(19) United States
(12) Patent Application Publication
(10) Pub. No.: US 2025/0120996 A1
(43) Pub. Date: Apr. 17, 2025

(54) T-CELL MODULATORY POLYPEPTIDES AND METHODS OF USE THEREOF

(71) Applicant: Cue Biopharma, Inc., Boston, MA (US)

(72) Inventors: Ronald D. Seidel, III, Boston, MA (US); Rodolfo J. Chaparro, Cambridge, MA (US); John F. Ross, Boston, MA (US); Saso Cemerski, Boston, MA (US)

(21) Appl. No.: 18/903,947

(22) Filed: Oct. 1, 2024

Related U.S. Application Data
(63) Continuation of application No. 17/584,133, filed on Jan. 25, 2022.
Application No. 17/584,133 is a continuation of application No. PCT/US2020/051255, filed on Sep. 17, 2020.
(60) Provisional application No. 62/903,441, filed on Sep. 20, 2019.
(60) Provisional application No. 62/990,693, filed on Mar. 17, 2020.
(60) Provisional application No. 63/048,561, filed on Jul. 6, 2020.

(51) Int. Cl. A61K 31/7088; A61K 38/00; A61P 35/00; C07K 14/55; C07K 14/74; C12N 9/14

(52) U.S. Cl. CPC A61K 31/7088; A61P 35/00; C07K 14/55; C07K 14/70539; C12N 9/14; A61K 38/00; C07K 2319/30

(57) ABSTRACT

The present disclosure provides T-cell modulatory polypeptides (TMPs) that comprise an immunomodulatory polypeptide, class I HLA polypeptides (a class I HLA heavy chain polypeptide and a β2 microglobulin polypeptide), and a KRAS peptide (e.g., a KRAS peptide comprising a cancer-associated mutation) that presents an epitope to a T-cell receptor. A TMP is useful for modulating the activity of a T cell, and for modulating an immune response in an individual.

### CROSS-REFERENCE

This application claims the benefit of U.S. Provisional Patent Application No. 62/903,441, filed Sep. 20, 2019, U.S. Provisional Patent Application No. 62/990,693, filed Mar. 17, 2020, and U.S. Provisional Patent Application No. 63/048,561, filed Jul. 6, 2020, each of which applications are incorporated herein by reference in their entirety.

### INCORPORATION BY REFERENCE OF SEQUENCE LISTING

A Sequence Listing is provided herewith as a Sequence Listing XML, “CUEB-126CON2_SEQLIST” created on Oct. 1, 2024 and having a size of 996,828 bytes. The contents of the Sequence Listing XML are incorporated herein by reference in their entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals—epitope/TCR binding and engagement of APC costimulatory proteins with T cell costimulatory proteins—are required to drive T cell specificity and activation or inhibition. The costimulatory proteins on the APC also are referred to as “immunomodulatory” proteins because they modulate the activity of the T cell when they bind the costimulatory protein on the T cell, with the specific modulation being a function of which immunomodulatory protein on the APC binds to which costimulatory protein on the T cell. The TCR is specific for a given epitope: however, the T cell's costimulatory protein is not epitope-specific and instead is generally expressed on all T cells or on large T cell subsets.

### SUMMARY

The present disclosure provides heterodimeric and single-chain T-cell modulatory polypeptides (TMPs), and dimers thereof, that comprise an immunomodulatory polypeptide, class I HLA polypeptides (a class I HLA heavy chain polypeptide and a β2 microglobulin polypeptide), and a KRAS peptide (e.g., a KRAS peptide comprising a cancer-associated mutation) that presents an epitope to a T-cell receptor. A TMP is useful for modulating the activity of a T cell, and for modulating an immune response in an individual.

### BRIEF DESCRIPTION OF THE DRAWINGS

FIGS. 1A-1F are schematic depictions of various exemplary TMPs of the present disclosure.

FIGS. 2A-2F are schematic depictions of various disulfide-linked TMPs of the present disclosure.

FIGS. 3A-3G provide amino acid sequences of immunoglobulin Fc polypeptides. The sequences are set forth in SEQ ID NO's 19-30.

FIG. 4 provides a multiple amino acid sequence alignment of beta-2 microglobulin (β2M) precursors (i.e., including the leader sequence) from Homo sapiens (NP_004039.1: SEQ ID NO:31), Pan troglodytes (NP_001009066.1: SEQ ID NO:31), Macaca mulatta (NP_001040602.1: SEQ ID NO:32), Bos taurus (NP_776318.1: SEQ ID NO:33) and Mus musculus (NP_033865.2: SEQ ID NO:34). Amino acids 1-20 are a signal peptide.

FIGS. 5A-5C provide amino acid sequences of full-length human HLA heavy chains of alleles A*0101 (SEQ ID NO: 35), A*1101 (SEQ ID NO: 36), A*2402 (SEQ ID NO: 37), and A*3303 (SEQ ID NO: 38) (FIG. 7A): full-length human HLA heavy chain of allele B*0702 (SEQ ID NO: 39) (FIG. 7B); and a full-length human HLA-C heavy chain (SEQ ID NO: 40) (FIG. 7C).

FIG. 6 provides an alignment of eleven mature MHC class I heavy chain amino acid sequences without their leader sequences, transmembrane domains, and intracellular domains. The sequences are set forth from top to bottom as follows: SEQ ID NOs: 41-51.

FIGS. 7A-7B provide an alignment of HLA-A heavy chain amino acid sequences (FIG. 7A; SEQ ID NO: 52-60, respectively) and a consensus sequence (FIG. 7B; SEQ ID NO: 61).

FIGS. 8A-8B provide an alignment of HLA-B heavy chain amino acid sequences (FIG. 8A; SEQ ID NOs: 62-68, respectively) and a consensus sequence (FIG. 8B; SEQ ID NO: 69).

FIGS. 9A-9B provide an alignment of HLA-C heavy chain amino acid sequences (FIG. 9A; SEQ ID NOs: 70-78, respectively) and a consensus sequence (FIG. 9B; SEQ ID NO: 79).

FIG. 10 provides a consensus amino acid sequence for each of HLA-E, -F, and -G heavy chains (SEQ ID NOs: 80-82, respectively). Variable amino acid (aa) positions are indicated as “X” residues sequentially numbered: the locations of amino acids 84, 139, and 236 are double underlined.

FIG. 11 provides an alignment of consensus amino acid sequences for HLA-A (SEQ ID No: 83), -B (SEQ ID NO: 84), -C(SEQ ID NO: 85), -E (SEQ ID NO: 86), -F (SEQ ID NO: 87), and -G (SEQ ID NO: 88).

FIG. 12A-12D provide schematic depictions of multiple disulfide-linked TMP of the present disclosure.

FIGS. 13A-13L provide amino acid sequences of examples of polypeptides suitable for inclusion in a TMP of the present disclosure. The sequences in FIG. 13A-13F are set forth in SEQ ID NOs: 255-260. The sequences in FIG. 13G-13L are set forth in SEQ ID NOs: 339-344.

FIGS. 14A-14JJ provide amino acid sequences of examples of polypeptides suitable for inclusion in a TMP of the present disclosure. The sequences are set forth in SEQ ID NOs: 261-296.

FIGS. 15A-15JJ provide amino acid sequences of examples of polypeptides suitable for inclusion in a TMP of the present disclosure. The sequences are set forth in SEQ ID NOs: 297-332.

FIGS. 16A-16C provide schematic depictions of examples of configurations of disulfide-linked TMPs of the present disclosure.

FIG. 17 provides schematic depictions of examples of positions of immunomodulatory polypeptides in “split chain” TMPs of the present disclosure.

FIG. 18 provides schematic depictions of examples of positions of immunomodulatory polypeptides in “single-chain” TMPs of the present disclosure.

FIGS. 19A-19P provide amino acid sequences of some examples of single-chain TMPs of the present disclosure. The sequences are set forth in SEQ ID NOs: 333-338 and 593-602.

FIG. 20 depicts the effect of KRAS G12V (7-16) HLA-A11 TMPs on expansion of KRAS G12V (7-16)-specific CD8⁺ T cells in vitro.

FIGS. 21A-21JJ provide amino acid sequences for HLA-A heavy chain amino acid sequences and β2M amino acid sequences of TMPs of the present disclosure.

FIGS. 22A-22BB provide amino acid sequences for HLA-A heavy chain amino acid sequences of TMPs of the present disclosure.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an MHC class I polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84C substitution is thus understood to be a substitution of Cys residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10. Unless otherwise stated, “sequence identity” as referred to herein is determined by BLAST (Basic Local Alignment Search Tool), as described in Altschul et al. (1990) J. Mol. Biol. 215:403.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001; 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to herein as a “MOD”), as used herein, means a polypeptide that specifically binds a cognate costimulatory polypeptide on a T cell, thereby providing a signal which, in addition to the primary signal provided by, for instance, binding of a TCR/CD3 complex with a major histocompatibility complex (MIHC) polypeptide loaded with peptide, mediates a T cell response, including, but not limited to, proliferation, activation, differentiation, and the like. As discussed herein, an immunomodulatory polypeptide can include, but is not limited to wild-type or variants of wild-type polypeptides such as a cytokine (e.g., IL-2), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor, and a ligand that specifically binds with B7-H3. An immunomodulatory domain or “MOD” of a TMP of the present disclosure can bind a cognate costimulatory polypeptide that is present on a target T cell.

As used herein the term “in vivo” refers to any process or procedure occurring inside of the body.

As used herein, “in vitro” refers to any process or procedure occurring outside of the body.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding,” as used herein (e.g. with reference to binding of a TMP to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ μM, less than 10⁻⁷ μM, less than 10⁻⁸ μM, less than 10⁻⁹ μM, less than 10⁻¹⁰ μM, less than 10⁻¹¹ μM, less than 10⁻¹² μM, less than 10⁻¹³ M, less than 10⁻¹⁴ μM, or less than 10⁻¹⁵ M. “Affinity” refers to the strength of non-covalent binding, increased binding affinity being correlated with a lower K_{D}. “Specific binding” generally refers to binding with an affinity of at least about 10⁻⁷ M or greater, e.g., 5×10⁻⁷ μM, 10⁻⁸ μM, 5×10⁻⁸ μM, 10⁻⁹ μM, and greater. “Non-specific binding” generally refers to binding (e.g., the binding of a ligand to a moiety other than its designated binding site or receptor) with an affinity of less than about 10⁻⁷ M (e.g., binding with an affinity of 10⁻⁶ μM, 10⁻⁵ μM, 10⁻⁴ M). However, in some contexts, e.g., binding between a TCR and a peptide/MHC complex, “specific binding” can be in the range of from 1 M to 100 μM, or from 100 μM to 1 mM. “Covalent binding” or “covalent bond,” as used herein, refers to the formation of one or more covalent chemical binds between two different molecules.

“Phosphate buffer saline” or “PBS”, as used herein, means a water-based buffer solution, typically available as a concentrated solution. Unless stated otherwise, the PBS solution used in this disclosure contains sodium chloride (500 mM), sodium phosphate dibasic (10 mM), potassium phosphate monobasic (2 mM), potassium chloride (2.7 mM), and the rest water. The pH of the PBS is 7.5±0.15. The buffer is prepared in 18.2 megohms DNase, and RNase free water and filtered through 0.22 micron filter.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely does not induce cell lysis.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

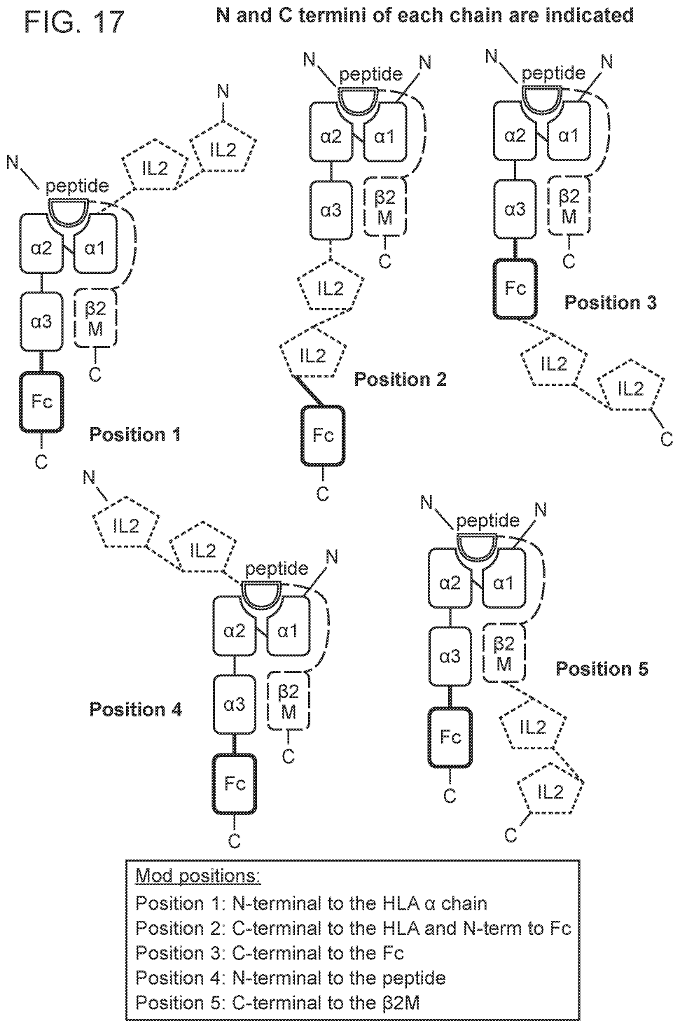

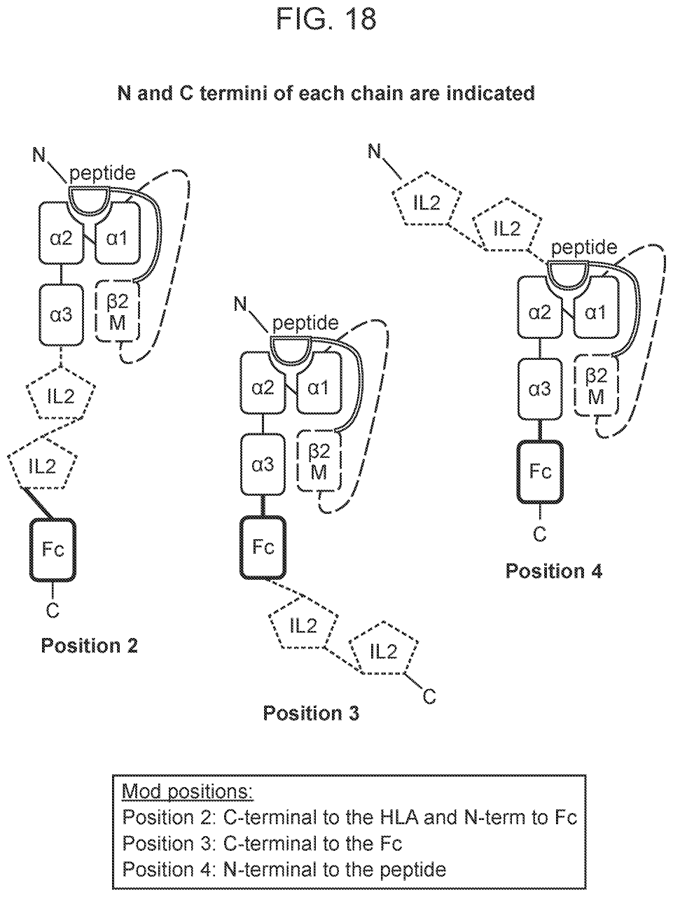

As used herein, the term “MHC heavy chain polypeptide” means collectively the domains of an MHC heavy chain polypeptide that are present in a TMP. For example, as illustrated in FIGS. 17 and 18, the MHC heavy chain polypeptide can comprise the α1, α2 and α3 domains.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to a “T-cell modulatory polypeptide” includes a plurality of such polypeptides and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication. Further, the dates of publication provided may be different from the actual publication dates which may need to be independently confirmed.

### DETAILED DESCRIPTION

The present disclosure provides T-cell modulatory polypeptides that comprise an immunomodulatory polypeptide and that comprise an epitope-presenting peptide. A TMP is useful for modulating the activity of a T cell, and for modulating an immune response in an individual.

### T-Cell Modulatory Polypeptides

The present disclosure provides a T-cell modulatory polypeptide (TMP), comprising: a) a first polypeptide; and b) a second polypeptide, wherein the TMP comprises: i) a KRAS peptide that, when bound to major histocompatibility complex (MHC) polypeptides, presents an epitope to a T-cell receptor (TCR); ii) a first MHC polypeptide; iii) a second MHC polypeptide; and iv) one or more immunomodulatory polypeptides; and optionally an immunoglobulin (Ig) Fc polypeptide or a non-Ig scaffold. A TMP comprising a first polypeptide and a second polypeptide also may be referred to herein as a “split-chain TMP” or a “heterodimeric TMP.” As discussed herein, the first and second polypeptides of such TMPs typically will be covalently linked to each other by one or more disulfide bonds, which can provide stability and/or improved expression to the TMP.

In some cases, however, a TMP of the present disclosure can comprise multiple different polypeptides that are linked together to form a single polypeptide chain. Such a single-chain TMP can comprise, e.g., i) a KRAS peptide that, when bound to major histocompatibility complex (MHC) polypeptides, presents an epitope to a T-cell receptor (TCR); ii) first and second MHC polypeptides; and iii) one or more immunomodulatory polypeptides; and optionally an immunoglobulin (Ig) Fc polypeptide or a non-Ig scaffold. As discussed below, heterodimeric TMPs and single-chain TMPs can self-assemble into dimers, e.g., when the TMP comprises an Ig Fc, e.g., an IgG1 Fc. In such cases, disulfide bonds will spontaneously form to bond the two TMPs.

As used herein, the term “KRAS peptide” means a peptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) that presents a KRAS epitope to a TCR when the KRAS peptide is bound to an MHC complex. As used herein, the term “KRAS epitope” means an epitope found on a KRAS protein. As used herein, the terms “KRAS” and “KRAS protein” are synonymous and mean a protein having an amino acid sequence present in one of the following: (i) a KRAS4A polypeptide; (ii) a KRAS4B; and (iii) variants of (i) and (ii) that occur in a human cancer, including, e.g., mutated forms. As used herein, the term “KRAS polypeptide” means a polypeptide having a sequence of amino acids found in all or a part of a KRAS protein, or where specified, a polypeptide having at least 75%, at least 80%, at least 85%, at least 90%, at least 95%, at least 98%, at least 99%, or 100% amino acid sequence identity to a sequence of amino acids found in all or a part of a KRAS protein or a variant that occurs in a human cancer, including, e.g., mutated forms.

KRAS (also known as “KRAS proto-oncogene, GTPase,” Kirsten rat sarcoma viral oncogene homolog,” and “K-Ras P21 protein”) is a GTPase that controls cell proliferation. When mutated, KRAS can fail to control cell proliferation, leading to cancer.

A wild-type (normal; non-cancer-associated) KRAS polypeptide can have the following amino acid sequence: MTEYKLVVVG AGGVGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:1).

A wild-type (normal; non-cancer-associated) KRAS polypeptide can have the following amino acid sequence: MTEYKLVVVG AGGVGKSALT IQLIQNHFVD EYDPTIEDSY RKQVVIDGET CLLDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ RVEDAFYTLV REIRQYRLKK ISKEEKTPGC VKIKKCIIM (SEQ ID NO:2).

Mutated forms of KRAS are associated with certain cancers; and at least a portion of the mutated form of KRAS is present on the surface of certain cancer cells. See, e.g., Prior et al. (2012) Cancer Res. 72:2457; and Warren and Holt (2010) Human Immunology 71:245. In SEQ ID NO:1 and SEQ ID NO:2, amino acids G12, G13, T35, I36, E49, Q61, K127, and A156 are in bold and underlined; substitutions of one or more of these residues can be present in a cancer-associated form of a KRAS polypeptide. A cancer-associated KRAS polypeptide can include one or more of: i) a substitution of G12 (e.g. G12C, G12V, G125, G12A, G12R, G12F, or G12D); ii) a substitution of G13 (e.g. G13C, G13D, G13R, G13V, G13S, or G13A); iii) a substitution of T35 (e.g., T35I); iv) a substitution of 136 (e.g., I36L or I36M); v) a substitution of E49 (e.g., E49K); vi) a substitution of Q61 (e.g. Q61H, Q61R, Q61P, Q61E, Q61K, Q61L, or Q61K); vii) a substitution of K117 (e.g., K117N); and viii) a substitution of A146 (e.g. A146T or A146V); where the amino acid numbering is as set out in SEQ ID NO:1 and SEQ ID NO:2. See, e.g., U.S. 2019/0194192.

For example, a cancer-associated, mutated form of a KRAS polypeptide can have one or more amino acid substitutions compared to the amino acid sequence set forth in SEQ ID NO:1 or SEQ ID NO:2. In some cases, a cancer-associated, mutated form of a KRAS polypeptide has only a single amino acid substitution compared to the amino acid sequence set forth in SEQ ID NO:1 or SEQ ID NO:2. In some cases, a cancer-associated, mutated form of a KRAS polypeptide has only two amino acid substitutions compared to the amino acid sequence set forth in SEQ ID NO:1 or SEQ ID NO:2. In some cases, a cancer-associated, mutated form of a KRAS polypeptide has only three amino acid substitutions compared to the amino acid sequence set forth in SEQ ID NO:1 or SEQ ID NO:2. In some cases, a cancer-associated, mutated form of a KRAS polypeptide has only four amino acid substitutions compared to the amino acid sequence set forth in SEQ ID NO:1 or SEQ ID NO:2. In some cases, a cancer-associated, mutated form of a KRAS polypeptide has only five amino acid substitutions compared to the amino acid sequence set forth in SEQ ID NO:1 or SEQ ID NO:2.

For example, KRAS(G12D) (a KRAS polypeptide having a G-to-D substitution at amino acid position 12, based on the amino acid numbering set forth in SEQ ID NO:1) is associated with pancreatic ductal adenocarcinoma (PDAC). KRAS(G12V) (a KRAS polypeptide having a G-to-V substitution at amino acid position 12, based on the amino acid numbering set forth in SEQ ID NO:1 or SEQ ID NO:2) is also associated with pancreatic cancer. KRAS(G12R) (a KRAS polypeptide having a G-to-R substitution at amino acid position 12, based on the amino acid numbering set forth in SEQ ID NO:1 or SEQ ID NO:2) is also associated with pancreatic cancer. See, e.g., Waters and Der (2018) Cold Spring Harb. Perspect. Med. 8:(9). pii: a031435. doi: 10.1101/cshperspect.a031435. As another example, KRAS(G12C) (a KRAS polypeptide having a G-to-C substitution at amino acid position 12, based on the amino acid numbering set forth in SEQ ID NO:1 or SEQ ID NO:2) is associated with lung cancer, e.g., non-small cell lung cancer. See, e.g., Román et al. (2018) Mol. Cancer 17:33. Other mutated forms of KRAS (e.g., G12A; G12C; G12D; G12R; G12S; G12V; G13A; G13C; G13D; G13R; G13S; G13V) are associated with various cancers; where such cancers include, e.g., bile duct carcinoma, gall bladder carcinoma, adenocarcinoma, rectal adenocarcinoma, endometrial carcinoma, hematopoietic neoplasms, and lung cancer. See, e.g., Prior et al. (20120 Cancer Res. 72:2457.

As another example, a cancer-associated, mutated form of a KRAS polypeptide can have an amino acid substitution at amino acid 61 of a KRAS polypeptide (e.g., a KRAS polypeptide having the amino acid sequence set forth in SEQ ID NO:1 or SEQ ID NO:2). For example, a cancer-associated, mutated form of a KRAS polypeptide can have an amino acid substitution such as Q61H, Q61L, Q61E, Q61R, or Q61K.

As discussed above, in some cases the present disclosure provides a TMP comprising a heterodimer, wherein the heterodimer comprises: a) a first polypeptide comprising a first MIHC polypeptide; and b) a second polypeptide comprising a second MHC polypeptide, wherein the first polypeptide or the second polypeptide comprises a KRAS peptide (e.g., a KRAS peptide comprising a cancer-associated mutation; where the KRAS peptide has a length of a least 4 amino acids (e.g., from 4 amino acids to about 25 amino acids); where the KRAS peptide, when bound to an MHC complex, presents an epitope to a T-cell receptor); wherein the first polypeptide and/or the second polypeptide comprises one or more immunomodulatory polypeptides that can be the same or different from one another; and optionally an Ig Fc polypeptide or a non-Ig scaffold. In some cases, at least one of the one or more immunomodulatory polypeptides is a variant immunomodulatory polypeptide that exhibits reduced affinity to a cognate costimulatory polypeptide compared to the affinity of a corresponding wild-type immunomodulatory polypeptide for the cognate costimulatory polypeptide.

The KRAS peptide/MHC complex present in a TMP of the present disclosure binds to a T-cell receptor (TCR) on a T cell with an affinity of at least 100 μM (e.g., at least 10 μM, at least 1 μM, at least 100 nM, at least 10 nM, or at least 1 nM). Generally speaking, a TMP of the present disclosure binds to a T cell having a cognate costimulatory polypeptide and a TCR that binds the KRAS peptide/MHC complex of the TMP with an affinity that is greater, e.g., 25% greater, than the affinity with which the same TMP binds a second T cell that has the same cognate costimulatory polypeptide but has a TCR that substantially does not bind the KRAS peptide/MHC complex, e.g., the KRAS peptide/MHC complex binds to the TCR with an affinity less than 10⁻⁷ M.

The present disclosure provides a TMP, wherein the TMP is a heterodimer comprising: a) a first polypeptide comprising a first MHC polypeptide; and b) a second polypeptide comprising a second MHC polypeptide, wherein the first polypeptide or the second polypeptide comprises a KRAS peptide; wherein the first polypeptide and/or the second polypeptide comprises one or more immunomodulatory polypeptides that can be the same or different, and wherein at least one of the one or more immunomodulatory polypeptides may be a wild-type immunomodulatory polypeptide or a variant of a wild-type immunomodulatory polypeptide, wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide; and wherein the first polypeptide or the second polypeptide optionally comprises an Ig Fc polypeptide or a non-Ig scaffold. In some cases, at least one of the one or more immunomodulatory domains is a variant immunomodulatory polypeptide that exhibits reduced affinity to a cognate costimulatory polypeptide compared to the affinity of a corresponding wild-type immunomodulatory polypeptide for the cognate costimulatory polypeptide, e.g., the ratio of (i) the binding affinity of a wild-type immunomodulatory polypeptide to a cognate costimulatory polypeptide to (ii) the binding affinity of the variant of the wild-type immunomodulatory polypeptide to the cognate costimulatory polypeptide, when measured by bio-layer interferometry, is in a range of from 1.5:1 to 10⁶:1; and wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide; and wherein the first polypeptide or the second polypeptide optionally comprises an Ig Fc polypeptide or a non-Ig scaffold.

In some cases, a heterodimeric TMP of this disclosure may comprise: a) a first polypeptide comprising, in order from N-terminus to C-terminus: i) a KRAS peptide; ii) a first MHC polypeptide; and b) a second polypeptide comprising, in order from N-terminus to C-terminus: i) a second MHC polypeptide; and ii) optionally an immunoglobulin (Ig) Fc polypeptide or a non-Ig scaffold, wherein the TMP comprises one or more immunomodulatory domains that can be the same or different, wherein at least one of the one or more immunomodulatory domain is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide, and wherein at least one of the one or more immunomodulatory domains may be a wild-type immunomodulatory polypeptide or a variant of a wild-type immunomodulatory polypeptide, wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide.

The present disclosure provides a TMP comprising, as a basic scaffold: a) a first polypeptide comprising, in order from N-terminus to C-terminus: i) a KRAS peptide; ii) a first MHC polypeptide; and b) a second polypeptide comprising, in order from N-terminus to C-terminus: i) a second MHC polypeptide; and ii) optionally an Ig Fc polypeptide or a non-Ig scaffold. A TMP of the present disclosure further comprises one or more immunomodulatory polypeptides, wherein at least one of the one or more immunomodulatory polypeptides is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide. In some cases, at least one of the one or more immunomodulatory polypeptides is a variant immunomodulatory polypeptide that exhibits reduced affinity to a cognate costimulatory polypeptide compared to the affinity of a corresponding wild-type immunomodulatory polypeptide for the cognate costimulatory polypeptide.

The KRAS peptide/MHC complex present in a TMP of the present disclosure binds to a T-cell receptor (TCR) on a T cell with an affinity of at least 100 μM (e.g., at least 10 μM, at least 1 μM, at least 100 nM, at least 10 nM, or at least 1 nM).

As noted above, in some cases, a TMP of the present disclosure comprises a single polypeptide chain. Such single-chain TMPs of the present disclosure comprise: i) a first MHC polypeptide; ii) a second MHC polypeptide; iii) a KRAS peptide that, when bound to MHC polypeptides, presents an epitope to a TCR; and iv) one or more immunomodulatory polypeptides; and optionally an Ig Fc polypeptide or a non-Ig scaffold.

In some cases, a single-chain TMP of the present disclosure comprises, in order from N-terminus to C-terminus: i) a KRAS peptide; ii) a first MHC polypeptide; iii) a second MIHC polypeptide; iv) one or more immunomodulatory polypeptides; and v) an Ig Fc polypeptide. In some cases, a single-chain TMP of the present disclosure comprises, in order from N-terminus to C-terminus: i) a KRAS peptide; ii) a β2M polypeptide; iii) a class I MHC heavy chain polypeptide; iv) one or more immunomodulatory polypeptides; and v) an Ig Fc polypeptide. This arrangement of components is referred to as MOD Position 2 in FIG. 18.

In some cases, a single-chain TMP of the present disclosure comprises, in order from N-terminus to C-terminus: i) a KRAS peptide; ii) a first MHC polypeptide; iii) a second MIHC polypeptide; iv) an Ig Fc polypeptide; and v) one or more immunomodulatory polypeptides. In some cases, a single-chain TMP of the present disclosure comprises, in order from N-terminus to C-terminus: i) a KRAS peptide; ii) a β2M polypeptide; iii) a class I MHC heavy chain polypeptide; iv) an Ig Fc polypeptide; and v) one or more immunomodulatory polypeptides. This arrangement of components is referred to as MOD Position 3 in FIG. 18.

In some cases, a single-chain TMP of the present disclosure comprises, in order from N-terminus to C-terminus: i) one or more immunomodulatory polypeptides; ii) a KRAS peptide; iii) a first MHC polypeptide; iv) a second MHC polypeptide; and v) an Ig Fc polypeptide. In some cases, a single-chain TMP of the present disclosure comprises, in order from N-terminus to C-terminus: i) one or more immunomodulatory polypeptides; ii) a KRAS peptide; iii) a first class I MHC polypeptide; iv) a second class I MHC polypeptide; and v) an Ig Fc polypeptide. In some cases, a single-chain TMP of the present disclosure comprises, in order from N-terminus to C-terminus: i) one or more immunomodulatory polypeptides; ii) a KRAS peptide; iii) a β2M polypeptide; iv) a class I MHC heavy chain polypeptide; and v) an Ig Fc polypeptide. This arrangement of components is referred to as MOD Position 4 in FIG. 18.

In some cases, the KRAS peptide/MHC complex present in a TMP of the present disclosure binds to a TCR on a T cell with an affinity of from about 10⁻⁴ μM to about 5×10⁻⁴ μM, from about 5×10⁻⁴ μM to about 10⁻⁵ μM, from about 10⁻⁵ M to 5×10⁻⁵ μM, from about 5×10⁻⁵ M to 10⁻⁶ μM, from about 10⁻⁶ μM to about 5×10⁻⁶ μM, from about 5×10⁻⁶ μM to about 10⁻⁷ μM, from about 10⁻⁷ μM to about 5×10⁻⁷ μM, from about 5×10⁻⁷ μM to about 10⁻⁸ μM, or from about 10⁻⁸ μM to about 10⁻⁹ M. Expressed another way, in some cases, the KRAS peptide/MHC complex present in a TMP of the present disclosure binds to a TCR on a T cell with an affinity of from about 1 nM to about 5 nM, from about 5 nM to about 10 nM, from about 10 nM to about 50 nM, from about 50 nM to about 100 nM, from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, from about 75 μM to about 100 μM.

In some cases, an immunomodulatory polypeptide present in a TMP of the present disclosure binds to its cognate costimulatory polypeptide with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type immunomodulatory polypeptide for the cognate costimulatory polypeptide.

In some cases, a variant immunomodulatory polypeptide present in a TMP of the present disclosure has a binding affinity for a cognate costimulatory polypeptide that is from 1 nM to 100 nM, or from 100 nM to 100 μM. For example, in some cases, a variant immunomodulatory polypeptide present in a TMP of the present disclosure has a binding affinity for a cognate costimulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM. In some cases, a variant immunomodulatory polypeptide present in a TMP of the present disclosure has a binding affinity for a cognate costimulatory polypeptide that is from about 1 nM to about 5 nM, from about 5 nM to about 10 nM, from about 10 nM to about 50 nM, from about 50 nM to about 100 nM.

The combination of the reduced affinity of the immunomodulatory polypeptide for its cognate costimulatory polypeptide, and the affinity of the KRAS peptide for a TCR, provides for enhanced selectivity of a TMP of the present disclosure. For example, a TMP of the present disclosure binds selectively to a first T cell that displays both: i) a TCR specific for the KRAS peptide present in the TMP; and ii) a costimulatory polypeptide that binds to the immunomodulatory polypeptide present in the TMP, compared to binding to a second T cell that displays:i) a TCR specific for an epitope other than the epitope present in the TMP; and ii) a costimulatory polypeptide that binds to the immunomodulatory polypeptide present in the TMP. For example, a TMP of the present disclosure binds to the first T cell with an affinity that is at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, at least 15-fold, at least 20-fold, at least 25-fold, at least 50-fold, at least 100-fold, or more than 100-fold, higher than the affinity to which it binds the second T cell.

In some cases, a TMP of the present disclosure, when administered to an individual in need thereof, induces both an epitope-specific T cell response and an epitope non-specific T cell response. In other words, in some cases, a TMP of the present disclosure, when administered to an individual in need thereof, induces an epitope-specific T cell response by modulating the activity of a first T cell that displays both: i) a TCR specific for the KRAS epitope present in the TMP; ii) a costimulatory polypeptide that binds to the immunomodulatory polypeptide present in the TMP; and induces an epitope non-specific T cell response by modulating the activity of a second T cell that displays:i) a TCR specific for an epitope other than the KRAS epitope present in the TMP; and ii) a costimulatory polypeptide that binds to the immunomodulatory polypeptide present in the TMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. “Modulating the activity” of a T cell can include one or more of: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin; a granzyme; a granulysin) by a cytotoxic (e.g., CD8⁺) T cell; iv) inducing proliferation of a cytotoxic (e.g., CD8⁺) T cell; v) inhibiting activity of an autoreactive T cell; and the like.

The combination of the reduced affinity of the immunomodulatory polypeptide for its cognate costimulatory polypeptide, and the affinity of the KRAS epitope for a TCR, provides for enhanced selectivity of a TMP of the present disclosure. Thus, for example, a TMP of the present disclosure binds with higher avidity to a first T cell that displays both: i) a TCR specific for the KRAS epitope present in the TMP; and ii) a costimulatory polypeptide that binds to the immunomodulatory polypeptide present in the TMP, compared to the avidity to which it binds to a second T cell that displays:i) a TCR specific for an epitope other than the epitope present in the TMP; and ii) a costimulatory polypeptide that binds to the immunomodulatory polypeptide present in the TMP.

Binding affinity between an immunomodulatory polypeptide and its cognate costimulatory polypeptide can be determined by bio-layer interferometry (BLI) using purified immunomodulatory polypeptide and purified cognate costimulatory polypeptide. Binding affinity between a TMP and its cognate costimulatory polypeptide can be determined by BLI using purified TMP and the cognate costimulatory polypeptide. BLI methods are well known to those skilled in the art. See, e.g., Lad et al. (2015) J. Biomol. Screen. 20(4):498-507; and Shah and Duncan (2014) J. Vis. Exp. 18:e51383.

A BLI assay can be carried out using an Octet RED 96 (Pal FortéBio) instrument, or a similar instrument, as follows. A TMP (e.g., a TMP of the present disclosure; a control TMP (where a control TMP comprises a wild-type immunomodulatory polypeptide)) is immobilized onto an insoluble support (a “biosensor”). The immobilized TMP is the “target.” Immobilization can be effected by immobilizing a capture antibody onto the insoluble support, where the capture antibody immobilizes the TMP. For example, immobilization can be effected by immobilizing anti-Fc (e.g., anti-human IgG Fc) antibodies onto the insoluble support, where the immobilized anti-Fc antibodies bind to and immobilize the TMP (where the TMP comprises an Ig Fc polypeptide). A costimulatory polypeptide is applied, at several different concentrations, to the immobilized TMP, and the instrument's response recorded. Assays are conducted in a liquid medium comprising 25 mM HEPES pH 6.8, 5% poly(ethylene glycol) 6000, 50 mM KCl, 0.10% bovine serum albumin, and 0.02% Tween 20 nonionic detergent. Binding of the costimulatory polypeptide to the immobilized TMP is conducted at 30° C. As a positive control for binding affinity, an anti-MHC class I monoclonal antibody can be used. For example, anti-HLA Class I monoclonal antibody W6/32 (American Type Culture Collection No. HB-95; Parham et al. (1979) J. Immunol. 123:342), which has a K_{D} of 7 nM, can be used. A standard curve can be generated using serial dilutions of the anti-MHC class I monoclonal antibody. The costimulatory polypeptide, or the anti-MHC class I mAb, is the “analyte.” BLI analyzes the interference pattern of white light reflected from two surfaces: i) from the immobilized polypeptide (“target”); and ii) an internal reference layer. A change in the number of molecules (“analyte”; e.g., costimulatory polypeptide; anti-HLA antibody) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d/a}) gives rise to the affinity constant K_{D}.

The BLI assay is carried out in a multi-well plate. To run the assay, the plate layout is defined, the assay steps are defined, and biosensors are assigned in Octet Data Acquisition software. The biosensor assembly is hydrated. The hydrated biosensor assembly and the assay plate are equilibrated for 10 minutes on the Octet instrument. Once the data are acquired, the acquired data are loaded into the Octet Data Analysis software. The data are processed in the Processing window by specifying method for reference subtraction, y-axis alignment, inter-step correction, and Savitzky-Golay filtering. Data are analyzed in the Analysis window by specifying steps to analyze (Association and Dissociation), selecting curve fit model (1:1), fitting method (global), and window of interest (in seconds). The quality of fit is evaluated. K_{D} values for each data trace (analyte concentration) can be averaged if within a 3-fold range. K_{D} error values should be within one order of magnitude of the affinity constant values; R² values should be above 0.95. See, e.g., Abdiche et al. (2008) J. Anal. Biochem. 377:209.

Unless otherwise stated herein, the affinity of a TMP of the present disclosure for a cognate costimulatory polypeptide, or the affinity of a control TMP (where a control TMP comprises a wild-type immunomodulatory polypeptide) for a cognate costimulatory polypeptide, is determined using BLI, as described above.

In some cases, the ratio of: i) the binding affinity of a control TMP (where the control comprises a wild-type immunomodulatory polypeptide) to a cognate costimulatory polypeptide to ii) the binding affinity of a TMP of the present disclosure comprising a variant of the wild-type immunomodulatory polypeptide to the cognate costimulatory polypeptide, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, the ratio of: i) the binding affinity of a control TMP (where the control comprises a wild-type immunomodulatory polypeptide) to a cognate costimulatory polypeptide to ii) the binding affinity of a TMP of the present disclosure comprising a variant of the wild-type immunomodulatory polypeptide to the cognate costimulatory polypeptide, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

As an example, where a control TMP comprises a wild-type IL-2 polypeptide, and where a TMP of the present disclosure comprises a variant IL-2 polypeptide (comprising from 1 to 10 amino acid substitutions relative to the amino acid sequence of the wild-type IL-2 polypeptide), e.g. the IL-2 variant comprising the H16A and F42A substitutions described herein, as the immunomodulatory polypeptide, the ratio of: i) the binding affinity of the control TMP to an IL-2 receptor (i.e., the cognate costimulatory polypeptide) to ii) the binding affinity of the TMP of the present disclosure to the IL-2 receptor, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, where a control TMP comprises a wild-type IL-2 polypeptide, and where a TMP of the present disclosure comprises a variant IL-2 polypeptide (comprising from 1 to 10 amino acid substitutions relative to the amino acid sequence of the wild-type IL-2 polypeptide) as the immunomodulatory polypeptide, the ratio of: i) the binding affinity of the control TMP to an IL-2 receptor (i.e., the cognate costimulatory polypeptide) to ii) the binding affinity of the TMP of the present disclosure to the IL-2 receptor, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

As another example, where a control TMP comprises a wild-type CD80 polypeptide, and where a TMP of the present disclosure comprises a variant CD80 polypeptide (comprising from 1 to 10 amino acid substitutions relative to the amino acid sequence of the wild-type CD80 polypeptide) as the immunomodulatory polypeptide, the ratio of: i) the binding affinity of the control TMP to a CTLA4 polypeptide (i.e., the cognate costimulatory polypeptide) to ii) the binding affinity of the TMP of the present disclosure to the CTLA4 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMP comprises a wild-type CD80 polypeptide, and where a TMP of the present disclosure comprises a variant CD80 polypeptide (comprising from 1 to 10 amino acid substitutions relative to the amino acid sequence of the wild-type CD80 polypeptide) as the immunomodulatory polypeptide, the ratio of: i) the binding affinity of the control TMP to a CD28 polypeptide (i.e., the cognate costimulatory polypeptide) to ii) the binding affinity of the TMP of the present disclosure to the CD28 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMP comprises a wild-type 4-1BBL polypeptide, and where a TMP of the present disclosure comprises a variant 4-1BBL polypeptide (comprising from 1 to 10 amino acid substitutions relative to the amino acid sequence of the wild-type 4-1BBL polypeptide) as the immunomodulatory polypeptide, the ratio of: i) the binding affinity of the control TMP to a 4-1BB polypeptide (i.e., the cognate costimulatory polypeptide) to ii) the binding affinity of the TMP of the present disclosure to the 4-1BB polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMP comprises a wild-type CD86 polypeptide, and where a TMP of the present disclosure comprises a variant CD86 polypeptide (comprising from 1 to 10 amino acid substitutions relative to the amino acid sequence of the wild-type CD86 polypeptide) as the immunomodulatory polypeptide, the ratio of: i) the binding affinity of the control TMP to a CD28 polypeptide (i.e., the cognate costimulatory polypeptide) to ii) the binding affinity of the TMP of the present disclosure to the CD28 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

Binding affinity of a TMP of the present disclosure to a target T cell can be measured in the following manner: A) contacting a TMP of the present disclosure with a target T-cell expressing on its surface: i) a cognate costimulatory polypeptide that binds the parental wild-type immunomodulatory polypeptide; and ii) a T-cell receptor that substantially binds to the epitope, where the TMP comprises an epitope tag, such that the TMP binds to the target T-cell; B) contacting the target T-cell-bound TMP with a fluorescently labeled binding agent (e.g., a fluorescently labeled antibody) that binds to the epitope tag, generating a TMP/target T-cell/binding agent complex; C) measuring the mean fluorescence intensity (MFI) of the TMP/target T-cell/binding agent complex using flow cytometry. The epitope tag can be, e.g., a FLAG tag, a hemagglutinin tag, a c-myc tag, a poly(histidine) tag, etc. The MFI measured over a range of concentrations of the TMP provides a measure of the affinity. The MFI measured over a range of concentrations of the TMP provides a half maximal effective concentration (EC₅₀) of the TMP. In some cases, the EC₅₀ of a TMP of the present disclosure for a target T cell is in the nM range; and the EC₅₀ of the TMP for a control T cell (where a control T cell expresses on its surface: i) a cognate costimulatory polypeptide that binds the parental wild-type immunomodulatory polypeptide; and ii) a T-cell receptor that does not bind to the epitope present in the TMP) is in the M range. In some cases, the ratio of the EC₅₀ of a TMP of the present disclosure for a control T cell to the EC₅₀ of the TMP for a target T cell is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at lease 10⁵:1, or at least 10⁶:1. The ratio of the EC₅₀ of a TMP of the present disclosure for a control T cell to the EC₅₀ of the TMP for a target T cell is an expression of the selectivity of the TMP.

In some cases, when measured as described in the preceding paragraph, a TMP of the present disclosure comprising a reduced-affinity MOD exhibits selective binding to target T-cell, compared to binding of the TMP to a control T cell that comprises: i) the cognate costimulatory polypeptide that binds the parental wild-type MOD; and ii) a T-cell receptor that substantially binds to an epitope other than the epitope present in the TMP.

### KRAS Peptides

As discussed above, a TMP of the present disclosure comprises a KRAS peptide that is typically at least about 4 amino acids in length, and presents a KRAS epitope to a T cell when in an MHC/peptide complex (e.g., an HLA/peptide complex).

A KRAS peptide present in a TMP of the present disclosure can have a length of at least 4 amino acids, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of 9-10 amino acids, from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length). In some cases, a KRAS peptide is 9 amino acids or 10 amino acids in length.

A KRAS epitope present in a TMP of the present disclosure is a peptide specifically bound by a T-cell, i.e., the epitope is specifically bound by an epitope-specific T cell, i.e., a T cell having a TCR that is specific for the KRAS epitope. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ μM, less than 10⁻⁵ μM, or less than 10⁻⁴ M. An epitope-specific T cell can bind an epitope for which it is specific with an affinity of at least 10⁻⁷ μM, at least 10⁻⁸ μM, at least 10⁻⁹ μM, or at least 10-W M.

In some cases, a suitable KRAS peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of 9-10 amino acids, from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a KRAS polypeptide comprising an amino acid sequence having at least 80%, at least 85%, at least 90%, at least 95%, at least 98%, or at least 99%, amino acid sequence identity to one of the following KRAS amino acid sequences:
(A) MTEYKLVVVG AGGVGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:1), where the KRAS polypeptide comprises one or more (e.g., 1, 2, 3, 4, or 5) amino acid substitutions compared to the amino acid sequence forth in SEQ ID NO:1, and where the one or more amino acid substitutions can include substitutions associated with cancer; e.g., substitutions that are found in a KRAS polypeptide in a cancer cell;
(B) MTEYKLVVVG AGGVGKSALT IQLIQNHFVD EYDPTIEDSY RKQVVIDGET CLLDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ RVEDAFYTLV REIRQYRLKK ISKEEKTPGC VKIKKCIIM (SEQ ID NO:2), where the KRAS polypeptide comprises one or more (e.g., 1, 2, 3, 4, or 5) amino acid substitutions compared to the amino acid sequence forth in SEQ ID NO:1, and where the one or more amino acid substitutions can include substitutions associated with cancer; e.g., substitutions that are found in a KRAS polypeptide in a cancer cell; and
(C) MTEY(X1)L(X2)(X3)(X4)GA(X5)(X6)VGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:89), where X1 is Lys, Phe, or Leu; X2 is Val or Leu; X3 is Val or Thr; X4 is Val or Thr; X5 is Gly, Asp, Cys, Val, or Ser; and X6 is Gly, Cys, or Asp; where one or both of X5 and X6 is not a Cys.

Non-limiting examples of suitable KRAS peptides include peptides comprising a sequence selected from the group consisting of VVGADGVGK (SEQ ID NO:176), VVGACGVGK (SEQ ID NO:177), VVGAVGVGK (SEQ ID NO:178), VVVGADGVGK (SEQ ID NO:179), VVVGAVGVGK (SEQ ID NO:180), VVVGACGVGK (SEQ ID NO:181), VTGADGVGK (SEQ ID NO:182), VTGAVGVGK (SEQ ID NO:183), VTGACGVGK (SEQ ID NO:184), VTVGADGVGK (SEQ ID NO:185), VTVGAVGVGK (SEQ ID NO:186), and VTVGACGVGK (SEQ ID NO:187); where the KRAS peptide has a length of 9 amino acids or 10 amino acids, or a length of at least 9 amino acids or 10 amino acids.

Additional non-limiting examples of suitable KRAS peptides include peptides comprising a sequence selected from the group consisting of: VVVGAGDVGK (SEQ ID NO:188); VVGAGDVGK (SEQ ID NO:189); VVVGARGVGK (SEQ ID NO:190); and VVGARGVGK (SEQ ID NO:191); where the KRAS peptide has a length of 9 amino acids or 10 amino acids, or a length of at least 9 amino acids or 10 amino acids.

Non-limiting examples of suitable KRAS peptides include peptides comprising a sequence selected from the group consisting of LVVVGADGV (SEQ ID NO:192), LVVVGAVGV (SEQ ID NO:193), LVVVGACGV (SEQ ID NO:194), KLVVVGADGV (SEQ ID NO:195), KLVVVGAVGV (SEQ ID NO:196), KLVVVGACGV (SEQ ID NO:197), LLVVGADGV (SEQ ID NO:198), LLVVGAVGV (SEQ ID NO:199), LLVVGACGV (SEQ ID NO:200), FLVVVGADGV (SEQ ID NO:201), FLVVVGAVGV (SEQ ID NO:202), and FLVVVGACGV (SEQ ID NO:203); where the KRAS peptide has a length of 9 amino acids or 10 amino acids, or a length of at least 9 amino acids or 10 amino acids.

Additional non-limiting examples of suitable KRAS peptides include peptides comprising a sequence selected from the group consisting of: KLVVVGAGDV (SEQ ID NO:204); and KLVVVGARGV (SEQ ID NO:205); where the KRAS peptide has a length of 9 amino acids or 10 amino acids, or a length of at least 9 amino acids or 10 amino acids.

Additional non-limiting examples of suitable KRAS peptides include peptides comprising a sequence selected from the group consisting of: GAGDVGKSAL (SEQ ID NO:206); AGDVGKSAL (SEQ ID NO:207); DVGKSALTI (SEQ ID NO:208); GAVGVGKSAL (SEQ ID NO:209); AVGVGKSAL (SEQ ID NO:210); YKLVVVGAV (SEQ ID NO:211); ARGVGKSAL (SEQ ID NO:212); GARGVGKSAL (SEQ ID NO:213); EYKLVVVGAR (SEQ ID NO:214); RGVGKSALTI (SEQ ID NO:215); LVVVGARGV (SEQ ID NO:216); GADGVGKSAL (SEQ ID NO:217); ACGVGKSAL (SEQ ID NO:218); and GACGVGKSAL (SEQ ID NO:219); where the KRAS peptide has a length of 9 amino acids or 10 amino acids, or a length of at least 9 amino acids or 10 amino acids.

In some cases, a TMP of the present disclosure modulates the activity of a T cell that comprises a TCR that is specific for a G12V form of a KRAS polypeptide, as described above. In such cases, the KRAS peptide present in a TMP of the present disclosure can comprise, e.g., one of the following amino acid sequences: VVGAVGVGK (SEQ ID NO: 178), VVVGAVGVGK (SEQ ID NO:180), VGAVGVGKS (SEQ ID NO:222), VGAVGVGKSA (SEQ ID NO:223), AVGVGKSAL (SEQ ID NO:210), AVGVGKSALT (SEQ ID NO:225), GAVGVGKSAL (SEQ ID NO:209), GAVGVGKSA (SEQ ID NO:227), LVVVGAVGVG (SEQ ID NO:228), LVVVGAVGV (SEQ ID NO:193), KLVVVGAVGV (SEQ ID NO:196), and KLVVVGAVG (SEQ ID NO:231); where the KRAS peptide has a length of 9 amino acids or 10 amino acids, or a length of at least 9 amino acids or 10 amino acids.

In some cases, the KRAS peptide present in a TMP of the present disclosure presents an epitope specific to an HLA-A, -B, -C, -E, -F, or -G allele. In an embodiment, the KRAS peptide present in a TMP presents an epitope restricted to HLA-A*0101, A*0201, A*0203, A*0301, A*1101, A*2301, A*2402, A*2407, A*3101, A*3303, A*3401, and/or A*6801. In an embodiment, the KRAS epitope peptide present in a TMP presents an epitope restricted to HLA-B*0702, B*0801, B*1502, B*2705, B*3802, B*3802, B*3901, B*3902, B*4001, B*4601, B*5101, and/or B*5301. In an embodiment, the KRAS epitope peptide present in a TMP presents an epitope restricted to C*0102, C*0303, C*0304, C*0401, C*0602, C*0701, C*702, C*0801, and/or C*1502.

As non-limiting examples, KRAS peptides comprising a peptide selected from the group consisting of VVGADGVGK (SEQ ID NO: 176), VVGACGVGK (SEQ ID NO:177), VVGAVGVGK (SEQ ID NO: 178), VVVGADGVGK (SEQ ID NO:179), VVVGAVGVGK (SEQ ID NO:180), VVVGACGVGK (SEQ ID NO:181), VTGADGVGK (SEQ ID NO:182), VTGAVGVGK (SEQ ID NO:183), VTGACGVGK (SEQ ID NO:184), VTVGADGVGK (SEQ ID NO:185), VTVGAVGVGK (SEQ ID NO:186), VTVGACGVGK (SEQ ID NO:187), VVVGAGDVGK (SEQ ID NO:188), VVGAGDVGK (SEQ ID NO:189), VVVGARGVGK (SEQ ID NO:190), and VVGARGVGK (SEQ ID NO:191), where the KRAS peptide has a length of 9 amino acids or 10 amino acids, or a length of at least 9 amino acids or 10 amino acids, present an epitope when bound to an HLA complex comprising a β2M polypeptide and an A*1101 HLA-A heavy chain. Such peptides may also be presented in complex with an HLA complex comprising a β2M polypeptide and an A*6801 HLA-A heavy chain.

As non-limiting examples, KRAS peptides comprising a peptide selected from the group consisting of LVVVGADGV (SEQ ID NO:192), LVVVGAVGV (SEQ ID NO:193), LVVVGACGV (SEQ ID NO:194), KLVVVGADGV (SEQ ID NO:195), KLVVVGAVGV (SEQ ID NO:196), KLVVVGACGV (SEQ ID NO:197), LLVVGADGV (SEQ ID NO:198), LLVVGAVGV (SEQ ID NO:199), LLVVGACGV (SEQ ID NO:200), FLVVVGADGV (SEQ ID NO:201), FLVVVGAVGV (SEQ ID NO:202), and FLVVVGACGV (SEQ ID NO:203) where the KRAS peptide has a length of 9 amino acids or 10 amino acids, or a length of at least 9 amino acids or 10 amino acids, present an epitope when bound to an HLA complex comprising a β2M polypeptide and an A*0201 HLA-A heavy chain.

As additional examples, the following KRAS peptides can present an epitope when bound to an HLA complex comprising a β2M polypeptide and an HLA-A heavy chain as follows: GAGDVGKSAL (SEQ ID NO:206), which can present an epitope when bound to an HLA complex comprising a β2M polypeptide and a B*3801 HLA-A heavy chain; AGDVGKSAL (SEQ ID NO:207), which can present an epitope when bound to an HLA complex comprising a β2M polypeptide and a B0702, a B*3801, or a B*3901 HLA-A heavy chain; DVGKSALTI (SEQ ID NO:208), which can present an epitope when bound to an HLA complex comprising a β2M polypeptide and a B*5101 HLA-A heavy chain; GAVGVGKSAL (SEQ ID NO:209), which can present an epitope when bound to an HLA complex comprising a β2M polypeptide and a B*0702 or a B*3801 HLA-A heavy chain; AVGVGKSAL (SEQ ID NO:210), which can present an epitope when bound to an HLA complex comprising a β2M polypeptide and a B*0702 HLA-A heavy chain; YKLVVVGAV (SEQ ID NO:211), which can present an epitope when bound to an HLA complex comprising a β2M polypeptide and an A*0203 or a B*3902 HLA-A heavy chain; ARGVGKSAL (SEQ ID NO:212), which can present an epitope when bound to an HLA complex comprising a β2M polypeptide and a B*0702, a B*2705, or a B*3901 HLA-A heavy chain; GARGVGKSAL (SEQ ID NO:213), which can present an epitope when bound to an HLA complex comprising a β2M polypeptide and a B*0702 HLA-A heavy chain; EYKLVVVGAR (SEQ ID NO:214), which can present an epitope when bound to an HLA complex comprising a β2M polypeptide and an A*3101 HLA-A heavy chain; RGVGKSALTI (SEQ ID NO:215), which can present an epitope when bound to an HLA complex comprising a β2M polypeptide and a B*0702 HLA-A heavy chain; LVVVGARGV (SEQ ID NO:216), which can present an epitope when bound to an HLA complex comprising a β2M polypeptide and an A*0203 HLA-A heavy chain; GADGVGKSAL (SEQ ID NO:217), which can present an epitope when bound to an HLA complex comprising a β2M polypeptide and a B*3801 HLA-A heavy chain; ACGVGKSAL (SEQ ID NO:218), which can present an epitope when bound to an HLA complex comprising a β2M polypeptide and a B*0702 HLA-A heavy chain; and GACGVGKSAL (SEQ ID NO:219), which can present an epitope when bound to an HLA complex comprising a β2M polypeptide and a B*3801 HLA-A heavy chain.

### MHC Polypeptides

As noted above, a TMP of the present disclosure includes MHC polypeptides. For the purposes of the instant disclosure, the term “major histocompatibility complex (MHC) polypeptides” is meant to include MHC polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., 3-2 microglobulin and MHC class I heavy chain).

In some cases, the first MHC polypeptide is an MHC class I β2M (β2M) polypeptide, and the second MHC polypeptide is an MHC class I heavy chain (H chain) (“MHC-H”)). In other instances, the first MHC polypeptide is an MHC class I heavy chain polypeptide; and the second MHC polypeptide is a β2M polypeptide. In some cases, both the β2M and MHC-H chain are of human origin; i.e., the MHC-H chain is an HLA heavy chain, or a variant thereof. Unless expressly stated otherwise, a TMP of the present disclosure does not include membrane anchoring domains (transmembrane regions) of an MHC class I heavy chain, or a part of MHC class I heavy chain sufficient to anchor the resulting TMP to a cell (e.g., eukaryotic cell such as a mammalian cell) in which it is expressed. In some cases, the MHC class I heavy chain present in a TMP of the present disclosure does not include a signal peptide, a transmembrane domain, or an intracellular domain (cytoplasmic tail) associated with a native MHC class I heavy chain. Thus, e.g., in some cases, the MHC class I heavy chain present in a TMP of the present disclosure includes only the α1, α2, and α3 domains of an MHC class I heavy chain. In some cases, the MHC class I heavy chain present in a TMP of the present disclosure has a length of from about 270 amino acids (aa) to about 290 aa. In some cases, the MHC class I heavy chain present in a TMP of the present disclosure has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, an MHC polypeptide of a TMP is a human MHC polypeptide, where human MHC polypeptides are also referred to as “human leukocyte antigen” (“HLA”) polypeptides. In some cases, an MHC polypeptide of a TMP is a Class I HLA polypeptide, e.g., a β2-microglobulin polypeptide, or a Class I HLA heavy chain polypeptide. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides.

### MHC Class I Heavy Chains

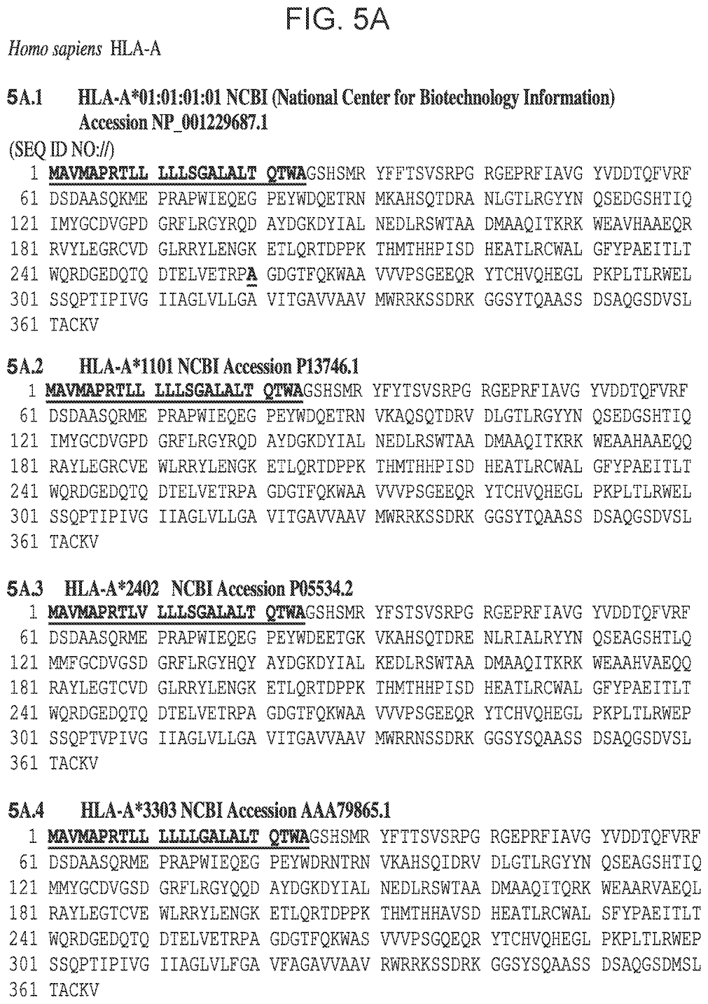

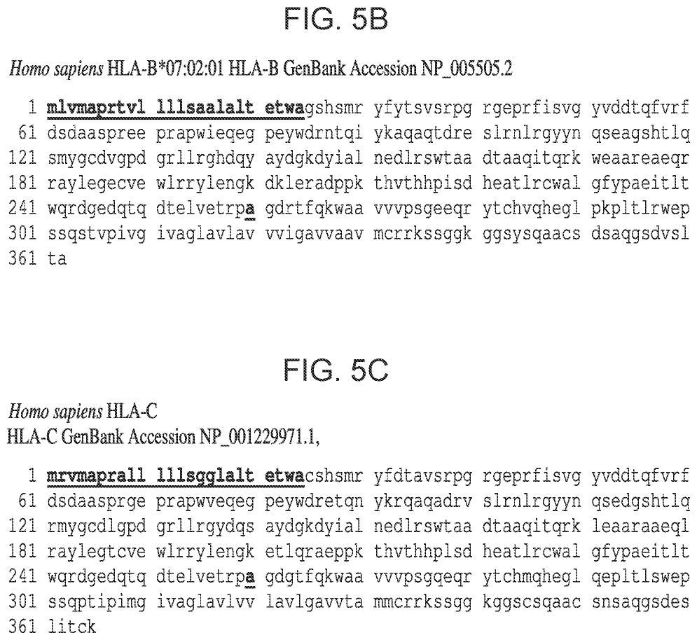

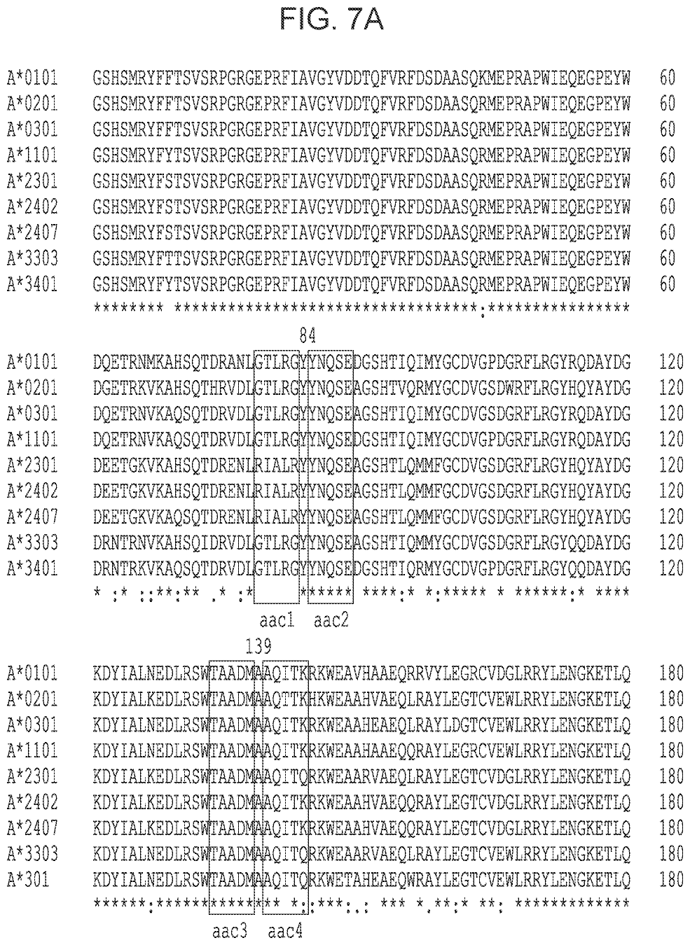

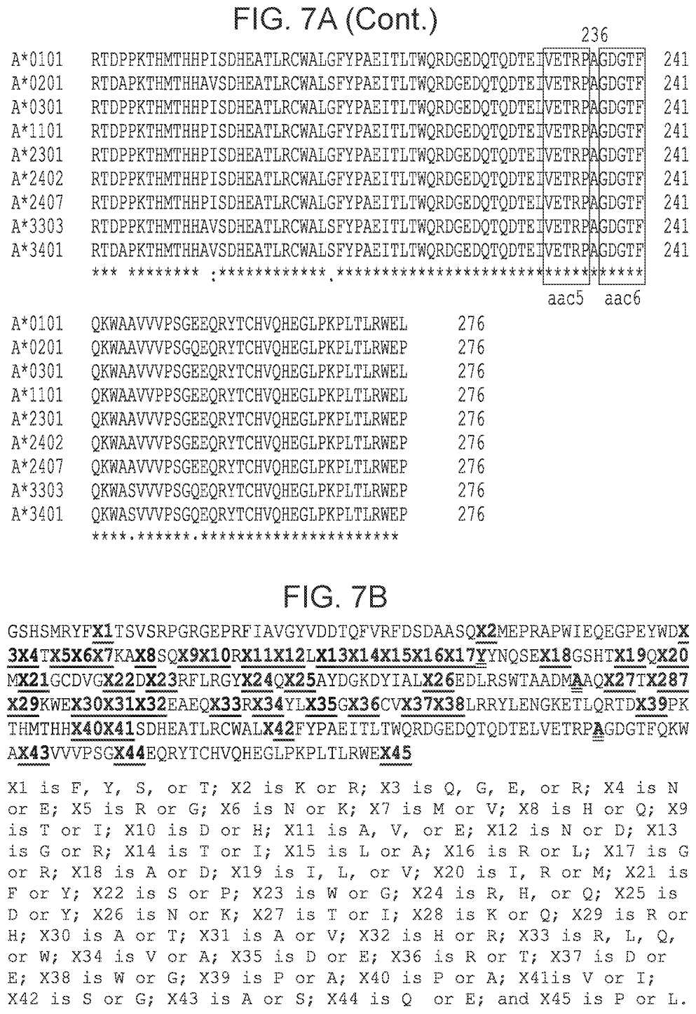

In some cases, an MHC class I heavy chain polypeptide present in a TMP of the present disclosure comprises an amino acid sequence having at least 75%, at least 80%, at least 85%, at least 90%, at least 95%, at least 98%, at least 99%, or 100%, amino acid sequence identity to all or part (e.g., 50, 75, 100, 150, 200, or 250 contiguous amino acids) of the amino acid sequence of any of the human HLA heavy chain polypeptides depicted in FIGS. 7-13. In some cases, the MIHC class I heavy chain has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa. In some cases, an MIHC class I heavy chain polypeptide present in a TMP of the present disclosure comprises 1-30, 1-5, 5-10, 10-15, 15-20, 20-25 or 25-30 amino acid insertions, deletions, and/or substitutions (in addition to those locations indicated as being variable in the heavy chain consensus sequences) of any one of the amino acid sequences depicted in FIGS. 7-13. In some cases, the MIHC class I heavy chain does not include transmembrane or cytoplasmic domains. As an example, a MHC class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to amino acids 25-300 (lacking all, or substantially all, of the leader, transmembrane and cytoplasmic sequence) or amino acids 25-365 (lacking the leader) of a human HLA-A heavy chain polypeptides depicted in any one of FIGS. 5A, 5B, and 5C.

FIGS. 5A, 5B and 5C provide amino acid sequences of human leukocyte antigen (HLA) Class I heavy chain polypeptides. Signal sequences, amino acids 1-24, are bolded and underlined. FIG. 5A entry: 3A.1 is the HLA-A heavy chain (HLA-A*01:01:01:01 or A*0101) (NCBI accession NP_001229687.1), SEQ ID NO:35; entry 3A.2 is from HLA-A*1101 SEQ ID NO:36; entry 3A.3 is from HLA-A*2402 SEQ ID NO:37 and entry 3A.4 is from HLA-A*3303 SEQ ID NO:38. FIG. 5B provides the sequence HLA-B*07:02:01 (HLA-B*0702) NCBI GenBank Accession NP_005505.2 (see also GenBank Accession AUV50118.1). FIG. 5C provides the sequence HLA-C*0701 (GenBank Accession NP_001229971.1) (HLA-C*07:01:01:01 or HLA-Cw*070101, HLA-Cw*07 see GenBank Accession CAO78194.1).

As mentioned above, the first and second polypeptides of a heterodimeric TMP typically will comprise one or more disulfide bonds to provide the TMP with improved stability and/or improved expression. The one or more disulfide bonds can be formed between Cys residues that are provided in the same polypeptide, i.e., intrachain disulfide bonds. Alternatively, or in addition to such intra-chain disulfide bonds, one or more interchain disulfide bonds can be formed between Cys residues that are provided in the first and second polypeptides, e.g., (i) non-naturally occurring Cys residues can be provided in both of the MHC class I polypeptides, i.e., the β2M polypeptide and MHC class I heavy chain polypeptide, and/or (ii) a linker comprising a Cys residue can be provided in both of the first and second polypeptides, and/or (iii) a linker comprising a Cys residue can be provided in one of the first and second polypeptides (e.g., between the epitope and β2M in the first polypeptide) and a non-naturally occurring Cys residue can be provided in the other MHC class I polypeptide (e.g., in the MHC class I heavy chain polypeptide). Exemplary configurations are discussed below.

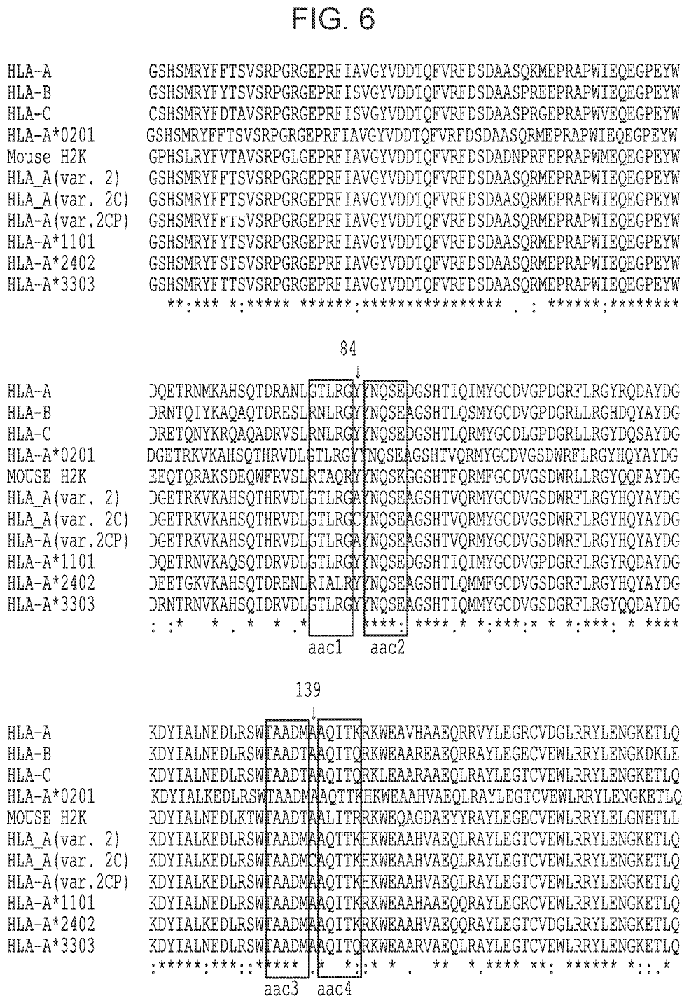

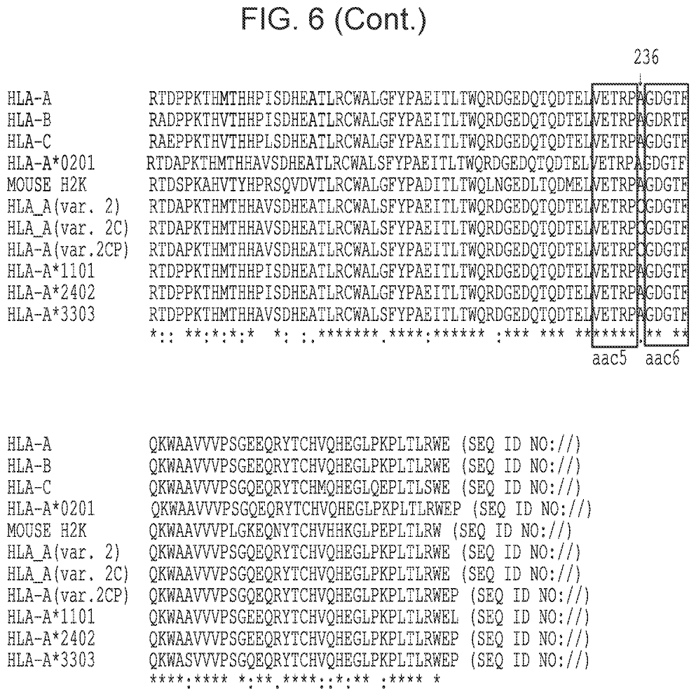

FIG. 6 provides an alignment of eleven mature MHC class I heavy chain amino acid sequences without their leader sequences or transmembrane domains or intracellular domains. The aligned sequences are human HLA-A, HLA-B, and HLA-C, a mouse H2K protein sequence, three variants of HLA-A (var.1, var.2C, and var.2CP), and 3 human HLA-A variants (HLA-A*1101; HLA-A*2402; and HLA-A*3303). Indicated in the alignment are the locations (84 and 139 of the mature proteins) where cysteine residues may be introduced (e.g., by substitution) for the formation of a disulfide bond to stabilize the MHC H chain—β2M complex. Also shown in the alignment is position 236 (of the mature polypeptide), which may be substituted by a cysteine residue that can form an inter-chain disulfide bond with β2M (e.g., at aa 12). An arrow appears above each of those locations and the residues are bolded. The seventh HLA-A sequence shown in the alignment (var.2c), shows the sequence of variant 2 substituted with C residues at positions 84, 139 and 236. The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

With regard to FIG. 6, in some cases: i) aac1 (amino acid cluster 1) may be the amino acid sequence GTLRG (SEQ ID NO:98) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO:99) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO:100) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO:101) or that sequence with one or two amino acids deleted or substituted with other naturally occurring amino acids (e.g., A replaced by G, Q replaced by N, or T replaced by S, and/or K replaced by R or Q); v) aac5 (amino acid cluster 5) may be the amino acid sequence VETRP (SEQ ID NO:102) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO:103) or that sequence with one or two amino acids deleted or substituted with other naturally occurring amino acids (e.g., D replaced by E, T replaced by S, or F replaced by L, W, or Y).

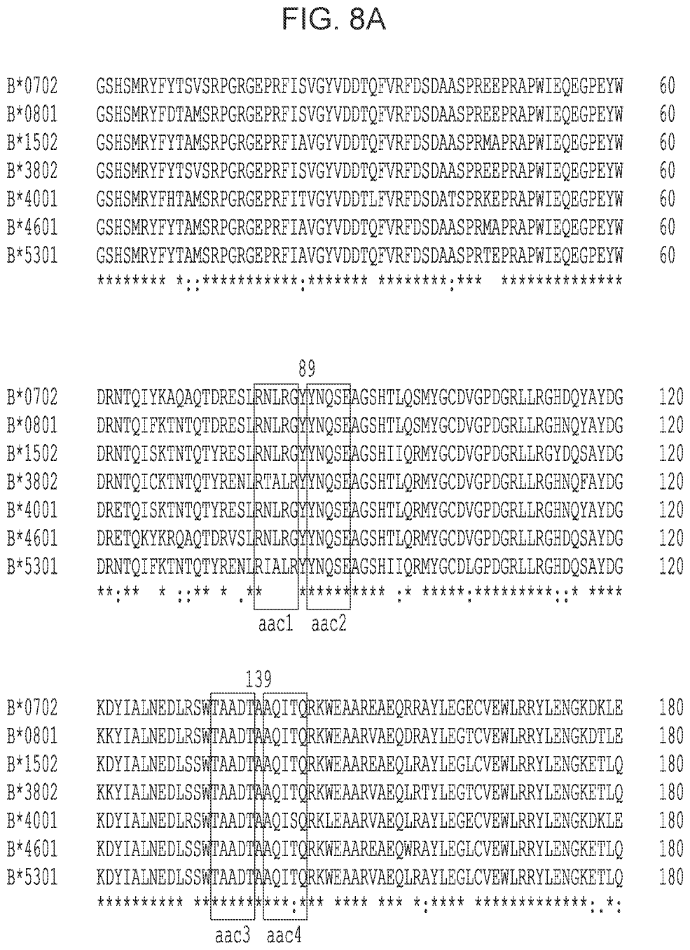

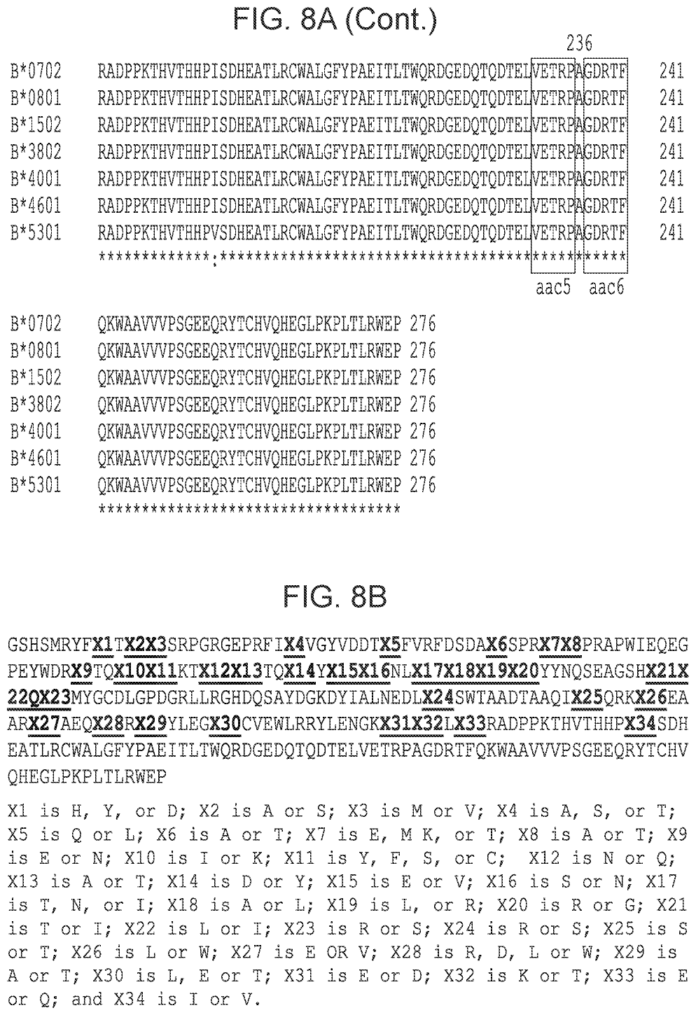

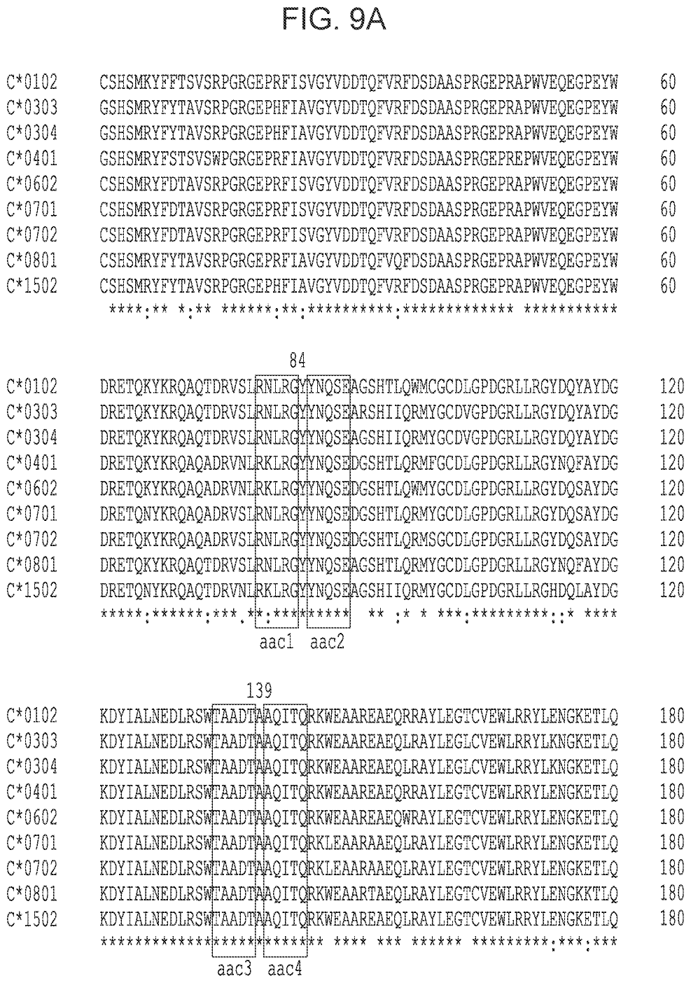

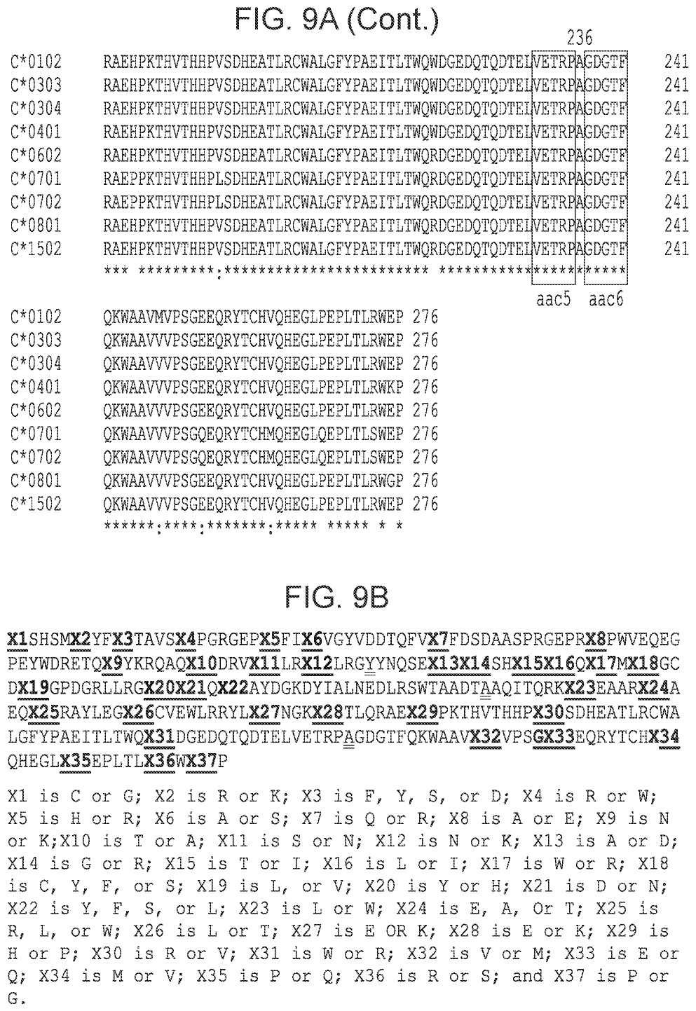

FIGS. 7-9 provide alignments of mature HLA class I heavy chain amino acid sequences (without leader sequences or transmembrane domains or intracellular domains). The aligned amino acid sequences in FIG. 7A are HLA-A class I heavy chains of the following alleles: A*0101, A*0201, A*0301, A*1101, A*2301, A*2402, A*2407, A*3303, and A*3401. The aligned amino acid sequences in FIG. 8A are HLA-B class I heavy chains of the following alleles: B*0702, B*0801, B*1502, B*3802, B*4001, B*4601, and B*5301. The aligned amino acid sequences in FIG. 9A are HLA-C class I heavy chains of the following alleles: C*0102, C*0303, C*0304, C*0401, C*0602, C*0701, C*0801, and C* 1502. Indicated in the alignments are the locations (84 and 139 of the mature proteins) where cysteine residues may be introduced (e.g., by substitution) for the formation of a disulfide bond to stabilize the HLA H chain—β2M complex. Also shown in the alignment is position 236 (of the mature polypeptide), which may be substituted by a cysteine residue that can form an inter-chain disulfide bond with β2M (e.g., at aa 12). The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

FIGS. 7A, 8A, and 9A provide alignments of the amino acid sequences of mature HLA-A, -B, and -C class I heavy chains, respectively. The sequences are provided for the extracellular portion of the mature protein (without leader sequences or transmembrane domains or intracellular domains). As described in FIG. 6, the positions of aa residues 84, 139, and 236 and their flanking residues (aac1 to aac6) that may be replaced by 1 to 5 amino acids selected independently from (i) any naturally occurring amino acid or (ii) any naturally occurring amino acid except proline or glycine ae also shown. FIGS. 7B, 8B, and 9B provide consensus amino acid sequences for the HLA-A, -B, and -C sequences, respectively, provide in FIGS. 7A, 8A, and 9A. The consensus sequences show the variable amino acid positions as “X” residues sequentially numbered and the locations of amino acids 84, 139 and 236 double underlined.

With regard to FIG. 7A, in some cases: i) aac1 (amino acid cluster 1) may be the amino acid sequence GTLRG (SEQ ID NO:98) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO:99) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO:100) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO:101) or that sequence with one or two amino acids deleted or substituted with other naturally occurring amino acids (e.g., A replaced by G, Q replaced by N, or T replaced by S, and or K replaced by R or Q); v) aac5 (amino acid cluster 5) may be the amino acid sequence VETRP (SEQ ID NO:102) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO:103) or that sequence with one or two amino acids deleted or substituted with other naturally occurring amino acids (e.g., D replaced by E, T replaced by S, or F replaced by L, W, or Y).

With regard to FIG. 8A, in some cases: i) aac1 (amino acid cluster 1) may be the amino acid sequence RNLRG (SEQ ID NO:104) or that sequence with one or two amino acids deleted or substituted with other naturally occurring amino acids (e.g., N replaced by T or I; and/or L replaced by A; and/or the second R replaced by L; and/or the G replaced by R); ii) aac2 (amino acid cluster 2) may be the amino acid sequence YNQSE (SEQ ID NO:99) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:105) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:106) or that sequence with one or two amino acids deleted or substituted with other naturally occurring amino acids (e.g., A replaced by G; and/or the first Q replaced by N; and/or I replaced by L or V; and/or the T replaced by S; and/or the second Q replaced by N); v) aac5 (amino acid cluster 5) may be the amino acid sequence VETRP (SEQ ID NO:102) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDRTF (SEQ ID NO:107) or that sequence with one or two amino acids deleted or substituted with other naturally occurring amino acids (e.g., D replaced by E; and/or T replaced by S; and/or R replaced by K or H; and/or F replaced by L, W, or Y).

With regard to FIG. 9A, in some cases: i) aac1 (amino acid cluster 1) may be the amino acid sequence RNLRG (SEQ ID NO:104) or that sequence with one or two amino acids deleted or substituted with other naturally occurring amino acids (e.g., N replaced by K; and/or L replaced by A or I; and/or the second R replaced by H; and/or the G replaced by T or S); ii) aac2 (amino acid cluster 2) may be the amino acid sequence YNQSE (SEQ ID NO:99) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:105) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:106) or that sequence with one or two amino acids deleted or substituted with other naturally occurring amino acids (e.g., A replaced by G; and/or the first Q replaced by N; and/or I replaced by L; and/or the second Q replaced by N or K); v) aac5 (amino acid cluster 5) may be the amino acid sequence VETRP (SEQ ID NO:102) or that sequence with one or two amino acids deleted or substituted with other naturally occurring amino acids (e.g., V replaced by I or L, E replaced by D, T replaced by S, and/or R replaced by K or H); and/or vi) aac6 (amino acid cluster 6) may be the amino acid sequence GDGTF (SEQ ID NO:103) or that sequence with one or two amino acids deleted or substituted with other naturally occurring amino acids (e.g., D replaced by E; and/or T replaced by S; and/or F replaced by L, W, or Y).

### HLA-A

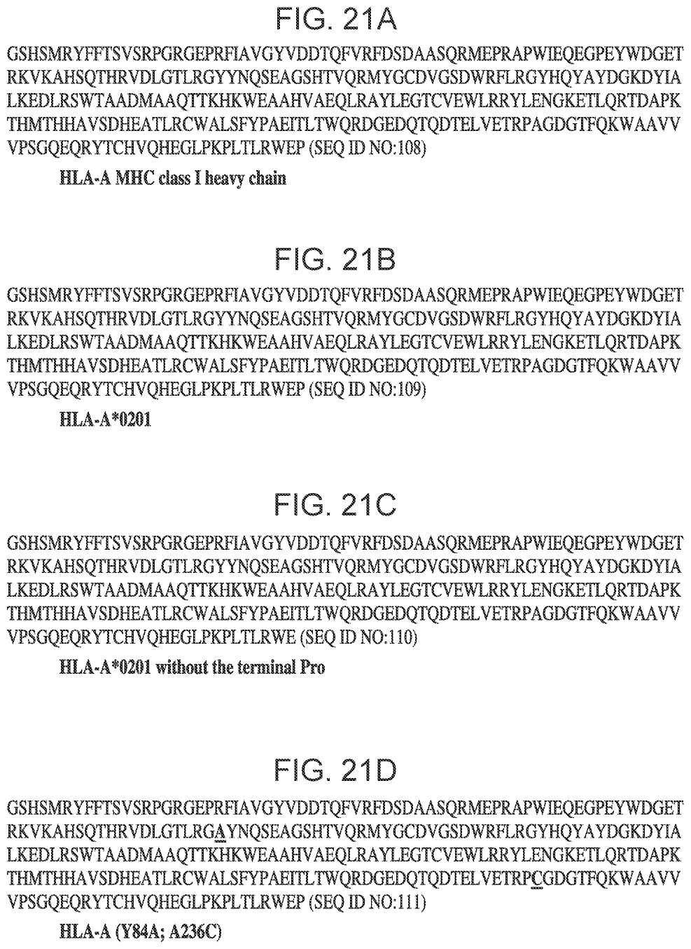

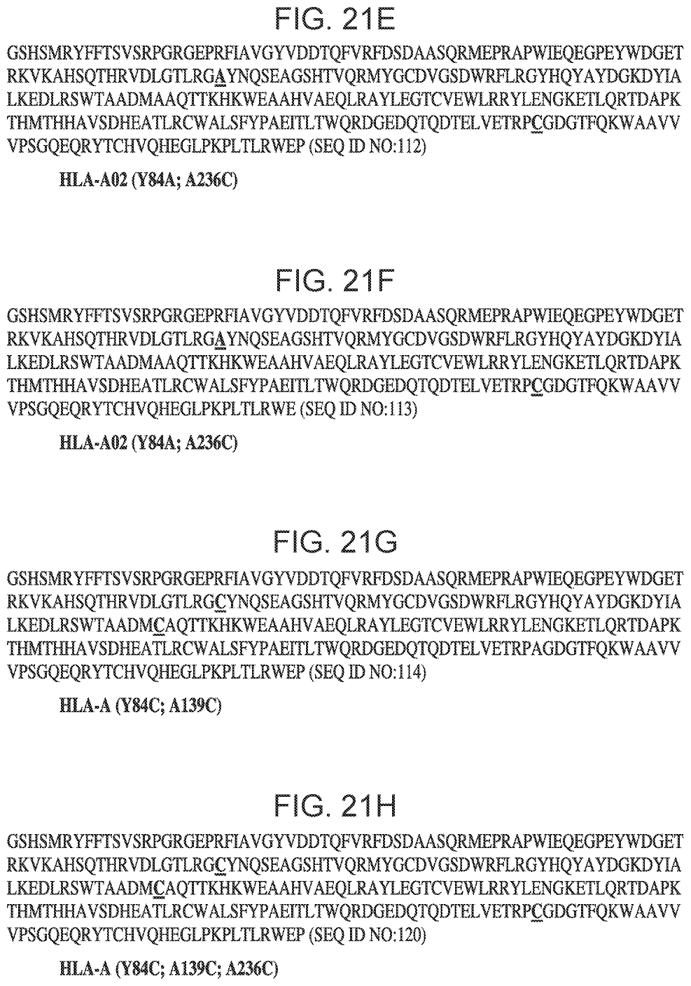

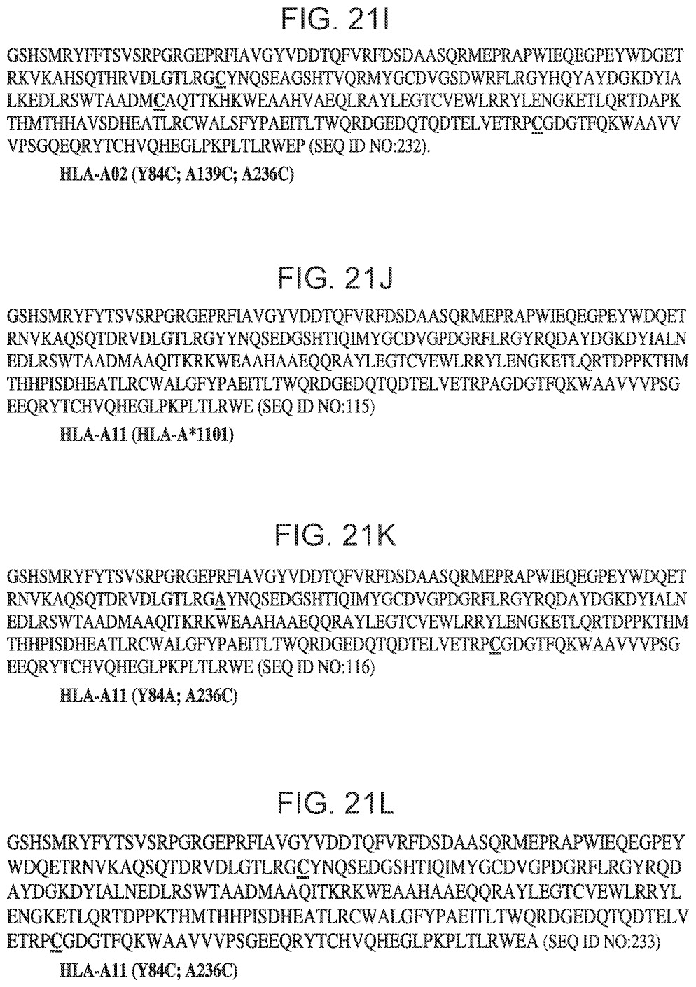

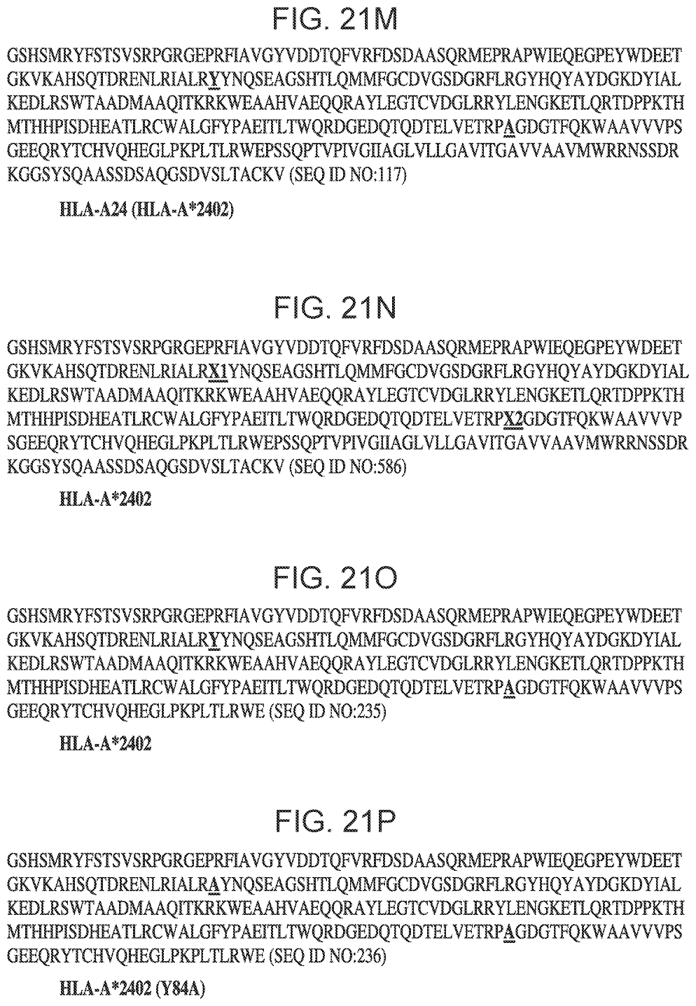

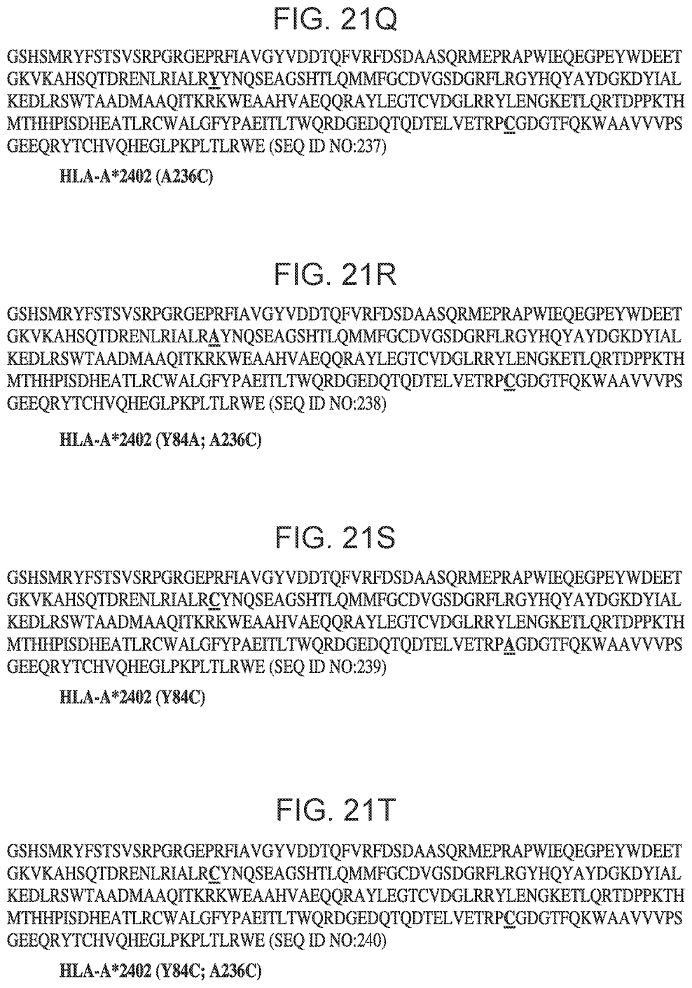

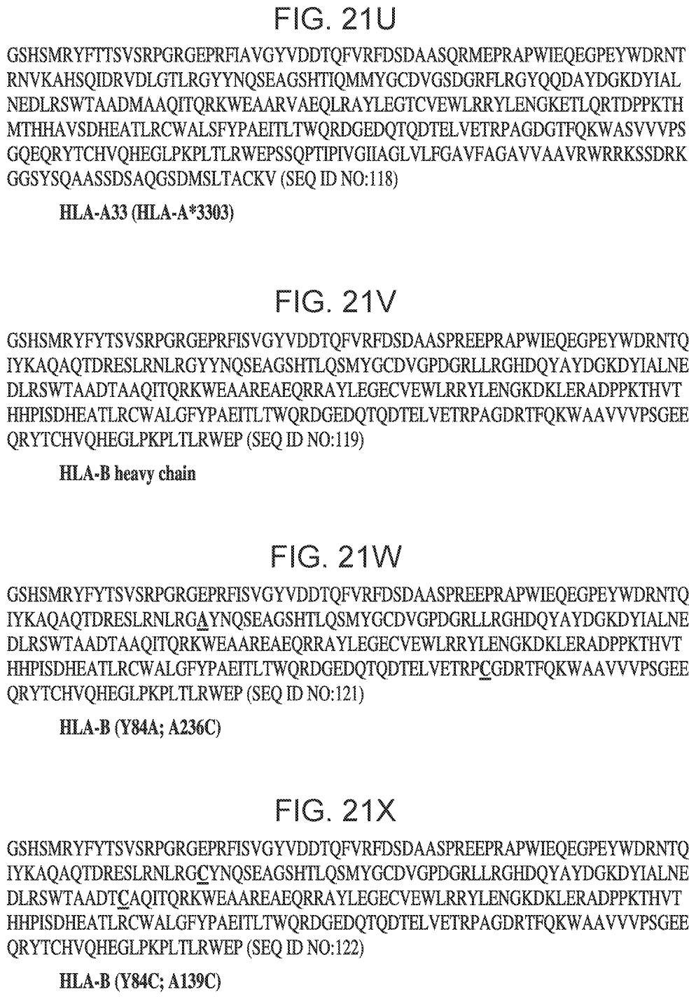

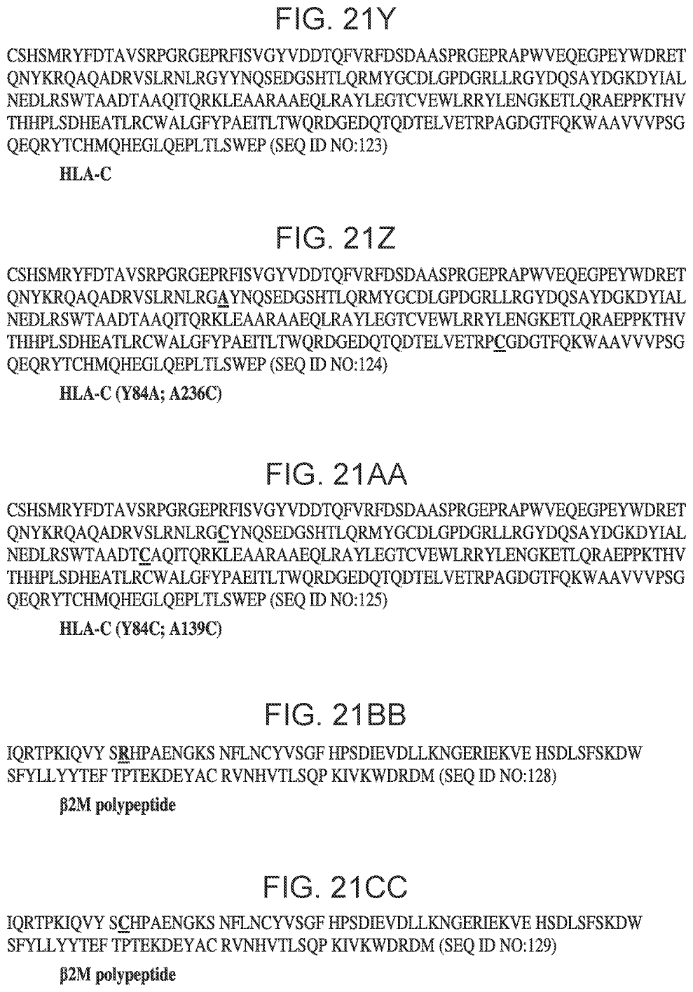

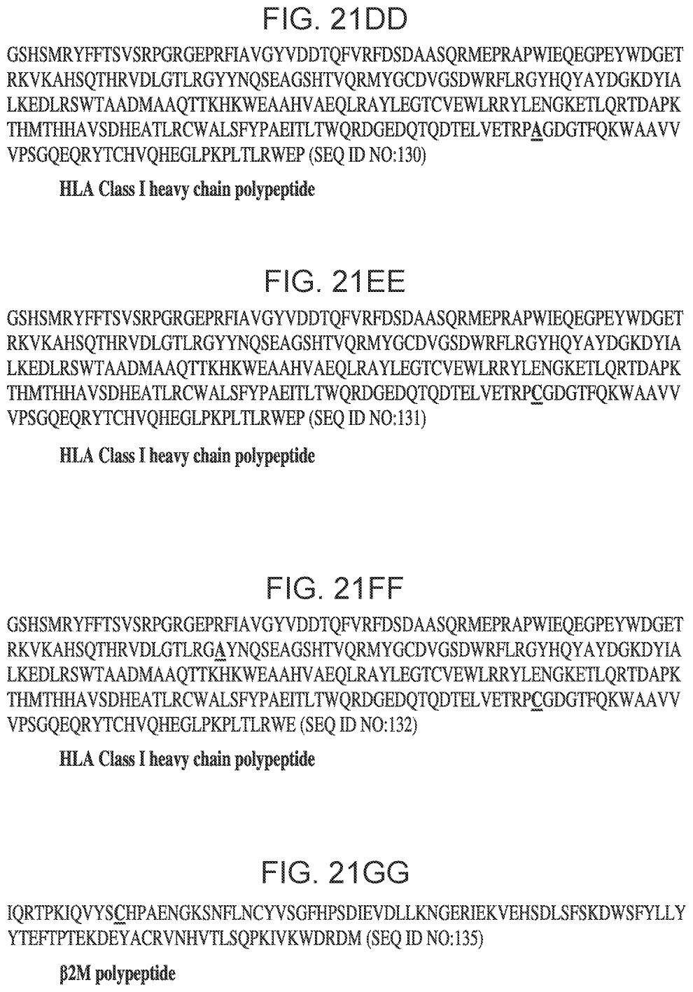

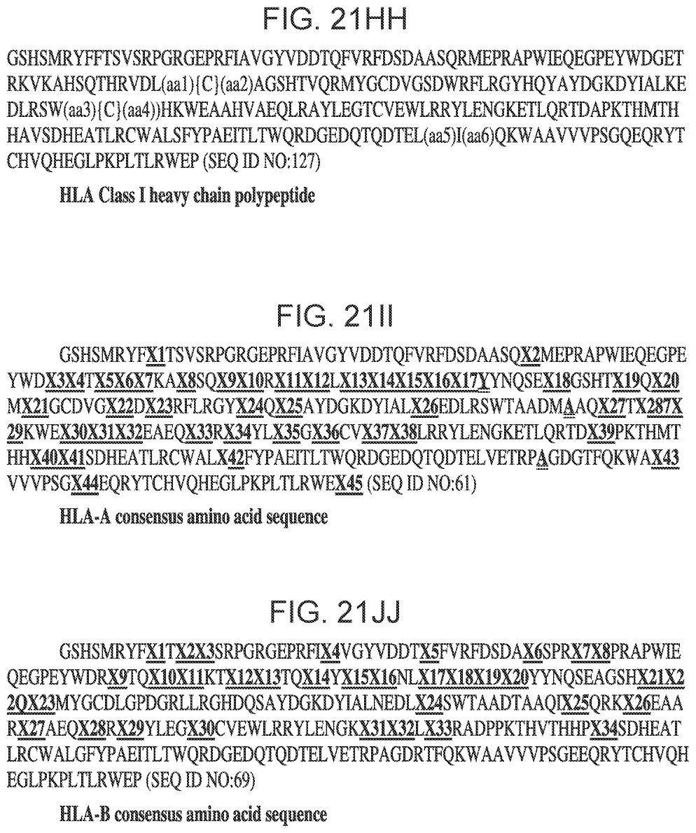

In some cases, a TMP of the present disclosure comprises an HLA-A heavy chain polypeptide. The HLA-A heavy chain peptide sequences, or portions thereof, that may be that may be incorporated into a TMP of the present disclosure include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 7A. Any of those alleles may comprise a mutation at one or more of positions 84, 139 and/or 236 (as shown in FIG. 7A) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, HLA-A sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-A alleles may also be employed (e.g., it may comprise 1-25, 1-5, 5-10, 10-15, 15-20, 20-25, or 25-30 amino acid insertions, deletions, and/or substitutions). A TMP of the present disclosure can comprise an MHC class I heavy chain comprising the amino acid sequence depicted in any one of FIGS. 21A-21AA, 21DD-21FF; 21HH, and 22A-22BB.

In some cases, a TMP of the present disclosure comprises an HLA-A heavy chain polypeptide comprising the HLA-A consensus amino acid sequence shown in FIG. 21II (SEQ ID NO:61), wherein X1 is F, Y, S, or T; X2 is K or R; X3 is Q, G, E, or R; X4 is N or E; X5 is R or G; X6 is N or K; X7 is M or V; X8 is H or Q; X9 is T or I; X10 is D or H; X11 is A, V, or E X12 is N or D; X13 is G or R; X14 is T or; X15 is L or A; X6 is R or L X17 is G or R; X18 is A or D; X19 is I, L, or V; X20 is I, R or M; X21 is F or Y; X22 is S or P; X23 is W or G; X24 is R, H, or Q; X25 is D or Y; X26 is N or K; X27 is T or 1; X28 is K or Q; X29 is R or 1H; X30 is A or T; X31 is A or V; X32 is 1H or R; X33 is R, L, Q, or W X34 is V or A; X35 is D or E; X36 is R or T; X37 is D or E; X38 is W or G; X39 is P or A; X40 is P or A; X4I is V or I; X42 is S or G; X43 is A or S; X44 is Q or E; and X45 is P or L.

As one example, an MHC class I heavy chain polypeptide of a TMP can comprise an amino acid sequence having at least 75%, at least 80%, at least 85%, at least 90%, at least 95%, at least 98%, at least 99%, or 100%, amino acid sequence identity to a human HLA-A heavy chain amino acid sequence shown in FIG. 21A (SEQ ID NO:108) or 21B (SEQ ID NO:109). The HLA-A heavy chain polypeptide of FIG. 21B is also referred to as “HLA-A*0201” or simply “HLA-A02.” In some cases, the C-terminal Pro is not included in a TMP of the present disclosure. For example, in some cases, an HLA-A02 polypeptide suitable for inclusion in a TMP of the present disclosure comprises at least 75%, at least 80%, at least 85%, at least 90%, at least 95%, at least 98%, at least 99%, or 100%, amino acid sequence identity to the amino acid sequence shown in FIG. 21C (SEQ ID NO:110).

### HLA-A (Y84A; A236C)

In some cases, the MHC class I heavy chain polypeptide comprises Y84A and A236C substitutions. For example, in some cases, the MHC class I heavy chain polypeptide comprises an amino acid sequence having at least 75%, at least 80%, at least 85%, at least 90%, at least 95%, at least 98%, at least 99%, or 100%, amino acid sequence identity to the human HLA-A heavy chain (Y84A; A236C) amino acid sequence shown in FIG. 21D (SEQ ID NO:111), where amino acid 84 is Ala and amino acid 236 is Cys. In some cases, the Cys-236 forms an interchain disulfide bond with Cys-12 of a variant β2M polypeptide that comprises an R12C substitution.

In some cases, an HLA-A heavy chain polypeptide suitable for inclusion in a TMP of the present disclosure is an HLA-A02 (Y84A; A236C) polypeptide comprising the amino acid sequence in FIG. 21E (SEQ ID NO:112). In some cases, an HLA-A heavy chain polypeptide suitable for inclusion in a TMP of the present disclosure is an HLA-A02 (Y84A; A236C) polypeptide comprising the amino acid sequence shown in FIG. 21F (SEQ ID NO:113).

### HLA-A (Y84C; A139C)

In some cases, the MHC class I heavy chain polypeptide comprises Y84C and A139C substitutions. For example, in some cases, the MHC class I heavy chain polypeptide comprises an amino acid sequence having at least 75%, at least 80%, at least 85%, at least 90%, at least 95%, at least 98%, at least 99%, or 100%, amino acid sequence identity to the human HLA-A heavy chain (Y84C; A139C) amino acid sequence shown in FIG. 21G (SEQ ID NO:114), where amino acid 84 is Cys and amino acid 139 is Cys. In some cases, Cys-84 forms an intrachain disulfide bond with Cys-139.

### HLA-A (Y84C; A139C; A236C)

In some cases, a MHC class I heavy chain polypeptide suitable for inclusion in a TMP of the present disclosure comprises an amino acid sequence having at least 75%, at least 80%, at least 85%, at least 90%, at least 95%, at least 98%, at least 99%, or 100%, amino acid sequence identity to the human HLA-A heavy chain (Y84C; A139C; A236C) amino acid sequence shown in FIG. 21H (SEQ ID NO:120), where amino acid 84 is Cys, amino acid 139 is Cys, and amino acid 236 is Cys. In some cases, Cys-84 forms an intrachain disulfide bond with Cys-139. The Cys at amino acid 236 can form a disulfide bond with a Cys residue in a second polypeptide chain. For example, the Cys at amino acid 236 can form a disulfide bond with the Cys-12 residue in a β2M polypeptide comprising an R12C substitution.

In some cases, an HLA-A heavy chain polypeptide suitable for inclusion in a TMP of the present disclosure is an HLA-A02 (Y84C; A139C; A236C) polypeptide comprising the amino acid sequence shown in FIG. 21I (SEQ ID NO:232).

### HLA-A11 (HLA-A*1101)

As one non-limiting example, an MHC class I heavy chain polypeptide of a TMP can comprise an amino acid sequence having at least 75%, at least 80%, at least 85%, at least 90%, at least 95%, at least 98%, at least 99%, or 100%, amino acid sequence identity to the human HLA-A11 heavy chain amino acid sequence shown in FIG. 21J (SEQ ID NO:115). Such an MHC class I heavy chain may be prominent in Asian populations, including populations of individuals of Asian descent.

### HLA-A11 (Y84A; A236C)

As one non-limiting example, in some cases, the MHC class I heavy chain polypeptide is an HLA-A11 allele that comprises Y84A and A236C substitutions. For example, in some cases, the MHC class I heavy chain polypeptide comprises an amino acid sequence having at least 75%, at least 80%, at least 85%, at least 90%, at least 95%, at least 98%, at least 99%, or 100%, amino acid sequence identity to the human HLA-A A11 heavy chain (Y84A; A236C) amino acid sequence shown in FIG. 21K (SEQ ID NO:116), where amino acid 84 is Ala and amino acid 236 is Cys. In some cases, the Cys-236 forms an interchain disulfide bond with Cys-12 of a variant β2M polypeptide that comprises an R12C substitution, as shown in FIG. 21L (SEQ ID NO:233), where amino acid 84 is Cys and amino acid 236 is Cys.

### HLA-A24 (HLA-A*2402)

As one non-limiting example, an MHC class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the human HLA-A24 heavy chain amino acid sequence shown in FIG. 21M (SEQ ID NO:117). Such an MHC class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is a Cys and amino acid 236 is a Cys.

As one non-limiting example, an MHC class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the human HLA-A24 (also referred to as HLA-A*2402) heavy chain amino acid sequence shown in FIG. 21N (SEQ ID NO:586). Such an MHC class I heavy chain may be prominent in Asian populations, including populations of individuals of Asian descent. In some cases, amino acid X1 at position 84 is an Ala. In some cases, amino acid 84 is a Cys. In some cases, amino acid X2 at position 236 is a Cys. In some cases, amino acid 84 is an Ala and amino acid 236 is a Cys. In some cases, amino acid 84 is a Cys and amino acid 236 is a Cys.

In some cases, an MHC class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the human HLA-A24 (also referred to as HLA-A*2402) heavy chain amino acid sequence shown in one of the following Figures:
(i) FIG. 21O (SEQ ID NO:235), where amino acid 84 is Tyr and amino acid 236 is Ala (amino acids 84 and 236 are bold and underlined); and where the MHC class I heavy chain has a length of about 275 amino acids;
(ii) TMPFIG. 21P (SEQ ID NO:236), where amino acid 84 is Ala and amino acid 236 is Ala (amino acids 84 and 236 are bold and underlined); and where the MHC class I heavy chain has a length of about 275 amino acids;
(iii) TMPFIG. 21Q (SEQ ID NO:237), where amino acid 84 is Tyr and amino acid 236 is Cys (amino acids 84 and 236 are bold and underlined); and where the MIHC class I heavy chain has a length of about 275 amino acids;
(iv) TMPFIG. 21R (SEQ ID NO:238), where amino acid 84 is Ala and amino acid 236 is Cys (amino acids 84 and 236 are bold and underlined); and where the MHC class I heavy chain has a length of about 275 amino acids;
(v) TMPFIG. 21S (SEQ ID NO:239), where amino acid 84 is Cys and amino acid 236 is Ala (amino acids 84 and 236 are bold and underlined); and where the MHC class I heavy chain has a length of about 275 amino acids; and
(vi) TMPFIG. 21T (SEQ ID NO:240), where amino acid 84 is Cys and amino acid 236 is Cys (amino acids 84 and 236 are bold and underlined); and where the MIHC class I heavy chain has a length of about 275 amino acids.

### HLA-A33 (HLA-A*3303)

As one non-limiting example, an MHC class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the human HLA-A33 heavy chain amino acid sequence shown in FIG. 21U (SEQ ID NO:118). Such an MHC class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is a Cys and amino acid 236 is a Cys.

### HLA-B

In some cases, a TMP of the present disclosure comprises an HLA-B heavy chain polypeptide. The HLA-B heavy chain peptide sequences, or portions thereof, that may be that may be incorporated into a TMP of the present disclosure include, but are not limited to, the alleles: B*0702, B*0801, B*1502, B*3802, B*4001, B*4601, and B*5301, which are aligned without all, or substantially all, of the leader, transmembrane and cytoplasmic sequences in FIG. 8A. Any of those alleles may comprise a mutation at one or more of positions 84, 139 and/or 236 (as shown in FIG. 8A) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, a HLA-B polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-B alleles may also be employed (e.g., it may comprise 1-25, 1-5, 5-10, 10-15, 15-20, 20-25, or 25-30 amino acid insertions, deletions, and/or substitutions).

In some cases, a TMP of the present disclosure comprises an HLA-B heavy chain polypeptide comprising the HLA-B consensus amino acid sequence shown in FIG. 21 JJ (SEQ ID NO:69), wherein X1 is H, Y, or D; X2 is A or S; X3 is M or V; X4 is A, S, or T; X5 is Q or L; X6 is A or T; X7 is E, M K, or T; X8 is A or T; X9 is E or N; X10 is I or K; X11 is Y, F, S, or C; X12 is N or Q; X13 is A or T; X14 is D or Y; X15 is E or V; X16 is S or N; X17 is T, N, or I; X18 is A or L; X19 is L, or R; X20 is R or G; X21 is T or I; X22 is L or I; X23 is R or S; X24 is R or S; X25 is S or T; X26 is L or W; X27 is E OR V; X28 is R, D, L or W; X29 is A or T; X30 is L, E or T; X31 is E or D; X32 is K or T; X33 is E or Q; and X34 is I or V.

As an example, an MHC class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the human HLA-B heavy chain amino acid sequence shown in FIG. 21V (SEQ ID NO:119).

### HLA-B (Y84A; A236C)

As one non-limiting example, in some cases, the MHC class I heavy chain polypeptide is an HLA-B polypeptide that comprises Y84A and A236C substitutions. For example, in some cases, the MHC class I heavy chain polypeptide comprises an amino acid sequence having at least 75%, at least 80%, at least 85%, at least 90%, at least 95%, at least 98%, at least 99%, or 100%, amino acid sequence identity to the human HLA-B heavy chain (Y84A; A236C) amino acid sequence shown in FIG. 21W (SEQ ID NO:121), where amino acid 84 is Ala and amino acid 236 is Cys. In some cases, the Cys-236 forms an interchain disulfide bond with Cys-12 of a variant β2M polypeptide that comprises an R12C substitution.

### HLA-B (Y84C; A139C)

In some cases, the MHC class I heavy chain polypeptide comprises Y84C and A139C substitutions. For example, in some cases, the MHC class I heavy chain polypeptide comprises an amino acid sequence having at least 75%, at least 80%, at least 85%, at least 90%, at least 95%, at least 98%, at least 99%, or 100%, amino acid sequence identity to the human HLA-B heavy chain (Y84C; A139C) amino acid sequence shown in FIG. 21X (SEQ ID NO:122), where amino acid 84 is Cys and amino acid 139 is Cys. In some cases, Cys-84 forms an intrachain disulfide bond with Cys-139.

### HLA-B*0702

As an example, in some cases, a MIHC class I heavy chain polypeptide present in a TMP of the present disclosure comprises an amino acid sequence of HLA-B*0702 (SEQ ID NO:62) in FIG. 8A, or a sequence having at least 75% (e.g., at least 80%, at least 85%, at least 90%, at least 95%, at least 98%, at least 99%) or 100%, amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the HLA-B heavy chain polypeptide of TMP of the present disclosure has less than 100% identity to the sequence labeled HLA-B in FIG. 6, or labeled “B*0702 in FIG. 8A, it may comprise a mutation at one or more of positions 84, 139 and/or 236 selected from: a tyrosine to alanine substitution at position 84 (Y84A); a tyrosine to cysteine substitution at position 84 (Y84C); an alanine to cysteine at position 139 (A139C); and an alanine to cysteine substitution at position 236 (A236C). In some cases, the HLA-B heavy chain polypeptide of TMP of the present disclosure comprises Y84A and A236C substitutions. In some cases, the HLA-B*0702 heavy chain polypeptide of TMP of the present disclosure comprises Y84C and A139C substitutions. In some cases, the HLA-B heavy chain polypeptide of TMP of the present disclosure comprises Y84C, A139C, and A236C substitutions.

### HLA-C

In some cases, a TMP of the present disclosure comprises an HLA-C heavy chain polypeptide. The HLA-C heavy chain polypeptide, or portions thereof, that may be that may be incorporated into a TMP of the present disclosure include, but are not limited to, the alleles: C*0102, C*0303, C*0304, C*0401, C*0602, C*0701, C*0801, and C*1502, which are aligned without all, or substantially all, of the leader, transmembrane and cytoplasmic sequences in FIG. 9A. Any of those alleles may comprise a mutation at one or more of positions 84, 139 and/or 236 (as shown in FIG. 9A) selected from: a tyrosine to alanine substitution at position 84 (Y84A); a tyrosine to cysteine substitution at position 84 (Y84C); an alanine to cysteine substitution at position 139 (A139C); and an alanine to cysteine substitution at position 236 (A236C). In addition, an HLA-C polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-C alleles may also be employed (e.g., it may comprise 1-25, 1-5, 5-10, 10-15, 15-20, 20-25, or 25-30 amino acid insertions, deletions, and/or substitutions).

In some cases, a TMP of the present disclosure comprises an HLA-C heavy chain polypeptide comprising the following HLA-C consensus amino acid sequence:

X1SHSMX2YFX3TAVSX4PGRGEPX5FIX6VGYVDDTQFVX7FDSDAASPRGEPR X8PWVEQEGPEYWDRETQX9YKRQAQX10DRVX11LRX12LRGYYNQSEX13X14SHX15X16QX 17MX18GCDX19GPDGRLLRGX20X21QX22AYDGKDYIALNEDLRSWTAADTAAQITQRKX23E AARX24AEQX25RAYLEGX26CVEWLRRYLX27NGKX28TLQRAEX29PKTHVTHHPX30SDHEA TLRCWALGFYPAEITLTWQX31DGEDQTQDTELVETRPAGDGTFQKWAAVX32VPSGX33EQR YTCHX34QHEGLX35EPLTLX36WX37P (SEQ ID NO:79), wherein X1 is C or G; X2 is R or K; X3 is F, Y, S, or D; X4 is R or W; X5 is H or R; X6 is A or S; X7 is Q or R; X8 is A or E; X9 is N or K; X10 is T or A; X11 is S or N; X12 is N or K; X13 is A or D; X14 is G or R; X15 is T or I; X16 is L or I; X17 is W or R; X18 is C, Y, F, or S; X19 is L, or V; X20 is Y or H; X21 is D or N; X22 is Y, F, S, or L; X23 is L or W; X24 is E, A, Or T; X25 is R, L, or W; X26 is L or T; X27 is E OR K; X28 is E or K; X29 is H or P; X30 is R or V; X31 is W or R; X32 is V or M; X33 is E or Q; X34 is M or V; X35 is P or Q; X36 is R or S; and X37 is P or G.

As an example, an MHC class I heavy chain polypeptide of a TMP of the present disclosure can comprise an amino acid sequence having at least 75%, at least 80%, at least 85%, at least 90%, at least 95%, at least 98%, at least 99%, or 100%, amino acid sequence identity to the human HLA-C heavy chain amino acid sequence shown in FIG. 21Y (SEQ ID NO:123).

### HLA-C(Y84A; A236C)

As one non-limiting example, in some cases, the MHC class I heavy chain polypeptide is an HLA-C polypeptide that comprises Y84A and A236C substitutions. For example, in some cases, the MHC class I heavy chain polypeptide comprises an amino acid sequence having at least 75%, at least 80%, at least 85%, at least 90%, at least 95%, at least 98%, at least 99%, or 100%, amino acid sequence identity to the human HLA-C heavy chain (Y84A; A236C) amino acid sequence shown in FIG. 21Z (SEQ ID NO:124), where amino acid 84 is Ala and amino acid 236 is Cys. In some cases, the Cys-236 forms an interchain disulfide bond with Cys-12 of a variant β2M polypeptide that comprises an R12C substitution.

### HLA-C(Y84C; A139C)

In some cases, the MHC class I heavy chain polypeptide comprises Y84C and A139C substitutions. For example, in some cases, the MHC class I heavy chain polypeptide comprises an amino acid sequence having at least 75%, at least 80%, at least 85%, at least 90%, at least 95%, at least 98%, at least 99%, or 100%, amino acid sequence identity to the human HLA-C heavy chain (Y84C; A139C) amino acid sequence shown in FIG. 21AA (SEQ ID NO:125), where amino acid 84 is Cys and amino acid 139 is Cys. In some cases, Cys-84 forms an intrachain disulfide bond with Cys-139.

### HLA-C*0701

In some cases, a MHC class I heavy chain polypeptide of a TMP of the present disclosure comprises an amino acid sequence of HLA-C*0701 of FIG. 9A (labeled HLA-C in FIG. 6), or an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the HLA-C heavy chain polypeptide of a TMP of the present disclosure has less than 100% identity to the sequence labeled HLA-C*0701 in FIG. 9A, it may comprise a mutation at one or more of positions 84, 139 and/or 236 selected from: a tyrosine to alanine substitution at position 84 (Y84A); a tyrosine to cysteine substitution at position 84 (Y84C); an alanine to cysteine at position 139 (A139C); and an alanine to cysteine substitution at position 236 (A236C). In some cases, the HLA-C heavy chain polypeptide of a TMP of the present disclosure comprises Y84A and A236C substitutions. In some cases, the HLA-C*0701 heavy chain polypeptide of a T-Cell-MMP or its epitope conjugate comprises Y84C and A139C substitutions. In some cases, the HLA-C heavy chain polypeptide of a TMP of the present disclosure comprises Y84C, A139C, and A236C substitutions.

### Non-Classical HLA-E, -F, and -G MHC Class I Heavy Chains

In some cases, a TMP of the present disclosure comprises a non-classical MHC class I heavy chain polypeptide. Among the non-classical HLA heavy chain polypeptides, or portions thereof, that may be that may be incorporated into a TMP of the present disclosure include, but are not limited to, those of HLA-E, -F, and -G alleles. Amino acid sequences for HLA-E, -F, and -G heavy chain polypeptides, (and the HLA-A, B and C alleles) may be found on the world wide web hla.alleles.org/nomenclature/index.html, the European Bioinformatics Institute (www(dot)ebi(dot)ac(dot)uk), which is part of the European Molecular Biology Laboratory (EMBL), and at the National Center for Biotechnology Information (www(dot)ncbi(dot)nlm(dot)nih(dot)gov).

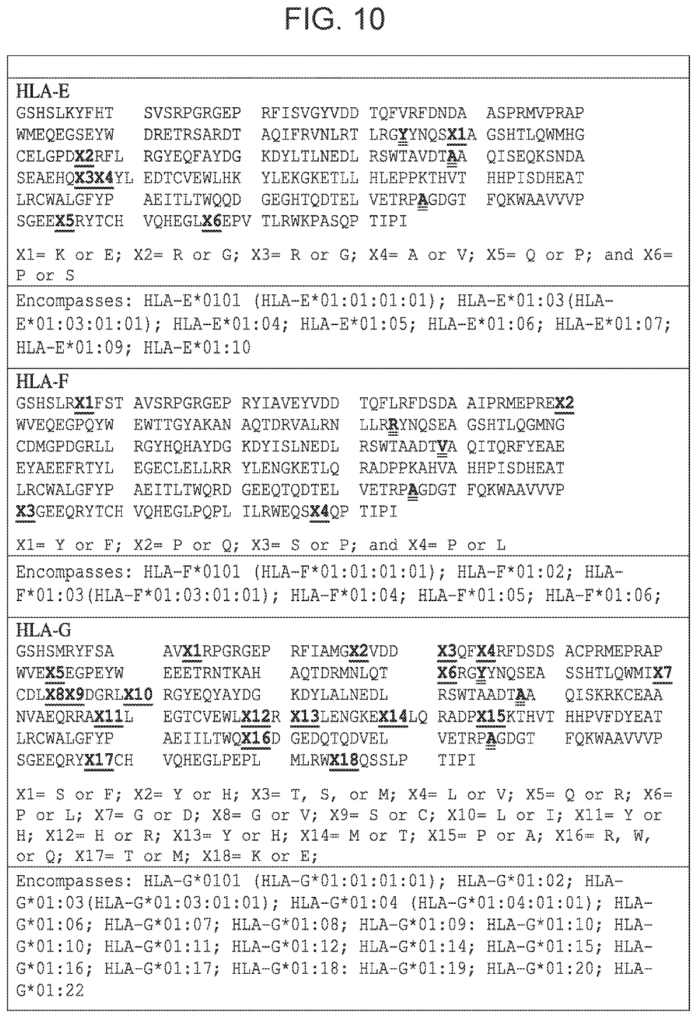

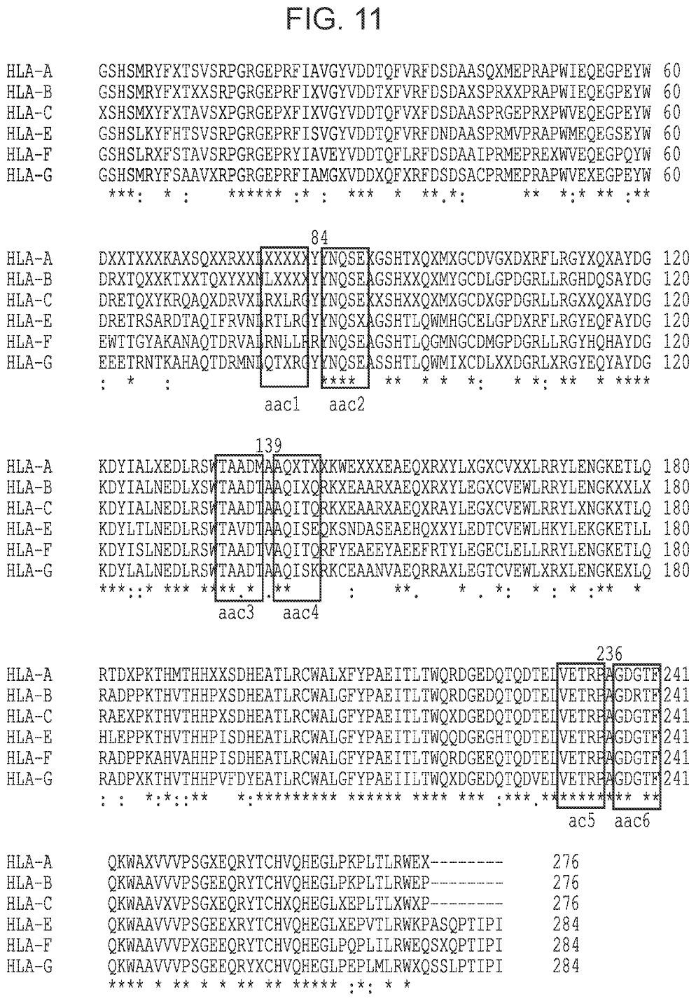

Non-limiting examples of suitable HLA-E alleles include, but are not limited to, HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Non-limiting examples of suitable HLA-F alleles include, but are not limited to, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06. Non-limiting examples of suitable HLA-G alleles include, but are not limited to, HLA-G*0101 (HLA-G*01:01:01:01), HLA-G*01:02, HLA-G*01:03 (HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA E, —F and -G alleles without all, or substantially all, of the leader, transmembrane and cytoplasmic sequences are provided in FIG. 10, and aligned with consensus sequences of the above-mentioned HLA-A, -B and -C alleles in FIG. 11.

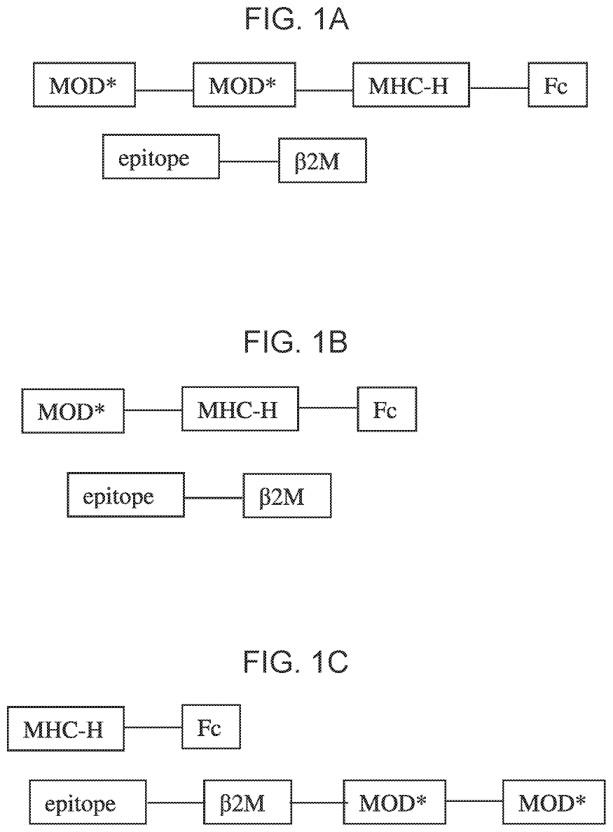

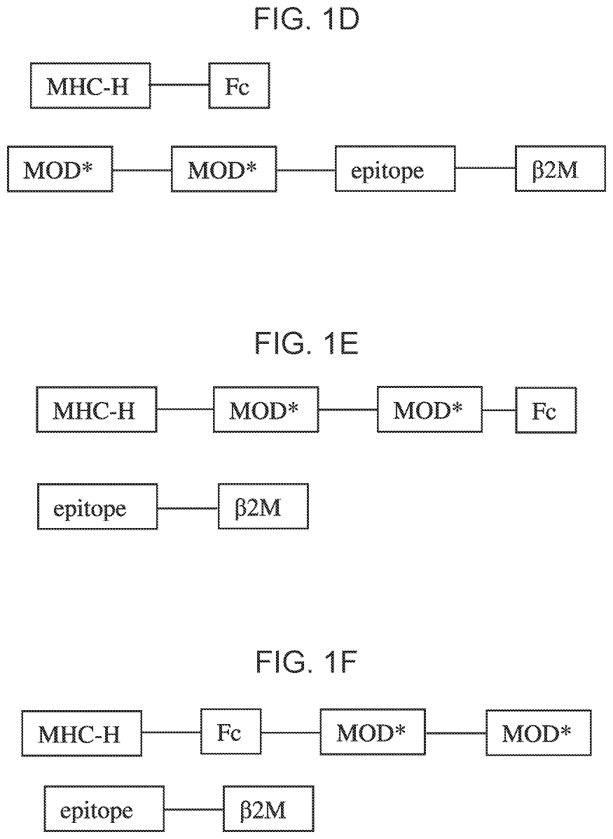

FIG. 1—provides a consensus sequence for each of HLA-E, -F, and -G with the variable aa positions indicated as “X” residues sequentially numbered and the locations of aas 84, 139 and 236 double underlined.

FIG. 11 provides an alignment of the consensus amino acid sequences for HLA-A, -B, -C, -E, -F, and -G, which are given in FIGS. 7-11. Variable residues in each sequence are listed as “X” with the sequential numbering removed. As indicated in FIG. 6, the locations of aas 84, 139 and 236 are indicated with their flanking five-amino acid clusters that may be replaced by 1 to 5 amino acids selected independently from (i) any naturally occurring amino acid or (ii) any naturally occurring amino acid except proline or glycine are also shown.

Any of the above-mentioned HLA-E, -F, and/or -G alleles may comprise a substitution at one or more of positions 84, 139 and/or 236 as shown in FIG. 11 for the consensus sequences. In some cases, the substitutions may be selected from a: position 84 tyrosine to alanine (Y84A) or cysteine (Y84C), or, in the case of HLA-F, an R84A or R84C substitution; a position 139 alanine to cysteine (A139C), or, in the case of HLA-F, a V139C; and an alanine to cysteine substitution at position 236 (A236C). In addition, an HLA-E, -F and/or -G sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of any of the consensus sequences of set forth in FIG. 11 may also be employed (e.g., the sequences may comprise 1-25, 1-5, 5-10, 10-15, 15-20, 20-25, or 25-30 amino acid insertions, deletions, and/or substitutions in addition to changes at variable residues listed therein).

### Mouse H2K

In some cases, a MHC class I heavy chain polypeptide present in a TMP of the present disclosure comprises an amino acid sequence of MOUSE H2K (SEQ ID NO:45) (MOUSE H2K in FIG. 6), or a sequence having at least 75%, at least 80%, at least 85%, at least 90%, at least 95%, at least 98%, at least 99%, or 100% amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the MOUSE H2K heavy chain polypeptide of a TMP of the present disclosure has less than 100% identity to the sequence labeled MOUSE H2K in FIG. 6, it may comprise a mutation at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In some cases, the MOUSE H2K heavy chain polypeptide of a TMP of the present disclosure comprises Y84A and A236C substitutions. In some cases, the MOUSE H2K heavy chain polypeptide of a TMP of the present disclosure comprises Y84C and A139C substitutions. In some cases, the MOUSE H2K heavy chain polypeptide of a TMP of the present disclosure comprises Y84C, A139C and A236C substitutions.

### Exemplary Combinations

Table 1, below, presents various combinations of MHC class I heavy chain sequence modifications that can be incorporated in a TMP of the present disclosure.

TABLE 1

| | | Sequence | Specific |
|---|---|---|---|
| | HLA Heavy | Identity | Substitutions at aa |
| Entry | Chain Sequence | Range [symbol] | positions 84, 139 and/or 236 |
| 1 | HLA-A | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 7B) | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions (not | (Y84C, A139C & |
| | | counting variable residues) | A236C) |
| 2 | A*0101, A*0201, | 75%-99.8%, 80%-99.8%, 85%-99.8%, | None; Y84C; Y84A; |
| | A*0301, A*1101, | 90%-99.8%, 95%-99.8%, 98%-99.8%, | A139C; A236C; |
| | A*2402, A*2301, | or 99%-99.8%; or 1-25, 1-5, 5-10, 10- | (Y84A & A236C); |
| | A*2402, A*2407, | 15, 15-20, or 20-25 aa insertions, | (Y84C & A139C); or |
| | A*3303, or | deletions, and/or substitutions | (Y84C, A139C & |
| | A*3401 | | A236C) |
| | (FIG. 7A) | | |
| 3 | HLA-B | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 8B) | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions (not | (Y84C, A139C & |
| | | counting variable residues) | A236C) |
| 4 | B*0702, B*0801, | 75%-99.8%, 80%-99.8%, 85%-99.8%, | None; Y84C; Y84A; |
| | B*1502, B*3802, | 90%-99.8%, 95%-99.8%, 98%-99.8%, | A139C; A236C; |
| | B*4001, B*4601, | or 99%-99.8%; or 1-25, 1-5, 5-10, 10- | (Y84A & A236C); |
| | or B*5301 | 15, 15-20, or 20-25 aa insertions, | (Y84C & A139C); or |
| | (FIG. 8A) | deletions, and/or substitutions | (Y84C, A139C & |
| | | | A236C) |
| 5 | HLA-C | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 9B) | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions (not | (Y84C, A139C & |
| | | counting variable residues) | A236C) |
| 6 | C*0102, C*0303, | 75%-99.8%, 80%-99.8%, 85%-99.8%, | None; Y84C; Y84A; |
| | C*0304, C*0401, | 90%-99.8%, 95%-99.8%, 98%-99.8%, | A139C; A236C; |
| | C*0602, C*0701, | or 99%-99.8%; or 1-25, 1-5, 5-10, 10- | (Y84A & A236C); |
| | C*0801, or | 15, 15-20, or 20-25 aa insertions, | (Y84C & A139C); or |
| | C*1502 | deletions, and/or substitutions | (Y84C, A139C & |
| | (FIG. 9A) | | A236C) |
| 7 | HLA-E, F, or G | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 10) | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions (not | (Y84C, A139C & |
| | | counting variable residues) | A236C) |
| 8 | MOUSE H2K | 75%-99.8%, 80%-99.8%, 85%-99.8%, | None; Y84C; Y84A; |
| | (FIG. 6) | 90%-99.8%, 95%-99.8%, 98%-99.8%, | A139C; A236C; |
| | | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions | (Y84C, A139C & |
| | | | A236C) |

[symbol]

The Sequence Identity Range is the permissible range in sequence identity of an MHC-H polypeptide sequence incorporated into a TMP relative to the corresponding portion of the sequences listed in FIG. 6-11 not counting the variable residues in the consensus sequences.

### Beta-2 Microglobulin

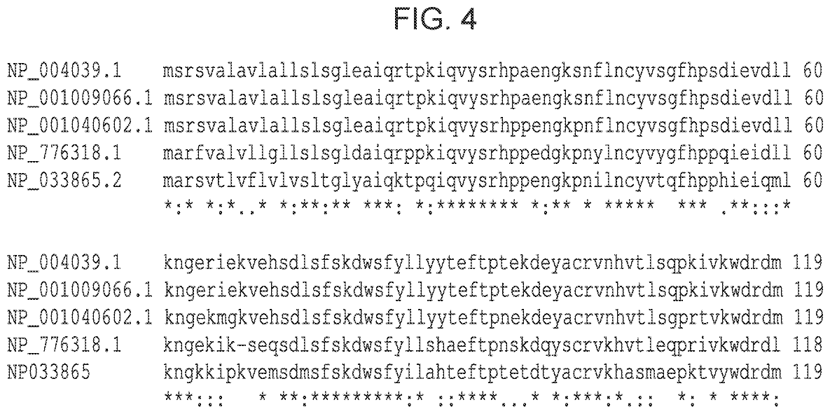

A β2-microglobulin (β2M) polypeptide of a TMP of the present disclosure can be a human β2M polypeptide, a non-human primate β2M polypeptide, a murine β2M polypeptide, and the like. In some instances, a β2M polypeptide comprises an amino acid sequence having at least 75%, at least 80%, at least 85%, at least 90%, at least 95%, at least 98%, at least 99%, or 100%, amino acid sequence identity to a β2M amino acid sequence depicted in FIG. 4. In some instances, a β2M polypeptide comprises an amino acid sequence having at least 75%, at least 80%, at least 85%, at least 90%, at least 95%, at least 98%, at least 99%, or 100%, amino acid sequence identity to amino acids 21 to 119 of a β2M amino acid sequence depicted in FIG. 4.

In some cases, a suitable β2M polypeptide comprises the amino acid sequence of FIG. 21CC (SEQ ID NO:126); and the HLA Class I heavy chain polypeptide comprises the amino acid sequence of FIG. 21HH (SEQ ID NO:127), where the cysteine residues indicated as {C}form an disulfide bond between the α1 and α2-1 helices and the residue forms a disulfide bond with the β2M polypeptide cysteine at position 12. In the sequence above, “aa1” is “amino acid cluster 1”; “aa2” is “amino acid cluster 2”; “aa3” is “amino acid cluster 3”; “aa4” is “amino acid cluster 4”; “aa5” is “amino acid cluster 5”; and “aa6” is “amino acid cluster 6”; see, e.g., FIG. 8. Each occurrence of aa1, aa2, aa3, aa4, aa5, and aa6 is and independently selected to be 1-5 amino acid residues, wherein the amino acid residues are i) selected independently from any naturally occurring (e.g., encoded) amino acid or ii) any naturally occurring amino acid except proline or glycine.

In some cases, an MHC polypeptide comprises a single amino acid substitution relative to a reference MHC polypeptide (where a reference MHC polypeptide can be a wild-type MHC polypeptide), where the single amino acid substitution substitutes an amino acid with a cysteine (Cys) residue. Such non-naturally cysteine residues, when present in an MHC polypeptide of a first polypeptide of a heterodimeric TMP or in an MHC polypeptide in a single-chain TMP of the present disclosure, can form a disulfide bond with a cysteine residue present in a second polypeptide chain of a TMP of the present disclosure or in another MHC polypeptide in a single-chain TMP.

In some cases, a first MHC polypeptide in a first polypeptide of a heterodimeric TMP of the present disclosure, and/or the second MHC polypeptide in the second polypeptide of a heterodimeric TMP of the present disclosure, includes an amino acid substitution to substitute an amino acid with a cysteine, where the non-naturally occurring cysteine in the first MHC polypeptide forms a disulfide bond with a cysteine in the second MHC polypeptide, where a cysteine in the first MHC polypeptide forms a disulfide bond with the non-naturally occurring cysteine in the second MHC polypeptide, or where the non-naturally occurring cysteine in the first MHC polypeptide forms a disulfide bond with the non-naturally occurring cysteine in the second MHC polypeptide. Similarly, one of the MHC polypeptides in a single-chain TMP of the present disclosure includes an amino acid substitution to substitute an amino acid with a cysteine, where the non-naturally occurring cysteine in the MHC polypeptide forms a disulfide bond with a naturally occurring or non-naturally occurring cysteine in a different MHC polypeptide in the TMP.

For example, in some cases, one of following pairs of residues in an HLA β2-microglobulin and an HLA Class I heavy chain is substituted with cysteines (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12) β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue Gln96; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The amino acid numbering of the MHC/HLA Class I heavy chain is in reference to the mature MHC/HLA Class I heavy chain, without a signal peptide. For example, in some cases, residue 236 of the mature HLA-A amino acid sequence is substituted with a Cys. In some cases, residue 236 of the mature HLA-B amino acid sequence is substituted with a Cys. In some cases, residue 236 of the mature HLA-C amino acid sequence is substituted with a Cys. In some cases, residue 32 (corresponding to Arg-12 of mature β2M) of an amino acid sequence depicted in FIG. 4 is substituted with a Cys.

In some cases, a β2M polypeptide comprises the amino acid sequence shown in FIG. 21BB (SEQ ID NO:128) or FIG. 21CC (SEQ ID NO:129).

In some cases, an HLA Class I heavy chain polypeptide comprises the amino acid sequence shown in FIG. 21DD (SEQ ID NO:130), 21EE (SEQ ID NO:131) or 21FF (SEQ ID NO:132).

In some cases, the β2M polypeptide of a TMP of this disclosure comprises the amino acid sequence of FIG. 21CC (SEQ ID NO:133) and the HLA Class I heavy chain polypeptide of the TMP comprises the amino acid sequence of FIG. 21EE (SEQ ID NO:134), where the Cys residues that are underlined and in bold form a disulfide bond with one another in the TMP.

In some cases, the β2M polypeptide comprises the amino acid sequence of FIG. 21GG (SEQ ID NO:135).

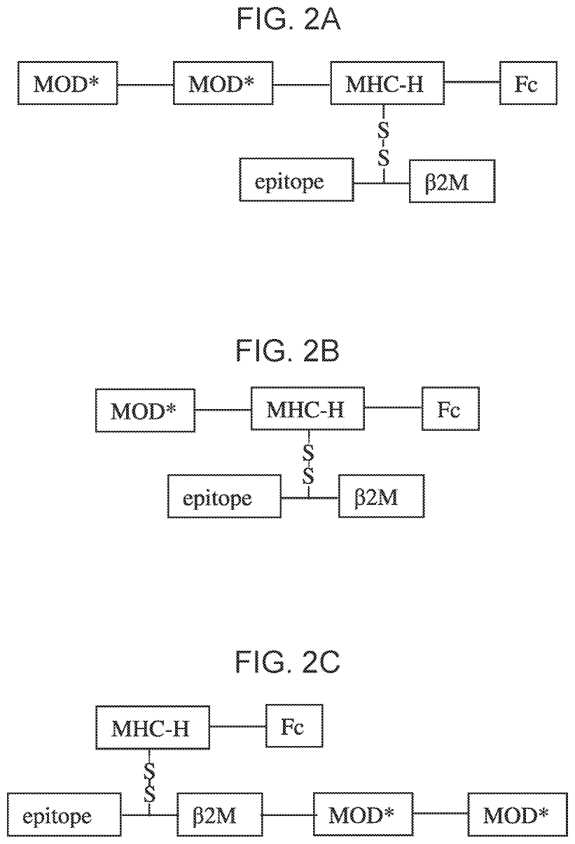

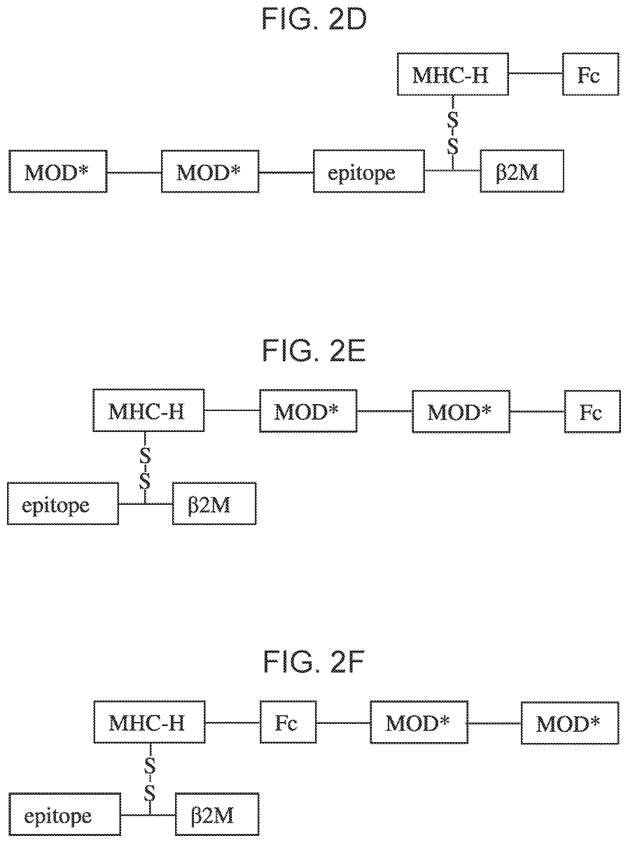

In some cases, MHC polypeptides in a heterodimeric or single-chain TMP of the present disclosure are disulfide linked to one another through: i) a Cys residue present in a linker connecting the peptide epitope and a β2M polypeptide; and ii) a Cys residue present in an MHC class I heavy chain. In some cases, the Cys residue present in the MHC class I heavy chain is a Cys introduce as a Y84C substitution. In some cases, the linker connecting the peptide epitope and the β2M polypeptide is GCGGS(GGGGS)n (SEQ ID NO:583), where n is 1, 2, 3, 4, 5, 6, 7, 8, or 9. For example, in some cases, the linker comprises the amino acid sequence GCGGSGGGGSGGGGSGGGGS (SEQ ID NO:137). As another example, the linker comprises the amino acid sequence GCGGSGGGGSGGGGS (SEQ ID NO:138). Examples of disulfide-linked first and second polypeptides of a heterodimeric TMP of the present disclosure are depicted schematically in FIG. 2A-2F.

### Multiple Disulfide Bonded TMPS

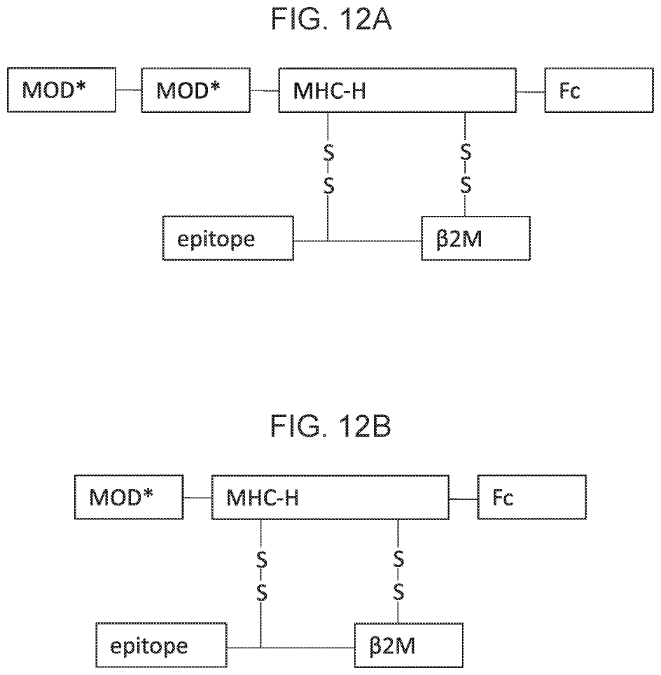

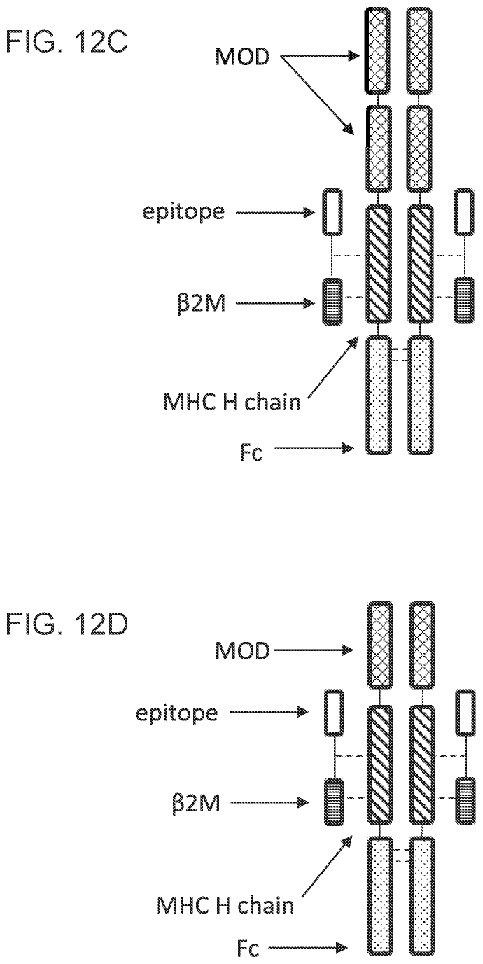

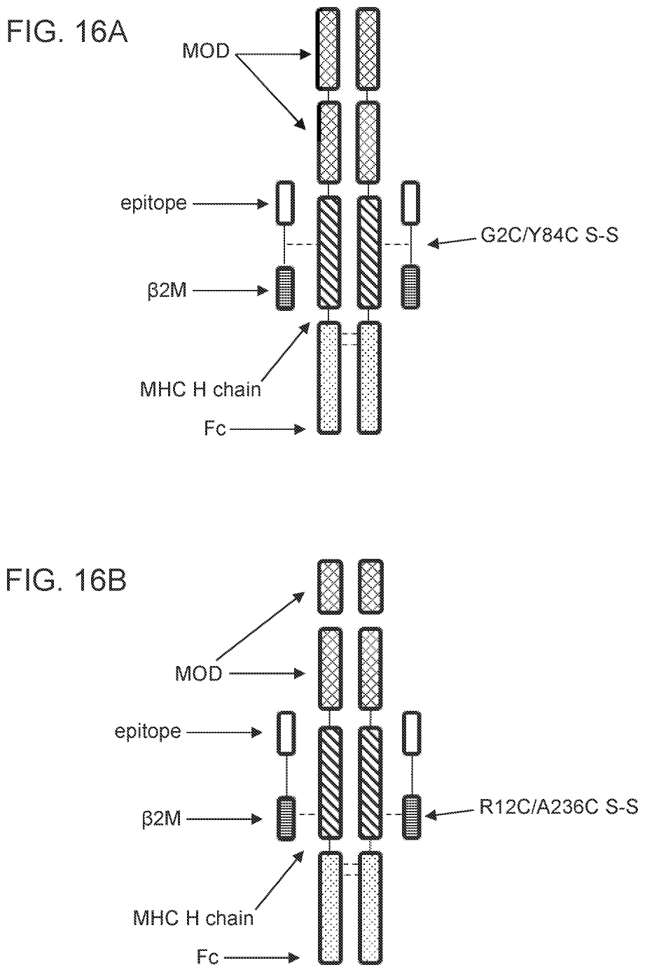

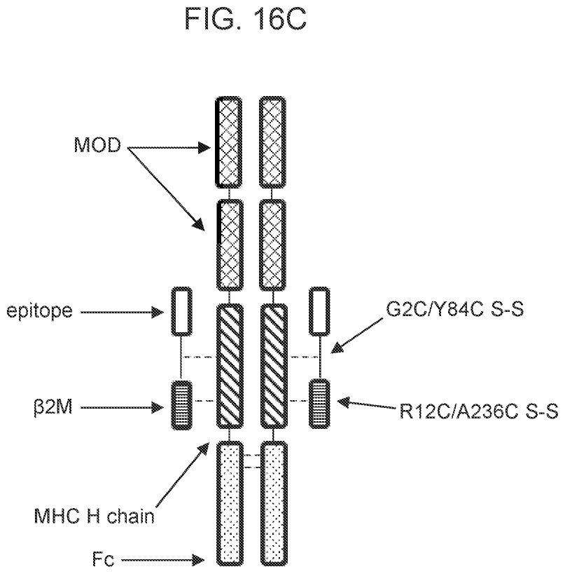

In some cases, the first polypeptide and the second polypeptide of a heterodimeric TMP of the present disclosure are linked to one another by at least two disulfide bonds (i.e., two interchain disulfide bonds). Examples of such multiple disulfide-linked TMP are depicted schematically in FIGS. 12A and 12B; and in FIG. 16C. In addition, where a heterodimeric or single-chain TMP of the present disclosure comprises an Ig Fc polypeptide, a heterodimeric TMP can be dimerized, such that disulfide bonds link the Ig Fc polypeptides in the two heterodimeric TMPs. Such an arrangement is depicted schematically in FIGS. 12C and 12D, where disulfide bonds are represented by dashed lines. Unless otherwise stated, the at least two disulfide bonds described in the multiple disulfide-linked TMPPs in this section are not referring to disulfide bonds linking Ig Fe polypeptides in dimerized TMPs.

For example, in some instances, the first polypeptide and the second polypeptide of a heterodimeric TMP of the present disclosure are linked to one another by 2 interchain disulfide bonds. As another example, in some instances, the first polypeptide and the second polypeptide of a TMP of the present disclosure are linked to one another by 3 interchain disulfide bonds. As another example, in some instances, the first polypeptide and the second polypeptide of a TMP of the present disclosure are linked to one another by 4 interchain disulfide bonds.

In some cases where a peptide epitope of a heterodimeric or single-chain TMP of the present disclosure is linked to a β2M polypeptide by a linker comprising a Cys, at least one of the at least two disulfide bonds links a Cys in the linker to a Cys in an MHC class I heavy chain in TMP. In some cases, where a peptide epitope of a TMP of the present disclosure is linked to an MHC class I heavy chain polypeptide by a linker, at least one of the at least two disulfide bonds links a Cys in the linker to a Cys in a β2M polypeptide present in the TMP.

In some cases, a multiple disulfide-linked TMP of the present disclosure (e.g., a double disulfide-linked TMP) exhibits increased stability and/or improved expression, compared to a control TMP that includes only one of the at least two disulfide bonds. In some cases, a multiple disulfide-linked TMP (e.g., a double disulfide-linked TMP) of the present disclosure exhibits increased in vitro stability, compared to a control TMP that includes only one of the at least two disulfide bonds. For example, in some cases, a multiple disulfide-linked TMP of the present disclosure (e.g., a double disulfide-linked TMP) exhibits at least 5%, at least 10%, at least 15%, at least 20%, at least 25%, at least 50%, at least 2-fold, at least 5-fold, or at least 10-fold, greater in vitro stability, compared to a control TMP that includes only one of the at least two disulfide bonds.

Stability is determined by measuring the percent of TMP remaining in the solution after a specified time in the solution at a specified temperature. Stability can be measured in the PBS buffer solution containing 500 mM NaCl (described above) in vitro over a specified period of time and at a specified temperature (e.g., in a solution at a temperature of 37° C. to 42° C. for a period of time of from 1 hour to 28 days; e.g., for 1 hour at 37° C.; 1 day at 37° C.; 5 days at 37° C.; 1 hour at 42° C.; 1 day at 42° C.; 5 days at 42° C.; 5 days at 37° C.; 10 days at 37° C.; 14 days at 37° C.; 28 days at 37° C.; and the like), compared to a control TMMP lacking the at least one disulfide bond between the first polypeptide and the second polypeptide of the heterodimer. The TMMP can be present in the PBS buffer solution in a concentration of from 0.1 mg/mL to 10 mg/mL, e.g., about 1 mL, about 2 mL, about 3 mL, about 4 mL, about 5 mL, about 6 mL, about 7 mL, about 8 mL, about 9 mL, or about 10 mL, and the buffer solution can be kept at 37° C. or 42° C. for 1 hour, 5 days, 10 days, 14 days, 21 days, or 28 days.

Whether a multiple disulfide-linked TMP of the present disclosure exhibits increased in vitro stability compared to a control TMP that includes only one of the at least two disulfide bonds, can be determined by measuring the amount of each TMP present in samples as discussed above, e.g., kept at 37° C. and/or 42° C. for 1 hour, 5 days, 10 days, 14 days, 21 days, or 28 days.

For example, in some cases, a multiple disulfide-linked TMP (e.g., a double disulfide-linked TMP) of the present disclosure exhibits at least 5%, at least 10%, at least 15%, at least 20%, at least 25%, at least 50%, at least 2-fold, at least 5-fold, or at least 10-fold, greater in vitro stability, compared to a control TMP that includes only one of the at least two disulfide bonds, when the TMP is stored at 37° C. for a period of time (e.g., for a period of time of from about 1 week to about 2 weeks, from about 2 weeks to about 4 weeks, or from about 4 weeks to about 2 months). For example, in some cases, the amount of disulfide-linked heterodimeric TMP remaining after storing a multiple disulfide-linked TMP (e.g., a double disulfide-linked TMP) of the present disclosure in vitro at 37° C. for 28 days is at least at least 5%, at least 10%, at least 15%, at least 20%, at least 25%, at least 50%, at least 2-fold, at least 5-fold, or at least 10-fold, greater than the amount of disulfide-linked heterodimeric TMP remaining after storing a control TMP (a TMP that includes only one of the at least two disulfide bonds present in the multiple disulfide-linked TMP) in vitro at 37° C. for 28 days.

In some cases, a multiple disulfide-linked IMP of the present disclosure exhibits increased in vivo stability, compared to a control TMP that includes only one of the at least two disulfide bonds. For example, in some cases, a multiple disulfide-linked TMP of the present disclosure exhibits at least 5%, at least 10%, at least 15%, at least 20%, at least 25%, at least 50%, at least 2-fold, at least 5-fold, or at least 10-fold, greater in vivo stability, compared to a control TMP that includes only one of the at least two disulfide bonds.

In some cases, the presence of two disulfide bonds in a multiple disulfide-linked TMP of the present disclosure (e.g., a double disulfide-linked TMP) provides for increased production of disulfide-linked heterodimeric or single-chain TMP, compared to the amount of disulfide-linked heterodimeric TMP produced when the IMP is a control TMP that includes only one of the at least two disulfide bonds. For example, a multiple disulfide-linked TMP of the present disclosure (e.g., a double disulfide-linked TMP) can be produced in a mammalian cell in in vitro cell culture, where the mammalian cell is cultured in a liquid cell culture medium. The TMP can be secreted into the cell culture medium. The cells can be lysed, generating a cell lysate, and the TMP can be present in the cell lysate. The TMP can be purified from the cell culture medium and/or the cell lysate. For example, where the TMP comprises an IgG1 Fe polypeptide, the cell culture medium and/or the cell lysate can be contacted with immobilized protein A (e.g., the cell culture medium and/or the cell lysate can be applied to a protein A column, where protein A is immobilized onto beads). TMP present in the cell culture medium and/or the cell lysate becomes bound to the immobilized protein A. After washing the column to remove unbound materials, the bound TMP is eluted, generating a protein A eluate. The amount of disulfide-linked heterodimeric or single-chain TMP present in the protein A eluate is a least 0.5%, at least 1%, at least 2%, at least 3%, at least 4%, at least 5%, at least 6%, at least 7%, at least 8%, at least 9%, or at least 10%, higher than the amount of disulfide-linked heterodimeric or single-chain TMP present in the protein A eluate when the TMP is a control TMP that includes only one of the at least two disulfide bonds present in the multiple disulfide-linked TMP (e.g., a double disulfide-linked TMP). In some cases, the percent of the total IMP protein in the eluate that is non-aggregated disulfide-linked heterodimeric or single-chain TMP is at least 70%, at least 75%, at least 80%, at least 85%, at least 90%, at least 95%, or at least 99%. The protein A eluate can be subjected to size exclusion chromatography (SEC) and/or one or more other additional purification steps.

In some cases, a T-cell modulatory polypeptide of the present disclosure comprises at least one heterodimer comprising: a) a first polypeptide comprising: i) a KRAS peptide, where the KRAS peptide has a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, and peptides within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length); and ii) first MHC polypeptide; b) a second polypeptide comprising a second MHC polypeptide, and c) at least one immunomodulatory polypeptide, where the first and/or the second polypeptide comprises the immunomodulatory polypeptide, and where the heterodimer comprises at least two disulfide bonds (e.g., two disulfide bonds) between the first polypeptide and the second polypeptide (e.g., the heterodimer comprises: i) a first disulfide bond linking the first polypeptide and the second polypeptide; and ii) a second disulfide bond linking the first polypeptide and the second polypeptide). Expressed another way, the first polypeptide comprises a first Cys residue that forms a disulfide bond (a first disulfide bond) with a first Cys residue in the second polypeptide; and the first polypeptide comprises a second Cys residue that forms a disulfide bond (a second disulfide bond) with a second Cys residue in the second polypeptide.

In some cases, a TMP of the present disclosure comprises: a) a first polypeptide comprising, in order from N-terminus to C-terminus: i) a KRAS peptide; ii a peptide linker; and iii) a β2M polypeptide; and b) a second polypeptide comprising an MHC class I heavy chain polypeptide, where one or both of the first and the second polypeptides comprises at least one immunomodulatory polypeptide, where the TMP comprises: a) a first disulfide linkage between: i) a Cys present in the linker between the KRAS peptide and the β2M polypeptide; and ii) a first Cys introduced into the MHC class I heavy chain polypeptide; and b) at least a second disulfide linkage between the first polypeptide and the second polypeptide, where the at least a second disulfide linkage is between: i) a Cys in the first polypeptide that is C-terminal to the Cys present in the linker; and ii) a Cys in the second polypeptide that is C-terminal to the first Cys introduced into the MHC class I heavy chain polypeptide.

Generally speaking, potential locations in the heterodimeric or single-chain TMP for disulfide bonds are where residues in the different polypeptides of the TMP are separated by a distance of 5 angstroms or less. Such locations represent potential locations where Cys residues, if not naturally present, can be substituted for the residues that exist in the polypeptides. For example, a first and second polypeptide of a heterodimeric TMP potentially can be linked via a disulfide bond between two Cys residues that are generally no more than about 5 angstroms apart from one another in the heterodimer. In some cases, one or both of the Cys residues are non-naturally occurring. An amino acid in the ß2M and MHC heavy chain of heterodimeric or single-chain TMPs that are no more than 5 angstroms from one another represent amino acids that, when substituted with a Cys, can form a disulfide bond in a TMP of the present disclosure. Similarly, a disulfide bond can be formed between a Cys residue in a linker and a naturally occurring or non-naturally occurring Cys residue in an MHC heavy chain where the two Cys residues are no more than about 5 angstroms apart from each other. Notably, however, not all pairs of residues separated by about 5 angstroms or less will be suitable for formation of a disulfide bond or provide a disulfide bond that stabilizes the resulting TMP or provides enhanced expression.

A multiple disulfide-linked heterodimeric TMP of the present disclosure (e.g., a double disulfide-linked TMP) can comprise, for example: a) a first polypeptide comprising: i) a KRAS peptide (e.g., a KRAS peptide of from 4 amino acids to about 25 amino acids in length, that is bound by a TCR when the peptide is complexed with MHC polypeptides); and ii) a first MHC polypeptide, where the first polypeptide comprises a peptide linker between the KRAS peptide and the first MIHC polypeptide, where the peptide linker comprises a Cys residue, and where the first MHC polypeptide is a β2M polypeptide that comprises an amino acid substitution that introduces a Cys residue; b) and a second polypeptide comprising a second MHC polypeptide, where the second MHC polypeptide is a Class I heavy chain comprising a Y84C substitution and an A236C substitution, based on the amino acid numbering of HLA-A*0201 (depicted in FIG. 7A), or at corresponding positions in another Class I heavy chain allele, where the TMP comprises a disulfide bond between the Cys residue in the peptide linker and the Cys residue at amino acid position 84 of the Class I heavy chain or corresponding position of another Class I heavy chain allele, and where the TMP comprises a disulfide bond between the introduced Cys residue in the β2M polypeptide and the Cys at amino acid position 236 of the Class I heavy chain or corresponding position of another Class I heavy chain allele; and c) at least one immunomodulatory polypeptide, where the first and/or the second polypeptide comprises the at least one immunomodulatory polypeptide. Examples are depicted schematically in FIG. 12A and FIG. 12B.

In some cases, the peptide linker comprises the amino acid sequence GCGGS (SEQ ID NO:139). In some cases, the peptide linker comprises the amino acid sequence GCGGS(GGGGS)n (SEQ ID NO: 140), where n is 1, 2, 3, 4, 5, 6, 7, 8, 9, or 10; e.g., 1, 2, or 3.

In some cases, the peptide linker comprises the amino acid sequence CGGGS (SEQ ID NO:141). In some cases, the peptide linker comprises the amino acid sequence CGGGS(GGGGS)n (SEQ ID NO: 142), where n is 1, 2, 3, 4, 5, 6, 7, 8, 9, or 10; e.g., 1, 2, or 3.

In some cases, the peptide linker comprises the amino acid sequence GGCGS (SEQ ID NO:587). In some cases, the peptide linker comprises the amino acid sequence GGCGS(GGGGS)n (SEQ ID NO:592), where n is 1, 2, 3, 4, 5, 6, 7, 8, 9 or 10, e.g., 1, 2, or 3.

In some cases, the peptide linker comprises the amino acid sequence GGGCS (SEQ ID NO:588). In some cases, the peptide linker comprises the amino acid sequence GGGCS(GGGGS)n (SEQ ID NO:589), where n is 1, 2, 3, 4, 5, 6, 7, 8, 9, or 10, e.g., 1, 2, or 3.

In some cases, the peptide linker comprises the amino acid sequence GGGGC (SEQ ID NO:590). In some cases, the peptide linker comprises the amino acid sequence GGGGC(GGGGS)n (SEQ ID NO:591), where n is 1, 2, 3, 4, 5, 6, 7, 8, 9, or 10, e.g., 1, 2, or 3.

The following are non-limiting examples of MHC class I heavy chain comprising a Y84C substitution and an A236C substitution, based on the amino acid numbering of HLA-A*0201 (depicted in FIG. 7A), or at corresponding positions in another Class I heavy chain allele.

### HLA-A

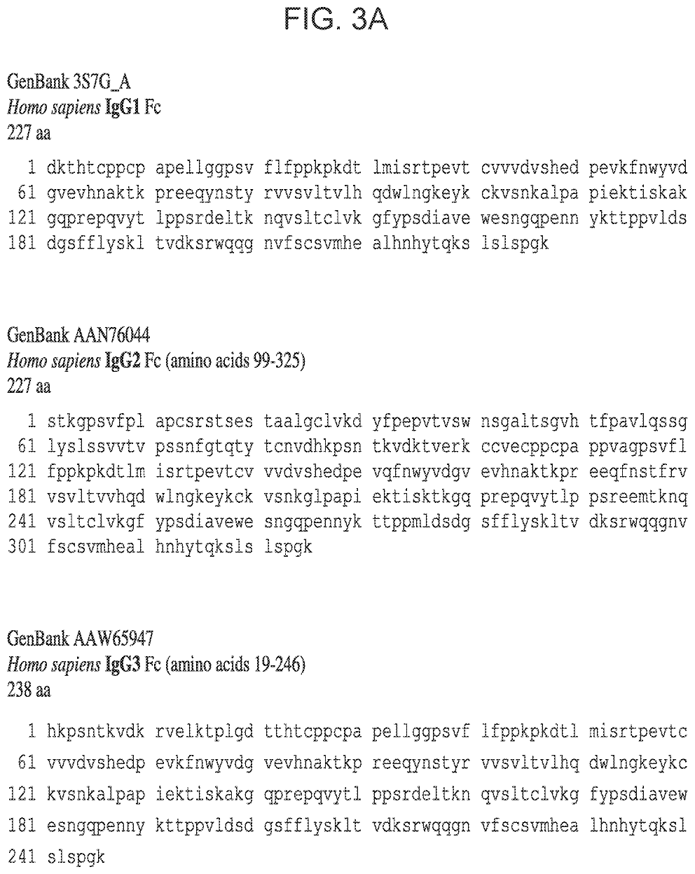

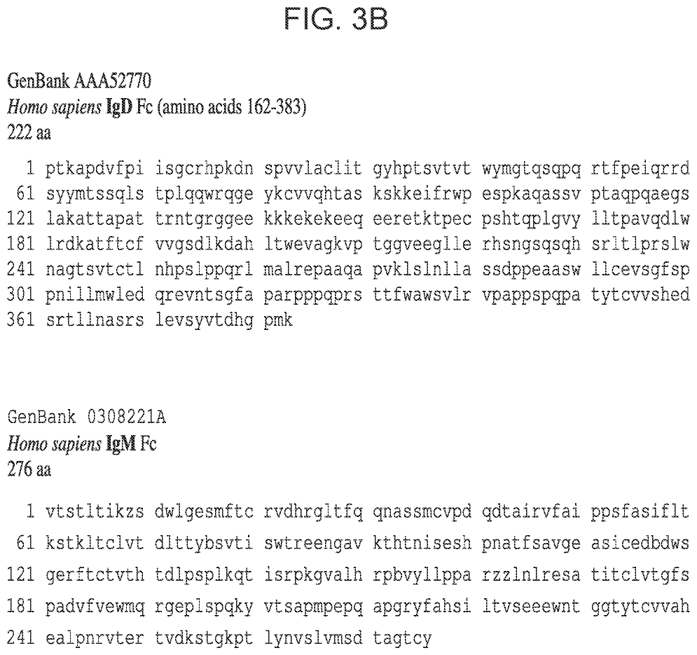

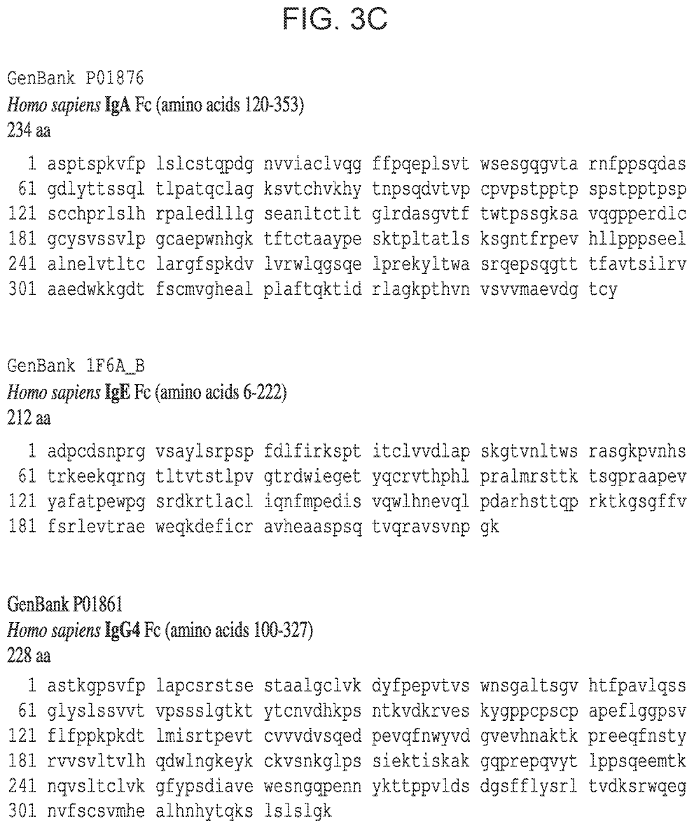

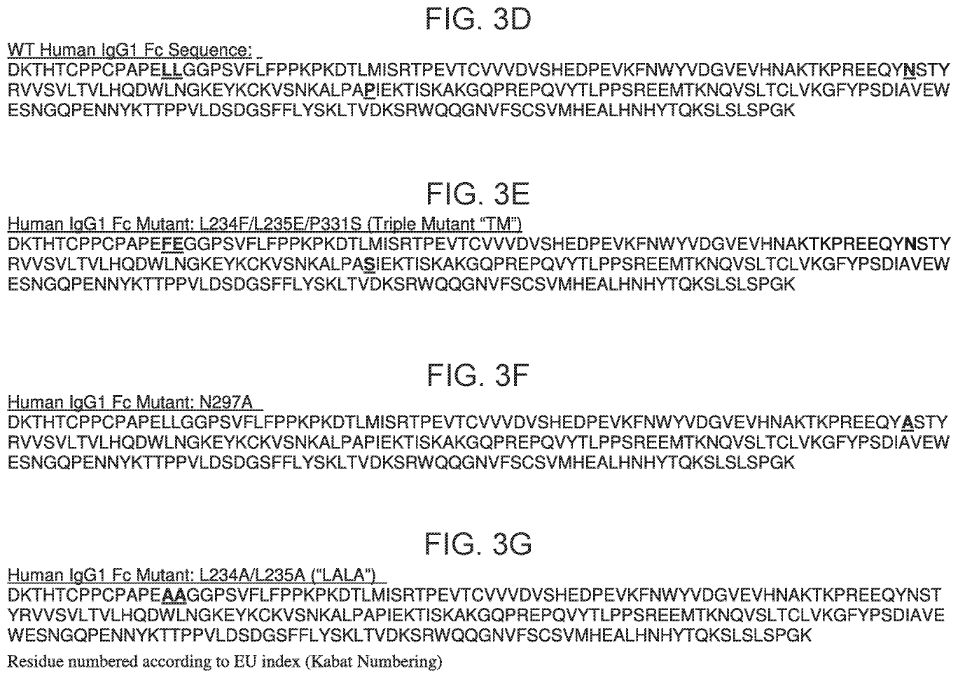

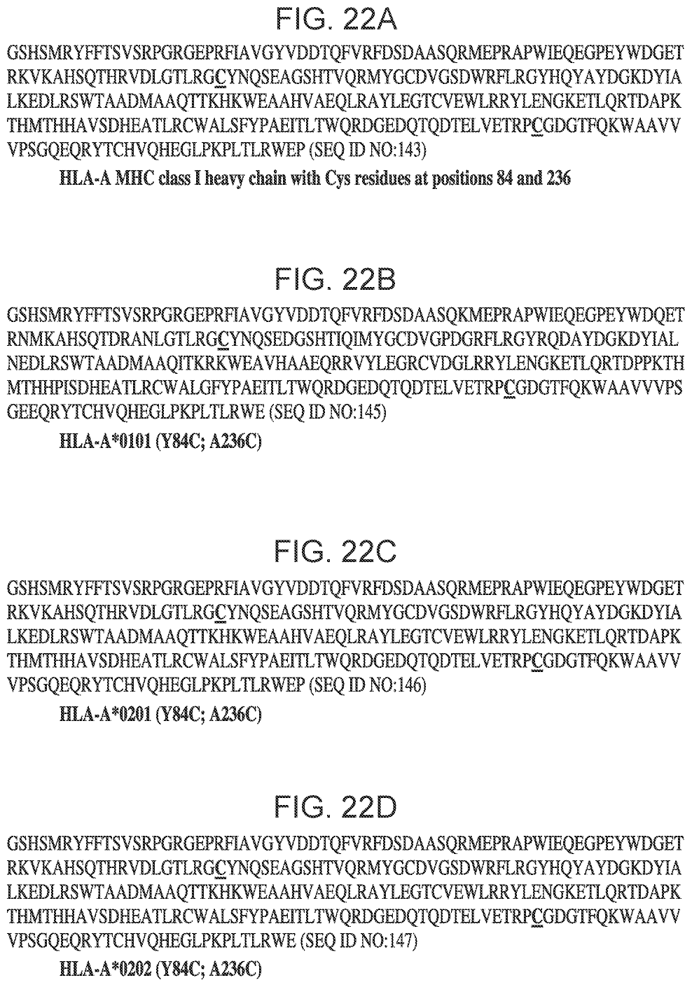

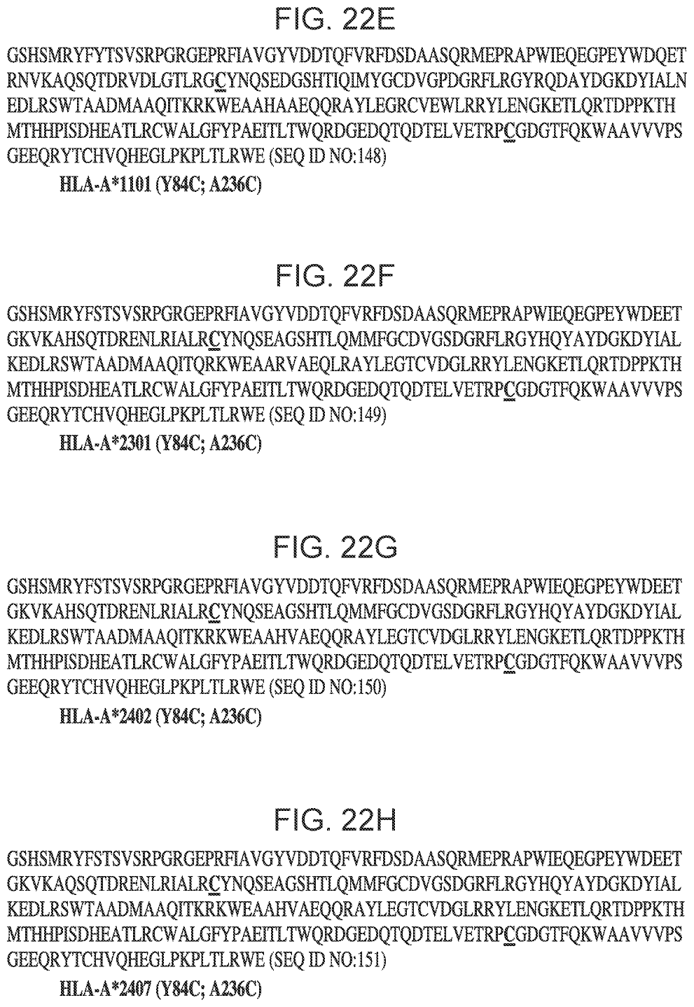

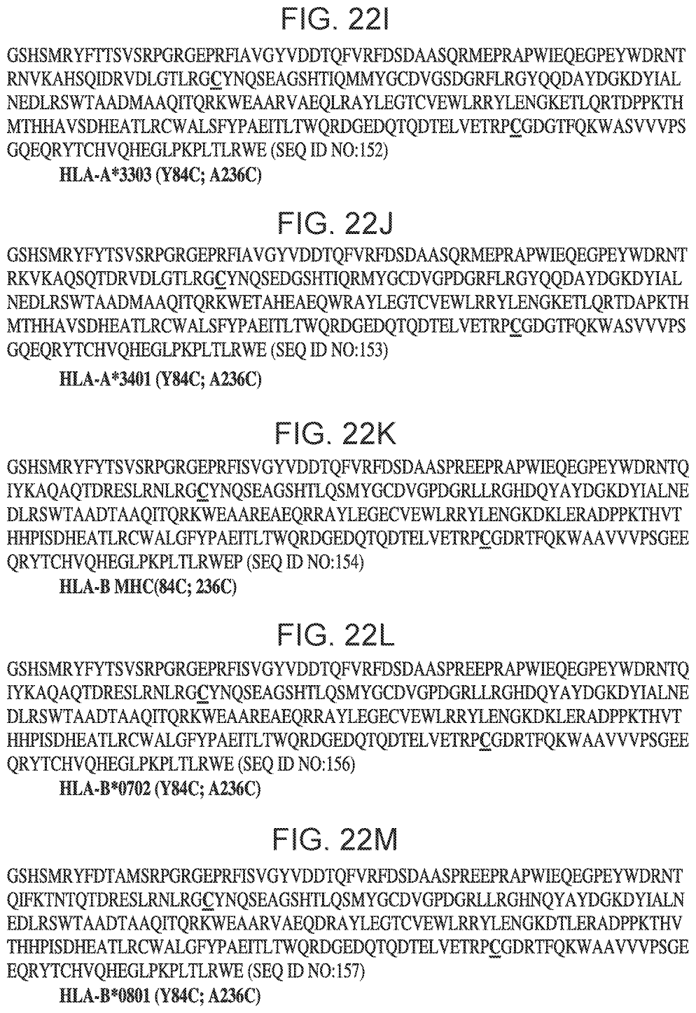

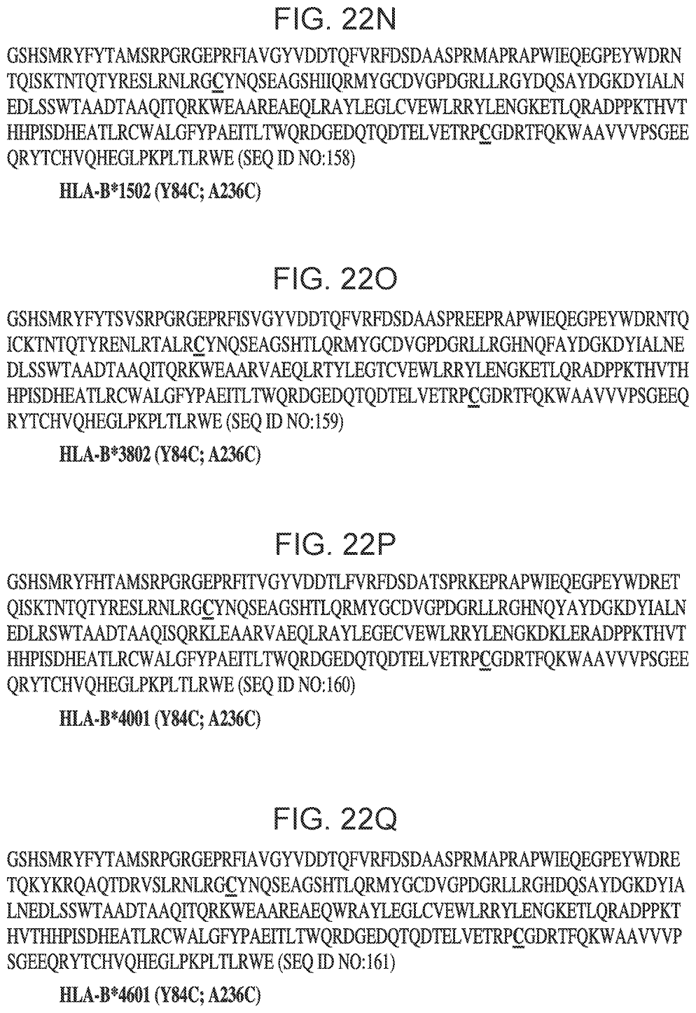

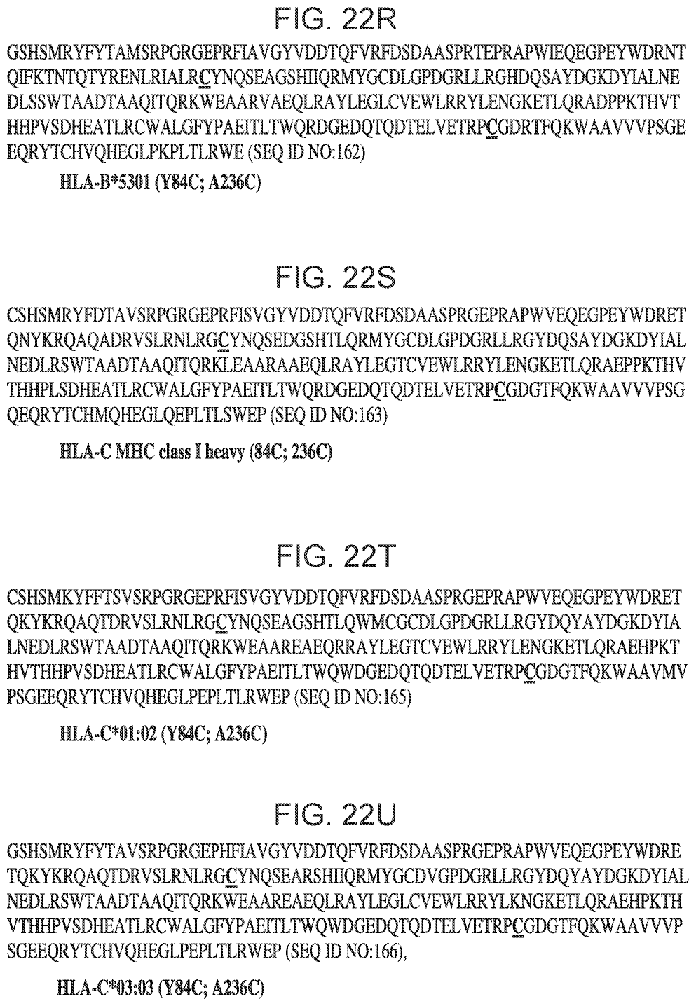

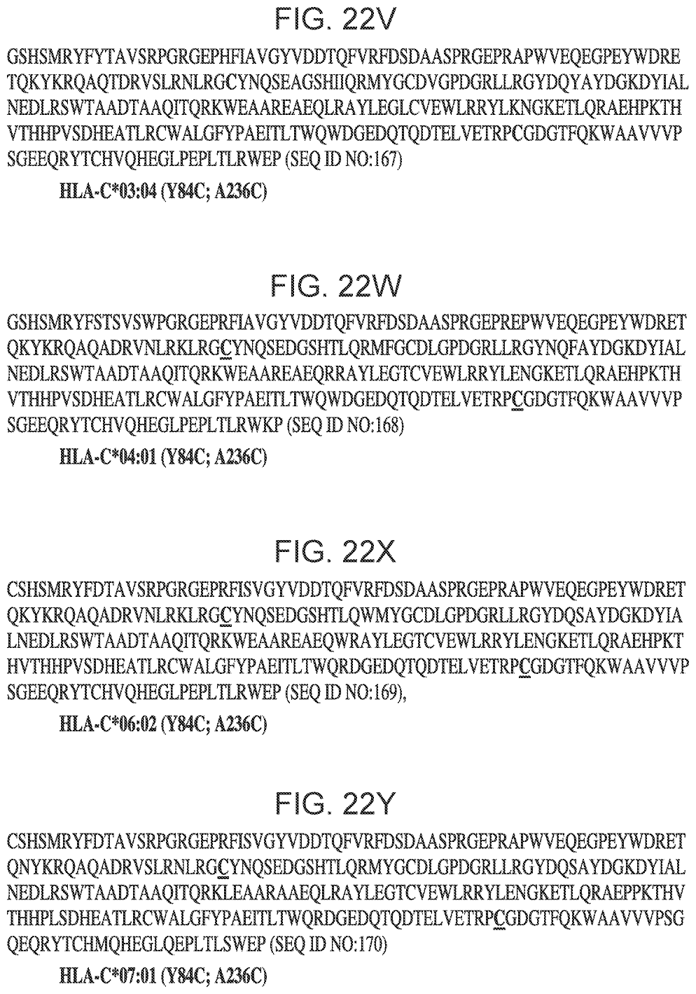

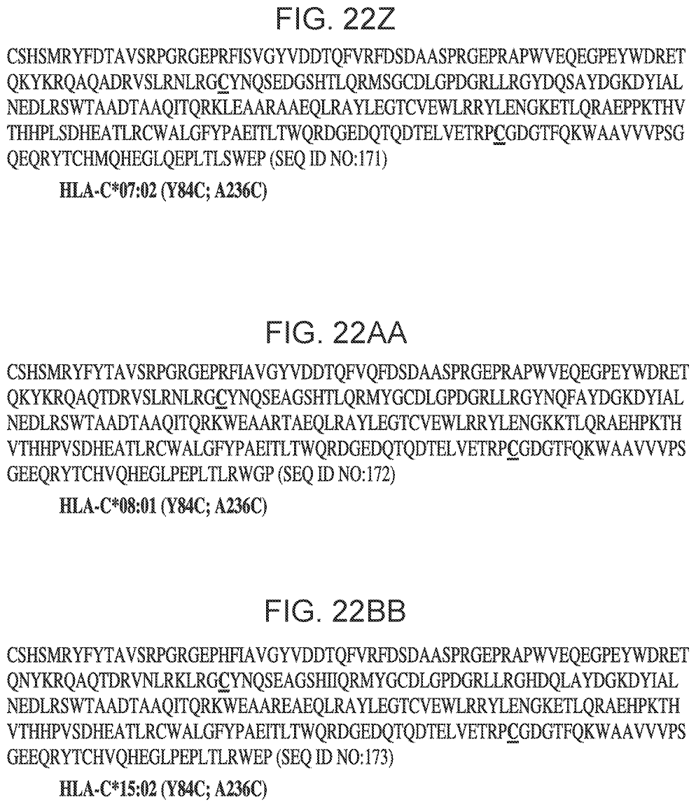

In some cases, a multiple disulfide-linked heterodimeric or single-chain TMP of the present disclosure (e.g., a double disulfide-linked TMP) comprises: i) a KRAS peptide (e.g., a KRAS peptide of from 4 amino acids to 25 amino acids in length, that is bound by a TCR when the peptide is complexed with MHC polypeptides; e.g., where the KRAS peptide comprises a cancer-associated mutation); ii) a first MHC polypeptide; iii) a peptide linker between the peptide and the first MHC polypeptide, where the peptide linker comprises a Cys residue, and where the first MIHC polypeptide is a β2M polypeptide that comprises an amino acid substitution that introduces a Cys residue; and iv) a second MHC polypeptide comprising an HLA-A MHC class I heavy chain comprising an amino acid sequence having at least 60%, at least 70%, at least 80%, at least 90%, at least 95%, at least 98%, at least 99%, or 100%, amino acid sequence identity to the amino acid sequence shown in FIG. 22A (SEQ ID NO:143), where amino acid 84 is a Cys and amino acid 236 is a Cys; and where the TMP comprises the at least one immunomodulatory polypeptide. In some cases, the peptide linker comprises the amino acid sequence GCGGS (SEQ ID NO:139). In some cases, the peptide linker comprises the amino acid sequence GCGGS(GGGGS)n (SEQ ID NO:140), where n is an integer from 1 to 10, e.g., 1, 2 or 3. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the amino acid sequence of FIG. 21CC (SEQ ID NO:144), where amino acid 12 is a Cys. The at least one immunomodulatory polypeptide can be a wild-type or variant of a immunomodulatory polypeptide, e.g., selected from the group consisting of cytokine (e.g., IL-2), a 4-1BBL polypeptide, a CD80 polypeptide, a CD86 polypeptide, or combinations thereof. In some cases, the at least one immunomodulatory polypeptide is a reduced affinity variant, e.g., a reduced-affinity variant of IL-2, as described elsewhere herein. In some cases, the TMP comprises an Ig Fc polypeptide, e.g., a human IgG1 Fc that substantially does not induce cell lysis such as shown in FIG. 3G.

In some cases, a multiple disulfide-linked heterodimeric or single-chain TMP of the present disclosure (e.g, a double disulfide-linked TMP) comprises an HLA-A Class I heavy chain polypeptide. In some cases, the HLA-A heavy chain polypeptide present in a multiple disulfide-linked TMP of the present disclosure (e.g., a double disulfide-linked TMP) comprises an amino acid sequence having at least 95%, at least 98%, or at least 99%, amino acid sequence identity to the HLA-A*0101, HLA-A*0201, HLA-A*0202, HLA-A*1101, HLA-A*2301, HLA-A*2402, HLA-A*2407, HLA-A*3303, or HLA-A*3401 amino acid sequence depicted in FIG. 7A, where the HLA-A heavy chain polypeptide comprises Y84C and A236C substitutions.

In some cases, the HLA-A heavy chain polypeptide present in a multiple disulfide-linked heterodimeric or single-chain TMP of the present disclosure (e.g., a double disulfide-linked IMP) comprises an amino acid sequence having at least 95%, at least 98%, at least 99%, or 100%, amino acid sequence identity to one of the following sequences:
(i) the HLA-A*0101 (Y84C; A236C) amino acid sequence shown in in FIG. 22B (SEQ ID NO:145), where amino acid 84 is a Cys and amino acid 236 is a Cys;
(ii) the HLA-A*0201 (Y84C; A236C) amino acid sequence shown in in FIG. 22C (SEQ ID NO:146), where amino acid 84 is a Cys and amino acid 236 is a Cys;
(iii) the HLA-A*0202 (Y84C; A236C) amino acid sequence shown in FIG. 22D (SEQ ID NO:147), where amino acid 84 is a Cys and amino acid 236 is a Cys;
(iv) the HLA-A*1101 (Y84C; A236C) amino acid sequence shown in FIG. 22E (SEQ ID NO:148), where amino acid 84 is a Cys and amino acid 236 is a Cys;
(v) the HLA-A*2301 (Y84C; A236C) amino acid sequence shown in FIG. 22F (SEQ ID NO:149), where amino acid 84 is a Cys and amino acid 236 is a Cys;
(vi) the HLA-A*2402 (Y84C; A236C) amino acid sequence shown in FIG. 22G (SEQ ID NO:150), where amino acid 84 is a Cys and amino acid 236 is a Cys;
(vii) the HLA-A*2407 (Y84C; A236C) amino acid sequence shown in FIG. 22H (SEQ ID NO:151), where amino acid 84 is a Cys and amino acid 236 is a Cys;
(viii) the HLA-A*3303 (Y84C; A236C) amino acid sequence shown in FIG. 22I (SEQ ID NO:152), where amino acid 84 is a Cys and amino acid 236 is a Cys;
(ix) the HLA-A*3401 (Y84C; A236C) amino acid sequence shown in FIG. 22J (SEQ ID NO:153), where amino acid 84 is a Cys and amino acid 236 is a Cys.

### HLA-B

In some cases, a multiple disulfide-linked heterodimeric or single-chain TMP of the present disclosure (e.g., a double disulfide-linked TMP) comprises i) a KRAS peptide that is bound by a TCR when the peptide is complexed with MHC polypeptides of the TMP, ii) a β2M polypeptide comprising a non-naturally occurring Cys residue, iii) a peptide linker between the KRAS peptide and the β2M polypeptide, and iv) an HLA-B MHC class I heavy chain polypeptide comprising an amino acid sequence having at least 60%, at least 70%, at least 80%, at least 90%, at least 95%, at least 98%, at least 99%, or 100%, amino acid sequence identity to the following amino acid sequence shown in FIG. 22K (SEQ ID NO:154), where amino acid 84 is a Cys and amino acid 236 is a Cys. In some cases, the peptide linker comprises the amino acid sequence GCGGS (SEQ ID NO:139). In some cases, the peptide linker comprises the amino acid sequence GCGGS(GGGGS)n (SEQ ID NO: 140), where n is an integer from 1 to 10, e.g., 1, 2 or 3. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the amino acid sequence of FIG. 21CC (SEQ ID NO:155), where amino acid 12 is a Cys. The at least one immunomodulatory polypeptide of the TMP can be a wild-type or variant immunomodulatory polypeptide, e.g., selected from the group consisting of cytokine (e.g., IL-2), 4-1BBL, CD80, CD86 and combinations thereof. In some cases, the at least one immunomodulatory polypeptide is a reduced affinity variant such as an IL-2 variant, as described elsewhere herein. In some cases, the TMP comprises an Ig Fc polypeptide, e.g. a variant human IgG1 Fc polypeptide that substantially does not induce cell lysis.

In some cases, a multiple disulfide-linked heterodimeric or single-chain TMP of the present disclosure comprises an HLA-B Class I heavy chain polypeptide. In some cases, the HLA-B heavy chain polypeptide present in a multiple disulfide-linked TMP of the present disclosure (e.g., a double disulfide-linked TMP) comprises an amino acid sequence having at least 95%, at least 98%, or at least 99%, amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A, where the HLA-B heavy chain polypeptide comprises Y84C and A236C substitutions.

### HLA-B*0702 (Y84C; A236C)

In some cases, the HLA-B heavy chain polypeptide present in a multiple disulfide-linked heterodimeric or single-chain TMP of the present disclosure (e.g., a double disulfide-linked TMP) comprises an amino acid sequence having at least 95%, at least 98%, at least 99%, or 100%, amino acid sequence identity to identity to one of the following sequences:
(i) the HLA-B*0702 (Y84C; A236C) amino acid sequence shown in FIG. 22L (SEQ ID NO:156), where amino acid 84 is a Cys and amino acid 236 is a Cys;
(ii) the HLA-B*0801 (Y84C; A236C) amino acid sequence shown in FIG. 22M (SEQ ID NO:157), where amino acid 84 is a Cys and amino acid 236 is a Cys;
(iii) the HLA-B*1502 (Y84C; A236C) amino acid sequence shown in FIG. 22N (SEQ ID NO:158), where amino acid 84 is a Cys and amino acid 236 is a Cys;
(iv) the HLA-B*3802 (Y84C; A236C) amino acid sequence shown in FIG. 22O (SEQ ID NO:159), where amino acid 84 is a Cys and amino acid 236 is a Cys;
(v) the HLA-B*4001 (Y84C; A236C) amino acid sequence shown in FIG. 22P (SEQ ID NO:160) where amino acid 84 is a Cys and amino acid 236 is a Cys;
(vi) the HLA-B*4601 (Y84C; A236C) amino acid sequence shown in FIG. 22Q (SEQ ID NO:161) where amino acid 84 is a Cys and amino acid 236 is a Cys;
(vii) the HLA-B*5301 (Y84C; A236C) amino acid sequence shown in FIG. 22R (SEQ ID NO:162) where amino acid 84 is a Cys and amino acid 236 is a Cys.

### HLA-C

In some cases, a multiple disulfide-linked heterodimeric or single-chain TMP of the present disclosure (e.g., a double disulfide-linked TMP) comprises i) a KRAS peptide that is bound by a TCR when the peptide is complexed with MHC polypeptides of the TMP, ii) a β2M polypeptide comprising a non-naturally occurring Cys residue, iii) a peptide linker between the KRAS peptide and the β2M polypeptide, and iv) an HLA-C MHC class I heavy chain comprising an amino acid sequence having at least 60%, at least 70%, at least 80%, at least 90%, at least 95%, at least 98%, at least 99%, or 100%, amino acid sequence identity to the amino acid sequence shown in FIG. 22S (SEQ ID NO:163), where amino acid 84 is a Cys and amino acid 236 is a Cys; and c) at least one immunomodulatory polypeptide, where the first and/or the second polypeptide comprises the at least one immunomodulatory polypeptide. In some cases, the peptide linker comprises the amino acid sequence GCGGS (SEQ ID NO:139). In some cases, the peptide linker comprises the amino acid sequence GCGGS(GGGGS)n (SEQ ID NO: 140), where n is an integer from 1 to 10, e.g., 1, 2 or 3. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the following amino acid sequence shown in FIG. 21CC (SEQ ID NO:164), where amino acid 12 is a Cys. The at least one immunomodulatory polypeptide can be a wild-type or variant polypeptide immunomodulatory polypeptide, e.g., selected from the group consisting of cytokine (e.g., IL-2), a 4-1BBL polypeptide, a CD80 polypeptide, a CD86 polypeptide, or combinations thereof. In some cases, the at least one immunomodulatory polypeptide is a reduced affinity variant, e.g., a reduced-affinity variant of IL-2, as described elsewhere herein. In some cases, the TMP comprises an Ig Fc polypeptide, e.g. a variant human IgG1 Fc polypeptide that substantially does not induce cell lysis.

In some cases, a multiple disulfide-linked TMP of the present disclosure (e.g., a double disulfide-linked TMP) comprises an HLA-C Class I heavy chain polypeptide. In some cases, the HLA-C heavy chain polypeptide present in a multiple disulfide-linked TMP of the present disclosure (e.g., a double disulfide-linked TMP) comprises an amino acid sequence having at least 95%, at least 98%, or at least 99%, amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 amino acid sequence depicted in FIG. 9A, where the HLA-C heavy chain polypeptide comprises Y84C and A236C substitutions.

In some cases, the HLA-C heavy chain polypeptide present in a multiple disulfide-linked TMP of the present disclosure (e.g., a double disulfide-linked TMP) comprises an amino acid sequence having at least 95%, at least 98%, at least 99%, or 100%, amino acid sequence identity to one of the following sequences:
(i) the HLA-C*01:02 (Y84C; A236C) amino acid sequence shown in FIG. 22T (SEQ ID NO:165), where amino acid 84 is a Cys and amino acid 236 is a Cys;
(ii) the HLA-C*03:03 (Y84C; A236C) amino acid sequence shown in FIG. 22U (SEQ ID NO:166), where amino acid 84 is a Cys and amino acid 236 is a Cys;
(iii) the HLA-C*03:04 (Y84C; A236C) amino acid sequence shown in FIG. 22V (SEQ ID NO:167), where amino acid 84 is a Cys and amino acid 236 is a Cys;
(iv) the HLA-C*04:01 (Y84C; A236C) amino acid sequence shown in FIG. 22W (SEQ ID NO:168), where amino acid 84 is a Cys and amino acid 236 is a Cys;
(v) the HLA-C*06:02 (Y84C; A236C) amino acid sequence shown in FIG. 22X (SEQ ID NO:169), where amino acid 84 is a Cys and amino acid 236 is a Cys;
(vi) the HLA-C*07:01 (Y84C; A236C) amino acid sequence shown in FIG. 22Y (SEQ ID NO:170), where amino acid 84 is a Cys and amino acid 236 is a Cys;
(vii) the HLA-C*07:02 (Y84C; A236C) amino acid sequence shown in FIG. 22Z (SEQ ID NO:171), where amino acid 84 is a Cys and amino acid 236 is a Cys;
(viii) the HLA-C*08:01 (Y84C; A236C) amino acid sequence shown in FIG. 22AA (SEQ ID NO:172), where amino acid 84 is a Cys and amino acid 236 is a Cys; and
(ix) the HLA-C*15:02 (Y84C; A236C) amino acid sequence shown in FIG. 22BB (SEQ ID NO: 173), where amino acid 84 is a Cys and amino acid 236 is a Cys.

### Antigen-Presenting, Disulfide-Linked TMPS (i.e., Mod-Less TMPS)

The present disclosure provides an antigen-presenting polypeptide (APP) that comprises a heterodimer or single-chain polypeptides (or a homodimer of two such polypeptides), where the APP comprises: i) a KRAS peptide that is bound by a TCR when the peptide is complexed with MHC polypeptides of the TMP, ii) a β2M polypeptide, optionally comprising a non-naturally occurring Cys residue, iii) a peptide linker between the KRAS peptide and the β2M polypeptide, wherein the linker optionally comprises a Cys residue, and iv) an HLA-C MHC class I heavy chain comprising an amino acid sequence having at least 60%, at least 70%, at least 80%, at least 90%, at least 95%, at least 98%, at least 99%, or 100%, amino acid sequence identity to any of the amino acid sequences shown in FIGS. 21A-21AA, 21DD-21FF, 21HH-21JJ, or 22A-22BB. The heterodimeric or single-chain APPs can be of the same configuration and can be singly or multiply disulfide linked in the same manner as any of the above singly of multiply disulfide-linked heterodimeric or single-chain TMPs; the only difference being that, unlike the TMPs discussed above, the APPs do not contain MODs. As discussed above, the disulfide linking referred to here is not intended to refer to disulfide linkages between the Ig Fc polypeptides of the APP, e.g., Ig Fc polypeptides that substantially do not induce cell lysis such as the human IgG1 Fc polypeptide of FIG. 3G.

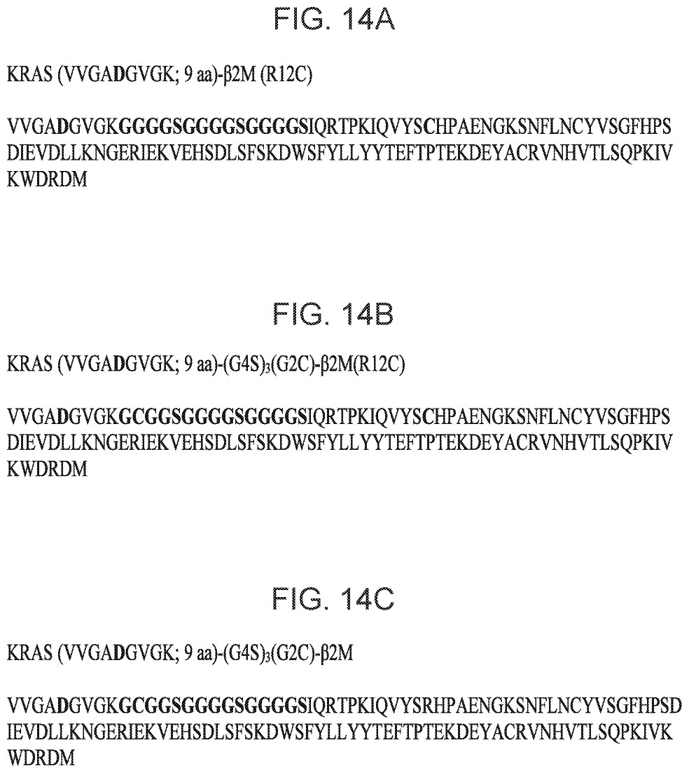

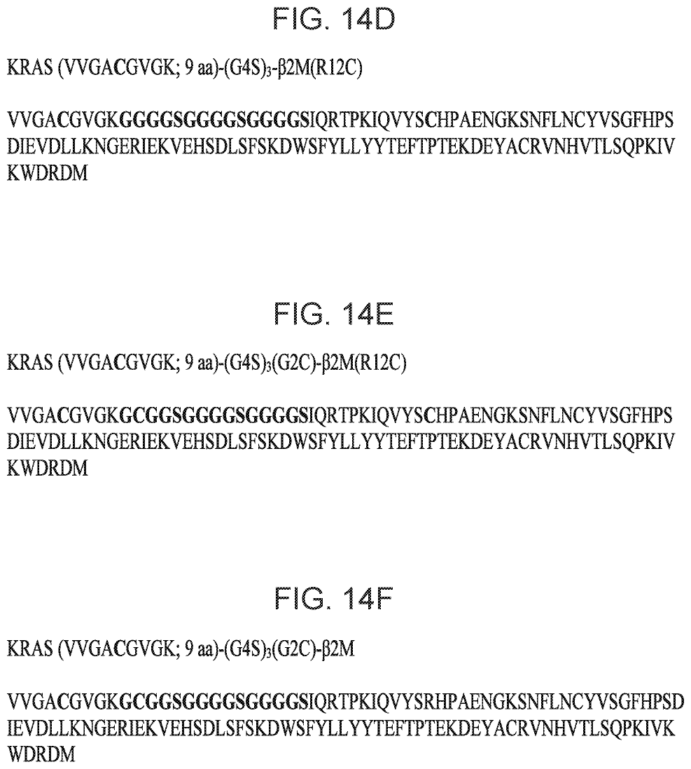

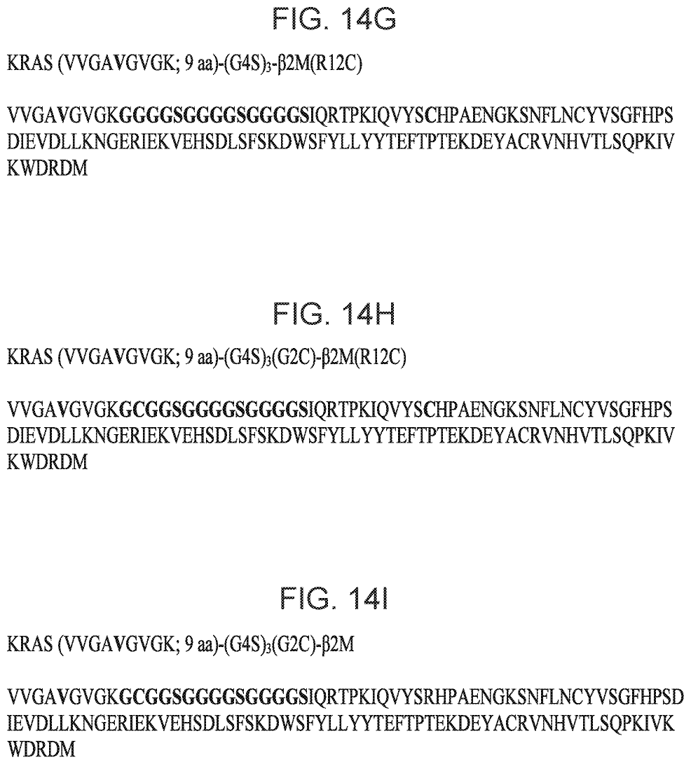

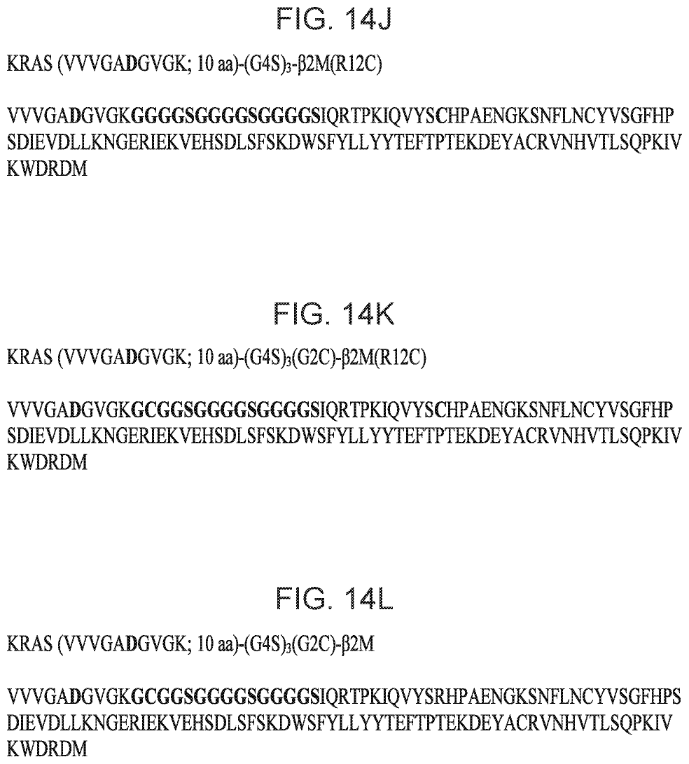

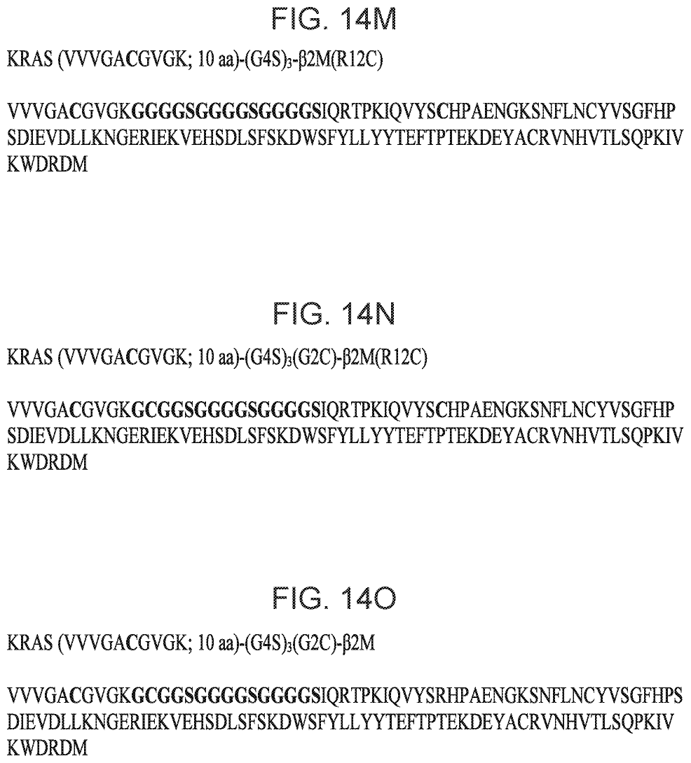

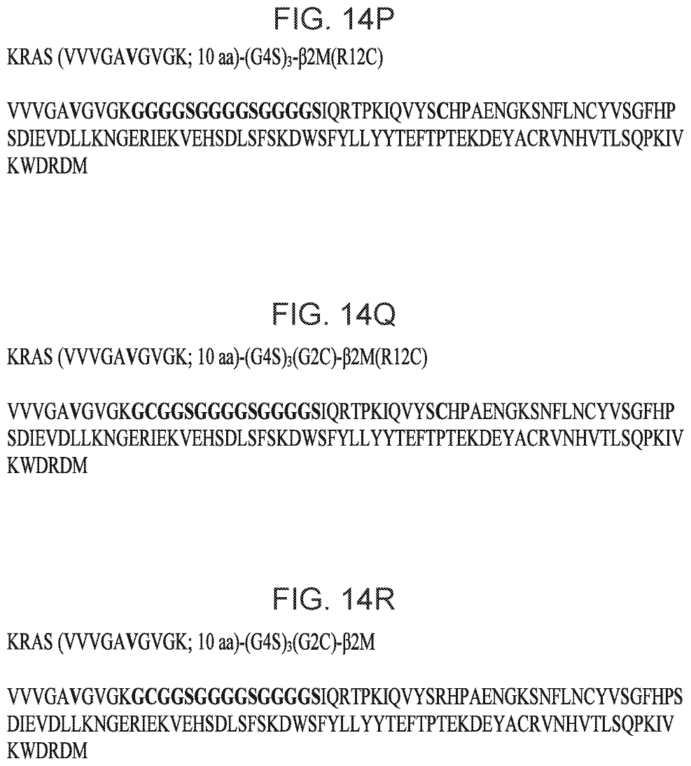

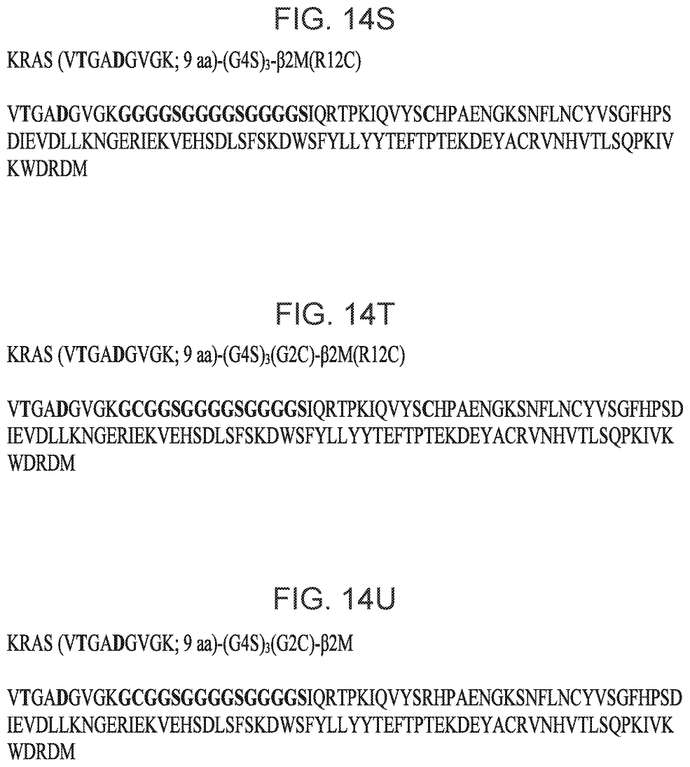

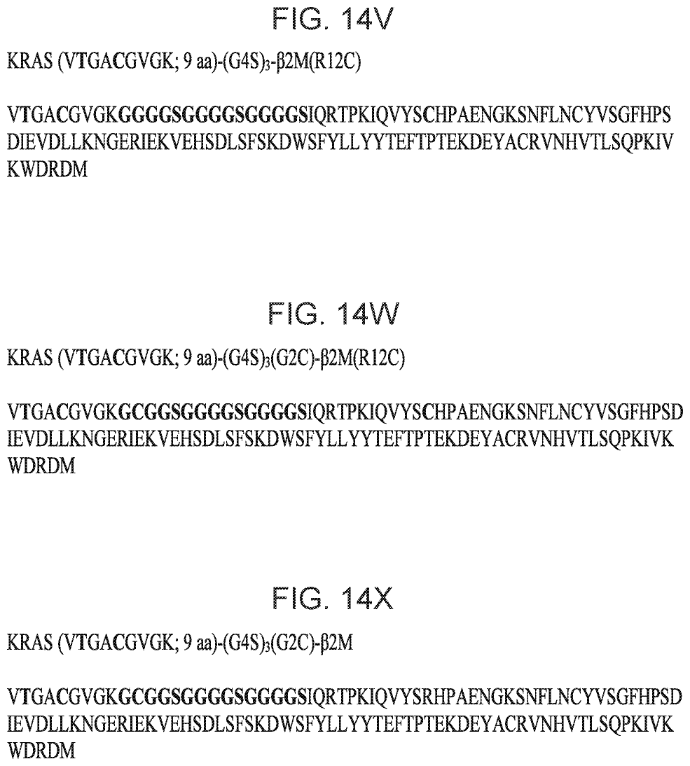

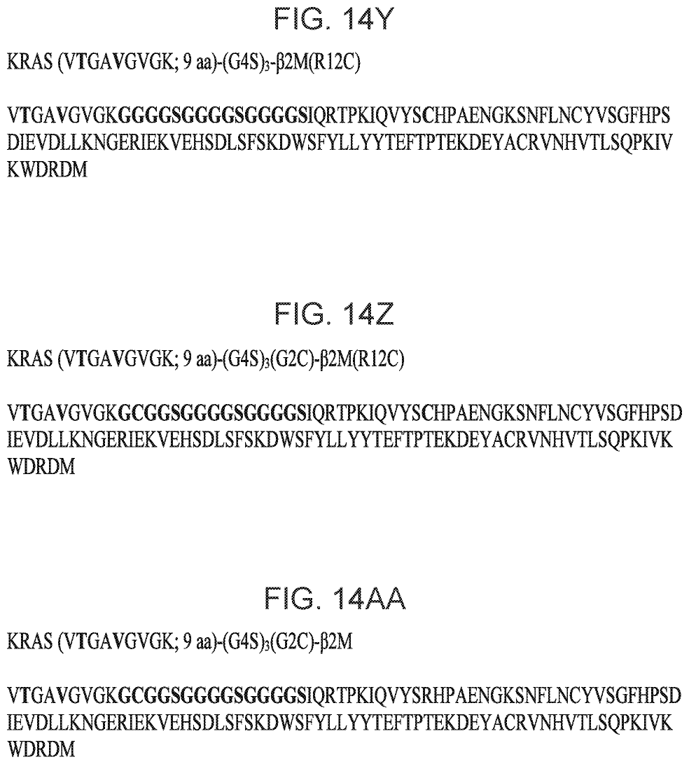

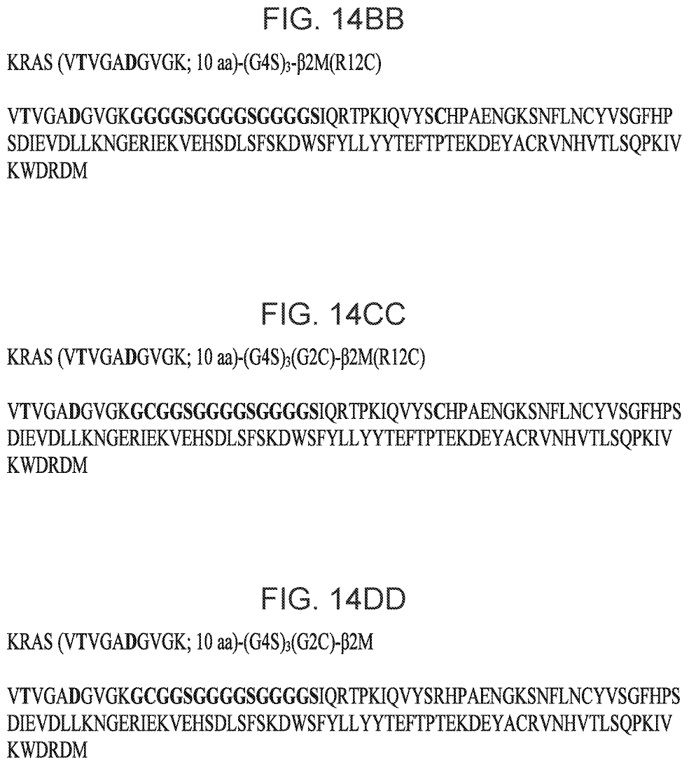

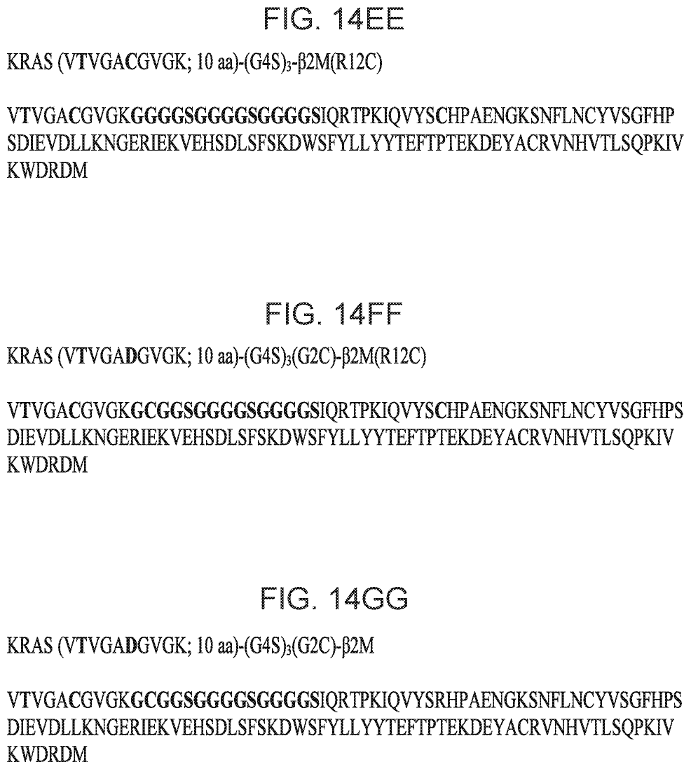

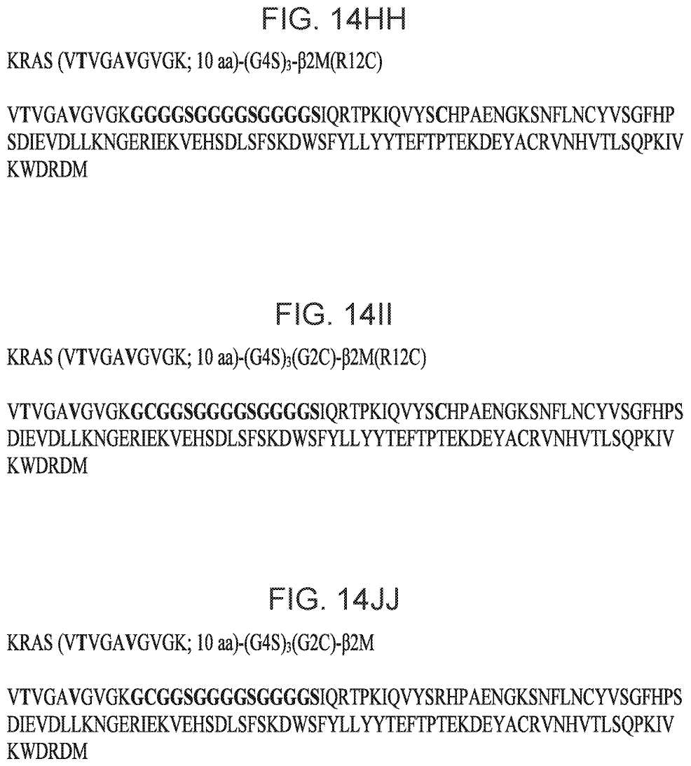

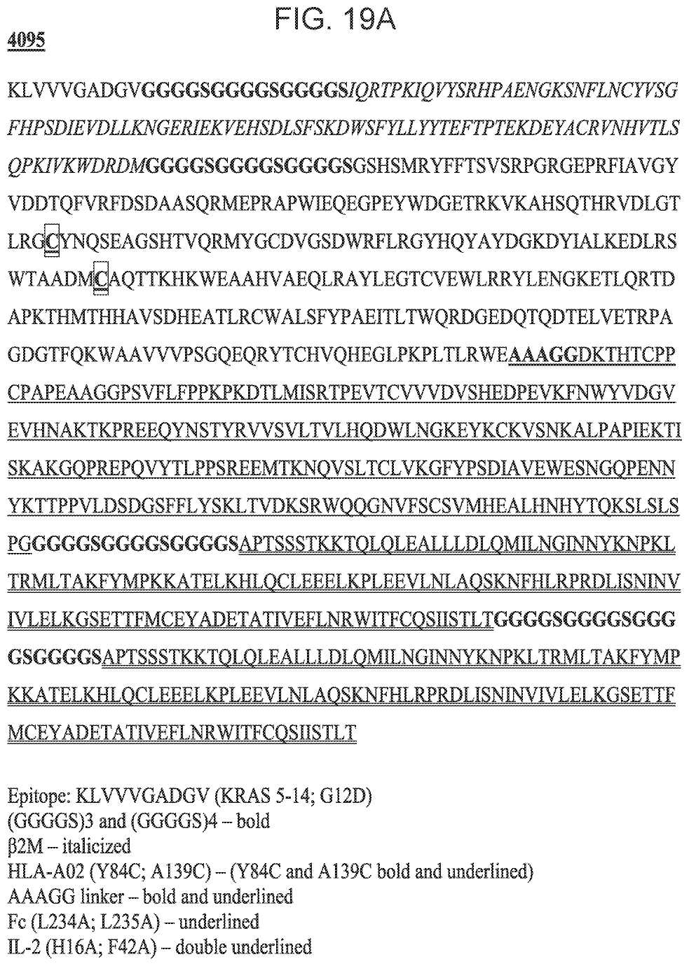

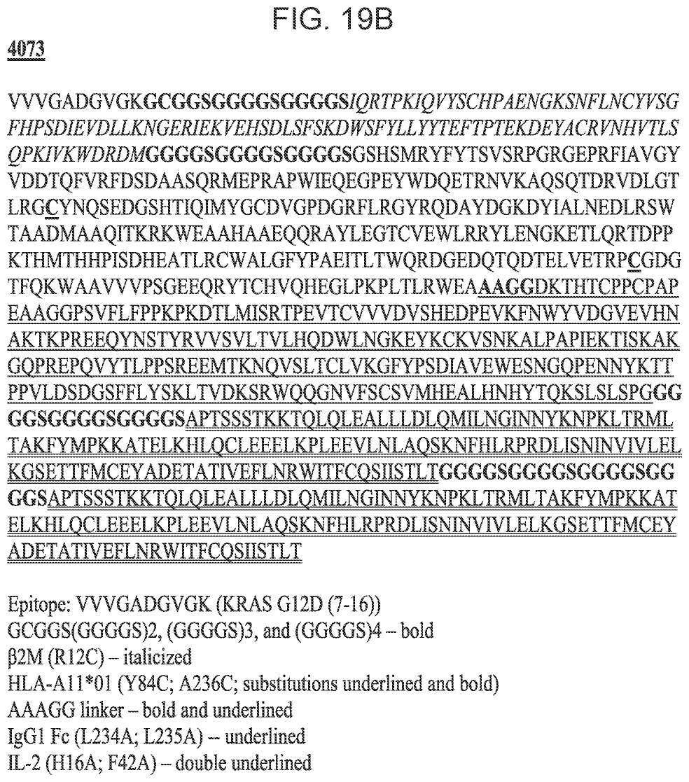

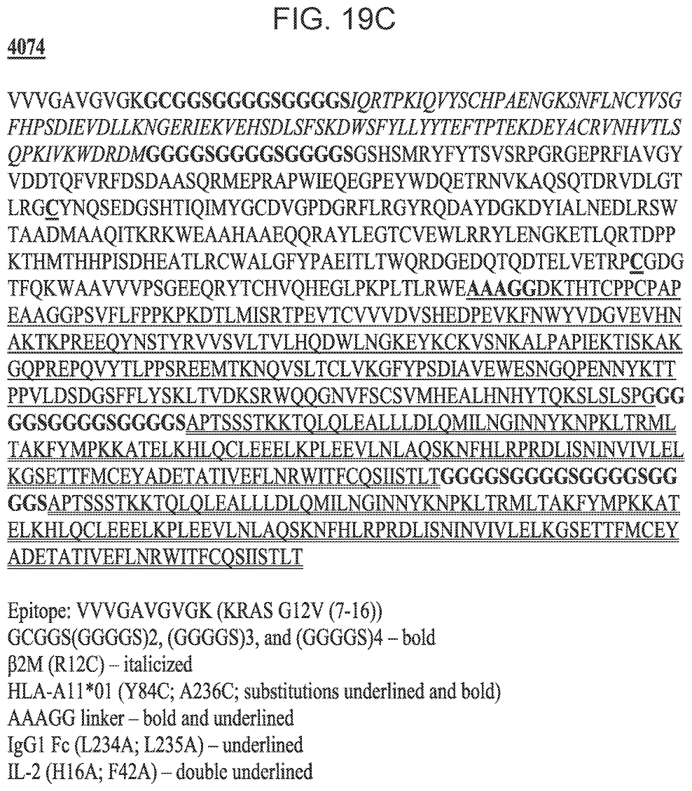

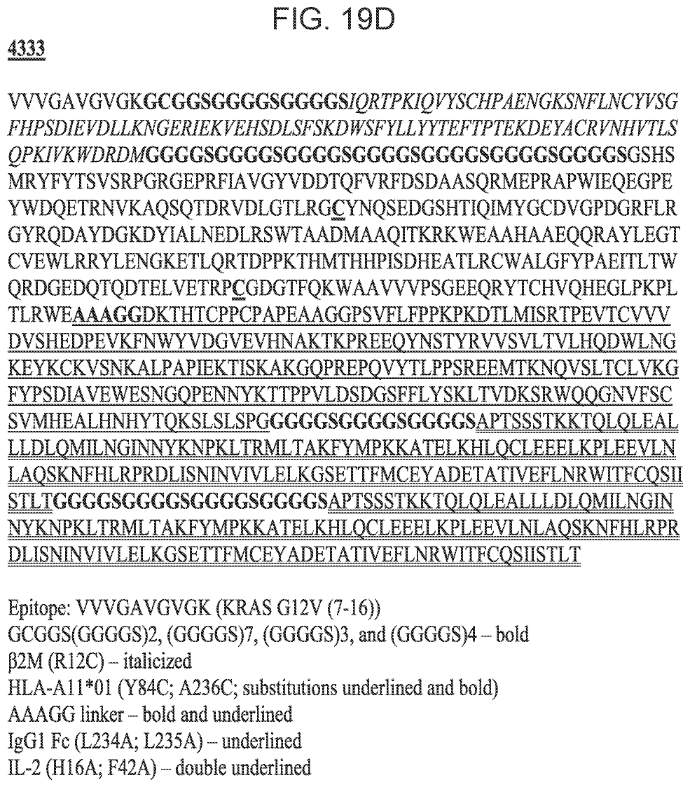

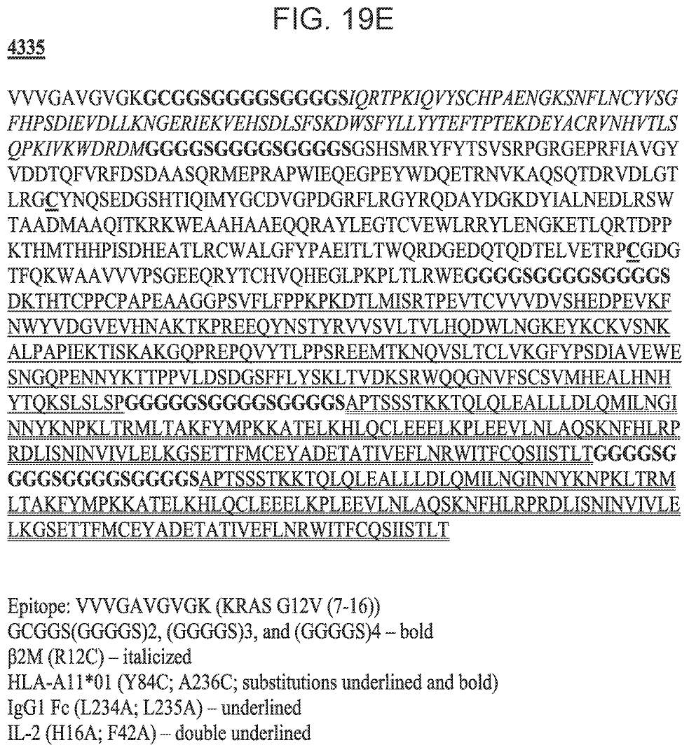

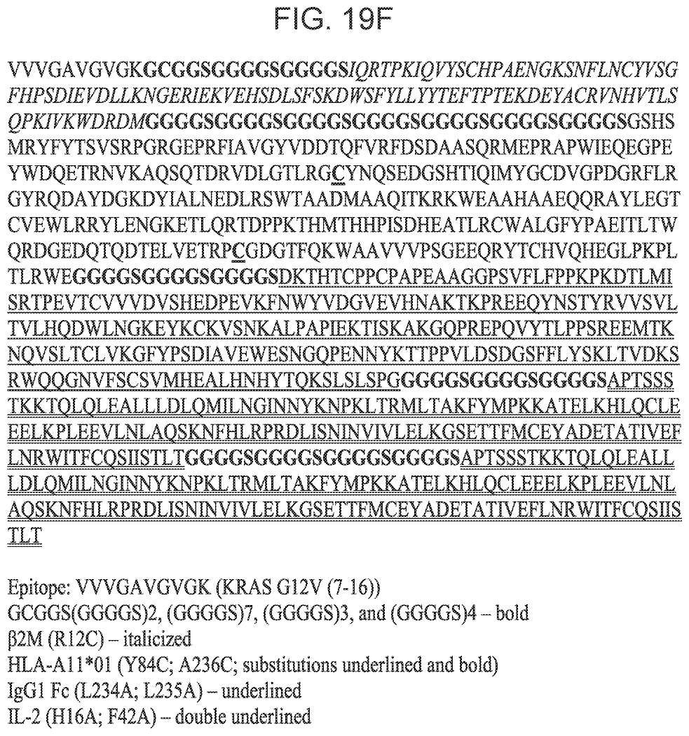

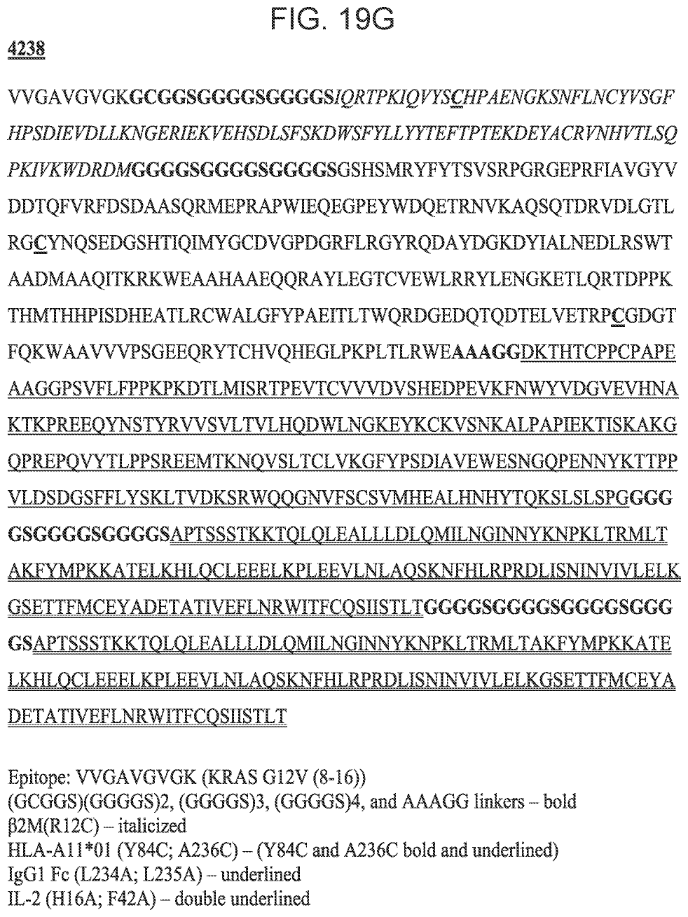

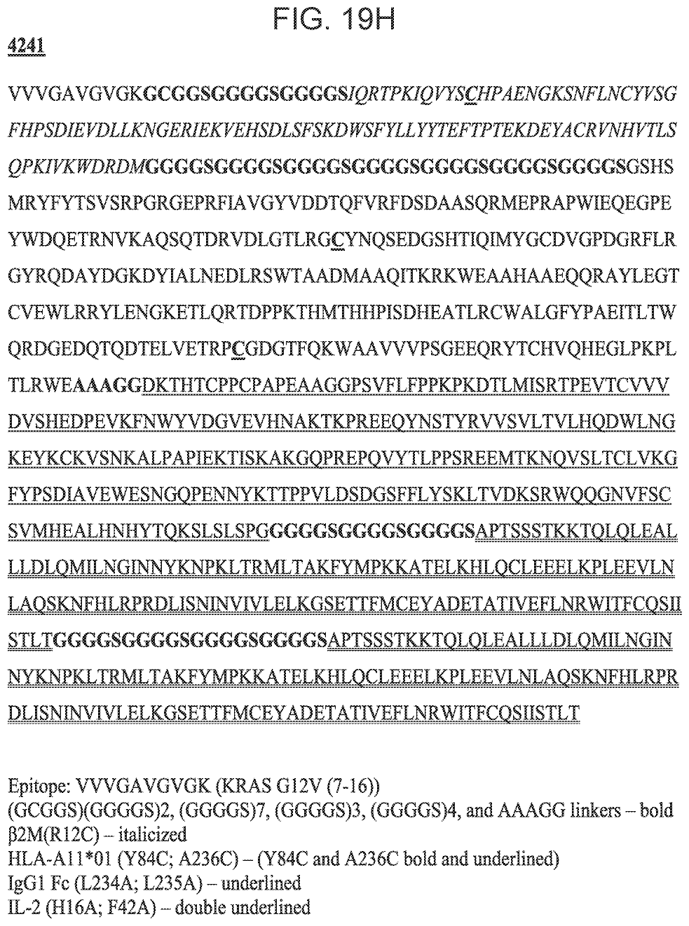

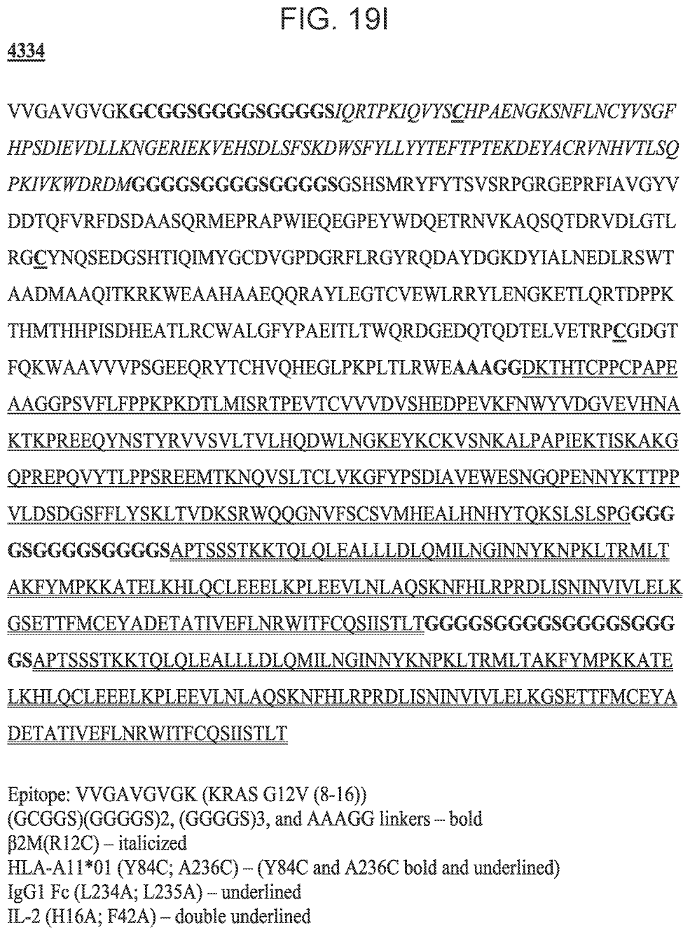

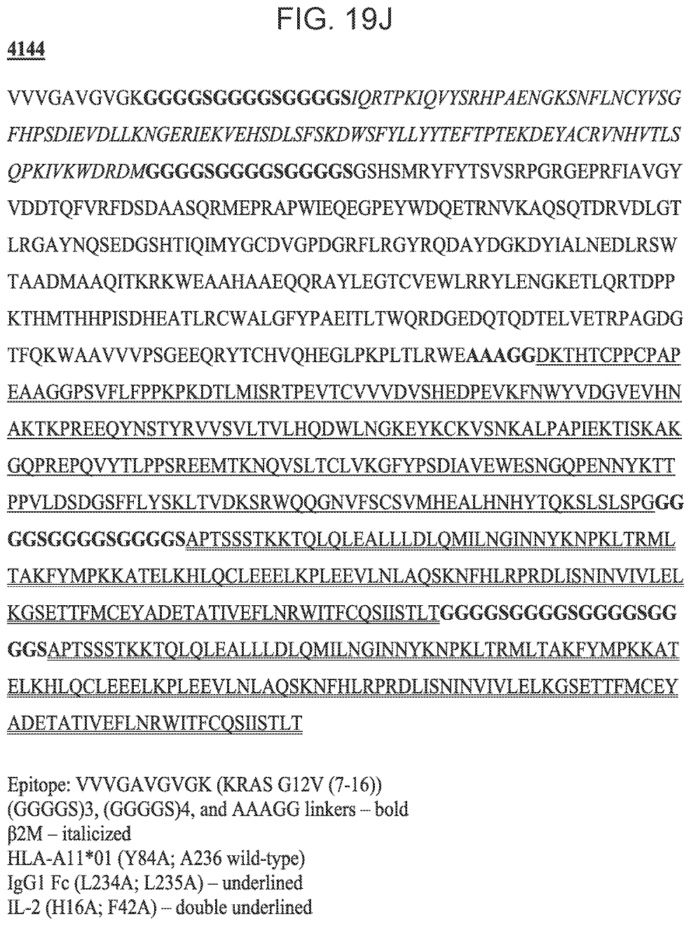

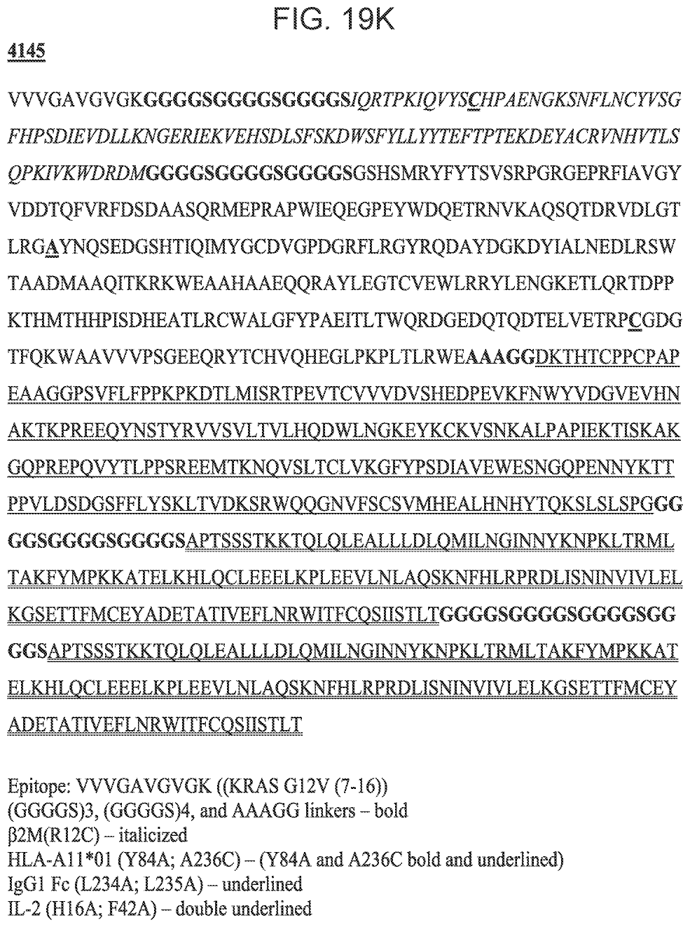

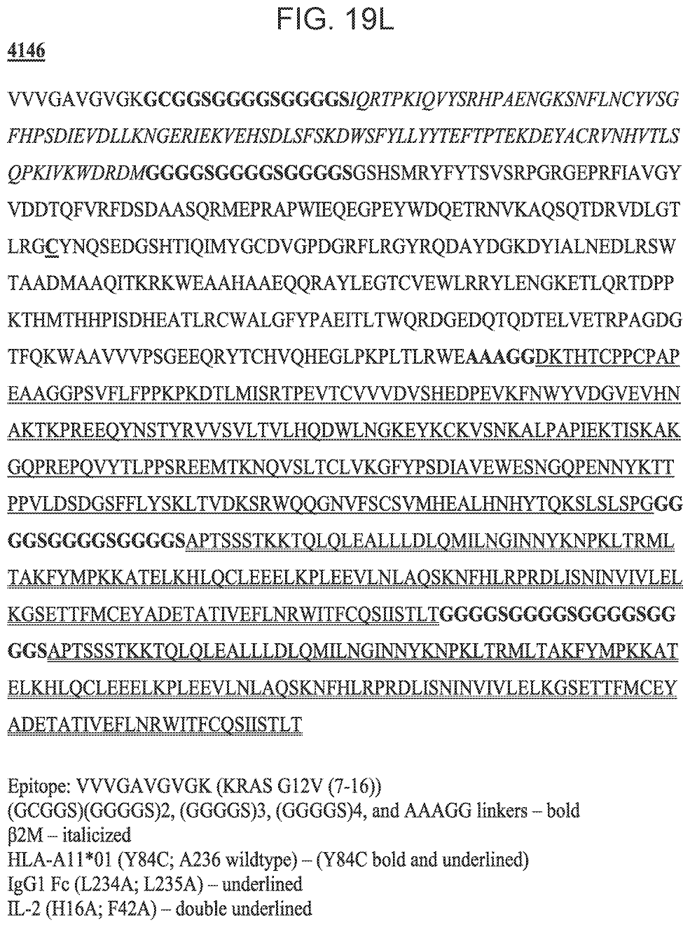

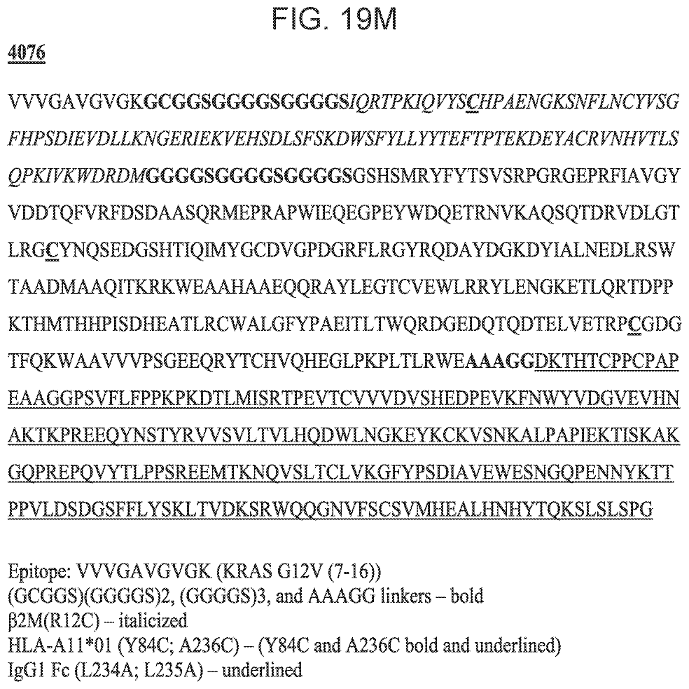

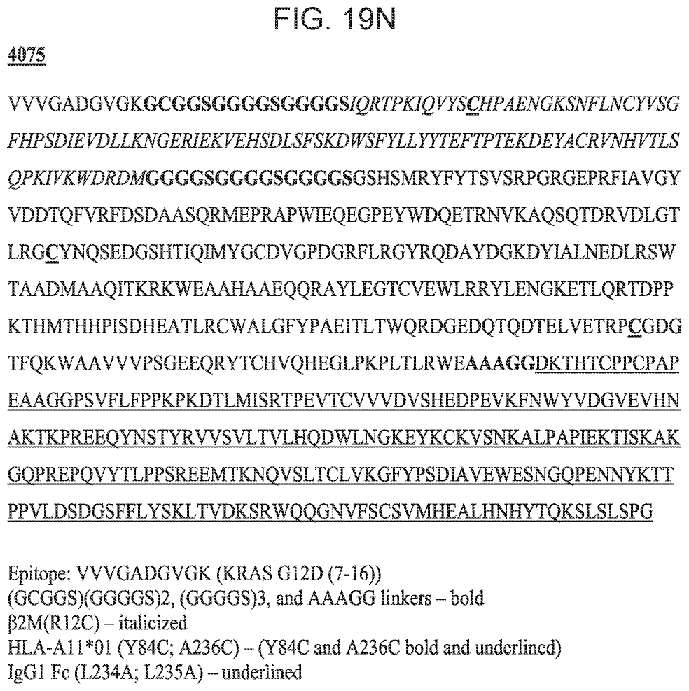

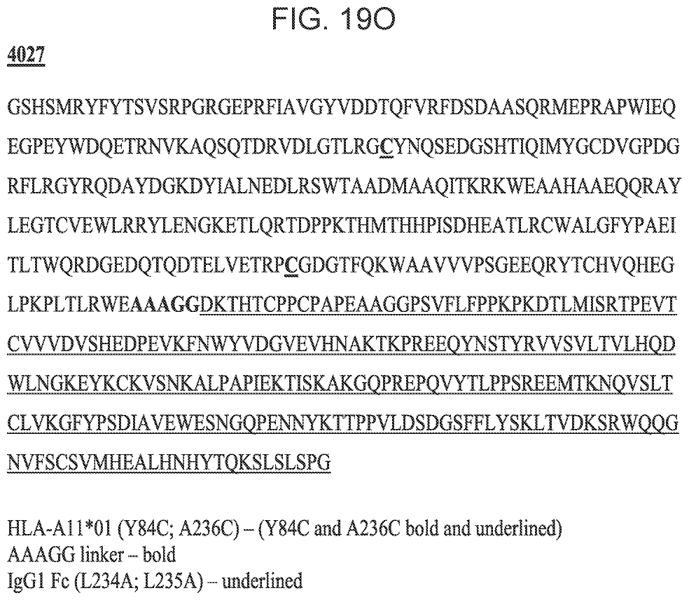

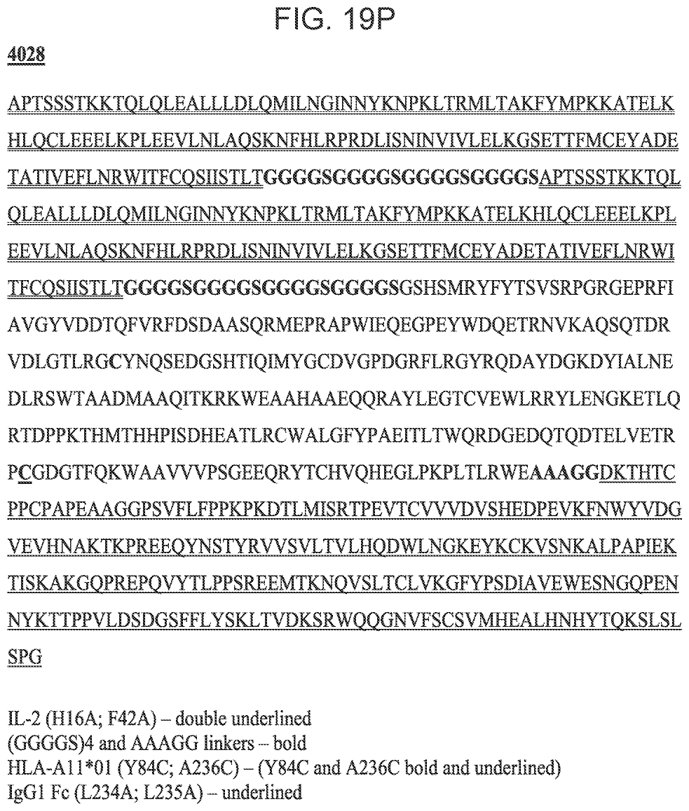

Examples of heterodimeric APPs include: a) an APP comprising: i) the “4027” polypeptide depicted in FIG. 190; and ii) the “4030” polypeptide depicted in FIG. 14Q; b) an APP comprising: i) the “4027” polypeptide depicted in FIG. 190; and ii) the “4029” polypeptide depicted in FIG. 14K. Examples of single-chain APPs include the polypeptides referred to as “4238” (FIG. 19G) “4241” (FIG. 19H), and “4334” (FIG. 19I).

An APP of the present disclosure is useful for diagnostic applications and therapeutic applications. As discussed below, when used for diagnostic applications, the APP also can comprise a detectable label so that binding of the APP to a target T cell is detected by detecting the detectable label.

The present disclosure thus provides a method of detecting an antigen-specific T-cell. The methods comprise contacting a T cell with an APP of the present disclosure; and detecting binding of the APP to the T cell. The present disclosure provides a method of detecting an antigen-specific T cell, the method comprising contacting a T cell with an APP of the present disclosure, wherein binding of the APP to the T cell indicates that the T cell is specific for the epitope present in the APP.

In some cases, the APP comprises a detectable label. Suitable detectable labels include, but are not limited to, a radioisotope, a fluorescent polypeptide, or an enzyme that generates a fluorescent product, and an enzyme that generates a colored product. Where the APP comprises a detectable label, binding of the APP to the T cell is detected by detecting the detectable label.

In some cases, an APP of the present disclosure comprises a detectable label suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacycl ododecane)), iron chelates, magnesium chelates, manganese chelates, copper chelates, chromium chelates, iodine-based materials, and radionuclides. Suitable radionuclides include, but are not limited to, ¹²³I, ¹²⁵I, ¹³⁰I, ¹³¹I, ¹³³I, ¹³⁵I, ⁴⁷Sc ⁷²As, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁴Cu, ⁶⁷Cu, ⁷⁵Br, ⁷⁷Br, ⁹⁹mTc, ¹⁴C, ¹³N, ¹⁵O, ³²P, ³³P, and ¹⁸F. In some cases, the detectable label is a positron-emitting isotope such as ¹¹C, ¹³N, ¹⁵O, ¹⁸F, ⁶⁴Cu, ⁶⁸Ga, ⁷⁸Br, ⁸²Rb, ⁸⁶Y, ⁹⁰Y, ²²Na, ²⁶Al, ⁴⁰K, ⁸³Sr, ⁸⁹Zr, or ¹²⁴I. In some cases, the detectable label is ⁶⁴Cu. See, e.g., Woodham, Andrew et al., In vivo detection of antigen-specific CD8+ T cells by immuno-positron emission tomography, Nature Methods Articles (2020) https://doi.org/10.1038/s41592-020-0934-5.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, 0-glucuronidase, invertase, Xanthine Oxidase, firefly luciferase, glucose oxidase (GO), and the like.

In some cases, binding of the APP to the T cell is detected using a detectably labeled antibody specific for the APP. An antibody specific for the APP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

### HLA/Peptide Binding Assays

Whether a given peptide (e.g., a KRAS peptide that comprises a KRAS epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays, including cell-based binding assays, biochemical binding assays, T-cell activation assays, ELISPOT assays, cytotoxicity assays and Detection of Antigen-specific T cells with peptide-HLA tetramers. Such assays are described in the published scientific literature as well as in published PCT application WO2020132138A1, the disclosure of which as it pertains to specific binding assays is expressly incorporated herein by reference, including specifically paragraphs [00217]-[00225].

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### Immunomodulatory Polypeptides

In some cases, an immunomodulatory polypeptide or “MOD” present in a TMP of the present disclosure is a wild-type immunomodulatory polypeptide. In other cases, an immunomodulatory polypeptide present in a TMP of the present disclosure is a variant immunomodulatory polypeptide that has reduced affinity for a costimulatory polypeptide, compared to the affinity of a corresponding wild-type immunomodulatory polypeptide for the costimulatory polypeptide. Suitable immunomodulatory domains that exhibit reduced affinity for a costimulatory domain can have from 1 amino acid (aa) to 20 aa differences from a wild-type immunomodulatory domain. For example, in some cases, a variant immunomodulatory polypeptide present in a TMP of the present disclosure differs in amino acid sequence by 1 aa, 2 aa, 3 aa, 4 aa, 5 aa, 6 aa, 7 aa, 8 aa, 9 aa, or 10 aa, from a corresponding wild-type immunomodulatory polypeptide. As another example, in some cases, a variant immunomodulatory polypeptide present in a TMP of the present disclosure differs in amino acid sequence by 11 aa, 12 aa, 13 aa, 14 aa, 15 aa, 16 aa, 17 aa, 18 aa, 19 aa, or 20 aa, from a corresponding wild-type immunomodulatory polypeptide.

Exemplary pairs of immunomodulatory polypeptides and their cognate costimulatory polypeptides include, but are not limited to those set out in Table 1, below:

TABLE 1

| Immunomodulatory | Cognate Costimulatory |
|---|---|
| Polypeptide (MOD) | Polypeptide |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

In some cases, a variant immunomodulatory polypeptide present in a TMP of the present disclosure has a binding affinity for a cognate costimulatory polypeptide that is from 100 nM to 100 μM. For example, in some cases, a variant immunomodulatory polypeptide present in a TMP of the present disclosure has a binding affinity for a cognate costimulatory polypeptide that is from about 100 nM to 150 nM, from about 150 nM to about 200 nM from about 200 nM to about 250 nM from about 250 nM to about 300 nM from about 300 nM to about 350 nM from about 350 nM to about 400 nM from about 400 nM to about 500 nM from about 500 nM to about 600 nM from about 600 nM to about 700 nM from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

A variant immunomodulatory polypeptide present in a TMP of the present disclosure exhibits reduced affinity for a cognate costimulatory polypeptide. Similarly, a TMP of the present disclosure that comprises a variant immunomodulatory polypeptide exhibits reduced affinity for a cognate costimulatory polypeptide. Thus, for example, a TMP of the present disclosure that comprises a variant immunomodulatory polypeptide has a binding affinity for a cognate costimulatory polypeptide that is from 100 nM to 100 μM. For example, in some cases, a TMP of the present disclosure that comprises a variant immunomodulatory polypeptide has a binding affinity for a cognate costimulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

As depicted schematically in FIG. 17, an immunomodulatory polypeptide (i.e., one or more immunomodulatory polypeptides) can be present in a heterodimeric TMP of the present disclosure at any of a variety of positions. FIG. 17 depicts the position of two copies of a variant IL-2 polypeptide; however, the immunomodulatory polypeptide can be any number of and any of a variety of immunomodulatory polypeptides, as described herein. As depicted in FIG. 17, an immunomodulatory polypeptide can be: 1) N-terminal to the MHC class I heavy chain; 2) C-terminal to the MHC class I heavy chain and N-terminal to the Ig Fc polypeptide; in other words, between the MHC class I heavy chain and the Ig Fc polypeptide; 3) C-terminal to the Ig Fc polypeptide; 4) N-terminal to the peptide epitope; or 5) C-terminal to the β2M polypeptide.

As depicted schematically in FIG. 18, an immunomodulatory polypeptide (i.e., one or more immunomodulatory polypeptides) can be present in a single-chain TMP of the present disclosure at any of a variety of positions. FIG. 18 depicts the position of two copies of a variant IL-2 polypeptide; however, the immunomodulatory polypeptide can be any number of and any of a variety of immunomodulatory polypeptides, as described herein. As depicted in FIG. 18, an immunomodulatory polypeptide can be: 1) C-terminal to the MHC class I heavy chain and N-terminal to the Ig Fc polypeptide; 2) C-terminal to the Ig Fc polypeptide; or 3) N-terminal to the peptide epitope.

Immunomodulatory polypeptides and variants, including reduced affinity variants, such as PD-L1, CD80, CD86, 4-1BBL and IL-2 are described in the published literature, e.g., published PCT application WO2020132138A1 and WO2019/051091, the disclosures of which as they pertain to immunomodulatory polypeptides and specific variant immunomodulatory polypeptides of PD-L1, CD80, CD86, 4-1BBL, IL-2 are expressly incorporated herein by reference, including specifically paragraphs [00260]-[00455] of WO2020132138A1 and paragraphs [00157]-[00352] of WO2019/051091.

Of specific interest are MODs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wt. Il-2 such as the drug Aldesleukin (trade name Proleukin®) are known to have severe side-effects when administered to humans for the treatment of cancer because the IL-2 indiscriminately activates both target and non-target T cells.

An IL-2 receptor is in some cases a heterotrimeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2 (SEQ ID NO:15), human IL-2Rα (SEQ ID NO:16), IL2Rβ (SEQ ID NO:17), and IL-2Rγ (SEQ ID NO:18) are known. See, e.g., published PCT applications WO2020132138A1 and WO2019/051091, discussed above.

In some cases, an IL-2 variant MOD of this disclosure exhibits substantially reduced or no binding to IL-2Rα, thereby minimizing or substantially reducing the activation of Tregs by the IL-2 variant. In some cases, an IL-2 variant MOD of this disclosure has reduced affinity to IL-2Rß and/or IL-2Rγ such that the IL-2 variant MOD exhibits an overall reduced affinity for IL-2R. In some cases, an IL-2 variant MOD of this disclosure exhibits both properties, i.e., it exhibits substantially reduced or no binding to IL-2Rα, and also has reduced affinity to IL-2Rß and/or IL-2Rγ such that the IL-2 variant polypeptide exhibits an overall reduced affinity for IL-2R. TMPs comprising such variants, including variants that substantially do not bind IL-2Rα and have reduced affinity to IL-2Rß, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically binds the peptide epitope on the TMP. That is, the binding of the IL-2 variant MOD to its costimulatory polypeptide on the T cell is substantially driven by the binding of the MHC-epitope moiety rather than by the binding of the Il-2.

Suitable IL-2 variant MODs thus include a polypeptide that comprises an amino acid sequence having at least 90%, at least 95%, at least 98%, at least 99% amino acid sequence identity to the amino acid sequence set forth in SEQ ID NO:15 for an IL-2R. In some cases, such a variant IL-2 polypeptide of this disclosure exhibits reduced binding affinity to IL-2R, compared to the binding affinity of a IL-2 polypeptide comprising the amino acid sequence set forth in SEQ ID NO:15. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the amino acid sequence set forth in SEQ ID NO:15 for an IL-2R (e.g., an IL-2R comprising polypeptides comprising the amino acid sequence set forth in SEQ ID NOs:16-18), when assayed under the same conditions. In some cases, such a variant IL-2 polypeptide has a binding affinity to IL-2R that is from 100 nM to 100 μM. As another example, in some cases, a variant IL-2 polypeptide has a binding affinity for IL-2R (e.g., an IL-2R comprising polypeptides comprising the amino acid sequence set forth in SEQ ID NOs:16-18) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO: 241), i.e., the variant IL-2 polypeptide has the amino acid sequence of wild-type IL-2 but with H16A and F42A substitutions (shown in bold). Alternatively, the foregoing sequence but substitutions other than Ala at H16 and/or F42 may be employed, e.g., H16T may be employed instead of H16A.

### Scaffold Polypeptides

A TMP of the present disclosure can comprise an Fc polypeptide or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:561), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the TMP, compared to a control TMP lacking the scaffold polypeptide. For example, in some cases, a scaffold polypeptide increases the in vivo half-life (e.g., the serum half-life) of the TMP, compared to a control TMP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the TMP, compared to a control TMP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

In some cases, the first and/or the second polypeptide chain of a TMP of the present disclosure comprises an Fc polypeptide. The Fc polypeptide of a TMP of the present disclosure can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 3A-3G. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 3A, optionally comprising a substitution of N77; e.g., the Fc polypeptide comprises an N77A substitution. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 3A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 3A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 3A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 3A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 3B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 3B. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 3C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 3C.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 3C. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fc polypeptide depicted in FIG. 3C.

In some cases, the IgG4 Fc polypeptide comprises the following amino acid sequence:

(SEQ ID NO: 175)
PPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSPG.

Typically, the Ig Fc employed in the TMP will comprise one or more substitutions for amino acids in the wild-type sequence the such that that Ig Fc that “substantially does not induce cell lysis. For example, in some cases the Fc polypeptide present in a TMP comprises the amino acid sequence depicted in FIG. 3A (human IgG1 Fc), except for a substitution of L234 (L14 of the amino acid sequence depicted in FIG. 3A) with an amino acid other than leucine, or a substitution of L235 (L15 of the amino acid sequence depicted in FIG. 3A) with an amino acid other than leucine.

In some cases, the Fc polypeptide present in a TMP comprises the amino acid sequence depicted in FIG. 3G (human IgG1 Fc comprising an L234A substitution and an L235A substitution, corresponding to positions 14 and 15 of the amino acid sequence depicted in FIG. 3G). In some cases, the Fc polypeptide present in a TMP comprises the amino acid sequence depicted in FIG. 3A (human IgG1 Fc), except for substitutions at L234 and L235 (L14 and L15 of the amino acid sequence depicted in FIG. 3A) with amino acids other than leucine. In some cases, the Fc polypeptide present in a TMP comprises the amino acid sequence depicted in FIG. 3A (human IgG1 Fc), except for substitutions at L234 and L235 (L14 and L15 of the amino acid sequence depicted in FIG. 3A) with amino acids other than leucine, and a substitution of P331 (P111 of the amino acid sequence depicted in FIG. 3A) with an amino acid other than proline. In some cases, the Fc polypeptide present in a TMP comprises the amino acid sequence depicted in FIG. 3E (human IgG1 Fc comprising L234F, L235E, and P331S substitutions (corresponding to amino acid positions 14, 15, and 111 of the amino acid sequence depicted in FIG. 3E). In some cases, the Fc polypeptide present in a TMP is an IgG1 Fc polypeptide that comprises L234A and L235A substitutions (substitutions of L14 and L15 of the amino acid sequence depicted in FIG. 3A with Ala), as depicted in FIG. 3G.

### Linkers

A TMP of the present disclosure can include one or more linkers, where the one or more linkers are between one or more of: i) an MHC class I polypeptide and an Ig Fc polypeptide, where such a linker is referred to herein as “L1”; ii) an immunomodulatory polypeptide and an MHC class I polypeptide, where such a linker is referred to herein as “L2”; iii) a first immunomodulatory polypeptide and a second immunomodulatory polypeptide, where such a linker is referred to herein as “L3”; iv) a peptide antigen (“epitope”) and an MIHC class I polypeptide; v) an MIHC class I polypeptide and a dimerization polypeptide (e.g., a first or a second member of a dimerizing pair); and vi) a dimerization polypeptide (e.g., a first or a second member of a dimerizing pair) and an Ig Fc polypeptide.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

Exemplary linkers include glycine polymers (G)ₙ, glycine-serine polymers (including, for example, (GS)ₙ, (GSGGS)ₙ (SEQ ID NO:366), and (GGGS)ₙ (SEQ ID NO:367), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:368), GGSGG (SEQ ID NO: 369), GSGSG (SEQ ID NO:370), GSGGG (SEQ ID NO:371), GGGSG (SEQ ID NO:372), GSSSG (SEQ ID NO:373), and the like. Exemplary linkers can include, e.g., Gly(Ser₄)n (SEQ ID NO: 374), where n is 1, 2, 3, 4, 5, 6, 7, 8, 9, or 10. In some cases, a linker comprises the amino acid sequence (GSSSS)n (SEQ ID NO:375), where n is 4. In some cases, a linker comprises the amino acid sequence (GSSSS)n (SEQ ID NO:376), where n is 5.

Exemplary linkers can include, e.g., (GGGGS)n (SEQ ID NO:377); also referred to as a “G4S” linker), where n is 1, 2, 3, 4, 5, 6, 7, 8, 9, or 10. In some cases, a linker comprises the amino acid sequence (GGGGS)n (SEQ ID NO:377), where n is 1, 2, 3, 4, 5, 6, 7, 8, 9 or 10. In some cases, a linker comprises the amino acid sequence AAAGG (SEQ ID NO:387). Also suitable is a linker having the amino acid sequence AAAGG (SEQ ID NO:387). In single-chain TMPs of this disclosure, the ß2M polypeptide can be connected to the MHC heavy chain polypeptide by a (GGGGS)n (SEQ ID NO:377) linker, where n is 1, 2, 3, 4, 5, 6, 7, 8, 9, or 10, e.g., with n=3 or 7.

In some cases, a linker polypeptide, present in a first polypeptide of a TMP of the present disclosure, includes a cysteine residue that can form a disulfide bond with a cysteine residue present in a second polypeptide of a TMP of the present disclosure. In some cases, for example, a suitable linker comprises the amino acid sequence GCGGSGGGGSGGGGS (SEQ ID NO:388). As another example, a suitable linker can comprise the amino acid sequence GCGGS(GGGGS)n (SEQ ID NO:389), where n is 1, 2, 3, 4, 5, 6, 7, 8, or 9. For example, in some cases, the linker comprises the amino acid sequence GCGGSGGGGSGGGGSGGGGS (SEQ ID NO:390). As another example, the linker comprises the amino acid sequence GCGGSGGGGSGGGGS (SEQ ID NO:391).

### Dimerized TMPs

In some cases, single-chain and heterodimeric TMPs of the present disclosure can form dimers; i.e., the present disclosure provides a polypeptide comprising a dimer of a TMP of the present disclosure. The present disclosure provides, e.g., a protein (a dimerized TMP of the present disclosure) comprising: A) a first heterodimer comprising: a) a first polypeptide comprising: i) a KRAS peptide; and ii) a first MHC polypeptide; and b) a second polypeptide comprising: i) a second MIHC polypeptide, wherein the first heterodimer comprises one or more immunomodulatory polypeptides; and B) a second heterodimer comprising: a) a first polypeptide comprising: i) a KRAS peptide; and ii) a first MHC polypeptide; and b) a second polypeptide comprising: i) a second MHC polypeptide, wherein the second heterodimer comprises one or more immunomodulatory polypeptides, and wherein the first heterodimer and the second heterodimer are covalently linked to one another. Alternatively, the dimerized TMP can comprise two single-chain TMPs that are covalently linked to each other. The covalent linkage of the dimer can be a disulfide bond between an Ig Fc polypeptide in the first single-chain or heterodimeric TMP and an Ig Fc polypeptide in the second single-chain or heterodimeric TMP. When the TMP comprises an Ig Fc polypeptide, e.g., a human IgG1 Fc polypeptide that substantially does not induce cell lysis (e.g., the polypeptide of FIG. 3G), the TMP typically will self-assemble into a dimer by spontaneously forming disulfide bonds with the IgG1 Fc polypeptide of another TMP. Thus, e.g., the Ig Fc polypeptides in the first single-chain or heterodimeric TMP and the second single-chain or heterodimer can be linked to one another by one or more disulfide bonds. In some cases, the two TMPs are identical to one another in amino acid sequence. In some cases, the first heterodimer and the second heterodimer are covalently linked to one another via a C-terminal region of the second polypeptide of the first heterodimer and a C-terminal region of the second polypeptide of the second heterodimer. In some cases, first heterodimer and the second heterodimer are covalently linked to one another via the C-terminal amino acid of the second polypeptide of the first heterodimer and the C-terminal region of the second polypeptide of the second heterodimer; for example, in some cases, the C-terminal amino acid of the second polypeptide of the first heterodimer and the C-terminal region of the second polypeptide of the second heterodimer are linked to one another, either directly or via a linker. The linker can be a peptide linker. The peptide linker can have a length of from 1 amino acid to 200 amino acids (e.g., from 1 amino acid (aa) to 5 aa, from 5 aa to 10 aa, from 10 aa to 25 aa, from 25 aa to 50 aa, from 50 aa to 100 aa, from 100 aa to 150 aa, or from 150 aa to 200 aa). In some cases, the peptide epitope of the first heterodimer and the peptide epitope of the second heterodimer comprise the same amino acid sequence. In some cases, the first MHC polypeptide of the first and the second heterodimer is an MHC class I β2-microglobulin, and wherein the second MHC polypeptide of the first and the second heterodimer is an MHC class I heavy chain. In some cases, the immunomodulatory polypeptide of the first heterodimer and the immunomodulatory polypeptide of the second heterodimer comprise the same amino acid sequence. In some cases, the immunomodulatory polypeptide of the first heterodimer and the immunomodulatory polypeptide of the second heterodimer are variant immunomodulatory polypeptides that comprise from 1 to 10 amino acid substitutions compared to a corresponding parental wild-type immunomodulatory polypeptide, and wherein the from 1 to 10 amino acid substitutions result in reduced affinity binding of the variant immunomodulatory polypeptide to a cognate costimulatory polypeptide. In some cases, the immunomodulatory polypeptide of the first heterodimer and the immunomodulatory polypeptide of the second heterodimer are each independently selected from the group consisting of wild-type and variant polypeptides of IL-2, 4-1BBL, PD-L1, CD80, CD86, ICOS-L, OX-40L, FasL, JAG1 (CD339), TGFβ, CD70, and ICAM. Examples, of suitable MHC polypeptides, immunomodulatory polypeptides, and peptide epitopes are described herein.

In some cases, a single-chain TMP of the present disclosure is dimerized. Thus, the present disclosure provides a protein comprising: a) a first single-chain TMP of the present disclosure; and b) a second single-chain TMP of the present disclosure, where the first and second single-chain TMPs are covalently linked to one another. The covalent linkage can be a disulfide bond between an Ig Fc polypeptide in the first single-chain TMP and an Ig Fc polypeptide in the second single-chain TMP.

### Additional Polypeptides

A polypeptide chain of a TMP of the present disclosure can include one or more polypeptides and conjugate drugs in addition to those described above. Suitable additional polypeptides, including epitope tags and affinity domains, and drug conjugates are described in in published PCT applications WO2020132138A1 and WO2019/051091, discussed above, the disclosures of which as they pertain to epitope tags, affinity domains and drug conjugates are expressly incorporated herein by reference, including specifically paragraphs [00498]-[00508] of WO2020132138A1 and paragraphs [00353]-[00363] of WO2019/051091. The one or more additional polypeptide can be included at the N-terminus of a polypeptide chain of a TMP, at the C-terminus of a polypeptide chain of a TMP, or internally within a polypeptide chain of a TMP.

### Exemplary TMPs

In some cases, a TMP of the present disclosure comprises at least one heterodimer comprising: a) a first polypeptide comprising: i) a KRAS peptide; and ii) first MHC polypeptide; b) a second polypeptide comprising a second MHC polypeptide, and c) at least one immunomodulatory polypeptide, where the first and/or the second polypeptide comprises the immunomodulatory polypeptide, and optionally comprises an Ig Fc. Thus, in some cases, a TMP of the present disclosure comprises at least one heterodimer comprising: a) a first polypeptide comprising: i) a KRAS peptide; ii) first MHC polypeptide; and iii) at least one immunomodulatory polypeptide; and b) a second polypeptide comprising a second MHC polypeptide and optionally comprises an Ig Fc. In other instances, a TMP of the present disclosure comprises at least one heterodimer comprising: a) a first polypeptide comprising: i) a KRAS peptide; and ii) first MHC polypeptide; and b) a second polypeptide comprising: i) a second MHC polypeptide; and ii) at least one immunomodulatory polypeptide, and optionally comprises an Ig Fc. In some cases, a TMP of the present disclosure comprises at least one heterodimer comprising: a) a first polypeptide comprising: i) a KRAS peptide; ii) first MHC polypeptide; and iii) at least one immunomodulatory polypeptide; and b) a second polypeptide comprising: i) a second MHC polypeptide; and ii) at least one immunomodulatory polypeptide, and optionally comprises an Ig Fc. In some cases, the at least one immunomodulatory polypeptide is a wild-type immunomodulatory polypeptide. In other cases, the at least one immunomodulatory polypeptide is a variant immunomodulatory polypeptide that exhibits reduced affinity for a costimulatory polypeptide, compared to the affinity of a corresponding wild-type immunomodulatory polypeptide for the costimulatory polypeptide.

As noted above, and as depicted schematically in FIG. 17, an immunomodulatory polypeptide (i.e., one or more immunomodulatory polypeptides) can be present in a TMP of the present disclosure at any of a variety of positions. FIG. 17 depicts the position of two copies of a variant IL-2 polypeptide; however, the immunomodulatory polypeptide can be any number of and any of a variety of immunomodulatory polypeptide, as described herein. As depicted in FIG. 17, an immunomodulatory polypeptide can be: 1) N-terminal to the MHC class I heavy chain (position 1); 2) C-terminal to the MHC class I heavy chain and N-terminal to the Ig Fc polypeptide; in other words, between the MHC class I heavy chain and the Ig Fc polypeptide (position 2); 3) C-terminal to the Ig Fc polypeptide (position 3); 4) N-terminal to the peptide epitope (position 4); or 5) C-terminal to the β2M polypeptide (position 5). In some cases, therefore, a TMP of the present disclosure comprises one of the following scaffolds having the defined arrangements of components, wherein the first MHC polypeptide is a β2M polypeptide; and the second MHC polypeptide is an HLA heavy chain polypeptide:
a) a first polypeptide comprising, in order from N-terminus to C-terminus: i) a KRAS peptide; and ii) a first MHC polypeptide; and b) a second polypeptide comprising, in order from N-terminus to C-terminus: i) at least one immunomodulatory polypeptide; ii) a second MHC polypeptide; and iii) an Ig Fc polypeptide (this arrangement being referred to as MOD Position 1);
a) a first polypeptide comprising, in order from N-terminus to C-terminus: i) a KRAS peptide; and ii) a first MHC polypeptide; and b) a second polypeptide comprising, in order from N-terminus to C-terminus: i) a second MHC polypeptide; ii) at least one immunomodulatory polypeptide; and iii) an Ig Fc polypeptide (this arrangement being referred to as MOD Position 2); TMP
a) a first polypeptide comprising, in order from N-terminus to C-terminus: i) a KRAS peptide; and ii) a first MHC polypeptide; and b) a second polypeptide comprising, in order from N-terminus to C-terminus: i) a second MHC polypeptide; ii) an Ig Fc polypeptide; and iii) at least one immunomodulatory polypeptide (this arrangement being referred to as MOD Position 3).
a) a first polypeptide comprising, in order from N-terminus to C-terminus: i) at least one immunomodulatory polypeptide; ii) a KRAS peptide; and iii) a first MHC polypeptide; and b) a second polypeptide comprising, in order from N-terminus to C-terminus: i) a second MIHC polypeptide; and ii) an Ig Fc polypeptide (this arrangement being referred to as MOD Position 4);
a) a first polypeptide comprising, in order from N-terminus to C-terminus: i) a KRAS peptide; ii) a first MHC polypeptide; and iii) at least one immunomodulatory polypeptide; and b) a second polypeptide comprising, in order from N-terminus to C-terminus: i) a second MHC polypeptide; and ii) an Ig Fc polypeptide (this arrangement being referred to as MOD Position 5);

In the above scaffolds, any of the components of the first and second polypeptides optionally may be joined to the next component in the polypeptide by a linker. In some cases, a peptide linker is between one or more of: i) the second MHC polypeptide and the Ig Fc polypeptide; ii) the epitope and the first MHC polypeptide; iii) the first MHC polypeptide and the immunomodulatory polypeptide; and (where the TMP comprises two immunomodulatory polypeptides on the first polypeptide chain) iv) between the two immunomodulatory polypeptides, v) the second MIHC polypeptide and the Ig Fc polypeptide; vi) the first MHC polypeptide and the immunomodulatory polypeptide(s). In some cases, the peptide linker comprises the amino acid sequence AAAGG (SEQ ID NO:387). In some cases, the peptide linker comprises the amino acid sequence (GGGGS)n, where n is an integer from 1 to 10 (e.g., where n is 2, 3, or 4).

In any of the above scaffolds, the KRAS peptide has an amino acid sequence selected from the following group: VVGADGVGK (SEQ ID NO:176); VVGACGVGK (SEQ ID NO:177); VVGAVGVGK (SEQ ID NO:178); VVVGADGVGK (SEQ ID NO:179); VVVGAVGVGK (SEQ ID NO:180); VVVGACGVGK (SEQ ID NO:181); VTGADGVGK (SEQ ID NO:182); VTGAVGVGK (SEQ ID NO:183); VTGACGVGK (SEQ ID NO:184); VTVGADGVGK (SEQ ID NO:185); VTVGAVGVGK (SEQ ID NO:186); VTVGACGVGK (SEQ ID NO:187); LVVVGADGV (SEQ ID NO:192); LVVVGAVGV (SEQ ID NO:193); LVVVGACGV (SEQ ID NO:194); KLVVVGADGV (SEQ ID NO:195); KLVVVGAVGV (SEQ ID NO:196); KLVVVGACGV (SEQ ID NO:197); LLVVGADGV (SEQ ID NO:198); LLVVGAVGV (SEQ ID NO:199); LLVVGACGV (SEQ ID NO:200); FLVVVGADGV (SEQ ID NO:201); FLVVVGAVGV (SEQ ID NO:202); FLVVVGACGV (SEQ ID NO:203).

In the above scaffolds, in some cases, the second MHC polypeptide is an HLA heavy chain that comprises an amino acid sequence having at least 95% amino acid sequence identity to an HLA-A*0201 polypeptide, an HLA-A*1101 polypeptide or an HLA-A24 polypeptide. In some cases, the HLA heavy chain polypeptide is an HLA-A*0201 polypeptide. In some cases, the HLA heavy chain polypeptide is an HLA-A*0201 polypeptide comprising an A236C substitution. In some cases, the HLA heavy chain polypeptide is an HLA-A*1101 polypeptide comprising an A236C substitution.

In some cases, the scaffolds comprise two immunomodulatory polypeptides, where the two immunomodulatory polypeptides have the same amino acid sequence, e.g., the immunomodulatory polypeptide is a variant IL-2 polypeptide comprising H16A and F42A substitutions, or a variant IL-2 polypeptide comprising H16T and F42A substitutions.

In some cases, the Ig Fc polypeptide is a variant of a human IgG1 Fc polypeptide that substantially does not induce cell lysis, e.g., an IgG1 Fc polypeptide comprising L234A and L235A substitutions such as is shown in FIG. 3G.

In some cases, the first and the second polypeptides are disulfide linked to one another.

In some cases, a TMP of the present disclosure comprises a scaffold with a MOD Position 1 or Position 3 arrangement, wherein the HLA heavy chain polypeptide is a wild-type or variant HLA-A*0201 polypeptide, e.g., an HLA-A*0201 polypeptide comprising an A236C substitution, or a sequence as shown in FIG. 22C, or a variant thereof. In some cases, the Ig Fc polypeptide is a human IgG1 Fc polypeptide that substantially does not cause cell lysis, e.g., a human IgG1 Fc polypeptide comprising L234A and L235A substitutions as shown in FIG. 3G. In some cases, the first and the second polypeptides are disulfide linked to one another. In some cases, the immunomodulatory polypeptide is a variant IL-2 polypeptide comprising H16A and F42A substitutions or H16T and F42A substitutions. In some cases, the KRAS peptide has an amino acid sequence selected from the following group:

(SEQ ID NO: 176)
VVGADGVGK;
(SEQ ID NO: 177)
VVGACGVGK;
(SEQ ID NO: 178)
VVGAVGVGK;
(SEQ ID NO: 179)
VVVGADGVGK;
(SEQ ID NO: 180)
VVVGAVGVGK;
(SEQ ID NO: 181)
VVVGACGVGK;
(SEQ ID NO: 182)
VTGADGVGK;
(SEQ ID NO: 183)
VTGAVGVGK;
(SEQ ID NO: 184)
VTGACGVGK;
(SEQ ID NO: 185)
VTVGADGVGK;
(SEQ ID NO: 186)
VTVGAVGVGK;
(SEQ ID NO: 187)
VTVGACGVGK;
(SEQ ID NO: 192)
LVVVGADGV;
(SEQ ID NO: 193)
LVVVGAVGV;
(SEQ ID NO: 194)
LVVVGACGV;
(SEQ ID NO: 195)
KLVVVGADGV;
(SEQ ID NO: 196)
KLVVVGAVGV;
(SEQ ID NO: 197)
KLVVVGACGV;
(SEQ ID NO: 198)
LLVVGADGV;
(SEQ ID NO: 199)
LLVVGAVGV;
(SEQ ID NO: 200)
LLVVGACGV;
(SEQ ID NO: 201)
FLVVVGADGV;
(SEQ ID NO: 202)
FLVVVGAVGV;
(SEQ ID NO: 203)
FLVVVGACGV.

In some cases, a TMP of the present disclosure comprises a scaffold with a MOD Position 1 or Position 3 arrangement, wherein the HLA heavy chain polypeptide is a wild-type or variant HLA-A24 polypeptide (also referred to as HLA-A*2402), e.g., an HLA-A*0201 polypeptide comprising an A236C substitution or an amino acid sequence shown in any one of FIG. 21O, 21P, 21Q, 21R, 21S or 21T, or a variant thereof. In some cases, the Ig Fc polypeptide is a human IgG1 Fc polypeptide that substantially does not cause cell lysis, e.g., a human IgG1 Fc polypeptide comprising L234A and L235A substitutions as shown in FIG. 3G. In some cases, the first and the second polypeptides are disulfide linked to one another. In some cases, the immunomodulatory polypeptide is a variant IL-2 polypeptide comprising H16A and F42A substitutions or H16T and F42A substitutions. In some cases, the KRAS peptide has an amino acid sequence selected from the following group: VVGADGVGK (SEQ ID NO:176); VVGACGVGK (SEQ ID NO:177); VVGAVGVGK (SEQ ID NO:178); VVVGADGVGK (SEQ ID NO:179); VVVGAVGVGK (SEQ ID NO:180); VVVGACGVGK (SEQ ID NO:181); VTGADGVGK (SEQ ID NO:182); VTGAVGVGK (SEQ ID NO:183); VTGACGVGK (SEQ ID NO:184); VTVGADGVGK (SEQ ID NO:185); VTVGAVGVGK (SEQ ID NO:186); VTVGACGVGK (SEQ ID NO:187); LVVVGADGV (SEQ ID NO:192); LVVVGAVGV (SEQ ID NO:193); LVVVGACGV (SEQ ID NO:194); KLVVVGADGV (SEQ ID NO:195); KLVVVGAVGV (SEQ ID NO:196); KLVVVGACGV (SEQ ID NO:197); LLVVGADGV (SEQ ID NO:198); LLVVGAVGV (SEQ ID NO:199); LLVVGACGV (SEQ ID NO:200); FLVVVGADGV (SEQ ID NO:201); FLVVVGAVGV (SEQ ID NO:202); FLVVVGACGV (SEQ ID NO:203).

In some cases, a TMP of the present disclosure comprises a scaffold with a MOD Position 1 or Position 3 arrangement, wherein the HLA heavy chain polypeptide is a wild-type or variant HLA-A*1101 polypeptide as disclosed herein, e.g. an HLA-A*1101 polypeptide comprising an A236C substitution or having an amino acid sequence as shown in one of FIG. 21J or 21K, or a variant thereof. In some cases, the Ig Fc polypeptide is a human IgG1 Fc polypeptide that substantially does not cause cell lysis, e.g., a human IgG1 Fc polypeptide comprising L234A and L235A substitutions as shown in FIG. 3G. In some cases, the first and the second polypeptides are disulfide linked to one another. In some cases, the immunomodulatory polypeptide is a variant IL-2 polypeptide comprising H16A and F42A substitutions or H16T and F42A substitutions. In some cases, the KRAS peptide has an amino acid sequence selected from the following group: VVGADGVGK (SEQ ID NO:176); VVGACGVGK (SEQ ID NO:177); VVGAVGVGK (SEQ ID NO:178); VVVGADGVGK (SEQ ID NO:179); VVVGAVGVGK (SEQ ID NO:180); VVVGACGVGK (SEQ ID NO:181); VTGADGVGK (SEQ ID NO:182); VTGAVGVGK (SEQ ID NO:183); VTGACGVGK (SEQ ID NO:184); VTVGADGVGK (SEQ ID NO:185); VTVGAVGVGK (SEQ ID NO:186); VTVGACGVGK (SEQ ID NO:187); LVVVGADGV (SEQ ID NO:192); LVVVGAVGV (SEQ ID NO:193); LVVVGACGV (SEQ ID NO:194); KLVVVGADGV (SEQ ID NO:195); KLVVVGAVGV (SEQ ID NO:196); KLVVVGACGV (SEQ ID NO:197); LLVVGADGV (SEQ ID NO:198); LLVVGAVGV (SEQ ID NO:199); LLVVGACGV (SEQ ID NO:200); FLVVVGADGV (SEQ ID NO:201); FLVVVGAVGV (SEQ ID NO:202); FLVVVGACGV (SEQ ID NO:203).

In some cases, a TMP of the present disclosure comprises a scaffold with a MOD Position 1 or Position 3 arrangement, wherein the HLA heavy chain polypeptide is a wild-type or variant HLA-A*1101 polypeptide as disclosed herein, e.g. an HLA-A*1101 polypeptide comprising an A236C substitution or having an amino acid sequence as shown in one of FIG. 21J or 21K, or a variant thereof. In some cases, the Ig Fc polypeptide is a human IgG1 Fc polypeptide that substantially does not cause cell lysis, e.g., a human IgG1 Fc polypeptide comprising L234A and L235A substitutions as shown in FIG. 3G. In some cases, the first and the second polypeptides are disulfide linked to one another. In some cases, the immunomodulatory polypeptide is a variant IL-2 polypeptide comprising H16A and F42A substitutions or H16T and F42A substitutions. In some cases, the KRAS peptide has an amino acid sequence selected from the following group: VVGADGVGK (SEQ ID NO:176); VVGACGVGK (SEQ ID NO:177); VVGAVGVGK (SEQ ID NO:178); VVVGADGVGK (SEQ ID NO:179); VVVGAVGVGK (SEQ ID NO:180); VVVGACGVGK (SEQ ID NO:181); VTGADGVGK (SEQ ID NO:182); VTGAVGVGK (SEQ ID NO:183); VTGACGVGK (SEQ ID NO:184); VTVGADGVGK (SEQ ID NO:185); VTVGAVGVGK (SEQ ID NO:186); VTVGACGVGK (SEQ ID NO:187); LVVVGADGV (SEQ ID NO:192); LVVVGAVGV (SEQ ID NO:193); LVVVGACGV (SEQ ID NO:194); KLVVVGADGV (SEQ ID NO:195); KLVVVGAVGV (SEQ ID NO:196); KLVVVGACGV (SEQ ID NO:197); LLVVGADGV (SEQ ID NO:198); LLVVGAVGV (SEQ ID NO:199); LLVVGACGV (SEQ ID NO:200); FLVVVGADGV (SEQ ID NO:201); FLVVVGAVGV (SEQ ID NO:202); FLVVVGACGV (SEQ ID NO:203). In the MOD Position 1 arrangement, in some cases, one or more independently selected peptide linkers is/are located between one or more of: i) the KRAS peptide and the first MIHC polypeptide; ii) the immunomodulatory polypeptide and the second MHC polypeptide (and where the TMP comprises two immunomodulatory polypeptides on the second polypeptide chain, between the two immunomodulatory polypeptides); and/or iii) the second MHC polypeptide and the Ig Fc polypeptide. In the MOD Position 3 arrangement, in some cases, one or more independently selected peptide linkers is/are located between one or more of: i) the KRAS peptide and the first MIHC polypeptide; ii) the second MHC polypeptide and the Ig Fc polypeptide and iii) the Ig Fc and the immunomodulatory polypeptide (and where the TMP comprises two immunomodulatory polypeptides on the second polypeptide chain, between the two immunomodulatory polypeptides) TMP. In some cases, the peptide linker comprises the amino acid sequence AAAGG (SEQ ID NO:387). In some cases, the peptide linker comprises the amino acid sequence (GGGGS)n (SEQ ID NO:377), where n is an integer from 1 to 10 (e.g., where n is 2, 3, or 4).

Furthermore, as discussed above and as depicted schematically in FIG. 16A-16C, the first polypeptide chain and the second polypeptide chain of a TMP of the present disclosure can be linked by one or more disulfide bonds. For example, a TMMP of the present disclosure can comprise: a) a first polypeptide chain comprising an β2M polypeptide having an R12C substitution; and b) a second polypeptide chain comprising a class I MHC heavy chain polypeptide having an A236C substitution; such that a disulfide bond forms between the Cys at position 12 of the β2M polypeptide in the first polypeptide chain and the Cys at position 236 of the class I MHC heavy chain polypeptide in the second polypeptide chain. As another example, a TMMP of the present disclosure can comprise: a) a first polypeptide comprising, in order from N-terminus to C-terminus: i) a peptide epitope; ii) a peptide linker comprising a GCGGS(GGGGS)ₙ (SEQ ID NO:582) sequence, where n is 1, 2, or 3; and iii) a β2M polypeptide; and b) a second polypeptide comprising a class I MHC heavy chain polypeptide having a Y84C substitution, such that a disulfide bond forms between the Cys in the peptide linker in the first polypeptide chain and the Cys at position 84 of the class I MIHC heavy chain polypeptide in the second polypeptide chain. In other examples, a TMP of the present disclosure can comprise: a) a first polypeptide comprising, in order from N-terminus to C-terminus: i) a peptide epitope; ii) a peptide linker comprising a GCGGS(GGGGS)ₙ (SEQ ID NO:582) sequence, where n is 1, 2, or 3; and iii) a β2M polypeptide having an R12C substitution; and b) a second polypeptide comprising a class I MIHC heavy chain polypeptide having a Y84C substitution and an A236C substitution; such that: i) a first disulfide bond forms between the Cys in the peptide linker in the first polypeptide chain and the Cys at position 84 of the class I MHC heavy chain polypeptide in the second polypeptide chain; and ii) a second disulfide bond forms between the Cys at position 12 of the β2M polypeptide in the first polypeptide chain and the Cys at position 236 of the class I MIHC heavy chain polypeptide in the second polypeptide chain. For simplicity, the first disulfide bond is referred to as “G2C/Y84C”; and the second disulfide bond is referred to as “R12C/A236C.” A TMP of the present disclosure can include: a) a G2C/Y84C disulfide bond and not an R12C/A236C disulfide bond; b) an R12C/A236C disulfide bond and not a G2C/Y84C disulfide bond; or c) a G2C/Y84C disulfide bond and an R12C/A236C disulfide bond.

A TMP of the present disclosure can include: a) a G2C/Y84C disulfide bond and not an R12C/A236C disulfide bond; and b) at least one immunomodulatory polypeptide at position 1 or 3. A TMP of the present disclosure can include: a) a G2C/Y84C disulfide bond and not an R12C/A236C disulfide bond; and b) at least one immunomodulatory polypeptide at position 2, 4 or 5.

A TMP of the present disclosure can include: a) an R12C/A236C disulfide bond and not a G2C/Y84C disulfide bond; and at least one immunomodulatory polypeptide at position 1 or 3. A TMP of the present disclosure can include: a) an R12C/A236C disulfide bond and not a G2C/Y84C disulfide bond; and at least one immunomodulatory polypeptide at position 2, 4 or 5.

A TMP of the present disclosure can include: a) a G2C/Y84C disulfide bond and an R12C/A236C disulfide bond; and b) and at least one immunomodulatory polypeptide at position 1 or 3. A TMP of the present disclosure can include: a) a G2C/Y84C disulfide bond and an R12C/A236C disulfide bond; and b) and at least one immunomodulatory polypeptide at position 2, 4 or 5.

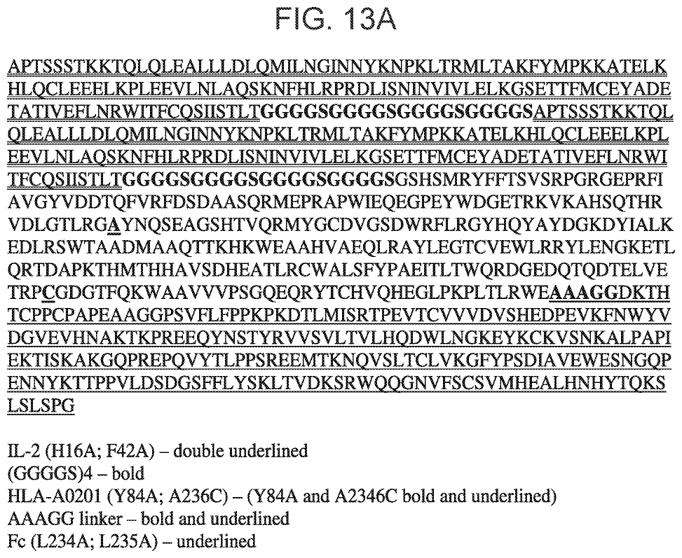

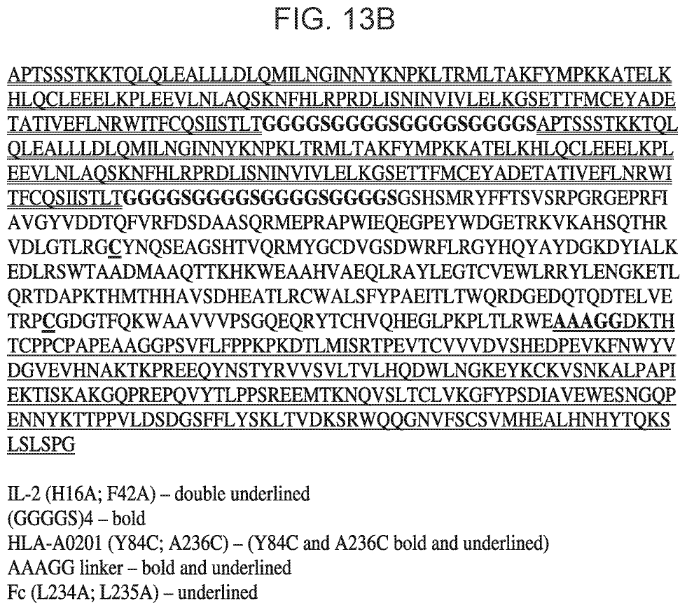

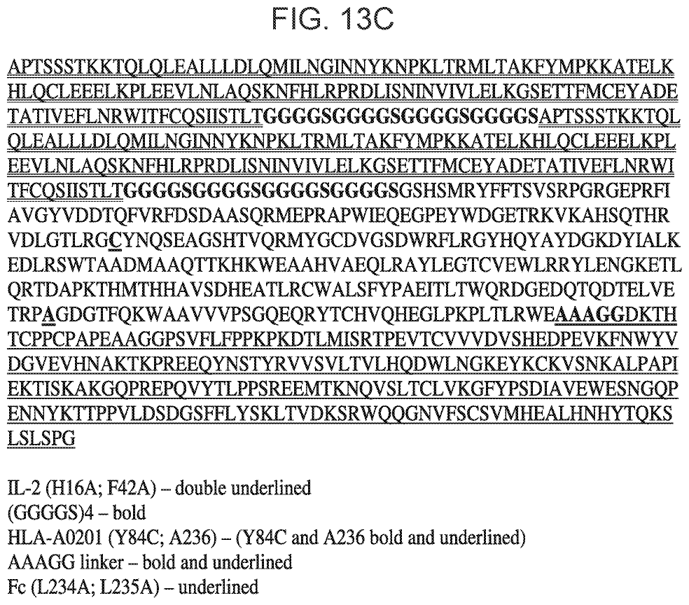

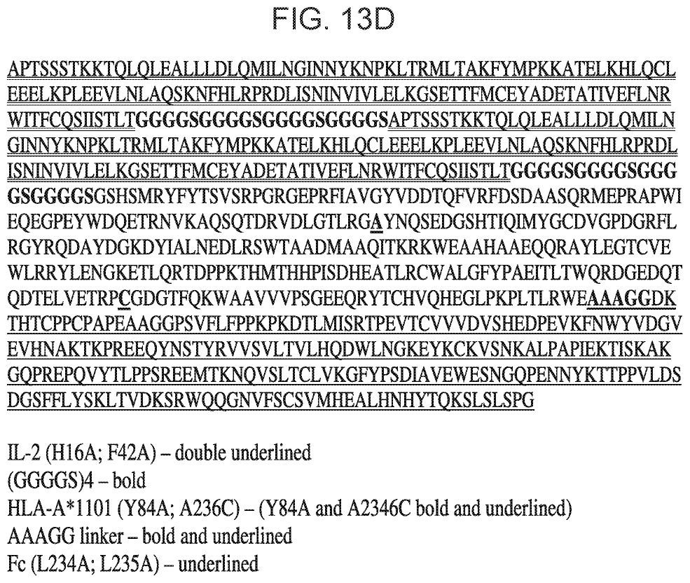

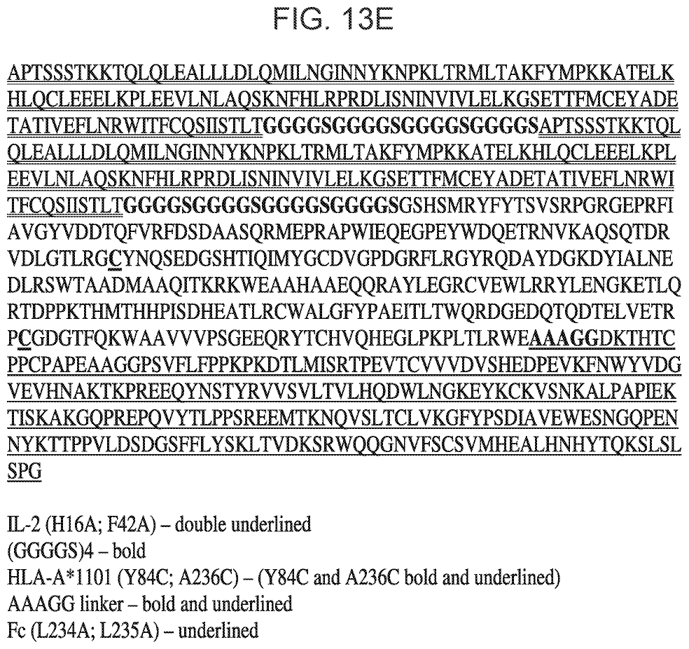

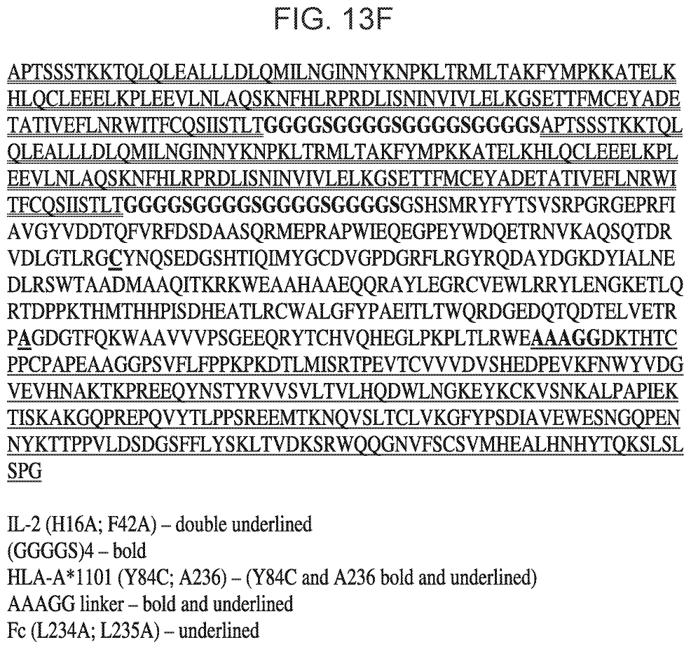

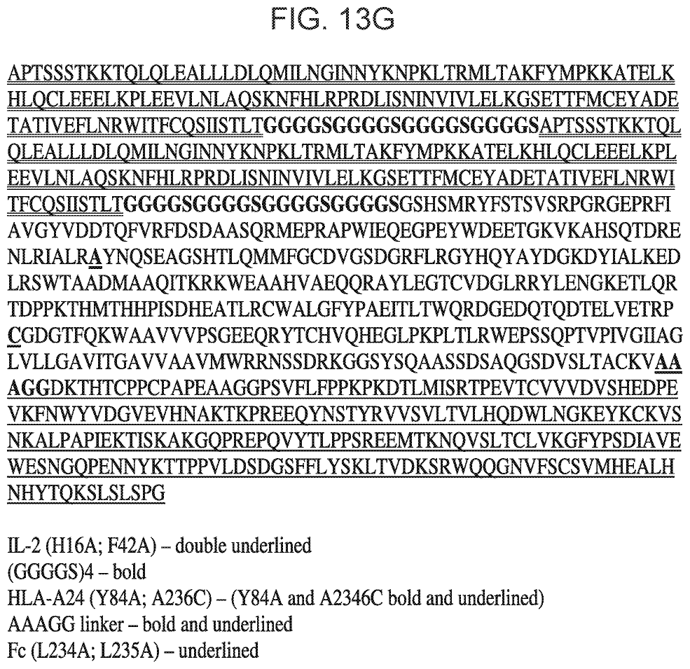

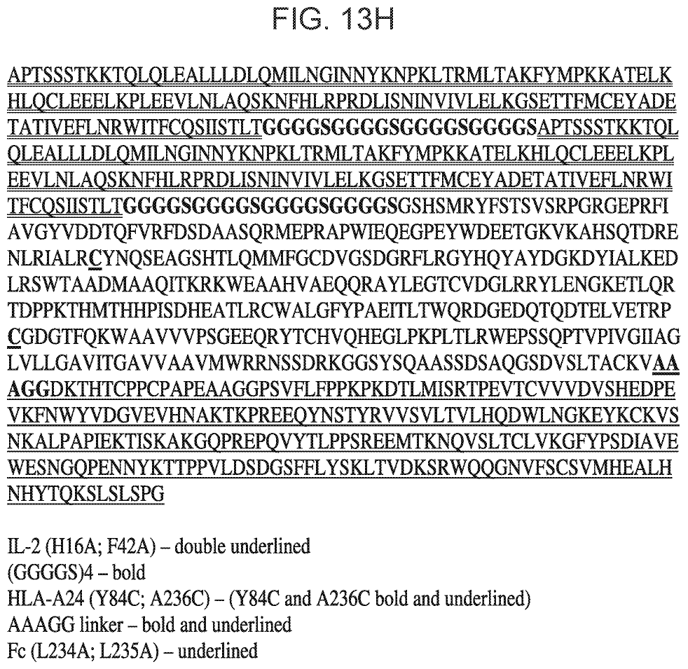

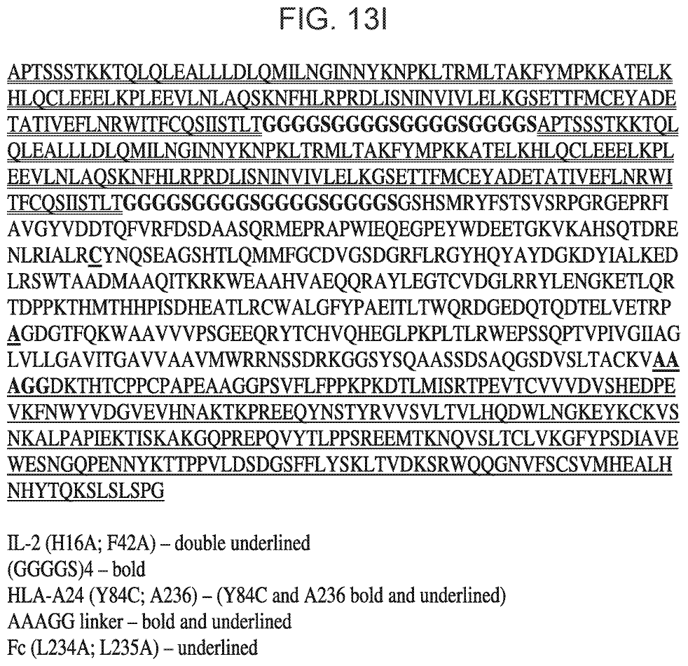

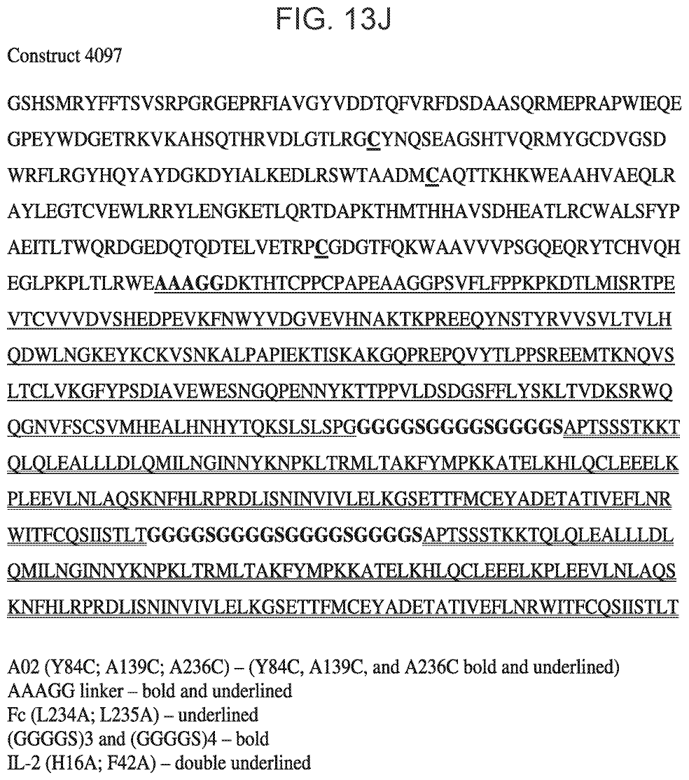

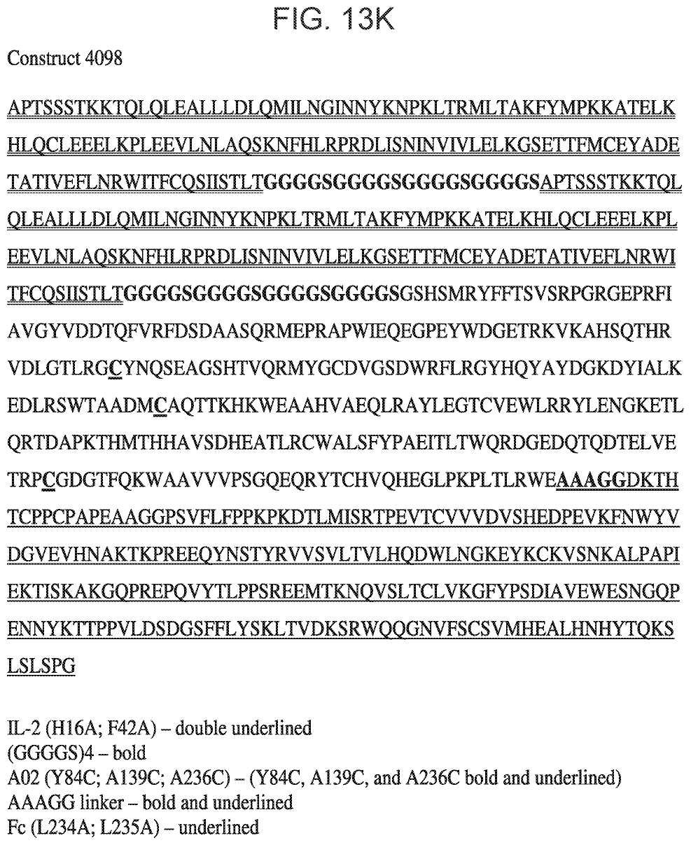

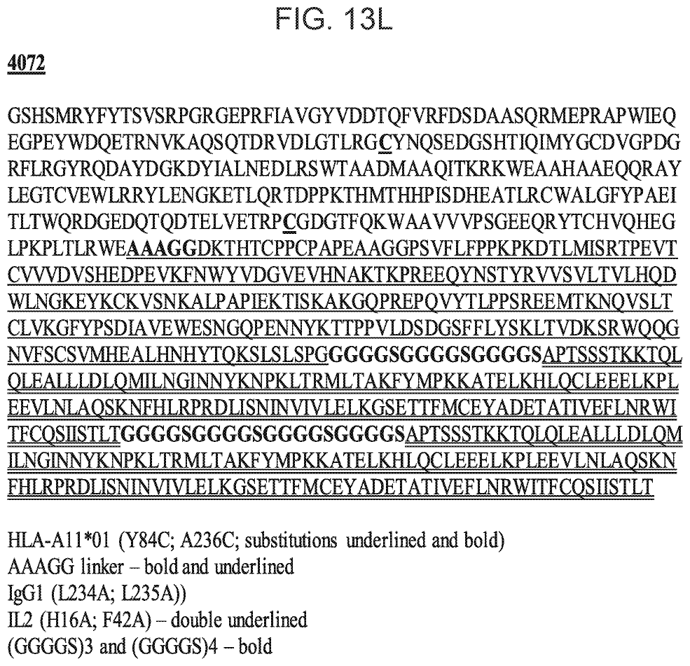

In some cases, a TMP of the present disclosure comprises a second polypeptide comprising (i) an HLA-A0201 (Y84A; A236C) polypeptide comprising an Ala at position 84 and a Cys at position 236, or (ii) an HLA-A0201 (Y84C; A139C) polypeptide comprising a Cys at positions 84 and 139, or (iii) an HLA-A0201 (Y84C; A236) polypeptide comprising a Cys at positions 84 and an alanine at position 236, e.g., as depicted in FIG. 13A, 13B or 13C, respectively.

In some cases, a single-chain TMP of the present disclosure comprises a MHC class I heavy chain polypeptide comprising (i) an HLA-A*1101 (Y84A; A236C) polypeptide comprising an Ala at position 84 and a Cys at position 236, or (ii) an HLA-A*1101 (Y84C; A236C) polypeptide comprising a Cys at positions 84 and 236, or (iii) an HLA-A*1101 (Y84C; A236) polypeptide comprising a Cys at position 84 and an alanine at position 236, e.g., as depicted in FIG. 13D, FIG. 13E, or FIG. 13F, respectively.

In some cases, a TMP of the present disclosure comprises a second polypeptide comprising (i) an HLA-A24 (Y84A; A236C) polypeptide comprising an Ala at position 84 and a Cys at position 236, or (ii) an HLA-A24 (Y84C; A236C) polypeptide comprising a Cys at positions 84 and 236, or (iii) an HLA-A24 (Y84C; A236) polypeptide comprising a Cys at position 84 and an alanine at position 236, e.g., as depicted in FIG. 13G, FIG. 13H or FIG. 13I.

As non-limiting examples, a TMP of the present disclosure can comprise one of the combinations of first and second polypeptides set out in Table 2, below:

TABLE 2

| First Polypeptide | Second Polypeptide |
|---|---|
| FIG. 14A | FIG. 13D, 13E or 13F |
| FIG. 14B | FIG. 13D, 13E or 13F |
| FIG. 14C | FIG. 13D, 13E or 13F |
| FIG. 14D | FIG. 13D, 13E or 13F |
| FIG. 14E | FIG. 13D, 13E or 13F |
| FIG. 14F | FIG. 13D, 13E or 13F |
| FIG. 14G | FIG. 13D, 13E or 13F |
| FIG. 14H | FIG. 13D, 13E or 13F |
| FIG. 14I | FIG. 13D, 13E or 13F |
| FIG. 14J | FIG. 13D, 13E or 13F |
| FIG. 14K | FIG. 13D, 13E, 13F, or 19P |
| FIG. 14L | FIG. 13D, 13E or 13F |
| FIG. 14M | FIG. 13D, 13E or 13F |
| FIG. 14N | FIG. 13D, 13E or 13F |
| FIG. 14O | FIG. 13D, 13E or 13F |
| FIG. 14P | FIG. 13D, 13E or 13F |
| FIG. 14Q | FIG. 13D, 13E, 13F, or 19P |
| FIG. 14R | FIG. 13D, 13E or 13F |
| FIG. 14S | FIG. 13D, 13E or 13F |
| FIG. 14T | FIG. 13D, 13E or 13F |
| FIG. 14U | FIG. 13D, 13E or 13F |
| FIG. 14V | FIG. 13D, 13E or 13F |
| FIG. 14W | FIG. 13D, 13E or 13F |
| FIG. 14X | FIG. 13D, 13E or 13F |
| FIG. 14Y | FIG. 13D, 13E or 13F |
| FIG. 14Z | FIG. 13D, 13E or 13F |
| FIG. 14AA | FIG. 13D, 13E or 13F |
| FIG. 14BB | FIG. 13D, 13E or 13F |
| FIG. 14CC | FIG. 13D, 13E or 13F |
| FIG. 14DD | FIG. 13D, 13E or 13F |
| FIG. 14EE | FIG. 13D, 13E or 13F |
| FIG. 14FF | FIG. 13D, 13E or 13F |
| FIG. 14GG | FIG. 13D, 13E or 13F |
| FIG. 14HH | FIG. 13D, 13E or 13F |
| FIG. 14II | FIG. 13D, 13E or 13F |
| FIG. 14JJ | FIG. 13D, 13E or 13F |

As non-limiting examples, a TMP of the present disclosure can comprise one of the combinations of first and second polypeptides set out in Table 3, below:

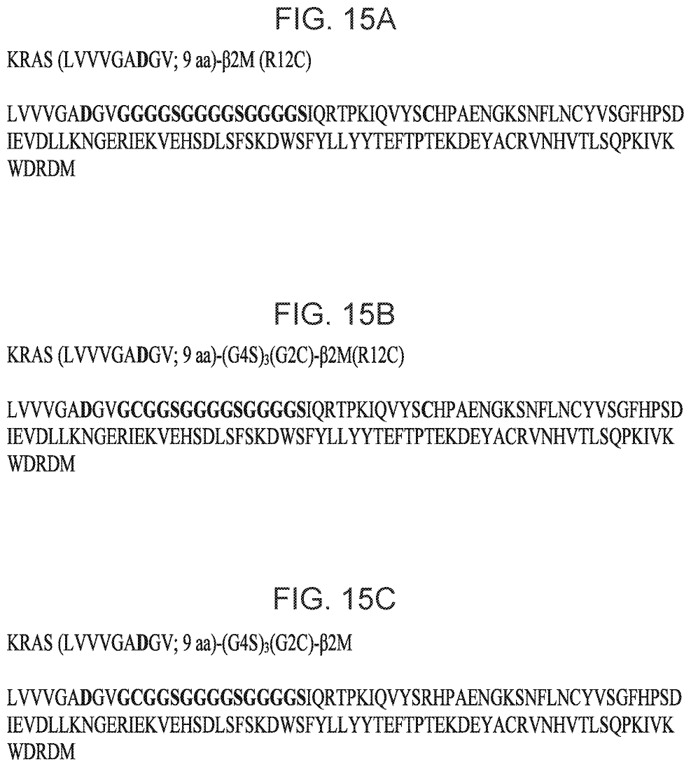

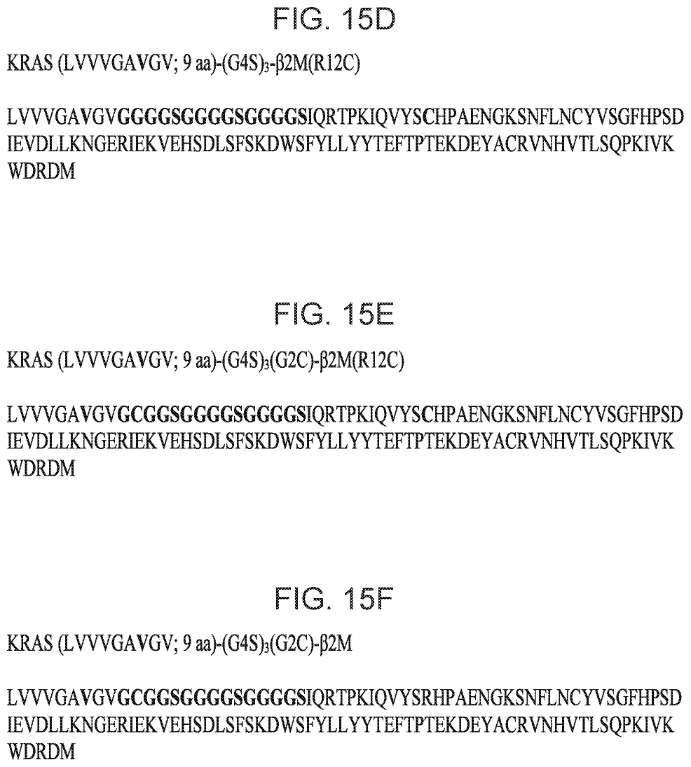

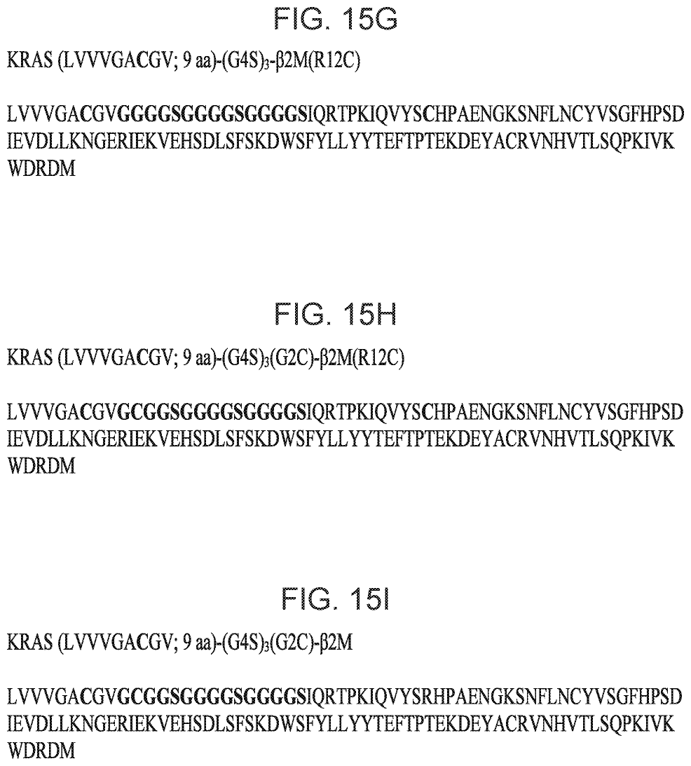

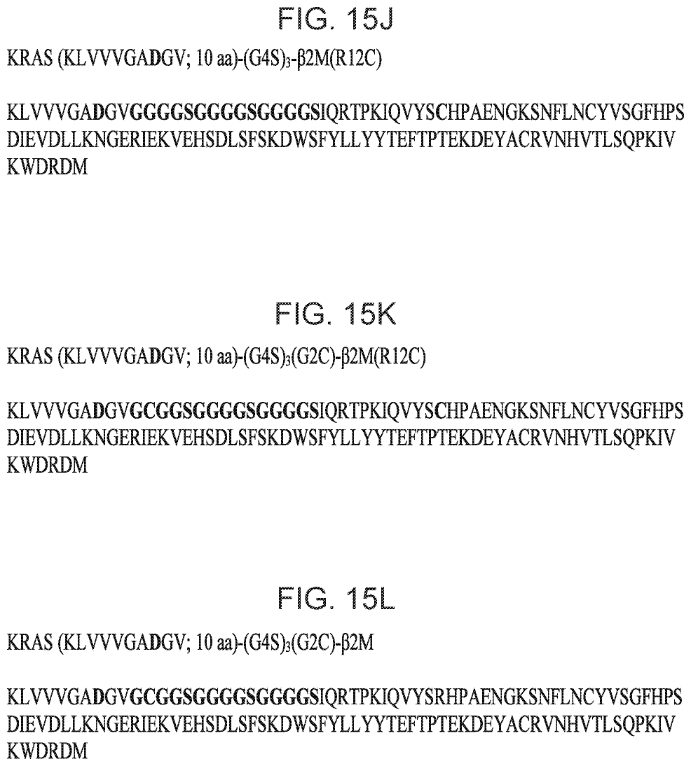

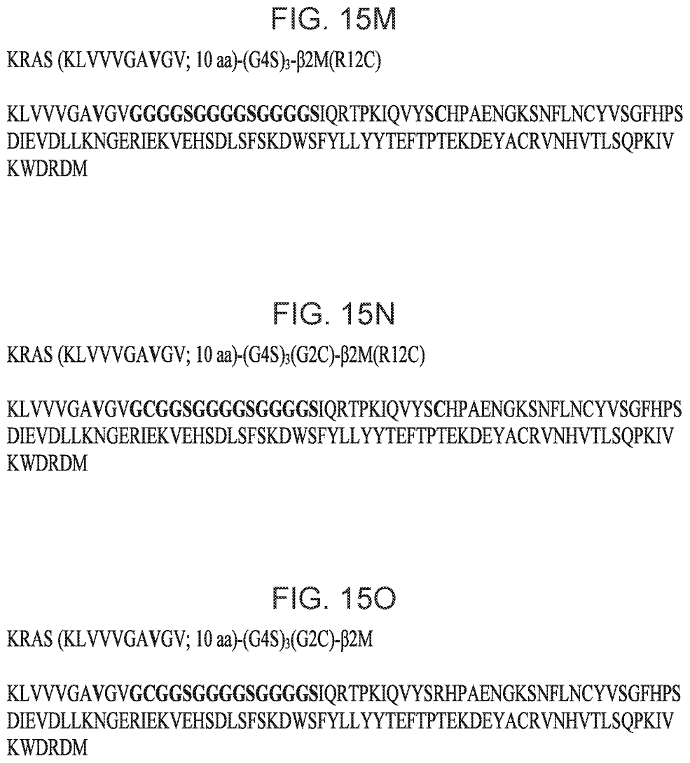

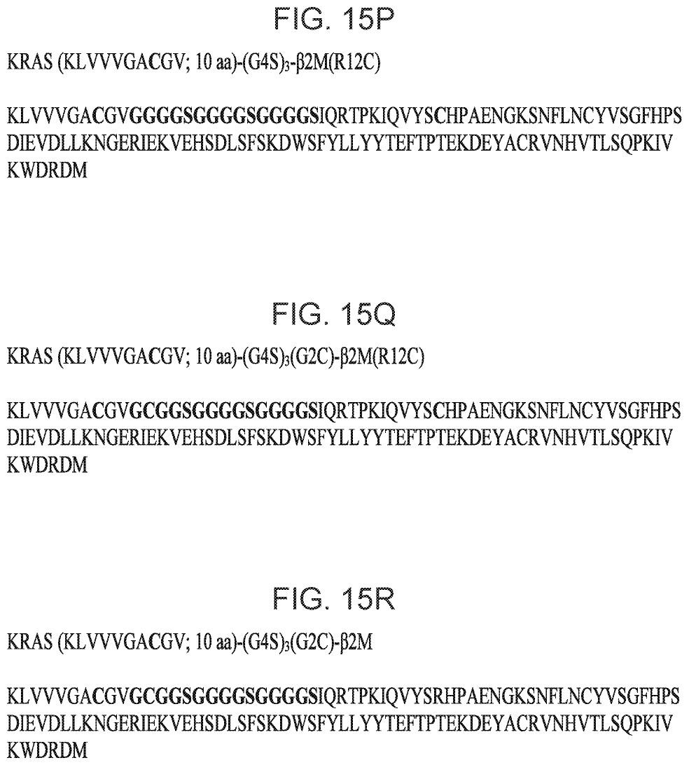

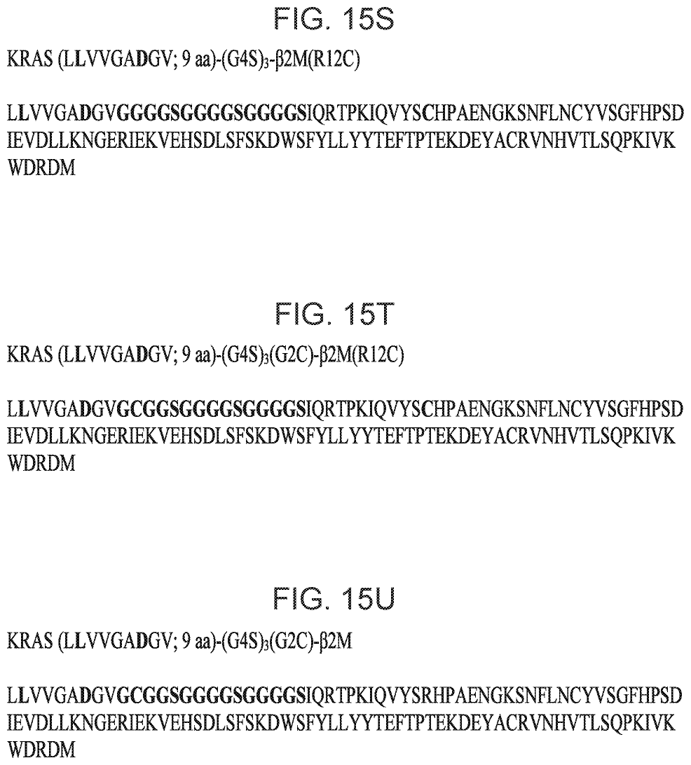

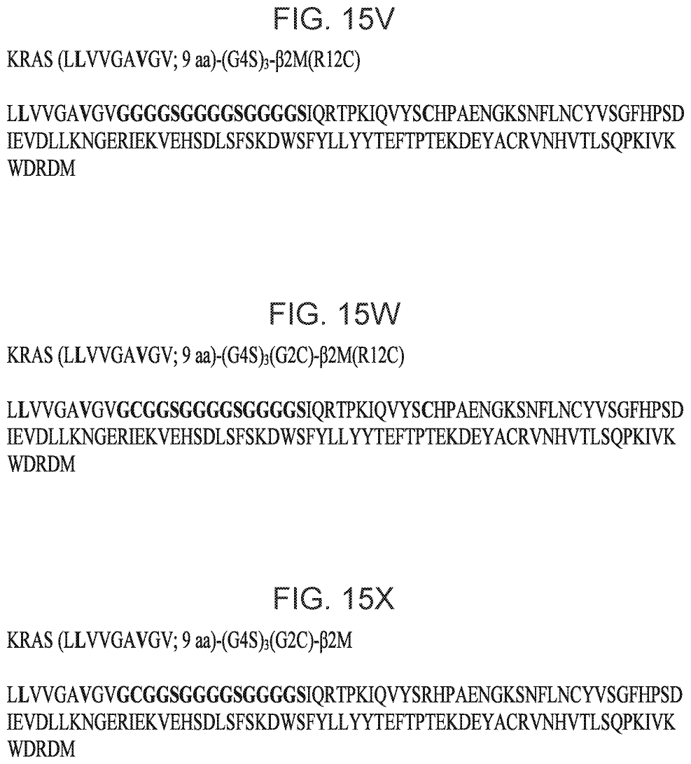

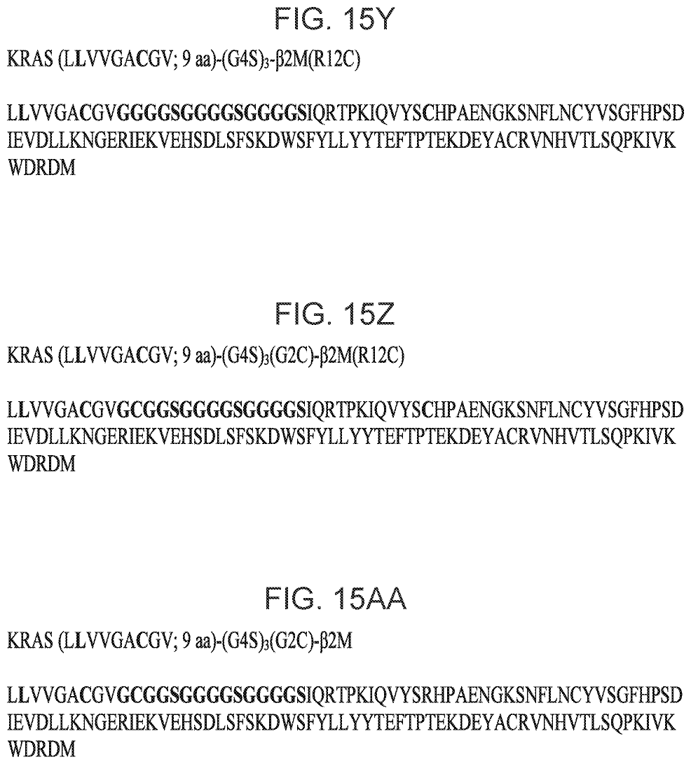

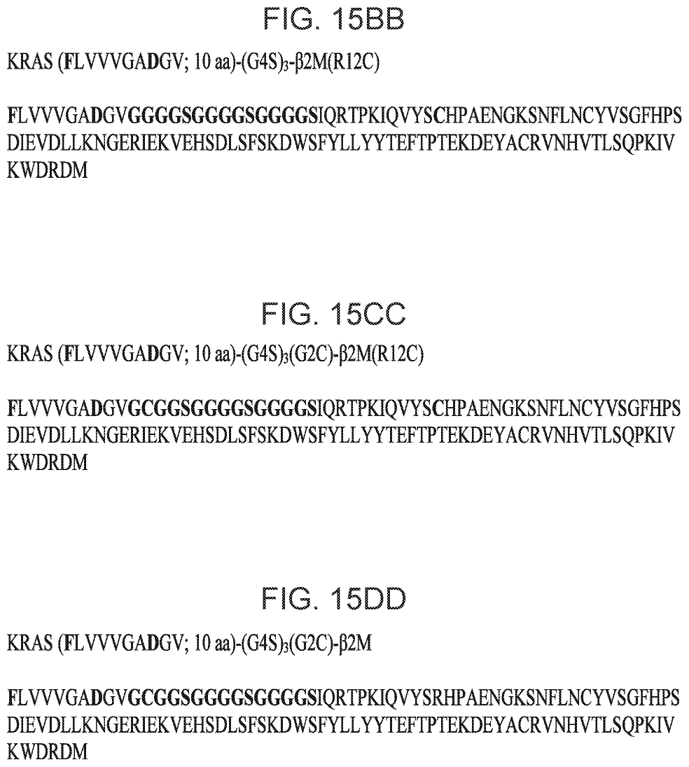

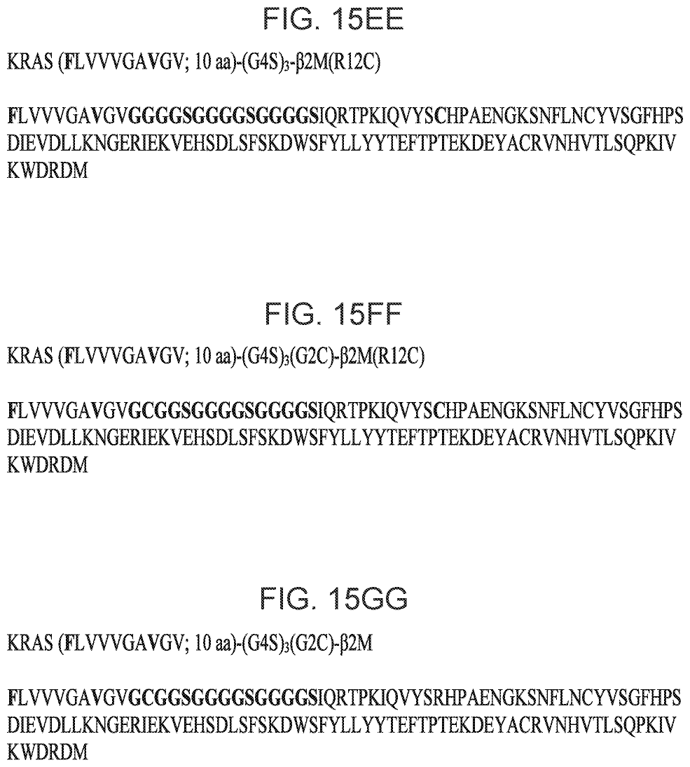

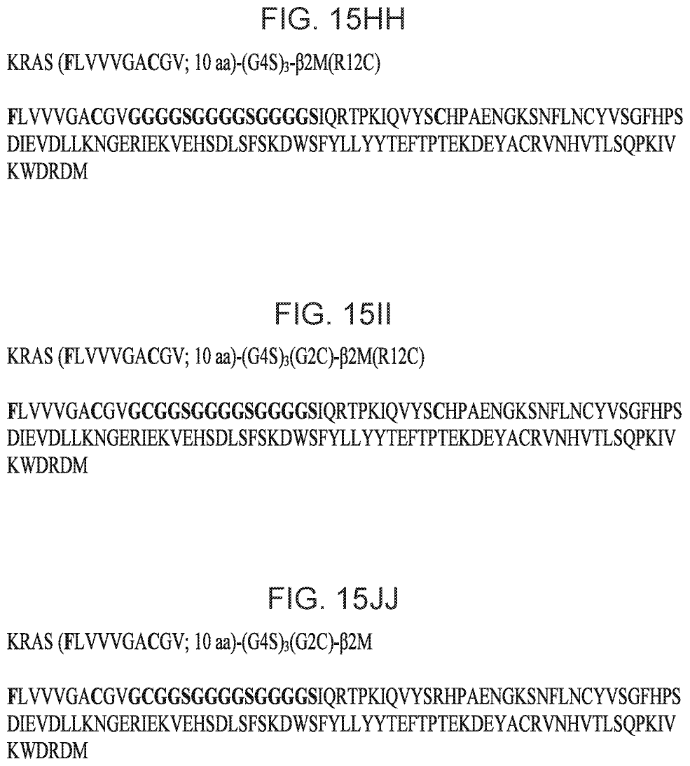

TABLE 3

| First Polypeptide | Second Polypeptide |
|---|---|
| FIG. 15A | FIG. 13A, 13B or 13C |
| FIG. 15B | FIG. 13A, 13B or 13C |
| FIG. 15C | FIG. 13A, 13B or 13C |
| FIG. 15D | FIG. 13A, 13B or 13C |
| FIG. 15E | FIG. 13A, 13B or 13C |
| FIG. 15F | FIG. 13A, 13B or 13C |
| FIG. 15G | FIG. 13A, 13B or 13C |
| FIG. 15H | FIG. 13A, 13B or 13C |
| FIG. 15I | FIG. 13A, 13B or 13C |
| FIG. 15J | FIG. 13A, 13B or 13C |
| FIG. 15K | FIG. 13A, 13B or 13C |
| FIG. 15L | FIG. 13A, 13B or 13C |
| FIG. 15M | FIG. 13A, 13B or 13C |
| FIG. 15N | FIG. 13A, 13B or 13C |
| FIG. 15O | FIG. 13A, 13B or 13C |
| FIG. 15P | FIG. 13A, 13B or 13C |
| FIG. 15Q | FIG. 13A, 13B or 13C |
| FIG. 15R | FIG. 13A, 13B or 13C |
| FIG. 15S | FIG. 13A, 13B or 13C |
| FIG. 15T | FIG. 13A, 13B or 13C |
| FIG. 15U | FIG. 13A, 13B or 13C |
| FIG. 15V | FIG. 13A, 13B or 13C |
| FIG. 15W | FIG. 13A, 13B or 13C |
| FIG. 15X | FIG. 13A, 13B or 13C |
| FIG. 15Y | FIG. 13A, 13B or 13C |
| FIG. 15Z | FIG. 13A, 13B or 13C |
| FIG. 15AA | FIG. 13A, 13B or 13C |
| FIG. 15BB | FIG. 13A, 13B or 13C |
| FIG. 15CC | FIG. 13A, 13B or 13C |
| FIG. 15DD | FIG. 13A, 13B or 13C |
| FIG. 15EE | FIG. 13A, 13B or 13C |
| FIG. 15FF | FIG. 13A, 13B or 13C |
| FIG. 15GG | FIG. 13A, 13B or 13C |
| FIG. 15HH | FIG. 13A, 13B or 13C |
| FIG. 15II | FIG. 13A, 13B or 13C |
| FIG. 15JJ | FIG. 13A, 13B or 13C |

In some cases, a heterodimeric TMP of the present disclosure comprises a class I MHC heavy chain that comprises an intrachain disulfide bond. For example, in some cases, a heterodimeric TMP of the present disclosure comprises a class I MHC heavy chain that comprises an intrachain disulfide bond formed between Cys residues resulting from Y84C and A139C substitutions. In some cases, such a heterodimeric TMP also comprises a class I MHC heavy chain that comprises an A236C substitution, where the Cys-236 can form a disulfide bond with a second polypeptide chain that comprises: i) a peptide epitope; ii) a β2M polypeptide comprising an R12C substitution, such that the Cys-12 forms a disulfide bond with the Cys-236 in the class I MHC heavy chain; and iii) a peptide linker between the peptide epitope and the β2M polypeptide, where the linker comprises the amino acid sequence (GGGGS)n, where n is an integer from 1 to 9 (e.g., n is 1, 2, or 3).

As examples, a TMP of the present disclosure can comprise one of the combinations of first and second polypeptides, set out in Table 4, below, to provide heterodimeric TMPs comprising a class I MHC A02 heavy allele with Y84C, A139C, and A236C substitutions; and where the immunomodulatory polypeptides are at position 1 or position 3 as depicted in FIG. 17.

TABLE 4

| | Second | Second |
|---|---|---|
| | Polypeptide | Polypeptide |
| | (MOD | (MOD |
| First Polypeptide | Position 1) | Position 3) |
| FIG. 14A or 15A | FIG. 13K | FIG. 13J |
| FIG. 14D or 15D | FIG. 13K | FIG. 13J |
| FIG. 14G or 15G | FIG. 13K | FIG. 13J |
| FIG. 14J or 15J | FIG. 13K | FIG. 13J |
| FIG. 14M or 15M | FIG. 13K | FIG. 13J |
| FIG. 14P or 15P | FIG. 13K | FIG. 13J |
| FIG. 14S or 15S | FIG. 13K | FIG. 13J |
| FIG. 14V or 15V | FIG. 13K | FIG. 13J |
| FIG. 14Y or 15Y | FIG. 13K | FIG. 13J |
| FIG. 14BB or 15BB | FIG. 13K | FIG. 13J |
| FIG. 14EE or 15EE | FIG. 13K | FIG. 13J |
| FIG. 14HH or 15HH | FIG. 13K | FIG. 13J |

As another example, a heterodimeric TMP of the present disclosure can comprise: a) a first polypeptide comprising the amino acid sequence depicted in FIG. 13L; and a second polypeptide comprises the amino acid sequence depicted in FIG. 14K. As another example, a heterodimeric TMP of the present disclosure can comprise: a) a first polypeptide comprising the amino acid sequence depicted in FIG. 13L; and a second polypeptide comprises the amino acid sequence depicted in FIG. 14Q.

As noted above, in some cases, a TMP of the present disclosure is a heterodimeric TMP comprising a first polypeptide and a second polypeptide, where the first polypeptide and the second polypeptide are linked by one or more disulfide bonds, e.g., a single disulfide bond or two disulfide bonds. For example, as discussed above, a TMP of the present disclosure can include: a) a G2C/Y84C disulfide bond and not an R12C/A236C disulfide bond; b) an R12C/A236C disulfide bond and not a G2C/Y84C disulfide bond; or c) a G2C/Y84C disulfide bond and an R12C/A236C disulfide bond. In some cases, e.g., where a TMP of the present disclosure includes a G2C/Y84C disulfide bond and not an R12C/A236C disulfide bond, the β2M polypeptide does not include an R12C substitution (and instead has an Arg at position 12) and the class I MHC heavy chain polypeptide does not include an A236C substitution (and instead has an Ala at position 236); in other words, the β2M polypeptide and the class I MHC heavy chain polypeptide do not include “free” (unpaired) Cys residues at position 12 of the β2M and at position 236 of the class I MHC heavy chain polypeptide. Similarly, in some cases, e.g., where a TMP of the present disclosure includes an R12C/A236C disulfide bond and not a G2C/Y84C disulfide bond, the linker between the peptide epitope and the β2M polypeptide does not include a Cys substitution (and instead, the linker is a (GGGGS)n linker, where n is an integer from 1 to 5) and the class I MHC polypeptide does not include a Y84C substitution (and instead has a Tyr at position 84); in other words, neither the linker between the peptide epitope and the β2M polypeptide nor the class I MHC polypeptide includes a “free” (unpaired) Cys residue in the linker or at position 84 of the class I MHC polypeptide.

### Single-Chain TMPs

As noted above and as depicted schematically in FIG. 18, an immunomodulatory polypeptide (i.e., one or more immunomodulatory polypeptides) can be present in a single-chain TMP of the present disclosure at any of a variety of positions. FIG. 18 depicts the position of two copies of a variant IL-2 polypeptide; however, the immunomodulatory polypeptide can be any number of and any of a variety of immunomodulatory polypeptides, as described herein. As depicted in FIG. 18, an immunomodulatory polypeptide can be: 1) C-terminal to the MHC class I heavy chain and N-terminal to the Ig Fc polypeptide; 2) C-terminal to the Ig Fc polypeptide; or 3) N-terminal to the peptide epitope.

The MHC class I polypeptides (i.e., the β2M and heavy chain polypeptides), immunomodulatory polypeptides, Ig Fc components and linkers of a single-chain TMP according to this disclosure are identical to those described above for the heterodimeric TMPs. Further, the single-chain TMPs can include the same disulfide bonds as in the heterodimeric TMPs, namely, intrachain in the MHC class I heavy chain polypeptide, e.g., between two Cys residues as discussed above (e.g., between the Cys-84 and the Cys-139 of the heavy chain), interchain between the β2M and heavy chain polypeptides (e.g., between R12C of the β2M and a Cys at residue 236 of the heavy chain), and/or a disulfide that joins a Cys in the MHC class I heavy chain to a Cys residue in the linker between the epitope and β2M polypeptide. In some cases, the MHC class I heavy chain is an A02 allele MHC class I heavy chain. In some cases, the MHC class I heavy chain comprises a Y84C and an A139C substitution, such that an intrachain disulfide bond forms between the Cys-84 and the Cys-139. In some cases, the MHC class I heavy chain is an A02 allele MHC class I heavy chain.

In some cases, a single-chain TMP of the present disclosure comprises a scaffold with a MOD Position 2 or Position 3 arrangement as shown in FIG. 18, wherein the HLA heavy chain polypeptide is a wild-type or variant HLA-A*0201 polypeptide, e.g., an HLA-A*0201 polypeptide comprising an A236C substitution, or a sequence as shown in FIG. 22C, or a variant thereof. In some cases, the Ig Fc polypeptide is a human IgG1 Fc polypeptide that substantially does not cause cell lysis, e.g., a human IgG1 Fc polypeptide comprising L234A and L235A substitutions as shown in FIG. 3G. In some cases, the single-chain TMP comprises an intrachain disulfide bond (i) between two Cys residues in the MHC class I heavy chain polypeptide, (ii) between the β2M and heavy chain polypeptides, and/or between a Cys in the MHC class I heavy chain and a Cys in the linker between the KRAS epitope and β2M polypeptide. In some cases, the immunomodulatory polypeptide is a variant IL-2 polypeptide comprising H16A and F42A substitutions or H16T and F42A substitutions. In some cases, the KRAS peptide has an amino acid sequence selected from the following group: VVGADGVGK (SEQ ID NO:176); VVGACGVGK (SEQ ID NO:177); VVGAVGVGK (SEQ ID NO:178); VVVGADGVGK (SEQ ID NO:179); VVVGAVGVGK (SEQ ID NO:180); VVVGACGVGK (SEQ ID NO:181); VTGADGVGK (SEQ ID NO:182); VTGAVGVGK (SEQ ID NO:183); VTGACGVGK (SEQ ID NO:184); VTVGADGVGK (SEQ ID NO:185); VTVGAVGVGK (SEQ ID NO:186); VTVGACGVGK (SEQ ID NO:187); LVVVGADGV (SEQ ID NO:192); LVVVGAVGV (SEQ ID NO:193); LVVVGACGV (SEQ ID NO:194); KLVVVGADGV (SEQ ID NO:195); KLVVVGAVGV (SEQ ID NO:196); KLVVVGACGV (SEQ ID NO:197); LLVVGADGV (SEQ ID NO:198); LLVVGAVGV (SEQ ID NO:199); LLVVGACGV (SEQ ID NO:200); FLVVVGADGV (SEQ ID NO:201); FLVVVGAVGV (SEQ ID NO:202); FLVVVGACGV (SEQ ID NO:203).

In some cases, a single-chain TMP of the present disclosure comprises a scaffold with a MOD Position 2 or Position 3 arrangement as shown in FIG. 18, wherein the HLA heavy chain polypeptide is a wild-type or variant HLA-A24 polypeptide (also referred to as HLA-A*2402), e.g., an HLA-A*0201 polypeptide comprising an A236C substitution or an amino acid sequence shown in any one of FIG. 21O, 21P, 21Q, 21R, 21S or 21T, or a variant thereof. In some cases, the Ig Fc polypeptide is a human IgG1 Fc polypeptide that substantially does not cause cell lysis, e.g., a human IgG1 Fc polypeptide comprising L234A and L235A substitutions as shown in FIG. 3G. In some cases, the single-chain TMP comprises an intrachain disulfide bond (i) between two Cys residues in the MHC class I heavy chain polypeptide, (ii) between the β2M and heavy chain polypeptides, and/or between a Cys in the MHC class I heavy chain and a Cys in the linker between the KRAS epitope and β2M polypeptide. In some cases, the immunomodulatory polypeptide is a variant IL-2 polypeptide comprising H16A and F42A substitutions or H16T and F42A substitutions. In some cases, the KRAS peptide has an amino acid sequence selected from the following group: VVGADGVGK (SEQ ID NO:176); VVGACGVGK (SEQ ID NO:177); VVGAVGVGK (SEQ ID NO:178); VVVGADGVGK (SEQ ID NO:179); VVVGAVGVGK (SEQ ID NO:180); VVVGACGVGK (SEQ ID NO:181); VTGADGVGK (SEQ ID NO:182); VTGAVGVGK (SEQ ID NO:183); VTGACGVGK (SEQ ID NO:184); VTVGADGVGK (SEQ ID NO:185); VTVGAVGVGK (SEQ ID NO:186); VTVGACGVGK (SEQ ID NO:187); LVVVGADGV (SEQ ID NO:192); LVVVGAVGV (SEQ ID NO:193); LVVVGACGV (SEQ ID NO:194); KLVVVGADGV (SEQ ID NO:195); KLVVVGAVGV (SEQ ID NO:196); KLVVVGACGV (SEQ ID NO:197); LLVVGADGV (SEQ ID NO:198); LLVVGAVGV (SEQ ID NO:199); LLVVGACGV (SEQ ID NO:200); FLVVVGADGV (SEQ ID NO:201); FLVVVGAVGV (SEQ ID NO:202); FLVVVGACGV (SEQ ID NO:203).

In some cases, a TMP of the present disclosure comprises a scaffold with a MOD Position 2 or Position 3 arrangement as shown in FIG. 18, wherein the HLA heavy chain polypeptide is a wild-type or variant HLA-A*1101 polypeptide as disclosed herein, e.g. an HLA-A*1101 polypeptide comprising an A236C substitution or having an amino acid sequence as shown in one of FIG. 21J or 21K, or a variant thereof. In some cases, the Ig Fc polypeptide is a human IgG1 Fc polypeptide that substantially does not cause cell lysis, e.g., a human IgG1 Fc polypeptide comprising L234A and L235A substitutions as shown in FIG. 3G. In some cases, the single-chain TMP comprises an intrachain disulfide bond (i) between two Cys residues in the MHC class I heavy chain polypeptide, (ii) between the β2M and heavy chain polypeptides, and/or between a Cys in the MHC class I heavy chain and a Cys in the linker between the KRAS epitope and β2M polypeptide. In some cases, the immunomodulatory polypeptide is a variant IL-2 polypeptide comprising H16A and F42A substitutions or H16T and F42A substitutions. In some cases, the KRAS peptide has an amino acid sequence selected from the following group: VVGADGVGK (SEQ ID NO:176); VVGACGVGK (SEQ ID NO:177); VVGAVGVGK (SEQ ID NO:178); VVVGADGVGK (SEQ ID NO:179); VVVGAVGVGK (SEQ ID NO:180); VVVGACGVGK (SEQ ID NO:181); VTGADGVGK (SEQ ID NO:182); VTGAVGVGK (SEQ ID NO:183); VTGACGVGK (SEQ ID NO:184); VTVGADGVGK (SEQ ID NO:185); VTVGAVGVGK (SEQ ID NO:186); VTVGACGVGK (SEQ ID NO:187); LVVVGADGV (SEQ ID NO:192); LVVVGAVGV (SEQ ID NO:193); LVVVGACGV (SEQ ID NO:194); KLVVVGADGV (SEQ ID NO:195); KLVVVGAVGV (SEQ ID NO:196); KLVVVGACGV (SEQ ID NO:197); LLVVGADGV (SEQ ID NO:198); LLVVGAVGV (SEQ ID NO:199); LLVVGACGV (SEQ ID NO:200); FLVVVGADGV (SEQ ID NO:201); FLVVVGAVGV (SEQ ID NO:202); FLVVVGACGV (SEQ ID NO:203).

As discussed above, the polypeptides of a single-chain TMP can be linked by one or more disulfide bonds. For example, a TMP of the present disclosure can comprise a β2M polypeptide having an R12C substitution and a class I MHC heavy chain polypeptide having an A236C substitution; such that a disulfide bond forms between the Cys at position 12 of the β2M polypeptide and the Cys at position 236 of the class I MHC heavy chain polypeptide. As another example, a single-chain TMP of the present disclosure can comprise i) a KRAS epitope and a β2M polypeptide that are joined by a peptide linker comprising a GCGGS(GGGGS)ₙ (SEQ ID NO:582) sequence, where n is 1, 2, or 3, and ii) a class I MHC heavy chain polypeptide having a Y84C substitution, such that a disulfide bond forms between the Cys in the peptide linker and the Cys at position 84 of the class I MHC heavy chain polypeptide. In other examples, a single-chain TMP of the present disclosure can comprise i) a KRAS epitope and a β2M polypeptide that are joined by a peptide linker comprising a GCGGS(GGGGS)˜(SEQ ID NO:582) sequence, where n is 1, 2, or 3, and where the β2M polypeptide comprises an R12C substitution; and ii) a class I MHC heavy chain polypeptide having a Y84C substitution and an A236C substitution, such that a) a first disulfide bond forms between the Cys in the peptide linker and the Cys at position 84 of the class I MHC heavy chain polypeptide, and b) a second disulfide bond forms between the Cys at position 12 of the β2M polypeptide and the Cys at position 236 of the class I MHC heavy chain polypeptide. For simplicity, the first disulfide bond is referred to as “G2C/Y84C”; and the second disulfide bond is referred to as “R12C/A236C.” A single-chain TMP of the present disclosure can include: a) a G2C/Y84C disulfide bond and not an R12C/A236C disulfide bond; b) an R12C/A236C disulfide bond and not a G2C/Y84C disulfide bond; or c) a G2C/Y84C disulfide bond and an R12C/A236C disulfide bond. In some cases, the MHC class I heavy chain comprises a non-naturally occurring Cys at position 84 and a non-naturally occurring residue at position 139, such that an intrachain disulfide bond forms between the Cys-84 and the Cys-139.

A single-chain TMP of the present disclosure can include: a) a G2C/Y84C disulfide bond and not an R12C/A236C disulfide bond; and b) at least one immunomodulatory polypeptide at position 2 or 3. A single-chain TMP of the present disclosure can include: a) an R12C/A236C disulfide bond and not a G2C/Y84C disulfide bond; and at least one immunomodulatory polypeptide at position 2 or 3. A single-chain TMP of the present disclosure can include: a) a G2C/Y84C disulfide bond and an R12C/A236C disulfide bond; and b) and at least one immunomodulatory polypeptide at position 2 or 3.

In some cases, a single-chain TMP of the present disclosure comprises a MHC class I heavy chain polypeptide comprising (i) an HLA-A0201 (Y84A; A236C) polypeptide comprising an Ala at position 84 and a Cys at position 236, or (ii) an HLA-A0201 (Y84C; A139C) polypeptide comprising a Cys at positions 84 and 139, or (iii) an HLA-A0201 (Y84C; A236) polypeptide comprising a Cys at position 84 and an alanine at position 236, e.g., as depicted in FIG. 13A, 13B or 13C, respectively.

In some cases, a single-chain TMP of the present disclosure comprises a MHC class I heavy chain polypeptide comprising (i) an HLA-A*1101 (Y84A; A236C) polypeptide comprising an Ala at position 84 and a Cys at position 236, or (ii) an HLA-A*1101 (Y84C; A236C) polypeptide comprising a Cys at positions 84 and 236, or (iii) an HLA-A*1101 (Y84C; A236) polypeptide comprising a Cys at position 84 and an alanine at position 236, e.g., as depicted in FIG. 13D, FIG. 13E, or FIG. 13F, respectively.

In some cases, a TMP of the present disclosure comprises a second polypeptide comprising (i) an HLA-A24 (Y84A; A236C) polypeptide comprising an Ala at position 84 and a Cys at position 236, or (ii) an HLA-A24 (Y84C; A236C) polypeptide comprising a Cys at positions 84 and 236, or (iii) an HLA-A24 (Y84C; A236) polypeptide comprising a Cys at position 84 and an alanine at position 236, e.g., as depicted in FIG. 13G, FIG. 13H or FIG. 13I, respectively.

As one non-limiting example, a single-chain TMP of the present disclosure can comprise the amino acid sequence depicted in FIG. 19A (TMP “4095”). As another non-limiting example, a single-chain TMP of the present disclosure can comprise the amino acid sequence depicted in FIG. 19B (TMP “4073”). As another non-limiting example, a single-chain TMP of the present disclosure can comprise the amino acid sequence depicted in FIG. 19C (TMP “4074”). As another non-limiting example, a single-chain TMP of the present disclosure can comprise the amino acid sequence depicted in FIG. 19D (TMP “4333”). As another non-limiting example, a single-chain TMP of the present disclosure can comprise the amino acid sequence depicted in FIG. 19E (TMP “4335”). As another non-limiting example, a single-chain TMP of the present disclosure can comprise the amino acid sequence depicted in FIG. 19F. As another non-limiting example, a single-chain TMP of the present disclosure can comprise the amino acid sequence depicted in FIG. 19G (TMP “4238”). As another non-limiting example, a single-chain TMP of the present disclosure can comprise the amino acid sequence depicted in FIG. 19H (TMP “4241”). As another non-limiting example, a single-chain TMP of the present disclosure can comprise the amino acid sequence depicted in FIG. 19I (TMP “4334”). As another non-limiting example, a single-chain TMP of the present disclosure can comprise the amino acid sequence depicted in FIG. 19J (TMP “4144”). As another non-limiting example, a single-chain TMP of the present disclosure can comprise the amino acid sequence depicted in FIG. 19K (TMP “4145”). As another non-limiting example, a single-chain TMP of the present disclosure can comprise the amino acid sequence depicted in FIG. 19L (TMP “4146”).

### Methods of Generating a Multimeric T-Cell Modulatory Polypeptide

Methods of obtaining a TMP comprising one or more variant immunomodulatory polypeptides that exhibit lower affinity for a cognate costimulatory polypeptide compared to the affinity of the corresponding parental wild-type immunomodulatory polypeptide for the costimulatory polypeptide are disclosed in published PCT application applications WO2020132138A1 and WO2019/051091, discussed above, the disclosures of which as they pertain to methods of generating TMPs are expressly incorporated herein by reference, including specifically paragraphs [00560]-[00583] of WO2020132138A1 and paragraphs [00364]-[00387] of WO2019/051091.

### Nucleic Acids

The present disclosure provides a nucleic acid comprising a nucleotide sequence encoding a TMP of the present disclosure. The present disclosure provides a nucleic acid comprising a nucleotide sequence encoding a TMP of the present disclosure.

The present disclosure provides nucleic acids comprising nucleotide sequences encoding a TMP of the present disclosure. In some cases, the individual polypeptide chains of a heterodimeric TMP of the present disclosure are encoded in separate nucleic acids. In some cases, all polypeptide chains of a heterodimeric or single-chain TMP of the present disclosure are encoded in a single nucleic acid. In some cases, a first nucleic acid comprises a nucleotide sequence encoding a first polypeptide of a heterodimeric TMP of the present disclosure; and a second nucleic acid comprises a nucleotide sequence encoding a second polypeptide of a heterodimeric TMP of the present disclosure. In some cases, single nucleic acid comprises a nucleotide sequence encoding a first polypeptide of a TMP of the present disclosure and a second polypeptide of a heterodimeric TMP of the present disclosure.

### Separate Nucleic Acids Encoding Individual Polypeptide Chains of a Polypeptide

TMPAs noted above, in some cases, the individual polypeptide chains of a heterodimeric TMP of the present disclosure are encoded in separate nucleic acids. In some cases, nucleotide sequences encoding the separate polypeptide chains of a TMP of the present disclosure are operably linked to transcriptional control elements, e.g., promoters, such as promoters that are functional in a eukaryotic cell, where the promoter can be a constitutive promoter or an inducible promoter.

Thus, for example, the present disclosure provides a first nucleic acid and a second nucleic acid, where the first nucleic acid comprises separate nucleotide sequences encoding the first polypeptide of a heterodimeric TMP and the second nucleic acid comprises the separate nucleotide sequences encoding the second polypeptide of the heterodimeric TMP of the present disclosure. For example, in MOD position 1 discussed above for heterodimeric TMPs (see FIG. 17), where the first MHC polypeptide comprises a β2M polypeptide and the second polypeptide comprises an HLA heavy chain polypeptide, the first nucleic acid encodes a first polypeptide comprising, in order from N-terminus to C-terminus: i) a KRAS peptide; and ii) a β2M polypeptide, and the second nucleic acid encodes a second polypeptide comprising, in order from N-terminus to C-terminus: i) at least one immunomodulatory polypeptide; ii) an HLA heavy chain polypeptide; and iii) an Ig Fc polypeptide. As noted above, linkers optionally may be included between the individual components of both the first and second polypeptides. Similarly, e.g., for MOD position 3, the first nucleic acid encodes a first polypeptide comprising, in order from N-terminus to C-terminus: i) a KRAS peptide; and ii) a β2M polypeptide, and the second nucleic acid encodes a second polypeptide comprising, in order from N-terminus to C-terminus: i) an HLA heavy chain polypeptide; ii) an Ig Fc polypeptide, and iii) i) at least one immunomodulatory polypeptide. Again, linkers optionally may be included between the individual components of both the first and second polypeptides.

In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Nucleic Acid Encoding Two or More Polypeptides Present in a Polypeptide

The present disclosure also provides a single nucleic acid comprising nucleotide sequences encoding at least the first polypeptide and the second polypeptide of a heterodimeric or single-chain TMP of the present disclosure. TMP Methods for preparing heterodimeric TMPs using a single nucleic acid are disclosed in published PCT application WO2020132138A1 and WO2019/051091, the disclosures of which as they pertain to nucleic acids encoding TMPs is expressly incorporated herein by reference, including specifically paragraphs [00507]-[00514] of WO2020132138A1 and paragraphs [00393]-[00400] of WO2019/051091.

### Recombinant Expression Vectors

The present disclosure provides recombinant expression vectors comprising nucleic acids of the present disclosure. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors are disclosed in published PCT application WO2020132138A1 and WO2019/051091, the disclosures of which as they pertain to such expression vectors are expressly incorporated herein by reference, including specifically paragraphs [00515]-[00520] of WO2020132138A1 and paragraphs [00401]-[00406] of WO2019/051091.

### Genetically Modified Host Cells

The present disclosure provides a genetically modified host cell, where the host cell is genetically modified with a nucleic acid of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC 02-M.

In some cases, the host cell is a mammalian cell that has been genetically modified such that it does not synthesize endogenous MHC class I heavy chain. In some cases, the host cell is a mammalian cell that has been genetically modified such that it does not synthesize endogenous MHC 02-M and such that it does not synthesize endogenous MHC class I heavy chain.

As noted above, a TMP is in some cases is a single-chain polypeptide (e.g., consists of a single polypeptide chain; or is a homodimer of a single polypeptide chain). It was observed that single-chain TMPs are produced intact and full-length, i.e., without cleavage of the polypeptide chain. A single-chain TMP can in some cases be produced in greater quantities than a heterodimeric TMP.

### Compositions

The present disclosure provides compositions, including pharmaceutical compositions, comprising a TMP (synTac) of the present disclosure. The present disclosure provides compositions, including pharmaceutical compositions, comprising a TMP of the present disclosure. The present disclosure provides compositions, including pharmaceutical compositions, comprising a nucleic acid or a recombinant expression vector of the present disclosure.

### Compositions Comprising a TMP

A composition of the present disclosure can comprise, in addition to a TMP of the present disclosure, one or more of: a salt, e.g., NaCl, MgCl₂, KCl, MgSO₄, etc.; a buffering agent, a solubilizing agent; a detergent, e.g., a non-ionic detergent such as Tween-20, etc.; a protease inhibitor; glycerol; and the like. The composition also may comprise a pharmaceutically acceptable excipient, a variety of which are known in the art and need not be discussed in detail herein. Examples of pharmaceutically acceptable salts, buffering agents, excipients, formulations, dosage forms, etc. are disclosed in published PCT applications WO2020132138A1 and WO2019/051091, the disclosures of which as they pertain to compositions comprising TMPs of this disclosure are expressly incorporated herein by reference, including specifically paragraphs [00526]-[00536] of WO2020132138A1 and paragraphs [00412]-[00422] of WO2019/051091.

Where a TMP of the present disclosure is administered as an injectable (e.g. subcutaneously, intraperitoneally, intramuscularly, and/or intravenously) directly into a tissue, a formulation can be provided as a ready-to-use dosage form that may be directly injected or infused into the patient or admixed with a saline solution for infusion, or possibly as a non-aqueous form (e.g. a reconstitutable storage-stable powder) or aqueous form, such as liquid composed of pharmaceutically acceptable carriers and excipients. Formulations may also be provided so as to enhance serum half-life of the TMP following administration. For example, the TMP may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

The concentration of a TMP of the present disclosure in a liquid composition formulation can vary widely (e.g., from less than about 0.1%, usually at or at least about 2% to as much as 20% to 50% or more by weight). Included within this range is a concentration of from about 5 to about 15 mg/mL, including about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL and about 15 mg/mL, The concentration may depend on numerous factors, including the stability of the TMP in the liquid composition.

In some cases, a TMP of the present disclosure is present in a liquid composition. TMP In some cases, a composition of the present disclosure comprises: a) a TMP of the present disclosure; and b) saline (e.g., 0.9% NaCl). In some cases, the composition is sterile and suitable for administration to a human subject.

### Compositions Comprising a Nucleic Acid or a Recombinant Expression Vector

The present disclosure provides compositions, e.g., pharmaceutical compositions, comprising a nucleic acid or a recombinant expression vector of the present disclosure. Published PCT applications WO2020132138A1 and WO2019/051091 disclose how to prepare such compositions. See, paragraphs [00537]-[00546] of WO2020132138A1 and paragraphs [00423]-[00432] of WO2019/051091, the disclosures of which are expressly incorporated herein by reference.

### Methods of Modulating T Cell Activity

The present disclosure provides a method of selectively modulating the activity of an epitope-specific T cell (e.g., a T cell specific for a KRAS epitope, such as a KRAS peptide comprising a cancer-associated mutation), the method comprising contacting the T cell with a TMP of the present disclosure, where contacting the T cell with a TMP of the present disclosure selectively modulates the activity of the epitope-specific T cell. In some cases, the contacting occurs in vitro. In some cases, the contacting occurs in vivo.

Where a TMP of the present disclosure includes an immunomodulatory polypeptide that is an activating polypeptide, contacting the T cell with the TMP activates the epitope-specific T cell. In some instances, the epitope-specific T cell is a T cell that is specific for an epitope present on a cancer cell, and contacting the epitope-specific T cell with the TMP increases cytotoxic activity of the T cell toward the cancer cell and/or increases the number of the epitope-specific T cells.

The present disclosure provides a method of modulating an immune response in an individual, the method comprising administering to the individual an effective amount of a TMP of the present disclosure. Administering the TMP induces an epitope-specific T cell response (e.g., cancer epitope-specific T-cell response) and an epitope-non-specific T cell response, where the ratio of the epitope-specific T cell response to the epitope-non-specific T cell response is at least 2:1. In some cases, the ratio of the epitope-specific T cell response to the epitope-non-specific T cell response is at least 5:1. In some cases, the ratio of the epitope-specific T cell response to the epitope-non-specific T cell response is at least 10:1. In some cases, the ratio of the epitope-specific T cell response to the epitope-non-specific T cell response is at least 25:1. In some cases, the ratio of the epitope-specific T cell response to the epitope-non-specific T cell response is at least 50:1. In some cases, the ratio of the epitope-specific T cell response to the epitope-non-specific T cell response is at least 100:1. In some cases, the individual is a human. In some cases, the modulating increases a cytotoxic T-cell response to a cancer cell, e.g., a cancer cell expressing an antigen that displays the same epitope displayed by the KRAS epitope present in the TMP and/or increases the number of T cells specific for the KRAS epitope. In some cases, the administering is intravenous, subcutaneous, intramuscular, systemic, intralymphatic, distal to a treatment site, local, or at or near a treatment site.

The present disclosure provides a method of delivering an immunomodulatory polypeptide selectively to target T cell, the method comprising contacting a mixed population of T cells with a TMP of the present disclosure, where the mixed population of T cells comprises the target T cell and non-target T cells, where the target T cell is specific for the epitope present within the TMP (e.g., where the target T cell is specific for the epitope present within the TMP), and where the contacting step delivers the one or more immunomodulatory polypeptides present within the TMP to the target T cell. In some cases, the population of T cells is in vitro. In some cases, the population of T cells is in vivo in an individual. In some cases, the method comprises administering the TMP to the individual. In some case, the T cell is a cytotoxic T cell. In some cases, the mixed population of T cells is an in vitro population of mixed T cells obtained from an individual, and the contacting step results in activation and/or proliferation of the target T cell, generating a population of activated and/or proliferated target T cells; in some of these instances, the method further comprises administering the population of activated and/or proliferated target T cells to the individual.

The present disclosure provides a method of detecting, in a mixed population of T cells obtained from an individual, the presence of a target T cell that binds an epitope of interest (e.g., a cancer epitope; a KRAS peptide comprising a cancer-associated mutation), the method comprising: a) contacting in vitro the mixed population of T cells with a TMP of the present disclosure, wherein the TMP comprises the KRAS epitope of interest; and b) detecting activation and/or proliferation of T cells in response to said contacting, wherein activated and/or proliferated T cells indicates the presence of the target T cell.

### Treatment Methods

The present disclosure provides a method of treatment of an individual, the method comprising administering to the individual an amount of a TMP of the present disclosure, or one or more nucleic acids encoding the TMP, effective to treat the individual. Also provided is a TMP of the present disclosure for use in a method of treatment of the human or non-human animal body. In some cases, a treatment method of the present disclosure comprises administering to an individual in need thereof one or more recombinant expression vectors comprising nucleotide sequences encoding a TMP of the present disclosure. In some cases, a treatment method of the present disclosure comprises administering to an individual in need thereof one or more mRNA molecules comprising nucleotide sequences encoding a TMP of the present disclosure. In some cases, a treatment method of the present disclosure comprises administering to an individual in need thereof a TMP of the present disclosure. Conditions that can be treated include, e.g., cancer, such as a cancer that expresses a KRAS polypeptide, e.g., a mutant KRAS polypeptide, as described above.

In some cases, a TMP of the present disclosure, when administered to an individual in need thereof, induces both an epitope-specific T cell response and an epitope non-specific T cell response. In other words, in some cases, a TMP of the present disclosure, when administered to an individual in need thereof, induces an epitope-specific T cell response by modulating the activity of a first T cell that displays both: i) a TCR specific for the epitope present in the TMP; ii) a costimulatory polypeptide that binds to the immunomodulatory polypeptide present in the TMP; and induces an epitope non-specific T cell response by modulating the activity of a second T cell that displays:i) a TCR specific for an epitope other than the epitope present in the TMP; and ii) a costimulatory polypeptide that binds to the immunomodulatory polypeptide present in the TMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. “Modulating the activity” of a T cell can include one or more of: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin; a granzyme; a granulysin) by a cytotoxic (e.g., CD8⁺) T cell; and iv) increasing the number of cytotoxic (e.g., CD8⁺) T cells.

Where a MOD is a reduced-affinity variant of a wild-type MOD, the combination of the reduced affinity of the MOD for its cognate costimulatory polypeptide, and the affinity of the epitope for a TCR, provides for enhanced selectivity of a TMP of the present disclosure. Thus, for example, a TMP of the present disclosure binds with higher avidity to a first T cell that displays both: i) a TCR specific for the epitope present in the TMP; and ii) a costimulatory polypeptide that binds to the immunomodulatory polypeptide present in the TMP, compared to the avidity to which it binds to a second T cell that displays:i) a TCR specific for an epitope other than the epitope present in the TMP; and ii) a costimulatory polypeptide that binds to the immunomodulatory polypeptide present in the TMP.

The present disclosure thus provides a method of selectively modulating the activity of an epitope-specific T cell in an individual, the method comprising administering to the individual an effective amount of a TMP of the present disclosure, or one or more nucleic acids (e.g., expression vectors; mRNA; etc.) comprising nucleotide sequences encoding the TMP, where the TMP selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a TMP of the present disclosure.

In some cases, the immunomodulatory polypeptide (“MOD”) is an activating polypeptide, and the TMP activates the epitope-specific T cell. In some cases, the TMP increases the activity of a T cell specific for the KRAS epitope. In some cases, the MOD is an activating polypeptide, and the TMP activates an epitope-specific T-cell (e.g., a T-cell specific for a KRAS epitope). In some cases, the T cells are T-helper cells (CD4⁺ cells), cytotoxic T-cells (CD8⁺ cells), or NK-T-cells. In some cases, the epitope is a cancer epitope, and the TMP increases the activity of a T-cell specific for a cancer cell expressing the KRAS cancer epitope (e.g., T-helper cells (CD4⁺ cells), cytotoxic T-cells (CD8⁺ cells), and/or NK-T-cells). Activation of CD4⁺ T cells can include increasing proliferation of CD4⁺ T cells and/or inducing or enhancing release cytokines by CD4⁺ T cells. Activation of NK-T-cells and/or CD8+ cells can include: increasing proliferation of NK-T-cells and/or CD8+ cells; and/or inducing release of cytokines such as interferon γ by NK-T-cells and/or CD8+ cells.

A TMP of the present disclosure can be administered to an individual in need thereof to treat a cancer in the individual, where the cancer expresses the KRAS peptide present in the TMP. For example, the cancer can be one in which the cancer cells express or over-express KRAS, e.g., a mutated form of KRAS, as described above. The present disclosure provides a method of treating cancer in an individual, the method comprising administering to the individual an effective amount of a TMP of the present disclosure, or one or more nucleic acids (e.g., expression vectors; mRNA; etc.) comprising nucleotide sequences encoding the TMP, where the TMP comprises a T-cell epitope that is a KRAS epitope, and where the TMP comprises a stimulatory immunomodulatory polypeptide. In some cases, an “effective amount” of a TMP of the present disclosure is an amount that, when administered in one or more doses to an individual in need thereof, reduces the number of cancer cells in the individual. For example, in some cases, an “effective amount” of a TMP of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the TMP, or in the absence of administration with the TMP. In some cases, an “effective amount” of a TMP of the present disclosure is an amount that, when administered in one or more doses to an individual in need thereof, reduces the number of cancer cells in the individual, including to substantially undetectable levels.

In some cases, an “effective amount” of a TMP of the present disclosure is an amount that, when administered in one or more doses to an individual in need thereof, reduces the tumor mass in the individual. For example, in some cases, an “effective amount” of a TMP of the present disclosure is an amount that, when administered in one or more doses to an individual in need thereof (an individual having a tumor), reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the TMP, or in the absence of administration with the TMP. In some cases, an “effective amount” of a TMP of the present disclosure is an amount that, when administered in one or more doses to an individual in need thereof (an individual having a tumor), reduces the tumor volume in the individual. For example, in some cases, an “effective amount” of a TMP of the present disclosure is an amount that, when administered in one or more doses to an individual in need thereof (an individual having a tumor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the TMP, or in the absence of administration with the TMP. In some cases, an “effective amount” of a TMP of the present disclosure is an amount that, when administered in one or more doses to an individual in need thereof, increases survival time of the individual. For example, in some cases, an “effective amount” of a TMP of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the TMP.

Cancers that can be treated with a method of the present disclosure include cancers in which the cancer cells express a mutated form of KRAS. Examples include adenocarcinomas and hematological malignancies. Examples of cancers that can be treated with a method of the present disclosure include multiple myeloma; B-cell lymphoma; breast cancer; lung cancer; ovarian carcinoma; pancreatic cancer; colorectal cancer; prostate cancer; renal cancer; acute myelogenous leukemia; mesothelioma; thyroid cancer; head and neck cancer; stomach cancer; urothelial cancer; cervical cancer; and ovarian endometrial cancer.

As noted above, in some cases, in carrying out a subject treatment method, a TMP of the present disclosure is administered to an individual in need thereof, as the TMP per se. In other instances, in carrying out a subject treatment method, one or more nucleic acids comprising nucleotide sequences encoding a TMP of the present disclosure is/are administering to an individual in need thereof. Thus, in other instances, one or more nucleic acids of the present disclosure, e.g., one or more recombinant expression vectors of the present disclosure, is/are administered to an individual in need thereof.

As discussed above, an APP of this disclosure also can be administered to a patient for therapeutic purposes in those instances where it is desired to engage the TCR of a T cell that is specific for the KRAS peptide of the APP. In such instances, the presence of a naturally occurring immunomodulatory polypeptide in the patient could effect modulation of the T cell when the APP is engaged with the TCR.

### Formulations

Suitable formulations are described above, where suitable formulations include a pharmaceutically acceptable excipient. In some cases, a suitable formulation comprises: a) a TMP of the present disclosure; and b) a pharmaceutically acceptable excipient. In some cases, a suitable formulation comprises: a) a nucleic acid comprising a nucleotide sequence encoding a TMP of the present disclosure; and b) a pharmaceutically acceptable excipient; in some instances, the nucleic acid is an mRNA. In some cases, a suitable formulation comprises: a) a first nucleic acid comprising a nucleotide sequence encoding the first polypeptide of a TMP of the present disclosure; b) a second nucleic acid comprising a nucleotide sequence encoding the second polypeptide of a TMP of the present disclosure; and c) a pharmaceutically acceptable excipient. In some cases, a suitable formulation comprises: a) a recombinant expression vector comprising a nucleotide sequence encoding a TMP of the present disclosure; and b) a pharmaceutically acceptable excipient. In some cases, a suitable formulation comprises: a) a first recombinant expression vector comprising a nucleotide sequence encoding the first polypeptide of a TMP of the present disclosure; b) a second recombinant expression vector comprising a nucleotide sequence encoding the second polypeptide of a TMP of the present disclosure; and c) a pharmaceutically acceptable excipient. Suitable pharmaceutically acceptable excipients are described above.

### Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A TMP of the present disclosure may be administered in amounts between 0.1 mg/kg body weight and 20 mg/kg body weight per dose, e.g. between 0.1 mg/kg body weight to 10 mg/kg body weight, e.g. between 0.5 mg/kg body weight to 5 mg/kg body weight, between 1 mg/kg body weight to 5 mg/kg body weight; between 5 mg/kg body weight to 10 mg/kg body weight; between 10 mg/kg body weight to 15 mg/kg body weight; between 15 mg/kg body weight to 20 mg/kg body weight, however, doses above this exemplary range are envisioned, especially considering the aforementioned factors. If the regimen is a continuous infusion, it can also be in the range of 1 g to 10 mg per kilogram of body weight per minute. A TMP of the present disclosure can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMP of the present disclosure is administered in maintenance doses, ranging from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, or amounts exceeding 20 mg/kg of body weight.

Those of skill will readily appreciate that dose levels can vary as a function of the specific TMP, the severity of the symptoms and the susceptibility of the subject to side effects. Preferred dosages for a given compound are readily determinable by those of skill in the art by a variety of means.

In some cases, multiple doses of a TMP of the present disclosure, a nucleic acid of the present disclosure, or a recombinant expression vector of the present disclosure are administered. The frequency of administration of a TMP of the present disclosure, a nucleic acid of the present disclosure, or a recombinant expression vector of the present disclosure can vary depending on any of a variety of factors, e.g., severity of the symptoms, etc. For example, in some cases, a TMP of the present disclosure, a nucleic acid of the present disclosure, or a recombinant expression vector of the present disclosure is administered once per month, twice per month, three times per month, every other week (qow), once per week (qw), once every two weeks, once every three weeks, once every four weeks, twice per week (biw), three times per week (tiw), four times per week, five times per week, six times per week, every other day (qod), daily (qd), twice a day (qid), or three times a day (tid).

The duration of administration of a TMP of the present disclosure, a nucleic acid of the present disclosure, or a recombinant expression vector of the present disclosure, e.g., the period of time over which a TMP of the present disclosure, a nucleic acid of the present disclosure, or a recombinant expression vector of the present disclosure is administered, can vary, depending on any of a variety of factors, e.g., patient response, etc. For example, a TMP of the present disclosure, a nucleic acid of the present disclosure, or a recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

An active agent (a TMP of the present disclosure, a nucleic acid of the present disclosure, or a recombinant expression vector of the present disclosure) is administered to an individual using any available method and route suitable for drug delivery, including in vivo and in vitro methods, as well as systemic and localized routes of administration.

A TMP of this disclosure typically will be delivered via intravenous administration, but other conventional and pharmaceutically acceptable routes of administration may be used, including intratumoral, peritumoral, intramuscular, intralymphatic, intratracheal, intracranial, subcutaneous, intradermal, topical application, intraarterial, rectal, nasal, oral, and other enteral and parenteral routes of administration. Routes of administration may be combined, if desired, or adjusted depending upon the TMP and/or the desired effect. A TMP of the present disclosure, or a nucleic acid or recombinant expression vector of the present disclosure, can be administered in a single dose or in multiple doses.

A TMP of the present disclosure, a nucleic acid of the present disclosure, or a recombinant expression vector of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

### Combination Therapy

A TMP of the present disclosure can be administered to an individual in need thereof in combination with one or more additional therapeutic agents or therapeutic treatment. A suitable dosage amount of the TMP will be the same as the dosage amount for monotherapy with the APP (described above) or may be less or more than the monotherapy dose. Suitable additional therapeutic agents include, e.g.:i) an immune checkpoint inhibitor; ii) a cancer chemotherapeutic agent; iii) an agent that inhibits a cancer-associated mutated form of KRAS; and iv) one or more additional TMPs. Suitable additional therapeutic treatments include, e.g., radiation, surgery (e.g., surgical resection of a tumor), and the like.

In some cases, the method comprises administering to an individual in need thereof:a) a first composition comprising a TMP of the present disclosure; and b) a second composition comprising an immune checkpoint inhibitor. In some cases, the method comprises administering to an individual in need thereof:a) a first composition comprising a TMP of the present disclosure; and b) a second composition comprising an agent that inhibits a cancer-associated mutated form of KRAS such as KRAS (G12C). In some cases, the method comprises administering to an individual in need thereof:a) a first composition comprising a TMP of the present disclosure; and b) a second composition comprising a second TMP.

A TMP of the present disclosure can be administered to an individual in need thereof at the same time, or at different times, as the one or more additional therapeutic agent is administered.

Thus, for example, a treatment method of the present disclosure can comprise co-administration of a TMP of the present disclosure and at least one additional therapeutic agent. By “co-administration” is meant that both a TMP of the present disclosure and at least one additional therapeutic agent are administered to an individual, although not necessarily at the same time, in order to achieve a therapeutic effect that is the result of having administered both the TMP and the at least one additional therapeutic agent. The administration of the TMP and the at least one additional therapeutic agent can be substantially simultaneous, e.g., the TMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a TMP of the present disclosure is administered to an individual who is undergoing treatment with, or who has undergone treatment with, the at least one additional therapeutic agent. The administration of the TMP and the at least one additional therapeutic agent can occur at different times and/or at different frequencies.

As another example, a treatment method of the present disclosure can comprise co-administration of a TMP of the present disclosure and an immune checkpoint inhibitor such as an antibody specific for an immune checkpoint. By “co-administration” is meant that both a TMP of the present disclosure and an antibody specific for an immune checkpoint are administered to an individual, although not necessarily at the same time, in order to achieve a therapeutic effect that is the result of having administered both the TMP and the immune checkpoint inhibitor. The administration of the TMP and the antibody specific for an immune checkpoint can be substantially simultaneous, e.g., the TMP can be administered to an individual within about 1 minute to about 24 hours (e.g., within about 1 minute, within about 5 minutes, within about 15 minutes, within about 30 minutes, within about 1 hour, within about 2 hours, within about 4 hours, within about 8 hours, within about 12 hours, or within about 24 hours) of administration of the antibody specific for an immune checkpoint. In some cases, a TMP of the present disclosure is administered to an individual who is undergoing treatment with, or who has undergone treatment with, an antibody specific for an immune checkpoint. The administration of the TMP and the antibody specific for an immune checkpoint can occur at different times and/or at different frequencies.

Exemplary immune checkpoint inhibitors include inhibitors that target immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122, and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT, and VISTA.

In some cases, the immune checkpoint inhibitor is an antibody specific for an immune checkpoint. Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J Clin. Oncol. 31:616-22. In some cases, the immune checkpoint inhibitor is an anti-PD-L1 antibody. In some cases, the anti-PD-L1 monoclonal antibody is BMS-935559, MED14736, MPDL3280A (also known as RG7446), KN035, or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149.

In some cases, the at least one additional therapeutic agent is an agent that selectively inhibits a mutant form of KRAS such as KRAS (G12C), KRAS (K117A), and the like. Examples of agents that selectively inhibit a mutant form of KRAS include ARS-1620; AMG510; KRA-533; and MRTX849.

AMG150 has the following structure:

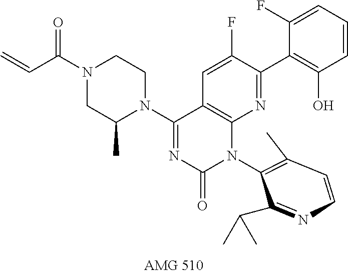

ARS-1620 has the following structure:

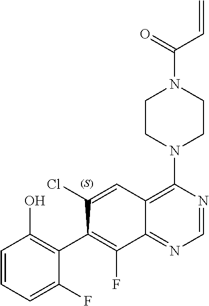

In some cases, the at least one additional therapeutic agent comprises one or more additional TMPs. In some cases, the method comprises administering to an individual in need thereof a) a first composition comprising a first TMP, where the first TMP is a TMP of the present disclosure; and b) a second composition comprising a second TMP, where the second TMP is a TMP of the present disclosure that is different from the first TMP of the present disclosure, e.g., comprising a different KRAS epitope and/or one or more different MODs. In addition, or alternatively, the one or more additional TMPs can comprise an epitope that is a cancer-associated peptide other than a KRAS peptide epitope.

### Subjects Suitable for Treatment

Subjects suitable for treatment with a method of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment with a method of the present disclosure include individuals having a cancer in which the cancer cells express a mutated form of KRAS, where the mutated form of KRAS is a cancer-associated mutated form. Subjects suitable for treatment with a method of the present disclosure include individuals having a cancer such as multiple myeloma; B-cell lymphoma; breast cancer; lung cancer; ovarian carcinoma; pancreatic cancer; colorectal cancer; prostate cancer; renal cancer; acute myelogenous leukemia; mesothelioma; thyroid cancer; head and neck cancer; stomach cancer; urothelial cancer; cervical cancer; and ovarian endometrial cancer.

In some cases, the subject is an individual who is undergoing treatment with an immune checkpoint inhibitor. In some cases, the subject is an individual who has undergone treatment with an immune checkpoint inhibitor, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory polypeptide (TMP) comprising:
i) a KRAS peptide comprising a KRAS epitope expressed on a cancer cell, wherein the KRAS peptide has a length of at least 4 amino acids;
ii) first major histocompatibility complex (MHC) polypeptide;
iii) a second MHC polypeptide, and
iv) at least one immunomodulatory polypeptide.

Aspect 2. A T-cell modulatory polypeptide according to aspect 1, further comprising in Ig Fc polypeptide.

Aspect 3. A T-cell modulatory polypeptide according to aspect 2, wherein the Ig Fc polypeptide is a human IgG1 Fc polypeptide that substantially does not induce cell lysis.

Aspect 4. A T-cell modulatory polypeptide of aspect 2 or 3, wherein IgG1 Fc polypeptide comprises one or more amino acid substitutions selected from N297A, L234A, L235A, L234F, L235E, and P331S.

Aspect 5. A T-cell modulatory polypeptide of aspect 4, wherein the IgG1 Fc polypeptide comprises the amino acid sequence of FIG. 3G.

Aspect 6. A T-cell modulatory polypeptide of any one of aspects 1-5, wherein the first major histocompatibility complex (MIHC) polypeptide is a β2-microglobulin polypeptide; and wherein the second MHC polypeptide is an MHC class I heavy chain polypeptide.

Aspect 7. A T-cell modulatory polypeptide of aspect 5, wherein the β2-microglobulin polypeptide is joined to the KRAS peptide by a first linker.

Aspect 8. A T-cell modulatory polypeptide of any one of aspects 1-7, wherein the β2-microglobulin polypeptide and MHC class I heavy chain polypeptide are covalently linked to one another.

Aspect 9. A T-cell modulatory polypeptide of aspect 8, wherein the covalent linkage is via one or more disulfide bonds.

Aspect 10. A T-cell modulatory polypeptide of aspect 9, wherein the β2M polypeptide and the MHC heavy chain polypeptide are joined by a disulfide bond that joins a Cys residue in the β2M polypeptide and a Cys residue in the MHC heavy chain polypeptide.

Aspect 11. A T-cell modulatory polypeptide of aspect 10, wherein a Cys at amino acid residue 12 of the β2M polypeptide is disulfide bonded to a Cys at amino acid residue 236 of the MHC heavy chain polypeptide.

Aspect 12. A T-cell modulatory polypeptide of any one of aspects 7-11, wherein the first linker comprises a Cys and wherein a disulfide bond links a Cys present in the first linker with a Cys present in the MIHC heavy chain polypeptide.

Aspect 13. A T-cell modulatory polypeptide of aspect 12, wherein the first linker comprises the sequence CGGGS (SEQ ID NO:141), GCGGS (SEQ ID NO:139), GGCGS (SEQ ID NO:587), GGGCS (SEQ ID NO:588) or GGGGC (SEQ ID NO:590) and a disulfide bond links the Cys in the linker with a Cys substituted for Tyr84 of the MHC heavy chain polypeptide.

Aspect 14. A T-cell modulatory polypeptide of aspect 13, wherein the first linker comprises the sequence CGGGS(GGGGS)n (SEQ ID NO:142) or GCGGS(GGGGS)n (SEQ ID NO:140), where n is an integer from 1-10, e.g., 2 or 3, and a disulfide bond links the Cys in the linker with a Cys substituted for Tyr84 of the MHC heavy chain polypeptide.

Aspect 15. A T-cell modulatory polypeptide of aspect 9-14, wherein the TMP comprises (i) a first disulfide bond between the Cys residue in the β2M polypeptide and a Cys residue in the MIHC heavy chain polypeptide, and (ii) a second disulfide bond that links a Cys present in the first linker with a Cys present in the MHC heavy chain polypeptide.

Aspect 16. A T-cell modulatory polypeptide of aspect 15, wherein (i) a Cys at amino acid residue 12 of the β2M polypeptide is disulfide bonded to a Cys at amino acid residue 236 of the MHC heavy chain polypeptide, and (ii) the first linker comprises the sequence CGGGS (SEQ ID NO:141), GCGGS (SEQ ID NO:139), GGCGS (SEQ ID NO:587), GGGCS (SEQ ID NO:588) or GGGGC (SEQ ID NO:590) and a disulfide bond links the Cys in the linker with a Cys substituted for Tyr84 of the MHC heavy chain polypeptide.

Aspect 17. A T-cell modulatory polypeptide of aspect 16, wherein the first linker comprises the sequence CGGGS(GGGGS)n (SEQ ID NO:142) or GCGGS(GGGGS)n (SEQ ID NO:140), where n is an integer from 1-10, e.g., 2 or 3.

Aspect 18. A T-cell modulatory polypeptide of any of aspects 1-17, wherein a disulfide bond joins two Cys residues present in the MHC heavy chain polypeptide.

Aspect 19. A T-cell modulatory polypeptide of any one of aspects 1-18, wherein the MHC heavy chain polypeptide comprises:
a) an amino acid sequence having at least 95% amino acid sequence identity to the HLA-A*0101, HLA-A*0201, HLA-A*1101, HLA-A*2301, HLA-A*2402, HLA-A*2407, HLA-A*3303, or HLA-A*3401 amino acid sequence depicted in FIG. 7A; or
b) an amino acid sequence having at least 95% amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A; or
c) an amino acid sequence having at least 95% amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIG. 9A.

Aspect 20. A T-cell modulatory polypeptide of aspect 19, wherein the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, an HLA-A*3303 polypeptide, and an HLA-A*2401 polypeptide.

Aspect 21. A T-cell modulatory polypeptide of any one of aspects 1-20, wherein the at least one immunomodulatory polypeptide is a wild-type or variant of an activating immunomodulatory polypeptide.

Aspect 22. A T-cell modulatory polypeptide of any one of aspects 1-21, wherein the at least one immunomodulatory polypeptide is a wild-type or variant of an activating immunomodulatory polypeptide selected from the group consisting of an IL-2 polypeptide, a 4-1BBL polypeptide, a CD80 polypeptide, a CD86 polypeptide, or combinations thereof.

Aspect 23. A T-cell modulatory polypeptide of any one of aspects 1-22, wherein at least one of the at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide,

Aspect 24. A T-cell modulatory polypeptide of any one of aspects 21-23, wherein the at least one immunomodulatory polypeptide is a variant of IL-2 that substantially does not bind to IL-2Rα and has reduced affinity for IL-2Rß.

Aspect 25. A T-cell modulatory polypeptide of aspect 24, wherein the variant IL-2 polypeptide comprises i) an H16A substitution and an F42A substitution; or ii) an H16T substitution and an F42A substitution.

Aspect 26. A T-cell modulatory polypeptide of any one of aspects 1-25, wherein the polypeptide comprises at least two immunomodulatory polypeptides, and wherein at least two of the immunomodulatory polypeptides are the same.

Aspect 27. A T-cell modulatory polypeptide of any one of aspects 25 or 26, wherein the 2 or more immunomodulatory polypeptides are in tandem.

Aspect 28. A T-cell modulatory polypeptide of aspect 27, comprising two variant IL-2 polypeptides in tandem, each one comprising i) an H16A substitution and an F42A substitution; or ii) an H16T substitution and an F42A substitution.

Aspect 29. A T-cell modulatory polypeptide of any one of aspects 1-28, wherein the KRAS peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 9-10 amino acids, from 10 to 20 amino acids, and from 15 to 25 amino acids in length).

Aspect 30. A T-cell modulatory polypeptide of aspect 29, wherein the KRAS peptide comprises a sequence selected from the group consisting of VVGADGVGK (SEQ ID NO:176), VVGACGVGK (SEQ ID NO:177), VVGAVGVGK (SEQ ID NO:178), VVVGADGVGK (SEQ ID NO:179), VVVGAVGVGK (SEQ ID NO:180), VVVGACGVGK (SEQ ID NO:181), VTGADGVGK (SEQ ID NO:182), VTGAVGVGK (SEQ ID NO:183), VTGACGVGK (SEQ ID NO:184), VTVGADGVGK (SEQ ID NO:185), VTVGAVGVGK (SEQ ID NO:186), and VTVGACGVGK (SEQ ID NO:187); and wherein the KRAS peptide has a length of 9 amino acids or 10 amino acids, or a length of at least 9 amino acids or 10 amino acids.

Aspect 31. A T-cell modulatory polypeptide of aspect 29, wherein the KRAS peptide comprises a sequence selected from the group consisting of: VVVGAGDVGK (SEQ ID NO:188); VVGAGDVGK (SEQ ID NO:189); VVVGARGVGK (SEQ ID NO:190); and VVGARGVGK (SEQ ID NO:191); and wherein the KRAS peptide has a length of 9 amino acids or 10 amino acids, or a length of at least 9 amino acids or 10 amino acids.

Aspect 32. A T-cell modulatory polypeptide of aspect 29, wherein the KRAS peptide comprises a sequence selected from the group consisting of LVVVGADGV (SEQ ID NO:192), LVVVGAVGV (SEQ ID NO:193), LVVVGACGV (SEQ ID NO:194), KLVVVGADGV (SEQ ID NO:195), KLVVVGAVGV (SEQ ID NO:196), KLVVVGACGV (SEQ ID NO:197), LLVVGADGV (SEQ ID NO:198), LLVVGAVGV (SEQ ID NO:199), LLVVGACGV (SEQ ID NO:200), FLVVVGADGV (SEQ ID NO:201), FLVVVGAVGV (SEQ ID NO:202), and FLVVVGACGV (SEQ ID NO:203); and wherein the KRAS peptide has a length of 9 amino acids or 10 amino acids, or a length of at least 9 amino acids or 10 amino acids.

Aspect 33. A T-cell modulatory polypeptide of aspect 29, wherein the KRAS peptide comprises a sequence selected from the group consisting of: KLVVVGAGDV (SEQ ID NO:204); and KLVVVGARGV (SEQ ID NO:205); wherein the KRAS peptide has a length of 9 amino acids or 10 amino acids, or a length of at least 9 amino acids or 10 amino acids.

Aspect 34. A T-cell modulatory polypeptide of aspect 29, wherein the KRAS peptide comprises a sequence selected from the group consisting: GAGDVGKSAL (SEQ ID NO:206); AGDVGKSAL (SEQ ID NO:207); DVGKSALTI (SEQ ID NO:208); GAVGVGKSAL (SEQ ID NO:209); AVGVGKSAL (SEQ ID NO:210); YKLVVVGAV (SEQ ID NO:211); ARGVGKSAL (SEQ ID NO:212); GARGVGKSAL (SEQ ID NO:213); EYKLVVVGAR (SEQ ID NO:214); RGVGKSALTI (SEQ ID NO:215); LVVVGARGV (SEQ ID NO:216); GADGVGKSAL (SEQ ID NO:217); ACGVGKSAL (SEQ ID NO:218); and GACGVGKSAL (SEQ ID NO:219); wherein the KRAS peptide has a length of 9 amino acids or 10 amino acids, or a length of at least 9 amino acids or 10 amino acids.

Aspect 35. A T-cell modulatory polypeptide of aspect 29, wherein the KRAS peptide comprises a sequence selected from the group consisting of: VVGAVGVGK (SEQ ID NO:178), VVVGAVGVGK (SEQ ID NO:180), VGAVGVGKS (SEQ ID NO:222), VGAVGVGKSA (SEQ ID NO:223), AVGVGKSAL (SEQ ID NO:210), AVGVGKSALT (SEQ ID NO:225), GAVGVGKSAL (SEQ ID NO:209), GAVGVGKSA (SEQ ID NO:227), LVVVGAVGVG (SEQ ID NO:228), LVVVGAVGV (SEQ ID NO:193), KLVVVGAVGV (SEQ ID NO:196), and KLVVVGAVG (SEQ ID NO:231); where the KRAS peptide has a length of 9 amino acids or 10 amino acids, or a length of at least 9 amino acids or 10 amino acids.

Aspect 36. A T-cell modulatory polypeptide of any one of aspects 29-35, wherein the KRAS peptide is a peptide of 9 or 10 amino acids in length.

Aspect 37. A T-cell modulatory polypeptide of aspect 36, wherein the KRAS peptide has the amino acid sequence VVGADGVGK (SEQ ID NO:176), VVGACGVGK (SEQ ID NO:177), or VVGAVGVGK (SEQ ID NO:178), and has a length of 9 amino acids.

Aspect 38. A T-cell modulatory polypeptide of aspect 36, wherein the KRAS peptide has the amino acid sequence VVVGADGVGK (SEQ ID NO:179), VVVGACGVGK (SEQ ID NO:181), or VVVGAVGVGK (SEQ ID NO:180), and has a length of 10 amino acids.

Aspect 39 A T-cell modulatory polypeptide of aspect 36, wherein the KRAS peptide has the amino acid sequence VTGADGVGK (SEQ ID NO:182), VTGACGVGK (SEQ ID NO:184), or VTGAVGVGK (SEQ ID NO:183), and has a length of 9 amino acids.

Aspect 40. A T-cell modulatory polypeptide of aspect 36, wherein the KRAS peptide has the amino acid sequence VTVGADGVGK (SEQ ID NO:185), VTVGACGVGK (SEQ ID NO:187), or VTVGAVGVGK (SEQ ID NO:186), and has a length of 10 amino acids.

Aspect 41. A T-cell modulatory polypeptide of aspect 32, wherein the KRAS peptide is KLVVVGADGV (SEQ ID NO:195) and the MHC heavy chain polypeptide comprises an amino acid sequence having at least 95% amino acid sequence identity to an HLA-A*0201 polypeptide.

Aspect 42. A T-cell modulatory polypeptide of aspect 32, wherein the KRAS peptide is VVVGADGVGK (SEQ ID NO:179) and the MHC heavy chain polypeptide comprises an amino acid sequence having at least 95% amino acid sequence identity to an HLA-A11*01 polypeptide.

Aspect 43. A T-cell modulatory polypeptide of aspect 37, wherein the KRAS peptide is VVGAVGVGK (SEQ ID NO:178) and the MHC heavy chain polypeptide comprises an amino acid sequence having at least 95% amino acid sequence identity to an HLA-A11*01 polypeptide.

Aspect 44. A heterodimeric T-cell modulatory polypeptide according to any one of aspects 1-43, comprising:
a) a first polypeptide comprising:
   i) a KRAS peptide comprising a KRAS epitope expressed on a cancer cell, wherein the KRAS peptide has a length of at least 4 amino acids; and
   ii) a β2-microglobulin polypeptide; and
b) an MIHC class I heavy chain polypeptide, and
c) at least one immunomodulatory polypeptide, wherein the first and/or the second polypeptide comprises the immunomodulatory polypeptide.

Aspect 45. A heterodimeric T-cell modulatory polypeptide of aspect 44, wherein:
a1) the first polypeptide comprises, in order from N-terminus to C-terminus:
   i) the KRAS peptide; and
   ii) a β2-microglobulin polypeptide; and
b1) the second polypeptide comprises, in order from N-terminus to C-terminus:
   i) at least one immunomodulatory polypeptide;
   ii) an MHC class I heavy chain polypeptide; and
   iii) an Ig Fc polypeptide; or
a2) the first polypeptide comprises, in order from N-terminus to C-terminus:
   i) the KRAS peptide; and
   ii) a β2-microglobulin polypeptide; and
b2) the second polypeptide comprises, in order from N-terminus to C-terminus:
   i) an MHC class I heavy chain polypeptide; and
   ii) at least one immunomodulatory polypeptide; and
   iii) an immunoglobulin (Ig) Fc polypeptide; or
a3) the first polypeptide comprises, in order from N-terminus to C-terminus:
   i) the KRAS peptide; and
   ii) a β2-microglobulin polypeptide; and
b3) the second polypeptide comprises, in order from N-terminus to C-terminus:
   i) an MHC class I heavy chain polypeptide; and
   ii) an Ig Fc polypeptide; and
   iii) at least one immunomodulatory polypeptide; or
a4) the first polypeptide comprises, in order from N-terminus to C-terminus:
   i) at least one immunomodulatory polypeptide;
   ii) the KRAS peptide; and
   iii) a β2-microglobulin polypeptide; and
b4) the second polypeptide comprises, in order from N-terminus to C-terminus:
   i) an MHC class I heavy chain polypeptide; and
   ii) an Ig Fc polypeptide; or
a5) the first polypeptide comprises, in order from N-terminus to C-terminus:
   i) the KRAS peptide;
   ii) a β2-microglobulin polypeptide; and
   iii) at least one immunomodulatory polypeptide; and
b5) the second polypeptide comprises:
   i) an MHC class I heavy chain polypeptide; and
   ii) an Ig Fc polypeptide.

Aspect 46. A heterodimeric T-cell modulatory polypeptide of aspect 45, wherein:
a1) the first polypeptide comprises, in order from N-terminus to C-terminus:
   i) the KRAS peptide; and
   ii) a β2-microglobulin polypeptide; and
b1) the second polypeptide comprises, in order from N-terminus to C-terminus:
   i) at least one immunomodulatory polypeptide;
   ii) an MHC class I heavy chain polypeptide; and
   iii) an Ig Fc polypeptide; or
a3) the first polypeptide comprises, in order from N-terminus to C-terminus:
   i) the KRAS peptide; and
   ii) a β2-microglobulin polypeptide; and
b3) the second polypeptide comprises, in order from N-terminus to C-terminus:
   i) an MHC class I heavy chain polypeptide; and
   ii) an Ig Fc polypeptide; and
   iii) at least one immunomodulatory polypeptide; and
   wherein the Ig Fc polypeptide is a human IgG1 Fc polypeptide that substantially does not induce cell lysis, optionally comprising the amino acid sequence of FIG. 3G,
   wherein the β2-microglobulin polypeptide is joined to the KRAS peptide by a first linker that comprises the sequence CGGGS(GGGGS)n (SEQ ID NO:142) or GCGGS(GGGGS)n (SEQ ID NO:140), where n is an integer from 1-10, e.g., 2 or 3,
   wherein the MIHC heavy chain polypeptide comprises a Cys at residue 84 and a Cys at residue 236,
   wherein the β2M polypeptide comprises a Cys at residue 12,
   wherein the Cys at amino acid residue 12 of the β2M polypeptide is disulfide bonded to a Cys at amino acid residue 236 of the MHC heavy chain polypeptide,
   wherein a disulfide bond links the Cys in the linker with a Cys substituted for Tyr84 of the MIHC heavy chain polypeptide,
   wherein the MIHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, .an HLA-A*3303 polypeptide, and an HLA-A*2401 polypeptide,
   wherein the at least one immunomodulatory polypeptide is a variant of IL-2 that comprises i) an H16A substitution and an F42A substitution; or ii) an H16T substitution and an F42A substitution, and
   wherein the polypeptide comprises two immunomodulatory polypeptides that are the same, are in tandem, and comprise a variant of IL-2 that comprises i) an H16A substitution and an F42A substitution; or ii) an H16T substitution and an F42A substitution.

Aspect 47. A heterodimeric T-cell modulatory polypeptide of aspect 46, wherein the KRAS peptide has the amino acid sequence VVGADGVGK (SEQ ID NO:176), VVGACGVGK (SEQ ID NO:177), or VVGAVGVGK (SEQ ID NO:178), and has a length of 9 amino acids.

Aspect 48. A heterodimeric T-cell modulatory polypeptide of aspect 46, wherein the KRAS peptide has the amino acid sequence VVVGADGVGK (SEQ ID NO:179), VVVGACGVGK (SEQ ID NO:181), or VVVGAVGVGK (SEQ ID NO:180), and has a length of 10 amino acids.

Aspect 49 A heterodimeric T-cell modulatory polypeptide of aspect 46, wherein the KRAS peptide has the amino acid sequence VTGADGVGK (SEQ ID NO:182), VTGACGVGK (SEQ ID NO:184), or VTGAVGVGK (SEQ ID NO:183), and has a length of 9 amino acids.

Aspect 50. A heterodimeric T-cell modulatory polypeptide of aspect 46, wherein the KRAS peptide has the amino acid sequence VTVGADGVGK (SEQ ID NO:185), VTVGACGVGK (SEQ ID NO:187), or VTVGAVGVGK (SEQ ID NO:186), and has a length of 10 amino acids.

Aspect 51. A heterodimeric T-cell modulatory polypeptide of aspect 46, wherein the KRAS peptide is KLVVVGADGV (SEQ ID NO:195) and the MHC heavy chain polypeptide comprises an amino acid sequence having at least 95% amino acid sequence identity to an HLA-A*0201 polypeptide.

Aspect 52. A heterodimeric T-cell modulatory polypeptide of aspect 46, wherein the KRAS peptide is VVVGADGVGK (SEQ ID NO:179) and the MHC heavy chain polypeptide comprises an amino acid sequence having at least 95% amino acid sequence identity to an HLA-A11*01 polypeptide.

Aspect 53. A heterodimeric T-cell modulatory polypeptide of aspect 46, wherein the KRAS peptide is VVGAVGVGK (SEQ ID NO: 178) and the MHC heavy chain polypeptide comprises an amino acid sequence having at least 95% amino acid sequence identity to an HLA-A11*01 polypeptide.

Aspect 54. A single chain T-cell modulatory polypeptide according to any one of aspects 1-43, comprising:
i) a KRAS peptide comprising a KRAS epitope expressed on a cancer cell, wherein the KRAS peptide has a length of at least 4 amino acids;
ii) a β2-microglobulin polypeptide;
iii) an MIHC class I heavy chain polypeptide, and
iv) at least one immunomodulatory polypeptide.

Aspect 55. A single chain T-cell modulatory polypeptide of aspect 54, comprising in order from N-terminus to C-terminus:
A) i) a KRAS peptide; ii) a β2M polypeptide; iii) a class I MHC heavy chain polypeptide; iv) one or more immunomodulatory polypeptides; and v) an Ig Fc polypeptide; or
B) i) a KRAS peptide; ii) a β2M polypeptide; iii) a class I MHC heavy chain polypeptide; iv) an Ig Fc polypeptide; and v) one or more immunomodulatory polypeptides; or
C) i) one or more immunomodulatory polypeptides; ii) a KRAS peptide; iii) a β2M polypeptide; iv) a class I MHC heavy chain polypeptide; and v) an Ig Fc polypeptide.

Aspect 56. A single chain T-cell modulatory polypeptide of aspect 55, comprising in order from N-terminus to C-terminus:
i) a KRAS peptide; ii) a β2M polypeptide; iii) a class I MIHC heavy chain polypeptide; iv) an Ig Fc polypeptide; and v) one or more immunomodulatory polypeptides,
   wherein the Ig Fc polypeptide T-substantially does not induce cell lysis, optionally comprising the amino acid sequence of FIG. 3G,
wherein the β2-microglobulin polypeptide is joined to the KRAS peptide by a first linker that comprises the sequence CGGGS(GGGGS)n (SEQ ID NO:142) or GCGGS(GGGGS)n (SEQ ID NO:140), where n is an integer from 1-10, e.g., 2 or 3,
wherein the MHC heavy chain polypeptide comprises a Cys at residue 84 and a Cys at residue 236,
wherein the β2M polypeptide comprises a Cys at residue 12,
wherein the Cys at amino acid residue 12 of the β2M polypeptide is disulfide bonded to a Cys at amino acid residue 236 of the MHC heavy chain polypeptide,
wherein a disulfide bond links the Cys in the linker with a Cys substituted for Tyr84 of the MHC heavy chain polypeptide,
wherein the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, .an HLA-A*3303 polypeptide, and an HLA-A*2401 polypeptide,
wherein the ß2M polypeptide is connected to the MIHC heavy chain polypeptide by a (GGGGS)n (SEQ ID NO:377) linker, where n is 1, 2, 3, 4, 5, 6, 7, 8, 9, or 10, e.g., with n=3 or 7,
wherein the at least one immunomodulatory polypeptide is a variant of IL-2 that comprises i) an H16A substitution and an F42A substitution; or ii) an H16T substitution and an F42A substitution, and
wherein the polypeptide comprises two immunomodulatory polypeptides that are the same, are in tandem, and comprise a variant of IL-2 that comprises i) an H16A substitution and an F42A substitution; or ii) an H16T substitution and an F42A substitution.

Aspect 57. A heterodimeric T-cell modulatory polypeptide of aspect 56, wherein the KRAS peptide has the amino acid sequence VVGADGVGK (SEQ ID NO:176), VVGACGVGK (SEQ ID NO:177), or VVGAVGVGK (SEQ ID NO:178), and has a length of 9 amino acids.

Aspect 58. A heterodimeric T-cell modulatory polypeptide of aspect 56, wherein the KRAS peptide has the amino acid sequence VVVGADGVGK (SEQ ID NO:179), VVVGACGVGK (SEQ ID NO:181), or VVVGAVGVGK (SEQ ID NO:180), and has a length of 10 amino acids.

Aspect 59 A heterodimeric T-cell modulatory polypeptide of aspect 56, wherein the KRAS peptide has the amino acid sequence VTGADGVGK (SEQ ID NO:182), VTGACGVGK (SEQ ID NO:184), or VTGAVGVGK (SEQ ID NO:183), and has a length of 9 amino acids.

Aspect 60. A heterodimeric T-cell modulatory polypeptide of aspect 56, wherein the KRAS peptide has the amino acid sequence VTVGADGVGK (SEQ ID NO:185), VTVGACGVGK (SEQ ID NO:187), or VTVGAVGVGK (SEQ ID NO:186), and has a length of 10 amino acids.

Aspect 61. A heterodimeric T-cell modulatory polypeptide of aspect 56, wherein the KRAS peptide is KLVVVGADGV (SEQ ID NO:195) and the MHC heavy chain polypeptide comprises an amino acid sequence having at least 95% amino acid sequence identity to an HLA-A*0201 polypeptide.

Aspect 62. A heterodimeric T-cell modulatory polypeptide of aspect 56, wherein the KRAS peptide is VVVGADGVGK (SEQ ID NO:179) and the MHC heavy chain polypeptide comprises an amino acid sequence having at least 95% amino acid sequence identity to an HLA-A11*01 polypeptide.

Aspect 63. A heterodimeric T-cell modulatory polypeptide of aspect 56, wherein the KRAS peptide is VVGAVGVGK (SEQ ID NO: 178) and the MHC heavy chain polypeptide comprises an amino acid sequence having at least 95% amino acid sequence identity to an HLA-A11*01 polypeptide.

Aspect 64. A T-cell modulatory polypeptide, wherein the TMP is a homodimer comprising a first and second heterodimer of any one of aspects 44-53,

wherein the first and second heterodimers are the same and are covalently bound by one or more disulfide bonds between the Ig Fc polypeptides of the first and second heterodimers.

Aspect 65. A T-cell modulatory polypeptide, wherein the TMP is a homodimer comprising a first and second single-chain TMP of any one of aspects 54-63,

wherein the first and second single-chain TMPs are the same and are covalently bound by one or more disulfide bonds between the Ig Fc polypeptides of the first and second TMPs.

Aspect 66. A nucleic acid comprising a nucleotide sequence encoding a first or second polypeptide of any one of aspects 44-53.

Aspect 67. A nucleic acid comprising a nucleotide sequence encoding a single-chain TMP of any one of aspects 54-63.

Aspect 68. An expression vector comprising a nucleic acid of aspect 66 or 67.

Aspect 69. A method of selectively modulating the activity of T cell specific for a KRAS peptide epitope, the method comprising contacting the T cell with a T-cell modulatory polypeptide according to any one of aspects 1-65, wherein said contacting selectively modulates the activity of the epitope-specific T cell.

Aspect 70. A method of treating a KRAS-associated cancer in a patient having the cancer, the method comprising administering to the patient an effective amount of a pharmaceutical composition comprising T-cell modulatory polypeptide according to any one of aspects 1-65.

Aspect 71. The method of aspect 70, wherein the cancer is non-small cell lung cancer, lung adenocarcinoma, mucinous adenoma, ductal carcinoma of the pancreas, colorectal cancer, or leukemia.

Aspect 72. The method of aspect 70 or 71, wherein said administering is intravenous.

Aspect 73. The method of any one of aspects 70-72, further comprising co-administering an immune checkpoint inhibitor to the patient.

Aspect 74. The method of aspect 73, wherein the immune checkpoint inhibitor is an antibody specific for PD-L1, PD-1, or CTLA4.

Aspect 75. The method of any one of aspects 70-74, further comprising administering a KRAS(G12C) inhibitor.

Aspect 76. A method of modulating an immune response in an individual, the method comprising administering to the individual an effective amount of the T-cell modulatory polypeptide of any one of aspects 1-65, wherein said administering induces an epitope-specific T cell response and an epitope-non-specific T cell response, wherein the ratio of the epitope-specific T cell response to the epitope-non-specific T cell response is at least 2:1.

Aspect 77. A method of delivering an immunomodulatory polypeptide selectively to a target T cell, the method comprising contacting a mixed population of T cells with a T-cell modulatory polypeptide of any one of aspects 1-65, wherein the mixed population of T cells comprises the target T cell and non-target T cells, wherein the target T cell is specific for the KRAS epitope present within the T-cell modulatory polypeptide, and wherein said contacting delivers the one or more immunomodulatory polypeptides present within the T-cell modulatory polypeptide to the target T cell.

Aspect 78. A method of detecting, in a mixed population of T cells obtained from an individual, the presence of a target T cell that binds a KRAS peptide comprising a cancer-associated mutation, the method comprising: a) contacting in vitro the mixed population of T cells with the T-cell modulatory polypeptide of any one of aspects 1-65, wherein the T-cell modulatory polypeptide comprises the peptide epitope; and b) detecting activation and/or proliferation of T cells in response to said contacting, wherein activated and/or proliferated T cells indicates the presence of the target T cell.

Aspect 79. A method of detecting the presence of KRAS-specific T cells, comprising the steps of
A) contacting a KRAS-specific T cell with an antigen presenting polypeptide (APP) comprising:
   i) a KRAS peptide comprising a KRAS epitope specific for the TCR on KRAS-specific T cell;
   ii) first major histocompatibility complex (MHC) polypeptide;
   iii) a second MHC polypeptide, and
   iv) a detectable label, and
B) detecting the presence of the label.

Aspect 80. A method of aspect 79, wherein the step of contacting is carried out in vivo.

Aspect 81. A method of aspect 79, wherein the step of contacting is carried out in vitro.

Aspect 82. A method according to any one of aspects 79-81, wherein the APP is a heterodimer comprising a first and second polypeptide, and wherein:
a1) the first polypeptide comprises, in order from N-terminus to C-terminus:
   i) the KRAS peptide; and
   ii) a β2-microglobulin polypeptide; and
b1) the second polypeptide comprises, in order from N-terminus to C-terminus:
   i) an MHC class I heavy chain polypeptide; and
   ii) an Ig Fc polypeptide; or
a3) the first polypeptide comprises, in order from N-terminus to C-terminus:
   i) the KRAS peptide; and
   ii) a β2-microglobulin polypeptide; and
b3) the second polypeptide comprises, in order from N-terminus to C-terminus:
   i) an MHC class I heavy chain polypeptide; and
   ii) an Ig Fc polypeptide,
   wherein the Ig Fc polypeptide is a human IgG1 Fc polypeptide that substantially does not induce cell lysis, optionally comprising the amino acid sequence of FIG. 3G.

Aspect 83. A method according to aspects 82,
wherein the β2-microglobulin polypeptide is joined to the KRAS peptide by a first linker that comprises the sequence CGGGS(GGGGS)n (SEQ ID NO:142) or GCGGS(GGGGS)n (SEQ ID NO:140), where n is an integer from 1-10, e.g., 2 or 3,
wherein the MHC heavy chain polypeptide comprises a Cys at residue 84 and a Cys at residue 236,
wherein the β2M polypeptide comprises a Cys at residue 12,
wherein the Cys at amino acid residue 12 of the β2M polypeptide is disulfide bonded to a Cys at amino acid residue 236 of the MHC heavy chain polypeptide,
wherein a disulfide bond links the Cys in the linker with a Cys substituted for Tyr84 of the MHC heavy chain polypeptide, and
wherein the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, .an HLA-A*3303 polypeptide, and an HLA-A*2401 polypeptide.

Aspect 84. A method according to any one of aspects 79-81, wherein the APP is a single chain polypeptide comprising:
i) a KRAS peptide;
ii) a β2-microglobulin polypeptide; and
iii) an MHC class I heavy chain polypeptide.

Aspect 85. A method according to aspect 84, wherein the single chain APP comprises in order from N-terminus to C-terminus: A) i) a KRAS peptide; ii) a β2M polypeptide; iii) a class I MHC heavy chain polypeptide; and iv) an Ig Fc polypeptide,

wherein the Ig Fc polypeptide is a human IgG1 Fc polypeptide that substantially does not induce cell lysis, optionally comprising the amino acid sequence of FIG. 3G.

Aspect 86. A method according to aspect 85,
wherein the β2-microglobulin polypeptide is joined to the KRAS peptide by a first linker that comprises the sequence CGGGS(GGGGS)n (SEQ ID NO:142) or GCGGS(GGGGS)n (SEQ ID NO:140), where n is an integer from 1-10, e.g., 2 or 3,
wherein the MHC heavy chain polypeptide comprises a Cys at residue 84 and a Cys at residue 236,
wherein the β2M polypeptide comprises a Cys at residue 12,
wherein the Cys at amino acid residue 12 of the β2M polypeptide is disulfide bonded to a Cys at amino acid residue 236 of the MHC heavy chain polypeptide,
wherein a disulfide bond links the Cys in the linker with a Cys substituted for Tyr84 of the MHC heavy chain polypeptide, and
wherein the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, .an HLA-A*3303 polypeptide, and an HLA-A*2401 polypeptide.

### EXAMPLES

The following examples are put forth so as to provide those of ordinary skill in the art with a complete disclosure and description of how to make and use the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Generation of Kras G12V-Specific HLA-A11+ T Cells and Assessment of their Expansion in Response to Kras G12V HLA-A11 TMPs

T-cell receptor (TCR)-transduced primary human T cells were generated from HLA-A11+ donors. The TCRs were responsive to KRAS G12V (7-16) peptide. The ability of TMPs comprising KRAS G12V (7-16) and HLA-A11 to expand KRAS G12V (7-16)-specific, TCR-transduced, CD8⁺ T cells was assessed.

### Materials and Methods

The following TMPs were used: (i) TMP 4074 (single chain; comprising G12V (7-16) as the peptide epitope); (ii) TMP 4073 (single chain; comprising G12D (7-16) as the peptide epitope); (iii) TMP 4072-4030 (split chain comprising heterodimers of construct 4072 and construct 4030; and comprising G12V (7-16) as the peptide epitope); and (iv) TMP 4072-4029 (split chain comprising heterodimers of construct 4072 and construct 4029; and comprising G12D (7-16) as the peptide epitope). The TMPs included an HLA-A*11:01 allele heavy chain; a β2M polypeptide; KRAS G12V (7-16) or KRAS G12D (7-16) as the peptide epitope; affinity-attenuated IL-2 variant (IL-2 (H16A; F42A)); and a human immunoglobulin G1 (IgG1) fragment crystallizable (Fc) polypeptide.

The G12D (7-16) peptide epitope has the amino acid sequence:VVVGADGVGK (SEQ ID NO:179). The G12V (7-16) peptide epitope has the amino acid sequence: VVVGAVGVGK (SEQ ID NO:180).

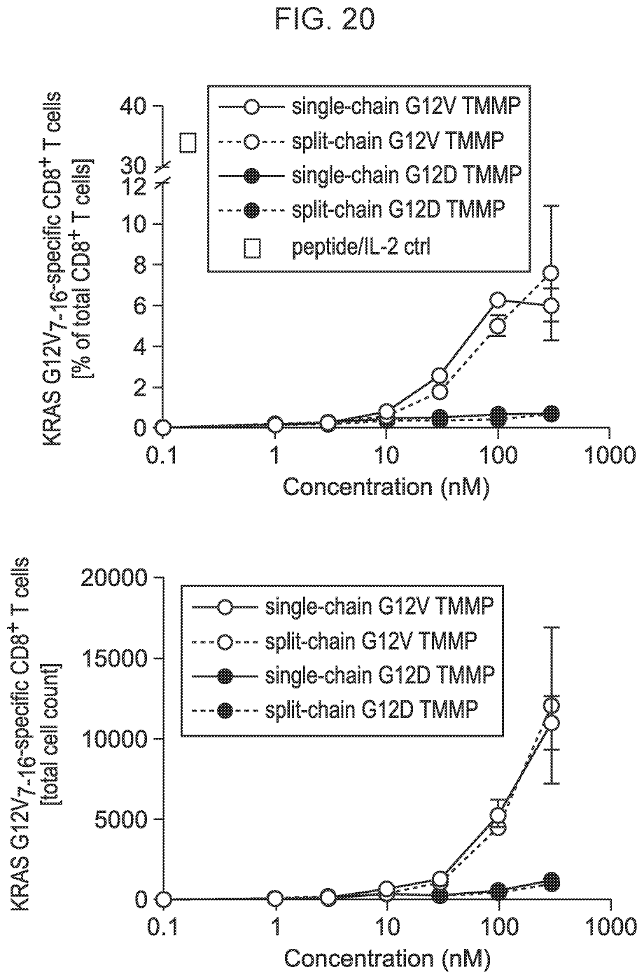

The 4072-4029 TMP is a homodimer of two heterodimers, each heterodimer comprising: a) the 4029 polypeptide (FIG. 14K), a “light chain” polypeptide which includes: i) the G12D (7-16) peptide; ii) a GCGGS(GGGS)2) (SEQ ID NO:584) linker; and iii) a β2M polypeptide comprising an R12C substitution; and b) the 4072 polypeptide (FIG. 13L), a “heavy chain” polypeptide which includes: i) an HLA-A*11:01 allele heavy chain with Y84C and A236C substitutions; ii) an IgG1 Fc polypeptide with L234A and L235A substitutions; and iii) two copies of IL2 (H16A; F42A). The heavy and light chain polypeptides are joined by 2 disulfide bonds formed between: i) the Cys in the GCGGS(GGGGS)2 (SEQ ID NO:585) linker and the Cys formed by the Y84C substitution in the HLA-A*11.01 heavy chain polypeptide; and ii) the Cys formed by the R12C substitution in the β2M polypeptide and the Cys formed by the A236C substitution in the HLA-A*11.01 heavy chain polypeptide. The 4072-4029 TMP is a homodimer of two copies of the heterodimer, joined by disulfide bonds formed between the respective IgG1 Fc regions. The 4072-4029 TMP is referred to in FIG. 20 as “split-chain G12D TMP.”

The 4072-4030 TMP is a homodimer of two heterodimers, each heterodimer comprising: a) the 4030 polypeptide (FIG. 14Q), which includes: i) the G12V (7-16) peptide; ii) a GCGGS(GGGS)2) (SEQ ID NO:584) linker; and iii) a β2M polypeptide comprising an R12C substitution; and b) the 4072 polypeptide (FIG. 13L), a “heavy chain” polypeptide which includes: i) an HLA-A*11:01 allele heavy chain with Y84C and A236C substitutions; ii) an IgG1 Fc polypeptide with L234A and L235A substitutions; and iii) two copies of IL2 (H16A; F42A). The heavy and light chain polypeptides are joined by 2 disulfide bonds formed between: i) the Cys in the GCGGS(GGGGS)2 (SEQ ID NO:585) linker and the Cys formed by the Y84C substitution in the HLA-A*11.01 heavy chain polypeptide; and ii) the Cys formed by the R12C substitution in the β2M polypeptide and the Cys formed by the A236C substitution in the HLA-A*11.01 heavy chain polypeptide. The 4072-4030 TMP is a homodimer of two copies of the heterodimer, joined by disulfide bonds formed between the respective IgG1 Fc regions. The 4072-4030 TMP is referred to in FIG. 20 as “split-chain G12V TMP.”

The 4073 TMP is a homodimer of two single-chain polypeptides, each single-chain polypeptide (depicted in FIG. 19B) comprising: i) the G12D (7-16) peptide; ii) a GCGGS(GGGGS)2 (SEQ ID NO:585) linker; iii) a β2M (R12C) polypeptide; iv) an HLA-A*11:01 allele heavy chain with Y84C and A236C substitutions; v) an IgG1 Fc polypeptide with L234A and L235A substitutions; and vi) two copies of IL2 (H16A; F42A). Each of the two single-chain polypeptides can include intrachain disulfide bonds formed between: i) the Cys in the GCGGS(GGGGS)2 (SEQ ID NO:585) linker and the Cys formed by the Y84C substitution in the HLA-A*11.01 heavy chain polypeptide; and ii) the Cys formed by the R12C substitution in the β2M polypeptide and the Cys formed by the A236C substitution in the HLA-A*11.01 heavy chain polypeptide. The two single-chain polypeptides are disulfide bonded to one another via disulfide bonds formed between the IgG1 Fc polypeptide in the respective single-chain polypeptides. The 4073 TMP is referred to in FIG. 20 as “single-chain G12D TMP.”

The 4074 TMP is a homodimer of two single-chain polypeptides, each single-chain polypeptide (depicted in FIG. 19C) comprising: i) the G12V (7-16) peptide; ii) a GCGGS(GGGGS)2 (SEQ ID NO:585) linker; iii) a β2M (R12C) polypeptide; iv) an HLA-A*11:01 allele heavy chain with Y84C and A236C substitutions; v) an IgG1 Fc polypeptide with L234A and L235A substitutions; and vi) two copies of IL2 (H16A; F42A). Each of the two single-chain polypeptides can include intrachain disulfide bonds formed between: i) the Cys in the GCGGS(GGGGS)2 (SEQ ID NO:585) linker and the Cys formed by the Y84C substitution in the HLA-A*11.01 heavy chain polypeptide; and ii) the Cys formed by the R12C substitution in the β2M polypeptide and the Cys formed by the A236C substitution in the HLA-A*11.01 heavy chain polypeptide. The two single-chain polypeptides are disulfide bonded to one another via disulfide bonds formed between the IgG1 Fc polypeptide in the respective single-chain polypeptides. The 4074 TMP is referred to in FIG. 20 as “single-chain G12V TMP.”

HLA-A11⁺ human healthy donor CD8⁺ T cells were transduced with a TCR specific for KRAS G12V (7-16). Transduced CD8⁺ T cells were stimulated with TMPs containing KRAS G12V (7-16) as the peptide epitope, or containing KRAS G12D (7-16) as the peptide epitope, in the presence of autologous peripheral blood mononuclear cells (PBMCs) as feeders. Expansion of the TCR-transduced T cells was monitored using antibodies specific for the mouse constant region of the TCR (engineered into the TCR) and assessed by flow cytometry.

### Results

As depicted in FIG. 20, KRAS G12V (7-16) HLA-A11 TMPs can expand KRAS G12V(7-16)-specific CD8⁺ T cells in a dose-dependent manner, both as a percent of CD8⁺ T cells (upper panel) and as the total number of antigen-specific cells (lower panel), in 10-day in vitro assays. The effect was epitope specific, as control TMPs comprising KRAS G12D (7-16) did not induce expansion in a dose-dependent manner.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-20. (canceled)

21. A T-cell modulatory polypeptide (TMP) comprising at least one heterodimeric polypeptide, wherein the at least one heterodimeric polypeptide comprises:
a) a first polypeptide comprising
i) a KRAS peptide epitope comprising a KRAS epitope expressed on a cancer cell,
ii) a β2-microglobulin (β2M) polypeptide, and
iii) a Cys-containing linker that joins the KRAS peptide epitope to the β2M polypeptide; and
b) a second polypeptide comprising
i) a major histocompatibility complex (MHC) class I heavy chain polypeptide, and
ii) one or more variant IL-2 polypeptides, wherein the one or more variant IL-2 comprise the amino acid sequence set forth in SEQ ID NO:241 and
iii) an immunoglobulin (Ig) Fc polypeptide,
wherein one or more independently selected linkers may be interposed between any two of the components of the second polypeptide, and
wherein the TMP comprises (a) a first disulfide bond formed between (i) the Cys residue in the Cys-containing linker, and (ii) a Cys residue in the MHC class I heavy chain polypeptide; and (b) a second disulfide bond formed between a Cys residue in the β2M polypeptide and a Cys residue in the MHC class I heavy chain polypeptide.

22. The TMP of claim 21, wherein the Ig Fc polypeptide is an IgG1 Fc polypeptide that substantially does not induce cell lysis, and wherein the Ig Fc polypeptide comprises one or more amino acid substitutions selected from N77A, L14A, L15A, L14F, L15F, and P111S, based on the number of the IgG1 Fc amino acid sequence set forth in SEQ ID NO:19.

23. The TMP of claim 22, wherein the β2M polypeptide and the MHC heavy chain polypeptide are joined by a disulfide bond that joins a Cys at amino acid residue 12 of the β2M polypeptide and a Cys at amino acid residue 236 of the MHC heavy chain polypeptide.

24. The TMP of claim 23, wherein the MHC heavy chain polypeptide comprises an amino acid sequence having at least 95% amino acid sequence identity to an HLA-A polypeptide having an amino acid sequence selected from SEQ ID NOs: 44 and 49-51.

25. The TMP of claim 24, wherein the KRAS peptide epitope comprises a sequence selected from the group consisting of:
A) VVGADGVGK (SEQ ID NO:176), VVGACGVGK (SEQ ID NO:177), VVGAVGVGK (SEQ ID NO:178), VVVGADGVGK (SEQ ID NO:179), VVVGAVGVGK (SEQ ID NO:180), VVVGACGVGK (SEQ ID NO:181), VTGADGVGK (SEQ ID NO:182), VTGAVGVGK (SEQ ID NO:183), VTGACGVGK (SEQ ID NO:184), VTVGADGVGK (SEQ ID NO:185), VTVGAVGVGK (SEQ ID NO:186), and VTVGACGVGK (SEQ ID NO:187); and wherein the KRAS peptide epitope has a length of 9 amino acids or 10 amino acids, or a length of at least 9 amino acids or 10 amino acids;
B) VVVGAGDVGK (SEQ ID NO:188); VVGAGDVGK (SEQ ID NO:189); VVVGARGVGK (SEQ ID NO:190); and VVGARGVGK (SEQ ID NO:191); and wherein the KRAS peptide epitope has a length of 9 amino acids or 10 amino acids, or a length of at least 9 amino acids or 10 amino acids;
C) LVVVGADGV (SEQ ID NO:192), LVVVGAVGV (SEQ ID NO:193), LVVVGACGV (SEQ ID NO:194), KLVVVGADGV (SEQ ID NO:195), KLVVVGAVGV (SEQ ID NO:196), KLVVVGACGV (SEQ ID NO:197), LLVVGADGV (SEQ ID NO:198), LLVVGAVGV (SEQ ID NO:199), LLVVGACGV (SEQ ID NO:200), FLVVVGADGV (SEQ ID NO:201), FLVVVGAVGV (SEQ ID NO:202), and FLVVVGACGV (SEQ ID NO:203); and wherein the KRAS peptide epitope has a length of 9 amino acids or 10 amino acids, or a length of at least 9 amino acids or 10 amino acids;
D) KLVVVGAGDV (SEQ ID NO:204); and KLVVVGARGV (SEQ ID NO:205); wherein the KRAS peptide epitope has a length of 9 amino acids or 10 amino acids, or a length of at least 9 amino acids or 10 amino acids;
E) GAGDVGKSAL (SEQ ID NO:206); AGDVGKSAL (SEQ ID NO:207); DVGKSALTI (SEQ ID NO:208); GAVGVGKSAL (SEQ ID NO:209); AVGVGKSAL (SEQ ID NO:210); YKLVVVGAV (SEQ ID NO:211); ARGVGKSAL (SEQ ID NO:212); GARGVGKSAL (SEQ ID NO:213); EYKLVVVGAR (SEQ ID NO:214); RGVGKSALTI (SEQ ID NO:215); LVVVGARGV (SEQ ID NO:216); GADGVGKSAL (SEQ ID NO:217); ACGVGKSAL (SEQ ID NO:218); and GACGVGKSAL (SEQ ID NO:219); wherein the KRAS peptide epitope has a length of 9 amino acids or 10 amino acids, or a length of at least 9 amino acids or 10 amino acids; and
F) VVGAVGVGK (SEQ ID NO:178), VVVGAVGVGK (SEQ ID NO:180), VGAVGVGKS (SEQ ID NO:222), VGAVGVGKSA (SEQ ID NO:223), AVGVGKSAL (SEQ ID NO:210), AVGVGKSALT (SEQ ID NO:225), GAVGVGKSAL (SEQ ID NO:209), GAVGVGKSA (SEQ ID NO:227), LVVVGAVGVG (SEQ ID NO:228), LVVVGAVGV (SEQ ID NO:193), KLVVVGAVGV (SEQ ID NO:196), and KLVVVGAVG (SEQ ID NO:231); where the KRAS peptide epitope has a length of 9 amino acids or 10 amino acids, or a length of at least 9 amino acids or 10 amino acids.

26. The TMP of claim 21, wherein the TMP comprises two identical heterodimeric polypeptides, and wherein the heterodimeric TMPs are covalently linked together by one or more disulfide bonds between the Ig Fc of the first heterodimeric polypeptide and the Ig Fc of the second heterodimeric polypeptide.

27. One or more nucleic acids comprising the nucleotide sequences encoding the first and second polypeptides of the TMP of claim 21.

28. A method of producing a TMP, the method comprising culturing host cells that are genetically modified with the one or more nucleic acids of claim 27 under conditions such that the genetically modified host cells produce the TMP.

29. A method of selectively modulating the activity of T cell specific for a KRAS peptide epitope, the method comprising contacting the T cell with the TMP of claim 21, wherein said contacting selectively modulates the activity of the epitope-specific T cell.

30. A method of treating a KRAS-associated cancer in a patient, the method comprising administering to the patient an effective amount of a pharmaceutical composition comprising the TMP of claim 26, wherein the patient may also be administered an immune checkpoint inhibitor.

31. A T-cell modulatory polypeptide (TMP), wherein the TMP comprises at least one single-chain polypeptide, wherein the at least one single-chain polypeptide comprises:
i) a KRAS peptide epitope comprising a KRAS epitope expressed on a cancer cell;
ii) a β2-microglobulin (β2M) polypeptide;
iii) a major histocompatibility complex (MHC) class I heavy chain polypeptide;
iv) an immunoglobulin (Ig) Fc polypeptide; and
v) one or more variant IL-2 polypeptides, wherein the one or more variant IL-2 comprise the amino acid sequence set forth in SEQ ID NO:241, and
wherein one or more independently selected linkers may be interposed between any two of the components of the single-chain polypeptide.

32. The TMP of claim 31, wherein:
i) a Cys-containing linker joins the β2M polypeptide to the KRAS peptide epitope;
ii) the MHC heavy chain polypeptide comprises a Cys at residue 84 and a Cys at residue 236;
iii) the β2M polypeptide comprises a Cys at residue 12;
iv) a disulfide bond links the Cys at amino acid residue 12 of the β2M polypeptide to the Cys at amino acid residue 236 of the MHC heavy chain polypeptide; and
v) a disulfide bond links the Cys in the Cys-containing linker with the Cys at residue 84 of the MHC heavy chain polypeptide.

33. The TMP of claim 32, wherein the Ig Fc polypeptide is an IgG1 Fc polypeptide that substantially does not induce cell lysis, and wherein the Ig Fc polypeptide comprises one or more amino acid substitutions selected from N77A, L14A, LISA, L14F, L15F, and P111S, based on the number of the IgG1 Fc amino acid sequence set forth in SEQ ID NO:19.

34. The TMP of claim 23, wherein the MHC heavy chain polypeptide comprises an amino acid sequence having at least 95% amino acid sequence identity to an HLA-A polypeptide having an amino acid sequence selected from SEQ ID NOs: 44 and 49-51.

35. The TMP of claim 34, wherein the KRAS peptide epitope comprises a sequence selected from the group consisting of:
A) VVGADGVGK (SEQ ID NO:176), VVGACGVGK (SEQ ID NO:177), VVGAVGVGK (SEQ ID NO:178), VVVGADGVGK (SEQ ID NO:179), VVVGAVGVGK (SEQ ID NO:180), VVVGACGVGK (SEQ ID NO:181), VTGADGVGK (SEQ ID NO:182), VTGAVGVGK (SEQ ID NO:183), VTGACGVGK (SEQ ID NO:184), VTVGADGVGK (SEQ ID NO:185), VTVGAVGVGK (SEQ ID NO:186), and VTVGACGVGK (SEQ ID NO:187); and wherein the KRAS peptide epitope has a length of 9 amino acids or 10 amino acids, or a length of at least 9 amino acids or 10 amino acids;
B) VVVGAGDVGK (SEQ ID NO:188); VVGAGDVGK (SEQ ID NO:189); VVVGARGVGK (SEQ ID NO:190); and VVGARGVGK (SEQ ID NO:191); and wherein the KRAS peptide epitope has a length of 9 amino acids or 10 amino acids, or a length of at least 9 amino acids or 10 amino acids;
C) LVVVGADGV (SEQ ID NO:192), LVVVGAVGV (SEQ ID NO:193), LVVVGACGV (SEQ ID NO:194), KLVVVGADGV (SEQ ID NO:195), KLVVVGAVGV (SEQ ID NO:196), KLVVVGACGV (SEQ ID NO:197), LLVVGADGV (SEQ ID NO:198), LLVVGAVGV (SEQ ID NO:199), LLVVGACGV (SEQ ID NO:200), FLVVVGADGV (SEQ ID NO:201), FLVVVGAVGV (SEQ ID NO:202), and FLVVVGACGV (SEQ ID NO:203); and wherein the KRAS peptide epitope has a length of 9 amino acids or 10 amino acids, or a length of at least 9 amino acids or 10 amino acids;
D) KLVVVGAGDV (SEQ ID NO:204); and KLVVVGARGV (SEQ ID NO:205); wherein the KRAS peptide epitope has a length of 9 amino acids or 10 amino acids, or a length of at least 9 amino acids or 10 amino acids;
E) GAGDVGKSAL (SEQ ID NO:206); AGDVGKSAL (SEQ ID NO:207); DVGKSALTI (SEQ ID NO:208); GAVGVGKSAL (SEQ ID NO:209); AVGVGKSAL (SEQ ID NO:210); YKLVVVGAV (SEQ ID NO:211); ARGVGKSAL (SEQ ID NO:212); GARGVGKSAL (SEQ ID NO:213); EYKLVVVGAR (SEQ ID NO:214); RGVGKSALTI (SEQ ID NO:215); LVVVGARGV (SEQ ID NO:216); GADGVGKSAL (SEQ ID NO:217); ACGVGKSAL (SEQ ID NO:218); and GACGVGKSAL (SEQ ID NO:219); wherein the KRAS peptide epitope has a length of 9 amino acids or 10 amino acids, or a length of at least 9 amino acids or 10 amino acids; and
F) VVGAVGVGK (SEQ ID NO:178), VVVGAVGVGK (SEQ ID NO:180), VGAVGVGKS (SEQ ID NO:222), VGAVGVGKSA (SEQ ID NO:223), AVGVGKSAL (SEQ ID NO:210), AVGVGKSALT (SEQ ID NO:225), GAVGVGKSAL (SEQ ID NO:209), GAVGVGKSA (SEQ ID NO:227), LVVVGAVGVG (SEQ ID NO:228), LVVVGAVGV (SEQ ID NO:193), KLVVVGAVGV (SEQ ID NO:196), and KLVVVGAVG (SEQ ID NO:231); where the KRAS peptide epitope has a length of 9 amino acids or 10 amino acids, or a length of at least 9 amino acids or 10 amino acids.

36. The TMP of claim 31, wherein the TMP comprises two single-chain polypeptides that have the same amino acid sequence, and wherein the single-chain polypeptides are covalently linked together by one or more disulfide bonds between the Ig Fc of the first single-chain polypeptide and the Ig Fc of the second single-chain polypeptide.

37. A nucleic acid encoding the single-chain polypeptide of the TMP of claim 31.

38. A method of producing a TMP, the method comprising culturing host cells that are genetically modified with the nucleic acid of claim 37 under conditions such that the genetically modified host cells produce the TMP.

39. A method of selectively modulating the activity of T cell specific for a KRAS peptide epitope, the method comprising contacting the T cell with the TMP of claim 31, wherein said contacting selectively modulates the activity of the epitope-specific T cell.

40. A method of treating a KRAS-associated cancer in a patient, the method comprising administering to the patient an effective amount of a pharmaceutical composition comprising the TMP of claim 36, wherein the patient may also be administered an immune checkpoint inhibitor.


SEQUENCE LISTING (602 sequences)

SEQ ID NO: 1 - protein, 188 aa, Homo sapiens [sequence omitted]

SEQ ID NO: 2 - protein, 189 aa, Homo sapiens [sequence omitted]

SEQ ID NO: 3 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - protein, 208 aa, Homo sapiens [sequence omitted]

SEQ ID NO: 5 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 9 - protein, 110 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 10 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 11 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 12 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 14 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 15 - protein, 133 aa, Homo sapiens [sequence omitted]

SEQ ID NO: 16 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 17 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 18 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 19 - protein, 227 aa, synthetic construct
  REGION 1..227: note = synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 325 aa, synthetic construct
  REGION 1..325: note = synthetic sequence
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 246 aa, synthetic construct
  REGION 1..246: note = synthetic sequence
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 383 aa, synthetic construct
  REGION 1..383: note = synthetic sequence
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 23 - protein, 276 aa, synthetic construct
  REGION 1..276: note = synthetic sequence
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 24 - protein, 353 aa, synthetic construct
  REGION 1..353: note = synthetic sequence
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 25 - protein, 222 aa, synthetic construct
  REGION 1..222: note = synthetic sequence
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 26 - protein, 327 aa, synthetic construct
  REGION 1..327: note = synthetic sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 227 aa, synthetic construct
  REGION 1..227: note = synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 227 aa, synthetic construct
  REGION 1..227: note = synthetic sequence
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 227 aa, synthetic construct
  REGION 1..227: note = synthetic sequence
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 227 aa, synthetic construct
  REGION 1..227: note = synthetic sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 32 - protein, 119 aa, Macaca mulatta
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 33 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 34 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 35 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 36 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 37 - protein, 365 aa, Homo sapiens [sequence omitted]

SEQ ID NO: 38 - protein, 365 aa, Homo sapiens [sequence omitted]

SEQ ID NO: 39 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 40 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 41 - protein, 275 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 42 - protein, 275 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWE

SEQ ID NO: 43 - protein, 275 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 44 - protein, 276 aa, Homo sapiens [sequence omitted]

SEQ ID NO: 45 - protein, 274 aa, Mus musculus
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRW

SEQ ID NO: 46 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 47 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 48 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 49 - protein, 276 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 50 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 51 - protein, 276 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 52 - protein, 276 aa, synthetic construct
  REGION 1..276: note = synthetic sequence
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 53 - protein, 276 aa, synthetic construct [sequence omitted]

SEQ ID NO: 54 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 55 - protein, 276 aa, synthetic construct; identical to SEQ ID NO: 49
  REGION 1..276: note = synthetic sequence

SEQ ID NO: 56 - protein, 276 aa, synthetic construct
  REGION 1..276: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 57 - protein, 276 aa, synthetic construct; identical to SEQ ID NO: 50
  REGION 1..276: note = synthetic sequence

SEQ ID NO: 58 - protein, 276 aa, synthetic construct
  REGION 1..276: note = synthetic sequence
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 59 - protein, 276 aa, synthetic construct; identical to SEQ ID NO: 51
  REGION 1..276: note = synthetic sequence

SEQ ID NO: 60 - protein, 276 aa, synthetic construct
  REGION 1..276: note = synthetic sequence
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 61 - protein, 276 aa, synthetic construct
  REGION 1..276: note = synthetic sequence
  VARIANT 9: note = X is phenylalanine, tyrosine, serine, or threonine
  VARIANT 44: note = X is lysine or arginine
  VARIANT 62: note = X is glutamine, glycine, glutamic acid, or arginine
  VARIANT 63: note = X is asparagine or glutamic acid
  VARIANT 65: note = X is arginine or glycine
  VARIANT 66: note = X is asparagine or lysine
  VARIANT 67: note = X is methionine or valine
  VARIANT 70: note = X is histidine or glutamine
  VARIANT 73: note = X is threonine or isoleucine
  VARIANT 74: note = X is aspartic acid or histidine
  VARIANT 76: note = X is alanine, valine, or glutamic acid
  VARIANT 77: note = X is asparagine or aspartic acid
  VARIANT 79: note = X is glycine or arginine
  VARIANT 80: note = X is threonine or arginine
  VARIANT 81: note = X is leucine or alanine
  VARIANT 82: note = X is arginine or leucine
  VARIANT 83: note = X is glycine ir arginine
  VARIANT 90: note = X is alanine or aspartic acid
  VARIANT 95: note = X is isoleucine, leucine, or valine
  VARIANT 97: note = X is isoleucine, arginine, or methionine
  VARIANT 99: note = X is phenylalanine or tyrosine
  VARIANT 105: note = X is serine or proline
  VARIANT 107: note = X is tryptophan or glycine
  VARIANT 114: note = X is arginine, histidine, or glutamine
  VARIANT 116: note = X is aspartic acid or tyrosine
  VARIANT 127: note = X is asparagine or lysine
  VARIANT 142: note = X is threonine or isoleucine
  VARIANT 144: note = X is lysine or glutamine
  VARIANT 145: note = X is arginine or histidine
  VARIANT 149: note = X is alanine or threonine
  VARIANT 150: note = X is alanine or valine
  VARIANT 151: note = X is histidine or arginine
  VARIANT 156: note = X is arginine, leucine, glutamine, or tryptophan
  VARIANT 158: note = X is valine or alanine
  VARIANT 161: note = X is aspartic acid or glutamic acid
  VARIANT 163: note = X is arginine or threonine
  VARIANT 166: note = X is aspartic acid or glutamic acid
  VARIANT 167: note = X is tryptophan or glycine
  VARIANT 184: note = X is proline or alanine
  VARIANT 193: note = X is proline or alanine
  VARIANT 194: note = X is valine or isoleucine
  VARIANT 207: note = X is serine or glycine
  VARIANT 246: note = X is alanine or serine
  VARIANT 253: note = X is glutamine or glutamic acid
  VARIANT 276: note = X is proline or leucine
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 62 - protein, 276 aa, synthetic construct [sequence omitted]

SEQ ID NO: 63 - protein, 276 aa, synthetic construct
  REGION 1..276: note = synthetic sequence
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 64 - protein, 276 aa, synthetic construct
  REGION 1..276: note = synthetic sequence
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 65 - protein, 276 aa, synthetic construct
  REGION 1..276: note = synthetic sequence
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 66 - protein, 276 aa, synthetic construct
  REGION 1..276: note = synthetic sequence
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 67 - protein, 276 aa, synthetic construct
  REGION 1..276: note = synthetic sequence
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 68 - protein, 276 aa, synthetic construct
  REGION 1..276: note = synthetic sequence
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 69 - protein, 276 aa, synthetic construct
  REGION 1..276: note = synthetic sequence
  VARIANT 9: note = X is histidine, tyrosine, or aspartic acid
  VARIANT 11: note = X is alanine or serine
  VARIANT 12: note = X is methionine or valine
  VARIANT 24: note = X is alanine, serine, or threonine
  VARIANT 32: note = X is glutamine or leucine
  VARIANT 41: note = X is alanine or threonine
  VARIANT 45: note = X is glutamic acid, methionine, lysine, or threonine
  VARIANT 46: note = X is alanine or threonine
  VARIANT 63: note = X is glutamic acid or asparagine
  VARIANT 66: note = X is isoleucine or lysine
  VARIANT 67: note = X is tyrosine, phenylalanie, serine, or cystine
  VARIANT 70: note = X is asparagine or glutamine
  VARIANT 71: note = X is alanine or threonine
  VARIANT 74: note = X is aspartic acid or tyrosine
  VARIANT 76: note = X is glutamic acid or valine
  VARIANT 77: note = X is serine or asparagine
  VARIANT 80: note = X is threonine, asparagine, or isoleucine
  VARIANT 81: note = X is alanine or leucine
  VARIANT 82: note = X is leucine or arginine
  VARIANT 83: note = X is arginine or glycine
  VARIANT 94: note = X is threonine or isoleucine
  VARIANT 95: note = X is leucine or isoleucine
  VARIANT 97: note = X is arginine or serine
  VARIANT 131: note = X is arginine or serine
  VARIANT 143: note = X is S or T
  VARIANT 147: note = X is L or W
  VARIANT 152: note = Xaa is E OR V
  VARIANT 156: note = Xaa is R, D, L
  VARIANT 158: note = Xaa A or T
  VARIANT 163: note = X is leucine, glutamic acid, or threonine
  VARIANT 177: note = X is glutamic acid or aspartic acid
  VARIANT 178: note = X is lysine or threonine
  VARIANT 180: note = X is glutamic acid or glutamine
  VARIANT 194: note = X is isoleucine or valine
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 70 - protein, 276 aa, synthetic construct
  REGION 1..276: note = synthetic sequence
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 71 - protein, 276 aa, synthetic construct
  REGION 1..276: note = synthetic sequence
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 72 - protein, 276 aa, synthetic construct
  REGION 1..276: note = synthetic sequence
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 73 - protein, 276 aa, synthetic construct
  REGION 1..276: note = synthetic sequence
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 74 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 75 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 76 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 77 - protein, 276 aa, synthetic construct
  REGION 1..276: note = synthetic sequence
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 78 - protein, 276 aa, synthetic construct
  REGION 1..276: note = synthetic sequence
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 79 - protein, 276 aa, synthetic construct
  REGION 1..276: note = synthetic sequence
  VARIANT 1: note = X is cysteine or glycine
  VARIANT 6: note = X is arginine or lysine
  VARIANT 9: note = X is phenylalanine, tyrosine, serine, or aspartic acid
  VARIANT 14: note = X is arginine or tryptophan
  VARIANT 21: note = X is histidine or arginine
  VARIANT 24: note = X is alanine or serine
  VARIANT 35: note = X is glutamine or arginine
  VARIANT 49: note = X is alanine or arginine
  VARIANT 66: note = X is asparagine, or lysine
  VARIANT 73: note = X is threonine or alanine
  VARIANT 77: note = X is serine or asparagine
  VARIANT 80: note = X is asparagine or lysine
  VARIANT 90: note = X is alanine or aspartic acid
  VARIANT 91: note = X is glycine or arginine
  VARIANT 94: note = X is threonine or isoleucine
  VARIANT 95: note = X is leucine or isoleucine
  VARIANT 97: note = X is tryptophan or arginine
  VARIANT 99: note = X is cysteine, tyrosine, phenylalanine, or serine
  VARIANT 103: note = X is leucine or valine
  VARIANT 113: note = X is tyrosine or histidine
  VARIANT 114: note = X is aspartic acid or asparagine
  VARIANT 116: note = X is tyrosine, phenylalanine, serine, or leucine
  VARIANT 147: note = X is leucine or tryptophan
  VARIANT 152: note = X is glutamic acid, alanine, or threonine
  VARIANT 156: note = X is arginine, leucine, or tryptophan
  VARIANT 163: note = X is leucine or threonine
  VARIANT 173: note = X is glutamic acid or lysine
  VARIANT 177: note = X is glutamic acid or lysine
  VARIANT 184: note = X is histidine or proline
  VARIANT 194: note = X is arginine or valine
  VARIANT 219: note = X is tryptophan or arginine
  VARIANT 248: note = X is valine or methionine
  VARIANT 253: note = X is glutamic acid or lysine
  VARIANT 261: note = X is methionine or valine
  VARIANT 267: note = X is proline or glutamine
  VARIANT 273: note = X is arginine or serine
  VARIANT 275: note = X is proline or glycine
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 80 - protein, 284 aa, synthetic construct
  REGION 1..284: note = synthetic sequence
  VARIANT 89: note = Xaa is K or E
  VARIANT 107: note = Xaa is R or G
  VARIANT 157: note = Xaa is R or G
  VARIANT 158: note = Xaa is A or V
  VARIANT 255: note = Xaa is Q or P
  VARIANT 267: note = Xaa is P or S
GSHSLKYFHTSVSRPGRGEPRFISVGYVDDTQFVRFDNDAASPRMVPRAPWMEQEGSEYWDRETRSARDTAQIFRVNLRTLRGYYNQSXAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXRYTCHVQHEGLXEPVTLRWKPASQPTIPI

SEQ ID NO: 81 - protein, 284 aa, synthetic construct
  REGION 1..284: note = synthetic sequence
  VARIANT 7: note = Xaa is Y or F
  VARIANT 50: note = Xaa is P or Q
  VARIANT 251: note = Xaa is S or P
  VARIANT 278: note = Xaa is P or L
GSHSLRXFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 82 - protein, 284 aa, synthetic construct
  REGION 1..284: note = synthetic sequence
  VARIANT 13: note = Xaa is S or F
  VARIANT 27: note = Xaa is Y or H
  VARIANT 31: note = Xaa is T, S, or M
  VARIANT 34: note = Xaa is L or V
  VARIANT 54: note = Xaa is Q or R
  VARIANT 81: note = Xaa is P or L
  VARIANT 100: note = Xaa is G or D
  VARIANT 104: note = Xaa is G or V
  VARIANT 105: note = Xaa is S or C
  VARIANT 110: note = Xaa is L or I
  VARIANT 159: note = Xaa is Y or H
  VARIANT 169: note = Xaa is H or R
  VARIANT 171: note = Xaa is Y or H
  VARIANT 178: note = Xaa is M or T
  VARIANT 185: note = Xaa is P or A
  VARIANT 219: note = Xaa is R, W, or Q
  VARIANT 258: note = Xaa is T or M
  VARIANT 275: note = Xaa is K or E
GSHSMRYFSAAVXRPGRGEPRFIAMGXVDDXQFXRFDSDSACPRMEPRAPWVEXEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 83 - protein, 276 aa, synthetic construct; identical to SEQ ID NO: 61
  REGION 1..276: note = synthetic sequence
  VARIANT 9: note = X is phenylalanine, tyrosine, serine, or threonine
  VARIANT 44: note = X is lysine or arginine
  VARIANT 62: note = X is glutamine, glycine, glutamic acid, or arginine
  VARIANT 63: note = X is asparagine or glutamic acid
  VARIANT 65: note = X is arginine or glycine
  VARIANT 66: note = X is asparagine or lysine
  VARIANT 67: note = X is methionine or valine
  VARIANT 70: note = X is histidine or glutamine
  VARIANT 73: note = X is threonine or isoleucine
  VARIANT 74: note = X is aspartic acid or histidine
  VARIANT 76: note = X is alanine, valine, or glutamic acid
  VARIANT 77: note = X is asparagine or aspartic acid
  VARIANT 79: note = X is glycine or arginine
  VARIANT 80: note = X is threonine or arginine
  VARIANT 81: note = X is leucine or alanine
  VARIANT 82: note = X is arginine or leucine
  VARIANT 83: note = X is glycine ir arginine
  VARIANT 90: note = X is alanine or aspartic acid
  VARIANT 95: note = X is isoleucine, leucine, or valine
  VARIANT 97: note = X is isoleucine, arginine, or methionine
  VARIANT 99: note = X is phenylalanine or tyrosine
  VARIANT 105: note = X is serine or proline
  VARIANT 107: note = X is tryptophan or glycine
  VARIANT 114: note = X is arginine, histidine, or glutamine
  VARIANT 116: note = X is aspartic acid or tyrosine
  VARIANT 127: note = X is asparagine or lysine
  VARIANT 142: note = X is threonine or isoleucine
  VARIANT 144: note = X is lysine or glutamine
  VARIANT 145: note = X is arginine or histidine
  VARIANT 149: note = X is alanine or threonine
  VARIANT 150: note = X is alanine or valine
  VARIANT 151: note = X is histidine or arginine
  VARIANT 156: note = X is arginine, leucine, glutamine, or tryptophan
  VARIANT 158: note = X is valine or alanine
  VARIANT 161: note = X is aspartic acid or glutamic acid
  VARIANT 163: note = X is arginine or threonine
  VARIANT 166: note = X is aspartic acid or glutamic acid
  VARIANT 167: note = X is tryptophan or glycine
  VARIANT 184: note = X is proline or alanine
  VARIANT 193: note = X is proline or alanine
  VARIANT 194: note = X is valine or isoleucine
  VARIANT 207: note = X is serine or glycine
  VARIANT 246: note = X is alanine or serine
  VARIANT 253: note = X is glutamine or glutamic acid
  VARIANT 276: note = X is proline or leucine

SEQ ID NO: 84 - protein, 552 aa, synthetic construct
  REGION 1..552: note = synthetic sequence
  VARIANT 9: note = X is histidinem tyrosine, or aspartic acid
  VARIANT 11: note = X is alanine or serine
  VARIANT 12: note = X is methionine or valine
  VARIANT 24: note = X is alanine, serine, or threonine
  VARIANT 32: note = X is glutamie or leucine
  VARIANT 41: note = X is alanine or threonine
  VARIANT 45: note = X is glutamic acid, methionine, lysine, or threonine
  VARIANT 46: note = X is alanine or threonine
  VARIANT 63: note = X is glutamic acid or asparagine
  VARIANT 66: note = X is isoleucine or lysine
  VARIANT 67: note = X is tyrosine, phenylalanie, serine, or cystine
  VARIANT 70: note = X is asparagine or glutamine
  VARIANT 71: note = X is alanine or threonine
  VARIANT 74: note = X is aspartic acid or tyrosine
  VARIANT 76: note = X is glutamic acid or valine
  VARIANT 77: note = X is serine or asparagine
  VARIANT 80: note = X is threonine, asparagine, or isoleucine
  VARIANT 81: note = X is alanine or leucine
  VARIANT 82: note = X is leucine or arginine
  VARIANT 83: note = X is arginine or glycine
  VARIANT 94: note = X is threonine or isoleucine
  VARIANT 95: note = X is leucine or isoleucine
  VARIANT 97: note = X is arginine or serine
  VARIANT 131: note = X is arginine or serine
  VARIANT 143: note = X is S or T
  VARIANT 147: note = X is L or W
  VARIANT 152: note = Xaa is E OR V
  VARIANT 156: note = Xaa is R, D, L
  VARIANT 158: note = Xaa A or T
  VARIANT 163: note = X is leucine, glutamic acid, or threonine
  VARIANT 177: note = X is glutamic acid or aspartic acid
  VARIANT 178: note = X is lysine or threonine
  VARIANT 180: note = X is glutamic acid or glutamine
  VARIANT 194: note = X is isoleucine or valine
  VARIANT 285: note = Xaa can be any naturally occurring amino acid
  VARIANT 287..288: note = Xaa can be any naturally occurring amino acid
  VARIANT 300: note = Xaa can be any naturally occurring amino acid
  VARIANT 308: note = Xaa can be any naturally occurring amino acid
  VARIANT 317: note = Xaa can be any naturally occurring amino acid
  VARIANT 321..322: note = Xaa can be any naturally occurring amino acid
  VARIANT 339: note = Xaa can be any naturally occurring amino acid
  VARIANT 342..343: note = Xaa can be any naturally occurring amino acid
  VARIANT 346..347: note = Xaa can be any naturally occurring amino acid
  VARIANT 350: note = Xaa can be any naturally occurring amino acid
  VARIANT 352..353: note = Xaa can be any naturally occurring amino acid
  VARIANT 356..359: note = Xaa can be any naturally occurring amino acid
  VARIANT 370..371: note = Xaa can be any naturally occurring amino acid
  VARIANT 373: note = Xaa can be any naturally occurring amino acid
  VARIANT 407: note = Xaa can be any naturally occurring amino acid
  VARIANT 419: note = Xaa can be any naturally occurring amino acid
  VARIANT 423: note = Xaa can be any naturally occurring amino acid
  VARIANT 428: note = Xaa can be any naturally occurring amino acid
  VARIANT 432: note = Xaa can be any naturally occurring amino acid
  VARIANT 434: note = Xaa can be any naturally occurring amino acid
  VARIANT 439: note = Xaa can be any naturally occurring amino acid
  VARIANT 453..454: note = Xaa can be any naturally occurring amino acid
  VARIANT 456: note = Xaa can be any naturally occurring amino acid
  VARIANT 470: note = Xaa can be any naturally occurring amino acid
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEPGSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 85 - protein, 276 aa, synthetic construct; identical to SEQ ID NO: 79
  REGION 1..276: note = synthetic sequence
  VARIANT 1: note = X is cysteine or glycine
  VARIANT 6: note = X is arginine or lysine
  VARIANT 9: note = X is phenylalanine, tyrosine, serine, or aspartic acid
  VARIANT 14: note = X is arginine or tryptophan
  VARIANT 21: note = X is histidine or arginine
  VARIANT 24: note = X is alanine or serine
  VARIANT 35: note = X is glutamine or arginine
  VARIANT 49: note = X is alanine or arginine
  VARIANT 66: note = X is asparagine, or lysine
  VARIANT 73: note = X is threonine or alanine
  VARIANT 77: note = X is serine or asparagine
  VARIANT 80: note = X is asparagine or lysine
  VARIANT 90: note = X is alanine or aspartic acid
  VARIANT 91: note = X is glycine or arginine
  VARIANT 94: note = X is threonine or isoleucine
  VARIANT 95: note = X is leucine or isoleucine
  VARIANT 97: note = X is tryptophan or arginine
  VARIANT 99: note = X is cysteine, tyrosine, phenylalanine, or serine
  VARIANT 103: note = X is leucine or valine
  VARIANT 113: note = X is tyrosine or histidine
  VARIANT 114: note = X is aspartic acid or asparagine
  VARIANT 116: note = X is tyrosine, phenylalanine, serine, or leucine
  VARIANT 147: note = X is leucine or tryptophan
  VARIANT 152: note = X is glutamic acid, alanine, or threonine
  VARIANT 156: note = X is arginine, leucine, or tryptophan
  VARIANT 163: note = X is leucine or threonine
  VARIANT 173: note = X is glutamic acid or lysine
  VARIANT 177: note = X is glutamic acid or lysine
  VARIANT 184: note = X is histidine or proline
  VARIANT 194: note = X is arginine or valine
  VARIANT 219: note = X is tryptophan or arginine
  VARIANT 248: note = X is valine or methionine
  VARIANT 253: note = X is glutamic acid or lysine
  VARIANT 261: note = X is methionine or valine
  VARIANT 267: note = X is proline or glutamine
  VARIANT 273: note = X is arginine or serine
  VARIANT 275: note = X is proline or glycine

SEQ ID NO: 86 - protein, 284 aa, synthetic construct; identical to SEQ ID NO: 80
  REGION 1..284: note = synthetic sequence
  VARIANT 89: note = Xaa is K or E
  VARIANT 107: note = Xaa is R or G
  VARIANT 157: note = Xaa is R or G
  VARIANT 158: note = Xaa is A or V
  VARIANT 255: note = Xaa is Q or P
  VARIANT 267: note = Xaa is P or S

SEQ ID NO: 87 - protein, 284 aa, synthetic construct; identical to SEQ ID NO: 81
  REGION 1..284: note = synthetic sequence
  VARIANT 7: note = Xaa is Y or F
  VARIANT 50: note = Xaa is P or Q
  VARIANT 251: note = Xaa is S or P
  VARIANT 278: note = Xaa is P or L

SEQ ID NO: 88 - protein, 284 aa, synthetic construct; identical to SEQ ID NO: 82
  REGION 1..284: note = synthetic sequence
  VARIANT 13: note = Xaa is S or F
  VARIANT 27: note = Xaa is Y or H
  VARIANT 31: note = Xaa is T, S, or M
  VARIANT 34: note = Xaa is L or V
  VARIANT 54: note = Xaa is Q or R
  VARIANT 81: note = Xaa is P or L
  VARIANT 100: note = Xaa is G or D
  VARIANT 104: note = Xaa is G or V
  VARIANT 105: note = Xaa is S or C
  VARIANT 110: note = Xaa is L or I
  VARIANT 159: note = Xaa is Y or H
  VARIANT 169: note = Xaa is H or R
  VARIANT 171: note = Xaa is Y or H
  VARIANT 178: note = Xaa is M or T
  VARIANT 185: note = Xaa is P or A
  VARIANT 219: note = Xaa is R, W, or Q
  VARIANT 258: note = Xaa is T or M
  VARIANT 275: note = Xaa is K or E

SEQ ID NO: 89 - protein, 188 aa, synthetic construct
  REGION 1..188: note = synthetic sequence
  VARIANT 5: note = Xaa is Lys, Phe, or Leu
  VARIANT 7: note = Xaa is Val or Leu
  VARIANT 8: note = Xaa is Val or Thr
  VARIANT 9: note = Xaa is Val or Thr
  VARIANT 12: note = Xaa is Gly, Asp, Cys, Val, or Ser where one or both of Xaa at position 12 and Xaa at position 13 is not a Cys
  VARIANT 13: note = Xaa is Gly, Cys, or Asp where one or both of Xaa at position 12 and Xaa at position 13 is not a Cys
MTEYXLXXXGAXXVGKSALTIQLIQNHFVDEYDPTIEDSYRKQVVIDGETCLWDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 90-97 - skipped (8 entries)

SEQ ID NO: 98 - protein, 5 aa, synthetic construct
  REGION 1..5: note = synthetic sequence
GTLRG

SEQ ID NO: 99 - protein, 5 aa, synthetic construct
  REGION 1..5: note = synthetic sequence
YNQSE

SEQ ID NO: 100 - protein, 5 aa, synthetic construct
  REGION 1..5: note = synthetic sequence
TAADM

SEQ ID NO: 101 - protein, 5 aa, synthetic construct
  REGION 1..5: note = synthetic sequence
AQTTK

SEQ ID NO: 102 - protein, 5 aa, synthetic construct
  REGION 1..5: note = synthetic sequence
VETRP

SEQ ID NO: 103 - protein, 5 aa, synthetic construct
  REGION 1..5: note = synthetic sequence
GDGTF

SEQ ID NO: 104 - protein, 5 aa, synthetic construct
  REGION 1..5: note = synthetic sequence
RNLRG

SEQ ID NO: 105 - protein, 5 aa, synthetic construct
  REGION 1..5: note = synthetic sequence
TAADT

SEQ ID NO: 106 - protein, 5 aa, synthetic construct
  REGION 1..5: note = synthetic sequence
AQITQ

SEQ ID NO: 107 - protein, 5 aa, synthetic construct
  REGION 1..5: note = synthetic sequence
GDRTF

SEQ ID NO: 108 - protein, 276 aa, synthetic construct [sequence omitted]

SEQ ID NO: 109 - protein, 276 aa, synthetic construct [sequence omitted]

SEQ ID NO: 110 - protein, 275 aa, synthetic construct [sequence omitted]

SEQ ID NO: 111 - protein, 276 aa, synthetic construct; identical to SEQ ID NO: 48
  REGION 1..276: note = synthetic sequence

SEQ ID NO: 112 - protein, 276 aa, synthetic construct; identical to SEQ ID NO: 48
  REGION 1..276: note = synthetic sequence

SEQ ID NO: 113 - protein, 275 aa, synthetic construct; identical to SEQ ID NO: 46
  REGION 1..275: note = synthetic sequence

SEQ ID NO: 114 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 115 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 116 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 117 - protein, 341 aa, synthetic construct
  REGION 1..341: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 118 - protein, 341 aa, synthetic construct
  REGION 1..341: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 119 - protein, 276 aa, synthetic construct [sequence omitted]

SEQ ID NO: 120 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 121 - protein, 276 aa, synthetic construct
  REGION 1..276: note = synthetic sequence
GSHSMRYFYTSVSRPGRGEPRFISVGYVDDTQFVRFDSDAASPREEPRAPWIEQEGPEYWDRNTQIYKAQAQTDRESLRNLRGAYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWEP

SEQ ID NO: 122 - protein, 276 aa, synthetic construct
  REGION 1..276: note = synthetic sequence
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTCAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 123 - protein, 276 aa, synthetic construct; identical to SEQ ID NO: 75
  REGION 1..276: note = synthetic sequence

SEQ ID NO: 124 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGAYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPCGDGTFQKWAAVVVPSGQEQRYTCHMQHEGLQEPLTLSWEP

SEQ ID NO: 125 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGCYNQSEDGSHTLQRMYGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 126 - protein, 99 aa, synthetic construct
  REGION 1..99: note = synthetic sequence
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 127 - protein, 246 aa, synthetic construct
  REGION 1..246: note = synthetic sequence
  VARIANT 78..79: note = 1-5 amino acid residues are present between the residues, wherein the amino acid residues are i) selected independently from any naturally occurring (e.g., encoded) amino acid or ii) any naturally occurring amino acid except proline or glycine
  VARIANT 79..80: note = 1-5 amino acid residues are present between the residues, wherein the amino acid residues are i) selected independently from any naturally occurring (e.g., encoded) amino acid or ii) any naturally occurring amino acid except proline or glycine
  VARIANT 123..124: note = 1-5 amino acid residues are present between the residues, wherein the amino acid residues are i) selected independently from any naturally occurring (e.g., encoded) amino acid or ii) any naturally occurring amino acid except proline or glycine
  VARIANT 124..125: note = 1-5 amino acid residues are present between the residues, wherein the amino acid residues are i) selected independently from any naturally occurring (e.g., encoded) amino acid or ii) any naturally occurring amino acid except proline or glycine
  VARIANT 210..211: note = 1-5 amino acid residues are present between the residues, wherein the amino acid residues are i) selected independently from any naturally occurring (e.g., encoded) amino acid or ii) any naturally occurring amino acid except proline or glycine
  VARIANT 211..212: note = 1-5 amino acid residues are present between the residues, wherein the amino acid residues are i) selected independently from any naturally occurring (e.g., encoded) amino acid or ii) any naturally occurring amino acid except proline or glycine
GSHSMRYFFTSVSRPGRGEPRFIAVGYVDDTQFVRFDSDAASQRMEPRAPWIEQEGPEYWDGETRKVKAHSQTHRVDLCAGSHTVQRMYGCDVGSDWRFLRGYHQYAYDGKDYIALKEDLRSWCHKWEAAHVAEQLRAYLEGTCVEWLRRYLENGKETLQRTDAPKTHMTHHAVSDHEATLRCWALSFYPAEITLTWQRDGEDQTQDTELIQKWAAVVVPSGQEQRYTCHVQHEGLPKPLTLRWEP

SEQ ID NO: 128 - protein, 99 aa, synthetic construct [sequence omitted]

SEQ ID NO: 129 - protein, 99 aa, synthetic construct; identical to SEQ ID NO: 126
  REGION 1..99: note = synthetic sequence

SEQ ID NO: 130 - protein, 276 aa, synthetic construct [sequence omitted]

SEQ ID NO: 131 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 132 - protein, 275 aa, synthetic construct; identical to SEQ ID NO: 46
  REGION 1..275: note = synthetic sequence

SEQ ID NO: 133 - protein, 99 aa, synthetic construct; identical to SEQ ID NO: 126
  REGION 1..99: note = synthetic sequence

SEQ ID NO: 134 - protein, 276 aa, synthetic construct; identical to SEQ ID NO: 131
  REGION 1..276: note = synthetic sequence

SEQ ID NO: 135 - protein, 99 aa, synthetic construct; identical to SEQ ID NO: 126
  REGION 1..99: note = synthetic sequence

SEQ ID NO: 136 - protein, 9 aa, synthetic construct
  REGION 1..9: note = synthetic sequence
GCGGSGGGS

SEQ ID NO: 137 - protein, 20 aa, synthetic construct
  REGION 1..20: note = synthetic sequence
GCGGSGGGGSGGGGSGGGGS

SEQ ID NO: 138 - protein, 15 aa, synthetic construct
  REGION 1..15: note = synthetic sequence
GCGGSGGGGSGGGGS

SEQ ID NO: 139 - protein, 5 aa, synthetic construct
  REGION 1..5: note = synthetic sequence
GCGGS

SEQ ID NO: 140 - protein, 10 aa, synthetic construct
  REGION 1..10: note = synthetic sequence
  VARIANT 6..10: note = The residues may be repated 1, 2, 3, 4, 5, 6, 7, 8, 9, or 10 times
GCGGSGGGGS

SEQ ID NO: 141 - protein, 5 aa, synthetic construct
  REGION 1..5: note = synthetic sequence
CGGGS

SEQ ID NO: 142 - protein, 10 aa, synthetic construct
  REGION 1..10: note = synthetic sequence
  VARIANT 6..10: note = The residues may be repeated 1,2,3,4,5,6,7,8,9, or 10 times
CGGGSGGGGS

SEQ ID NO: 143 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 144 - protein, 99 aa, synthetic construct; identical to SEQ ID NO: 126
  REGION 1..99: note = synthetic sequence

SEQ ID NO: 145 - protein, 275 aa, synthetic construct
  REGION 1..275: note = synthetic sequence
GSHSMRYFFTSVSRPGRGEPRFIAVGYVDDTQFVRFDSDAASQKMEPRAPWIEQEGPEYWDQETRNMKAHSQTDRANLGTLRGCYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 146 - protein, 276 aa, synthetic construct; identical to SEQ ID NO: 143
  REGION 1..276: note = synthetic sequence

SEQ ID NO: 147 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 148 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 149 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 150 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 151 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAQSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 152 - protein, 275 aa, synthetic construct
  REGION 1..275: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 153 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDRNTRKVKAQSQTDRVDLGTLRGCYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 154 - protein, 276 aa, synthetic construct
  REGION 1..276: note = synthetic sequence
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWEP

SEQ ID NO: 155 - protein, 99 aa, synthetic construct; identical to SEQ ID NO: 126
  REGION 1..99: note = synthetic sequence

SEQ ID NO: 156 - protein, 275 aa, synthetic construct
  REGION 1..275: note = synthetic sequence
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 157 - protein, 275 aa, synthetic construct
  REGION 1..275: note = synthetic sequence
GSHSMRYFDTAMSRPGRGEPRFISVGYVDDTQFVRFDSDAASPREEPRAPWIEQEGPEYWDRNTQIFKTNTQTDRESLRNLRGCYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 158 - protein, 275 aa, synthetic construct
  REGION 1..275: note = synthetic sequence
GSHSMRYFYTAMSRPGRGEPRFIAVGYVDDTQFVRFDSDAASPRMAPRAPWIEQEGPEYWDRNTQISKTNTQTYRESLRNLRGCYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 159 - protein, 275 aa, synthetic construct
  REGION 1..275: note = synthetic sequence
GSHSMRYFYTSVSRPGRGEPRFISVGYVDDTQFVRFDSDAASPREEPRAPWIEQEGPEYWDRNTQICKTNTQTYRENLRTALRCYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 160 - protein, 275 aa, synthetic construct
  REGION 1..275: note = synthetic sequence
GSHSMRYFHTAMSRPGRGEPRFITVGYVDDTLFVRFDSDATSPRKEPRAPWIEQEGPEYWDRETQISKTNTQTYRESLRNLRGCYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 161 - protein, 275 aa, synthetic construct
  REGION 1..275: note = synthetic sequence
GSHSMRYFYTAMSRPGRGEPRFIAVGYVDDTQFVRFDSDAASPRMAPRAPWIEQEGPEYWDRETQKYKRQAQTDRVSLRNLRGCYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPCGDRTFQKWAAVVVPSGEEQRYTCHVQHEGLPKPLTLRWE

SEQ ID NO: 162 - protein, 275 aa, synthetic construct
  REGION 1..275: note = synthetic sequence
GSHSMRYFYTAMSRPGRGEPRFIAVGYVDDTQFVRFDSDAASPRTEPRAPWIEQEGPEYWDRNTQIFKTNTQTYRENLRIALRCYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPCGDRTFQKWAAVVVPSGEEQRYTCHVQHEGLPKPLTLRWE

SEQ ID NO: 163 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGCYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPCGDGTFQKWAAVVVPSGQEQRYTCHMQHEGLQEPLTLSWEP

SEQ ID NO: 164 - protein, 99 aa, synthetic construct; identical to SEQ ID NO: 126
  REGION 1..99: note = synthetic sequence

SEQ ID NO: 165 - protein, 276 aa, synthetic construct
  REGION 1..276: note = synthetic sequence
CSHSMKYFFTSVSRPGRGEPRFISVGYVDDTQFVRFDSDAASPRGEPRAPWVEQEGPEYWDRETQKYKRQAQTDRVSLRNLRGCYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPCGDGTFQKWAAVMVPSGEEQRYTCHVQHEGLPEPLTLRWEP

SEQ ID NO: 166 - protein, 276 aa, synthetic construct
  REGION 1..276: note = synthetic sequence
GSHSMRYFYTAVSRPGRGEPHFIAVGYVDDTQFVRFDSDAASPRGEPRAPWVEQEGPEYWDRETQKYKRQAQTDRVSLRNLRGCYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPCGDGTFQKWAAVVVPSGEEQRYTCHVQHEGLPEPLTLRWEP

SEQ ID NO: 167 - protein, 276 aa, synthetic construct
  REGION 1..276: note = synthetic sequence
GSHSMRYFYTAVSRPGRGEPHFIAVGYVDDTQFVRFDSDAASPRGEPRAPWVEQEGPEYWDRETQKYKRQAQTDRVSLRNLRGCYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPCGDGTFQKWAAVVVPSGEEQRYTCHVQHEGLPEPLTLRWEP

SEQ ID NO: 168 - protein, 276 aa, synthetic construct
  REGION 1..276: note = synthetic sequence
GSHSMRYFSTSVSWPGRGEPRFIAVGYVDDTQFVRFDSDAASPRGEPREPWVEQEGPEYWDRETQKYKRQAQADRVNLRKLRGCYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPCGDGTFQKWAAVVVPSGEEQRYTCHVQHEGLPEPLTLRWKP

SEQ ID NO: 169 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQKYKRQAQADRVNLRKLRGCYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPCGDGTFQKWAAVVVPSGEEQRYTCHVQHEGLPEPLTLRWEP

SEQ ID NO: 170 - protein, 276 aa, synthetic construct; identical to SEQ ID NO: 163
  REGION 1..276: note = synthetic sequence

SEQ ID NO: 171 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPCGDGTFQKWAAVVVPSGQEQRYTCHMQHEGLQEPLTLSWEP

SEQ ID NO: 172 - protein, 276 aa, synthetic construct
  REGION 1..276: note = synthetic sequence
CSHSMRYFYTAVSRPGRGEPRFIAVGYVDDTQFVQFDSDAASPRGEPRAPWVEQEGPEYWDRETQKYKRQAQTDRVSLRNLRGCYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPCGDGTFQKWAAVVVPSGEEQRYTCHVQHEGLPEPLTLRWGP

SEQ ID NO: 173 - protein, 276 aa, synthetic construct
  REGION 1..276: note = synthetic sequence
CSHSMRYFYTAVSRPGRGEPHFIAVGYVDDTQFVRFDSDAASPRGEPRAPWVEQEGPEYWDRETQNYKRQAQTDRVNLRKLRGCYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPCGDGTFQKWAAVVVPSGEEQRYTCHVQHEGLPEPLTLRWEP

SEQ ID NO: 174 - skipped

SEQ ID NO: 175 - protein, 223 aa, synthetic construct
  REGION 1..223: note = synthetic sequence
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 176 - protein, 9 aa, synthetic construct
  REGION 1..9: note = synthetic sequence
VVGADGVGK

SEQ ID NO: 177 - protein, 9 aa, synthetic construct
  REGION 1..9: note = synthetic sequence
VVGACGVGK

SEQ ID NO: 178 - protein, 9 aa, synthetic construct
  REGION 1..9: note = synthetic sequence
VVGAVGVGK

SEQ ID NO: 179 - protein, 10 aa, synthetic construct
  REGION 1..10: note = synthetic sequence
VVVGADGVGK

SEQ ID NO: 180 - protein, 10 aa, synthetic construct
  REGION 1..10: note = synthetic sequence
VVVGAVGVGK

SEQ ID NO: 181 - protein, 10 aa, synthetic construct
  REGION 1..10: note = synthetic sequence
VVVGACGVGK

SEQ ID NO: 182 - protein, 9 aa, synthetic construct
  REGION 1..9: note = synthetic sequence
VTGADGVGK

SEQ ID NO: 183 - protein, 9 aa, synthetic construct
  REGION 1..9: note = synthetic sequence
VTGAVGVGK

SEQ ID NO: 184 - protein, 9 aa, synthetic construct
  REGION 1..9: note = synthetic sequence
VTGACGVGK

SEQ ID NO: 185 - protein, 10 aa, synthetic construct
  REGION 1..10: note = synthetic sequence
VTVGADGVGK

SEQ ID NO: 186 - protein, 10 aa, synthetic construct
  REGION 1..10: note = synthetic sequence
VTVGAVGVGK

SEQ ID NO: 187 - protein, 10 aa, synthetic construct
  REGION 1..10: note = synthetic sequence
VTVGACGVGK

SEQ ID NO: 188 - protein, 10 aa, synthetic construct
  REGION 1..10: note = synthetic sequence
VVVGAGDVGK

SEQ ID NO: 189 - protein, 9 aa, synthetic construct
  REGION 1..9: note = synthetic sequence
VVGAGDVGK

SEQ ID NO: 190 - protein, 10 aa, synthetic construct
  REGION 1..10: note = synthetic sequence
VVVGARGVGK

SEQ ID NO: 191 - protein, 9 aa, synthetic construct
  REGION 1..9: note = synthetic sequence
VVGARGVGK

SEQ ID NO: 192 - protein, 9 aa, synthetic construct
  REGION 1..9: note = synthetic sequence
LVVVGADGV

SEQ ID NO: 193 - protein, 9 aa, synthetic construct
  REGION 1..9: note = synthetic sequence
LVVVGAVGV

SEQ ID NO: 194 - protein, 9 aa, synthetic construct
  REGION 1..9: note = synthetic sequence
LVVVGACGV

SEQ ID NO: 195 - protein, 10 aa, synthetic construct
  REGION 1..10: note = synthetic sequence
KLVVVGADGV

SEQ ID NO: 196 - protein, 10 aa, synthetic construct
  REGION 1..10: note = synthetic sequence
KLVVVGAVGV

SEQ ID NO: 197 - protein, 10 aa, synthetic construct
  REGION 1..10: note = synthetic sequence
KLVVVGACGV

SEQ ID NO: 198 - protein, 9 aa, synthetic construct
  REGION 1..9: note = synthetic sequence
LLVVGADGV

SEQ ID NO: 199 - protein, 9 aa, synthetic construct
  REGION 1..9: note = synthetic sequence
LLVVGAVGV

SEQ ID NO: 200 - protein, 9 aa, synthetic construct
  REGION 1..9: note = synthetic sequence
LLVVGACGV

SEQ ID NO: 201 - protein, 10 aa, synthetic construct
  REGION 1..10: note = synthetic sequence
FLVVVGADGV

SEQ ID NO: 202 - protein, 10 aa, synthetic construct
  REGION 1..10: note = synthetic sequence
FLVVVGAVGV

SEQ ID NO: 203 - protein, 10 aa, synthetic construct
  REGION 1..10: note = synthetic sequence
FLVVVGACGV

SEQ ID NO: 204 - protein, 10 aa, synthetic construct
  REGION 1..10: note = synthetic sequence
KLVVVGAGDV

SEQ ID NO: 205 - protein, 10 aa, synthetic construct
  REGION 1..10: note = synthetic sequence
KLVVVGARGV

SEQ ID NO: 206 - protein, 10 aa, synthetic construct
  REGION 1..10: note = synthetic sequence
GAGDVGKSAL

SEQ ID NO: 207 - protein, 9 aa, synthetic construct
  REGION 1..9: note = synthetic sequence
AGDVGKSAL

SEQ ID NO: 208 - protein, 9 aa, synthetic construct
  REGION 1..9: note = synthetic sequence
DVGKSALTI

SEQ ID NO: 209 - protein, 10 aa, synthetic construct
  REGION 1..10: note = synthetic sequence
GAVGVGKSAL

SEQ ID NO: 210 - protein, 9 aa, synthetic construct
  REGION 1..9: note = synthetic sequence
AVGVGKSAL

SEQ ID NO: 211 - protein, 9 aa, synthetic construct
  REGION 1..9: note = synthetic sequence
YKLVVVGAV

SEQ ID NO: 212 - protein, 9 aa, synthetic construct
  REGION 1..9: note = synthetic sequence
ARGVGKSAL

SEQ ID NO: 213 - protein, 10 aa, synthetic construct
  REGION 1..10: note = synthetic sequence
GARGVGKSAL

SEQ ID NO: 214 - protein, 10 aa, synthetic construct
  REGION 1..10: note = synthetic sequence
EYKLVVVGAR

SEQ ID NO: 215 - protein, 10 aa, synthetic construct
  REGION 1..10: note = synthetic sequence
RGVGKSALTI

SEQ ID NO: 216 - protein, 9 aa, synthetic construct
  REGION 1..9: note = synthetic sequence
LVVVGARGV

SEQ ID NO: 217 - protein, 10 aa, synthetic construct
  REGION 1..10: note = synthetic sequence
GADGVGKSAL

SEQ ID NO: 218 - protein, 9 aa, synthetic construct
  REGION 1..9: note = synthetic sequence
ACGVGKSAL

SEQ ID NO: 219 - protein, 10 aa, synthetic construct
  REGION 1..10: note = synthetic sequence
GACGVGKSAL

SEQ ID NO: 220 - protein, 9 aa, synthetic construct
  REGION 1..9: note = synthetic sequence
VVGAVGVGK

SEQ ID NO: 221 - protein, 10 aa, synthetic construct
  REGION 1..10: note = synthetic sequence
VVVGAVGVGK

SEQ ID NO: 222 - protein, 9 aa, synthetic construct
  REGION 1..9: note = synthetic sequence
VGAVGVGKS

SEQ ID NO: 223 - protein, 10 aa, synthetic construct
  REGION 1..10: note = synthetic sequence
VGAVGVGKSA

SEQ ID NO: 224 - protein, 9 aa, synthetic construct
  REGION 1..9: note = synthetic sequence
AVGVGKSAL

SEQ ID NO: 225 - protein, 10 aa, synthetic construct
  REGION 1..10: note = synthetic sequence
AVGVGKSALT

SEQ ID NO: 226 - protein, 10 aa, synthetic construct
  REGION 1..10: note = synthetic sequence
GAVGVGKSAL

SEQ ID NO: 227 - protein, 9 aa, synthetic construct
  REGION 1..9: note = synthetic sequence
GAVGVGKSA

SEQ ID NO: 228 - protein, 10 aa, synthetic construct
  REGION 1..10: note = synthetic sequence
LVVVGAVGVG

SEQ ID NO: 229 - protein, 9 aa, synthetic construct
  REGION 1..9: note = synthetic sequence
LVVVGAVGV

SEQ ID NO: 230 - protein, 10 aa, synthetic construct
  REGION 1..10: note = synthetic sequence
KLVVVGAVGV

SEQ ID NO: 231 - protein, 9 aa, synthetic construct
  REGION 1..9: note = synthetic sequence
KLVVVGAVG

SEQ ID NO: 232 - protein, 276 aa, synthetic construct; identical to SEQ ID NO: 120
  REGION 1..276: note = synthetic sequence

SEQ ID NO: 233 - protein, 276 aa, synthetic construct
  REGION 1..276: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEA

SEQ ID NO: 234 - protein, 341 aa, synthetic construct; identical to SEQ ID NO: 117
  REGION 1..341: note = synthetic sequence

SEQ ID NO: 235 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 236 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 237 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 238 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 239 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 240 - protein, 275 aa, synthetic construct; identical to SEQ ID NO: 150
  REGION 1..275: note = synthetic sequence

SEQ ID NO: 241 - protein, 133 aa, synthetic construct
  REGION 1..133: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 242 - protein, 10 aa, synthetic construct
  REGION 1..10: note = synthetic sequence
GCGGSGGGGS

SEQ ID NO: 243 - protein, 15 aa, synthetic construct
  REGION 1..15: note = synthetic sequence
GCGGSGGGGSGGGGS

SEQ ID NO: 244 - protein, 20 aa, synthetic construct
  REGION 1..20: note = synthetic sequence
GCGGSGGGGSGGGGSGGGGS

SEQ ID NO: 245 - protein, 25 aa, synthetic construct
  REGION 1..25: note = synthetic sequence
GCGGSGGGGSGGGGSGGGGSGGGGS

SEQ ID NO: 246 - protein, 30 aa, synthetic construct
  REGION 1..30: note = synthetic sequence
GCGGSGGGGSGGGGSGGGGSGGGGSGGGGS

SEQ ID NO: 247 - protein, 35 aa, synthetic construct
  REGION 1..35: note = synthetic sequence
GCGGSGGGGSGGGGSGGGGSGGGGSGGGGSGGGGS

SEQ ID NO: 248 - protein, 40 aa, synthetic construct
  REGION 1..40: note = synthetic sequence
GCGGSGGGGSGGGGSGGGGSGGGGSGGGGSGGGGSGGGGS

SEQ ID NO: 249 - protein, 45 aa, synthetic construct
  REGION 1..45: note = synthetic sequence
GCGGSGGGGSGGGGSGGGGSGGGGSGGGGSGGGGSGGGGSGGGGS

SEQ ID NO: 250 - protein, 50 aa, synthetic construct
  REGION 1..50: note = synthetic sequence
GCGGSGGGGSGGGGSGGGGSGGGGSGGGGSGGGGSGGGGSGGGGSGGGGS

SEQ ID NO: 251 - protein, 55 aa, synthetic construct
  REGION 1..55: note = synthetic sequence
GCGGSGGGGSGGGGSGGGGSGGGGSGGGGSGGGGSGGGGSGGGGSGGGGSGGGGS

SEQ ID NO: 252 - protein, 10 aa, synthetic construct
  REGION 1..10: note = synthetic sequence
  VARIANT 6..10: note = The amino acids may be repeated 1, 2, or 3 times.
GCGGSGGGGS

SEQ ID NO: 253 - skipped

SEQ ID NO: 254 - protein, 5 aa, synthetic construct
  REGION 1..5: note = synthetic sequence
  VARIANT 1..5: note = The residues may be repeated 1, 2, 3, 4, 5, 6, 7, 8, 9, or 10 times.
GGGGS

SEQ ID NO: 255 - protein, 812 aa, synthetic construct
  REGION 1..812: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 256 - protein, 812 aa, synthetic construct
  REGION 1..812: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 257 - protein, 812 aa, synthetic construct
  REGION 1..812: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 258 - protein, 812 aa, synthetic construct
  REGION 1..812: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 259 - protein, 812 aa, synthetic construct
  REGION 1..812: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 260 - protein, 812 aa, synthetic construct
  REGION 1..812: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 261 - protein, 123 aa, synthetic construct
  REGION 1..123: note = synthetic sequence
VVGADGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 262 - protein, 123 aa, synthetic construct
  REGION 1..123: note = synthetic sequence
VVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 263 - protein, 123 aa, synthetic construct
  REGION 1..123: note = synthetic sequence
VVGADGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 264 - protein, 123 aa, synthetic construct
  REGION 1..123: note = synthetic sequence
VVGACGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 265 - protein, 123 aa, synthetic construct
  REGION 1..123: note = synthetic sequence
VVGACGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 266 - protein, 123 aa, synthetic construct
  REGION 1..123: note = synthetic sequence
VVGACGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 267 - protein, 123 aa, synthetic construct
  REGION 1..123: note = synthetic sequence
VVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 268 - protein, 123 aa, synthetic construct
  REGION 1..123: note = synthetic sequence
VVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 269 - protein, 123 aa, synthetic construct
  REGION 1..123: note = synthetic sequence
VVGAVGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 270 - protein, 124 aa, synthetic construct
  REGION 1..124: note = synthetic sequence
VVVGADGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 271 - protein, 124 aa, synthetic construct
  REGION 1..124: note = synthetic sequence
VVVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 272 - protein, 124 aa, synthetic construct
  REGION 1..124: note = synthetic sequence
VVVGADGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 273 - protein, 124 aa, synthetic construct
  REGION 1..124: note = synthetic sequence
VVVGACGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 274 - protein, 124 aa, synthetic construct
  REGION 1..124: note = synthetic sequence
VVVGACGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 275 - protein, 124 aa, synthetic construct
  REGION 1..124: note = synthetic sequence
VVVGACGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 276 - protein, 124 aa, synthetic construct
  REGION 1..124: note = synthetic sequence
VVVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 277 - protein, 124 aa, synthetic construct
  REGION 1..124: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 278 - protein, 124 aa, synthetic construct
  REGION 1..124: note = synthetic sequence
VVVGAVGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 279 - protein, 123 aa, synthetic construct
  REGION 1..123: note = synthetic sequence
VTGADGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 280 - protein, 123 aa, synthetic construct
  REGION 1..123: note = synthetic sequence
VTGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 281 - protein, 123 aa, synthetic construct
  REGION 1..123: note = synthetic sequence
VTGADGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 282 - protein, 123 aa, synthetic construct
  REGION 1..123: note = synthetic sequence
VTGACGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 283 - protein, 123 aa, synthetic construct
  REGION 1..123: note = synthetic sequence
VTGACGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 284 - protein, 123 aa, synthetic construct
  REGION 1..123: note = synthetic sequence
VTGACGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 285 - protein, 123 aa, synthetic construct
  REGION 1..123: note = synthetic sequence
VTGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 286 - protein, 123 aa, synthetic construct
  REGION 1..123: note = synthetic sequence
VTGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 287 - protein, 123 aa, synthetic construct
  REGION 1..123: note = synthetic sequence
VTGAVGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 288 - protein, 124 aa, synthetic construct
  REGION 1..124: note = synthetic sequence
VTVGADGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 289 - protein, 124 aa, synthetic construct
  REGION 1..124: note = synthetic sequence
VTVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 290 - protein, 124 aa, synthetic construct
  REGION 1..124: note = synthetic sequence
VTVGADGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 291 - protein, 124 aa, synthetic construct
  REGION 1..124: note = synthetic sequence
VTVGACGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 292 - protein, 124 aa, synthetic construct
  REGION 1..124: note = synthetic sequence
VTVGACGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 293 - protein, 124 aa, synthetic construct
  REGION 1..124: note = synthetic sequence
VTVGACGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 294 - protein, 124 aa, synthetic construct
  REGION 1..124: note = synthetic sequence
VTVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 295 - protein, 124 aa, synthetic construct
  REGION 1..124: note = synthetic sequence
VTVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 296 - protein, 124 aa, synthetic construct
  REGION 1..124: note = synthetic sequence
VTVGAVGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 297 - protein, 123 aa, synthetic construct
  REGION 1..123: note = synthetic sequence
LVVVGADGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 298 - protein, 123 aa, synthetic construct
  REGION 1..123: note = synthetic sequence
LVVVGADGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 299 - protein, 123 aa, synthetic construct
  REGION 1..123: note = synthetic sequence
LVVVGADGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 300 - protein, 123 aa, synthetic construct
  REGION 1..123: note = synthetic sequence
LVVVGAVGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 301 - protein, 123 aa, synthetic construct
  REGION 1..123: note = synthetic sequence
LVVVGAVGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 302 - protein, 123 aa, synthetic construct
  REGION 1..123: note = synthetic sequence
LVVVGAVGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 303 - protein, 123 aa, synthetic construct
  REGION 1..123: note = synthetic sequence
LVVVGACGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 304 - protein, 123 aa, synthetic construct
  REGION 1..123: note = synthetic sequence
LVVVGACGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 305 - protein, 123 aa, synthetic construct
  REGION 1..123: note = synthetic sequence
LVVVGACGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 306 - protein, 124 aa, synthetic construct
  REGION 1..124: note = synthetic sequence
KLVVVGADGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 307 - protein, 124 aa, synthetic construct
  REGION 1..124: note = synthetic sequence
KLVVVGADGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 308 - protein, 124 aa, synthetic construct
  REGION 1..124: note = synthetic sequence
KLVVVGADGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 309 - protein, 124 aa, synthetic construct
  REGION 1..124: note = synthetic sequence
KLVVVGAVGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 310 - protein, 124 aa, synthetic construct
  REGION 1..124: note = synthetic sequence
KLVVVGAVGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 311 - protein, 124 aa, synthetic construct
  REGION 1..124: note = synthetic sequence
KLVVVGAVGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 312 - protein, 124 aa, synthetic construct
  REGION 1..124: note = synthetic sequence
KLVVVGACGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 313 - protein, 124 aa, synthetic construct
  REGION 1..124: note = synthetic sequence
KLVVVGACGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 314 - protein, 124 aa, synthetic construct
  REGION 1..124: note = synthetic sequence
KLVVVGACGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 315 - protein, 123 aa, synthetic construct
  REGION 1..123: note = synthetic sequence
LLVVGADGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 316 - protein, 123 aa, synthetic construct
  REGION 1..123: note = synthetic sequence
LLVVGADGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 317 - protein, 123 aa, synthetic construct
  REGION 1..123: note = synthetic sequence
LLVVGADGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 318 - protein, 123 aa, synthetic construct
  REGION 1..123: note = synthetic sequence
LLVVGAVGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 319 - protein, 123 aa, synthetic construct
  REGION 1..123: note = synthetic sequence
LLVVGAVGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 320 - protein, 123 aa, synthetic construct
  REGION 1..123: note = synthetic sequence
LLVVGAVGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 321 - protein, 123 aa, synthetic construct
  REGION 1..123: note = synthetic sequence
LLVVGACGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 322 - protein, 123 aa, synthetic construct
  REGION 1..123: note = synthetic sequence
LLVVGACGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 323 - protein, 123 aa, synthetic construct
  REGION 1..123: note = synthetic sequence
LLVVGACGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 324 - protein, 124 aa, synthetic construct
  REGION 1..124: note = synthetic sequence
FLVVVGADGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 325 - protein, 124 aa, synthetic construct
  REGION 1..124: note = synthetic sequence
FLVVVGADGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 326 - protein, 124 aa, synthetic construct
  REGION 1..124: note = synthetic sequence
FLVVVGADGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 327 - protein, 124 aa, synthetic construct
  REGION 1..124: note = synthetic sequence
FLVVVGAVGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 328 - protein, 124 aa, synthetic construct
  REGION 1..124: note = synthetic sequence
FLVVVGAVGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 329 - protein, 124 aa, synthetic construct
  REGION 1..124: note = synthetic sequence
FLVVVGAVGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 330 - protein, 124 aa, synthetic construct
  REGION 1..124: note = synthetic sequence
FLVVVGACGVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 331 - protein, 124 aa, synthetic construct
  REGION 1..124: note = synthetic sequence
FLVVVGACGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 332 - protein, 124 aa, synthetic construct
  REGION 1..124: note = synthetic sequence
FLVVVGACGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 333 - protein, 946 aa, synthetic construct
  REGION 1..946: note = synthetic sequence
KLVVVGADGVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 334 - protein, 946 aa, synthetic construct
  REGION 1..946: note = synthetic sequence
VVVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 335 - protein, 946 aa, synthetic construct
  REGION 1..946: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 336 - protein, 966 aa, synthetic construct
  REGION 1..966: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 337 - protein, 956 aa, synthetic construct
  REGION 1..956: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 338 - protein, 976 aa, synthetic construct
  REGION 1..976: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 339 - protein, 878 aa, synthetic construct
  REGION 1..878: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKVAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 340 - protein, 878 aa, synthetic construct
  REGION 1..878: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKVAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 341 - protein, 878 aa, synthetic construct
  REGION 1..878: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKVAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 342 - protein, 807 aa, synthetic construct
  REGION 1..807: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 343 - protein, 812 aa, synthetic construct
  REGION 1..812: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 344 - protein, 807 aa, synthetic construct
  REGION 1..807: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 345-365 - skipped (21 entries)

SEQ ID NO: 366 - protein, 5 aa, synthetic construct
  REGION 1..5: note = synthetic sequence
  VARIANT 1..5: note = The residues may be repeated
GSGGS

SEQ ID NO: 367 - protein, 4 aa, synthetic construct
  REGION 1..4: note = synthetic sequence
  VARIANT 1..4: note = The residues may be repeated
GGGS

SEQ ID NO: 368 - protein, 4 aa, synthetic construct
  REGION 1..4: note = synthetic sequence
GGSG

SEQ ID NO: 369 - protein, 5 aa, synthetic construct
  REGION 1..5: note = synthetic sequence
GGSGG

SEQ ID NO: 370 - protein, 5 aa, synthetic construct
  REGION 1..5: note = synthetic sequence
GSGSG

SEQ ID NO: 371 - protein, 5 aa, synthetic construct
  REGION 1..5: note = synthetic sequence
GSGGG

SEQ ID NO: 372 - protein, 5 aa, synthetic construct
  REGION 1..5: note = synthetic sequence
GGGSG

SEQ ID NO: 373 - protein, 5 aa, synthetic construct
  REGION 1..5: note = synthetic sequence
GSSSG

SEQ ID NO: 374 - protein, 5 aa, synthetic construct
  REGION 1..5: note = synthetic sequence
  VARIANT 2..5: note = The residues may be repeated 1, 2, 3, 4, 5, 6, 7, 8, 9, or 10 times.
GSSSS

SEQ ID NO: 375 - protein, 20 aa, synthetic construct
  REGION 1..20: note = synthetic sequence
GSSSSGSSSSGSSSSGSSSS

SEQ ID NO: 376 - protein, 25 aa, synthetic construct
  REGION 1..25: note = synthetic sequence
GSSSSGSSSSGSSSSGSSSSGSSSS

SEQ ID NO: 377 - protein, 5 aa, synthetic construct
  REGION 1..5: note = synthetic sequence
GGGGS

SEQ ID NO: 378-386 - skipped (9 entries)

SEQ ID NO: 387 - protein, 5 aa, synthetic construct
  REGION 1..5: note = synthetic sequence
AAAGG

SEQ ID NO: 388 - protein, 15 aa, synthetic construct
  REGION 1..15: note = synthetic sequence
GCGGSGGGGSGGGGS

SEQ ID NO: 389 - protein, 10 aa, synthetic construct
  REGION 1..10: note = synthetic sequence
  VARIANT 6..10: note = The residues may be repeated 1, 2, 3, 4, 5, 6, 7, 8, or 9 times.
GCGGSGGGGS

SEQ ID NO: 390 - protein, 20 aa, synthetic construct
  REGION 1..20: note = synthetic sequence
GCGGSGGGGSGGGGSGGGGS

SEQ ID NO: 391 - protein, 15 aa, synthetic construct
  REGION 1..15: note = synthetic sequence
GCGGSGGGGSGGGGS

SEQ ID NO: 392-397 - skipped (6 entries)

SEQ ID NO: 398 - protein, 219 aa, synthetic construct
  REGION 1..219: note = synthetic sequence
  VARIANT 8: note = Xaa is any aminoa cid other than Asp.
FTVTVPKXLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 399 - protein, 219 aa, synthetic construct
  REGION 1..219: note = synthetic sequence
  VARIANT 36: note = Xaa is any amino acid other than Ile
FTVTVPKDLYVVEYGSNMTIECKFPVEKQLDLAALXVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 400 - protein, 219 aa, synthetic construct
  REGION 1..219: note = synthetic sequence
  VARIANT 54: note = Xaa can be any amino acid other than glutamine
FTVTVPKDLYVVEYGSNMTIECKFPVEKQLDLAALIVYWEMEDKNIIQFVHGEXDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 401 - protein, 208 aa, synthetic construct
  REGION 1..208: note = synthetic sequence
  VARIANT 19: note = Xaa can be any amino acid other than asparagine
VIHVTKEVKEVATLSCGHXVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 402 - protein, 208 aa, synthetic construct
  REGION 1..208: note = synthetic sequence
  VARIANT 63: note = Xaa can be any amino acid other than asparagine
VIHVTKEVKEVATLSCGHNVSVEELAQTRIYWQKEKKMVLTMMSGDMNIWPEYKNRTIFDITXNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 403 - protein, 208 aa, synthetic construct
  REGION 1..208: note = synthetic sequence
  VARIANT 67: note = Xaa can be any amino acid other than isoleucine
VIHVTKEVKEVATLSCGHNVSVEELAQTRIYWQKEKKMVLTMMSGDMNIWPEYKNRTIFDITNNLSXVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 404 - protein, 208 aa, synthetic construct
  REGION 1..208: note = synthetic sequence
  VARIANT 86: note = Xaa can be any amino acid other than lysine
VIHVTKEVKEVATLSCGHNVSVEELAQTRIYWQKEKKMVLTMMSGDMNIWPEYKNRTIFDITNNLSIVILALRPSDEGTYECVVLXYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 405 - protein, 208 aa, synthetic construct
  REGION 1..208: note = synthetic sequence
  VARIANT 157: note = any amino acid other than glutamine
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSXDPETELYAVSSKLDFNMTTNHSFMCLIKYGHLRVNQTFNWNTTKQEHFPDN

SEQ ID NO: 406 - protein, 208 aa, synthetic construct
  REGION 1..208: note = synthetic sequence
  VARIANT 158: note = any amino acid other than aspartic acid
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQXPETELYAVSSKLDFNMTTNHSFMCLIKYGHLRVNQTFNWNTTKQEHFPDN

SEQ ID NO: 407 - protein, 208 aa, synthetic construct
  REGION 1..208: note = synthetic sequence
  VARIANT 25: note = Xaa can be any amino acid other than leucine
VIHVTKEVKEVATLSCGHNVSVEEXAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 408 - protein, 208 aa, synthetic construct
  REGION 1..208: note = synthetic sequence
  VARIANT 31: note = Xaa can be any amino acid other than tyrosine
VIHVTKEVKEVATLSCGHNVSVEELAQTRIXWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 409 - protein, 208 aa, synthetic construct
  REGION 1..208: note = synthetic sequence
  VARIANT 33: note = Xaa can be any amino acid other than glutamine
VIHVTKEVKEVATLSCGHNVSVEELAQTRIYWXKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 410 - protein, 208 aa, synthetic construct
  REGION 1..208: note = synthetic sequence
  VARIANT 38: note = Xaa can be any amino acid other than methionine
VIHVTKEVKEVATLSCGHNVSVEELAQTRIYWQKEKKXVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 411 - protein, 208 aa, synthetic construct
  REGION 1..208: note = synthetic sequence
  VARIANT 39: note = Xaa can be any amino acid other than valine
VIHVTKEVKEVATLSCGHNVSVEELAQTRIYWQKEKKMXLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 412 - protein, 208 aa, synthetic construct
  REGION 1..208: note = synthetic sequence
  VARIANT 49: note = Xaa can be any amino acid other than isoleucine
VIHVTKEVKEVATLSCGHNVSVEELAQTRIYWQKEKKMVLTMMSGDMNXWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 413 - protein, 208 aa, synthetic construct
  REGION 1..208: note = synthetic sequence
  VARIANT 53: note = Xaa can be any amino acid other than aspartic acid
VIHVTKEVKEVATLSCGHNVSVEELAQTRIYWQKEKKMVLTMMSGDMNIWPEXKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 414 - protein, 208 aa, synthetic construct
  REGION 1..208: note = synthetic sequence
  VARIANT 60: note = any amino acid other than aspartic acid
VIHVTKEVKEVATLSCGHNVSVEELAQTRIYWQKEKKMVLTMMSGDMNIWPEYKNRTIFXITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 415 - protein, 208 aa, synthetic construct
  REGION 1..208: note = synthetic sequence
  VARIANT 108: note = Xaa can be any amino acid other than phenylalanine
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADXPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 416 - protein, 208 aa, synthetic construct
  REGION 1..208: note = synthetic sequence
  VARIANT 156: note = Xaa can be any amino acid other than serine
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVXQDPETELYAVSSKLDFNMTTNHSFMCLIKYGHLRVNQTFNWNTTKQEHFPDN

SEQ ID NO: 417 - protein, 208 aa, synthetic construct
  REGION 1..208: note = synthetic sequence
  VARIANT 111: note = Xaa can be any amino acid other than proline
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTXSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 418 - protein, 224 aa, synthetic construct
  REGION 1..224: note = synthetic sequence
  VARIANT 61: note = Xaa can be any amino acid other than asparagine
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 419 - protein, 224 aa, synthetic construct
  REGION 1..224: note = synthetic sequence
  VARIANT 66: note = Xaa can be any amino acid other than aspartic acid
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 420 - protein, 224 aa, synthetic construct
  REGION 1..224: note = synthetic sequence
  VARIANT 70: note = Xaa can be any amino acid other than tryptophan
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 421 - protein, 224 aa, synthetic construct
  REGION 1..224: note = synthetic sequence
  VARIANT 91: note = Xaa can be any amino acid other than histidine
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 422 - protein, 110 aa, synthetic construct
  REGION 1..110: note = synthetic sequence
  VARIANT 61: note = Xaa can be any amino acid other than asparagine
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVL

SEQ ID NO: 423 - protein, 110 aa, synthetic construct
  REGION 1..110: note = synthetic sequence
  VARIANT 66: note = Xaa can be any amino acid other than aspartic acid
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVL

SEQ ID NO: 424 - protein, 110 aa, synthetic construct
  REGION 1..110: note = synthetic sequence
  VARIANT 70: note = Xaa can be any amino acid other than tryptophan
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVL

SEQ ID NO: 425 - protein, 110 aa, synthetic construct
  REGION 1..110: note = synthetic sequence
  VARIANT 91: note = Xaa can be any amino acid other than histidine
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVL

SEQ ID NO: 426 - protein, 224 aa, synthetic construct
  REGION 1..224: note = synthetic sequence
  VARIANT 41: note = Xaa can be any amino acid other than valine
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 427 - protein, 110 aa, synthetic construct
  REGION 1..110: note = synthetic sequence
  VARIANT 41: note = Xaa can be any amino acid other than valine
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVL

SEQ ID NO: 428 - protein, 224 aa, synthetic construct
  REGION 1..224: note = synthetic sequence
  VARIANT 35: note = Xaa can be any amino acid other than glutamine
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 429 - protein, 110 aa, synthetic construct
  REGION 1..110: note = synthetic sequence
  VARIANT 35: note = Xaa can be any amino acid other than glutamine
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVL

SEQ ID NO: 430 - protein, 224 aa, synthetic construct
  REGION 1..224: note = synthetic sequence
  VARIANT 33: note = Xaa can be any amino acid other than phenylalanine
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 431 - protein, 110 aa, synthetic construct
  REGION 1..110: note = synthetic sequence
  VARIANT 33: note = Xaa can be any amino acid other than phenylalanine
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVL

SEQ ID NO: 432 - protein, 224 aa, synthetic construct
  REGION 1..224: note = synthetic sequence
  VARIANT 72: note = Xaa can be any amino acid other than leucine
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 433 - protein, 110 aa, synthetic construct
  REGION 1..110: note = synthetic sequence
  VARIANT 72: note = Xaa can be any amino acid other than leucine
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVL

SEQ ID NO: 434 - protein, 224 aa, synthetic construct
  REGION 1..224: note = synthetic sequence
  VARIANT 59: note = Xaa can be any amino acid other than tyrosine
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 435 - protein, 110 aa, synthetic construct
  REGION 1..110: note = synthetic sequence
  VARIANT 59: note = Xaa can be any amino acid other than tyrosine
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVL

SEQ ID NO: 436 - protein, 224 aa, synthetic construct
  REGION 1..224: note = synthetic sequence
  VARIANT 61: note = Xaa can be any amino acid other than asparagine
  VARIANT 91: note = Xaa can be any amino acid other than histidine
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 437 - protein, 110 aa, synthetic construct
  REGION 1..110: note = synthetic sequence
  VARIANT 61: note = Xaa can be any amino acid other than asparagine
  VARIANT 91: note = Xaa can be any amino acid other than histidine
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVL

SEQ ID NO: 438 - protein, 224 aa, synthetic construct
  REGION 1..224: note = synthetic sequence
  VARIANT 66: note = Xaa can be any amino acid other than aspartic acid
  VARIANT 91: note = Xaa can be any amino acid other than histidine
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 439 - protein, 110 aa, synthetic construct
  REGION 1..110: note = synthetic sequence
  VARIANT 66: note = Xaa can be any amino acid other than asparagine
  VARIANT 91: note = Xaa can be any amino acid other than His
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVL

SEQ ID NO: 440 - protein, 224 aa, synthetic construct
  REGION 1..224: note = synthetic sequence
  VARIANT 61: note = Xaa can be any amino acid other than asparagine
  VARIANT 66: note = Xaa can be any amino acid other than aspartic acid
  VARIANT 91: note = Xaa can be any amino acid other than histidine
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 441 - protein, 110 aa, synthetic construct
  REGION 1..110: note = synthetic sequence
  VARIANT 61: note = Xaa can be any amino acid other than asparagine
  VARIANT 66: note = Xaa can be any amino acid other than aspartic acid
  VARIANT 91: note = Xaa can be any amino acid other than histidine
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVL

SEQ ID NO: 442 - protein, 174 aa, synthetic construct
  REGION 1..174: note = synthetic sequence
  VARIANT 47: note = Xaa can be any amino acid other than lysine
PAGLLDLRQGMFAQLVAQNVLLIDGPLSWYSDPGLAGVSLTGGLSYXEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 443 - protein, 174 aa, synthetic construct
  REGION 1..174: note = synthetic sequence
  VARIANT 147: note = Xaa can be any amino acid other than glutamine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWXLTQGATVLGLFRVTPEIPAGLPSPRSE

SEQ ID NO: 444 - protein, 174 aa, synthetic construct
  REGION 1..174: note = synthetic sequence
  VARIANT 11: note = Xaa can be any amino acid other than methionine
PAGLLDLRQGXFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 445 - protein, 174 aa, synthetic construct
  REGION 1..174: note = synthetic sequence
  VARIANT 12: note = Xaa can be any amino acid other than phenylalanine
PAGLLDLRQGMXAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 446 - protein, 174 aa, synthetic construct
  REGION 1..174: note = synthetic sequence
  VARIANT 14: note = Xaa can be any amino acid other than glutamine
PAGLLDLRQGMFAXLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 447 - protein, 174 aa, synthetic construct
  REGION 1..174: note = synthetic sequence
  VARIANT 15: note = Xaa can be any amino acid other than leucine
PAGLLDLRQGMFAQXVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 448 - protein, 174 aa, synthetic construct
  REGION 1..174: note = synthetic sequence
  VARIANT 16: note = Xaa can be any amino acid other than valine
PAGLLDLRQGMFAQLXAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 449 - protein, 174 aa, synthetic construct
  REGION 1..174: note = synthetic sequence
  VARIANT 18: note = Xaa can be any amino acid other than glutamine
PAGLLDLRQGMFAQLVAXNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 450 - protein, 174 aa, synthetic construct
  REGION 1..174: note = synthetic sequence
  VARIANT 19: note = Xaa can be any amino acid other than asparagine
PAGLLDLRQGMFAQLVAQXVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 451 - protein, 174 aa, synthetic construct
  REGION 1..174: note = synthetic sequence
  VARIANT 20: note = Xaa can be any amino acid other than valine
PAGLLDLRQGMFAQLVAQNXLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 452 - protein, 174 aa, synthetic construct
  REGION 1..174: note = synthetic sequence
  VARIANT 21: note = Xaa can be any amino acid other than leucine
PAGLLDLRQGMFAQLVAQNVXLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 453 - protein, 174 aa, synthetic construct
  REGION 1..174: note = synthetic sequence
  VARIANT 22: note = Xaa can be any amino acid other than leucine
PAGLLDLRQGMFAQLVAQNVLXIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 454 - protein, 174 aa, synthetic construct
  REGION 1..174: note = synthetic sequence
  VARIANT 23: note = Xaa can be any amino acid other than isoleucine
PAGLLDLRQGMFAQLVAQNVLLXDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 455 - protein, 174 aa, synthetic construct
  REGION 1..174: note = synthetic sequence
  VARIANT 24: note = Xaa can be any amino acid other than aspartic acid
PAGLLDLRQGMFAQLVAQNVLLIXGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 456 - protein, 174 aa, synthetic construct
  REGION 1..174: note = synthetic sequence
  VARIANT 25: note = Xaa can be any amino acid other than glycine
PAGLLDLRQGMFAQLVAQNVLLIDXPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 457 - protein, 174 aa, synthetic construct
  REGION 1..174: note = synthetic sequence
  VARIANT 26: note = Xaa can be any amino acid other than proline
PAGLLDLRQGMFAQLVAQNVLLIDGXLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 458 - protein, 174 aa, synthetic construct
  REGION 1..174: note = synthetic sequence
  VARIANT 27: note = Xaa can be any amino acid other than leucine
PAGLLDLRQGMFAQLVAQNVLLIDGPXSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 459 - protein, 174 aa, synthetic construct
  REGION 1..174: note = synthetic sequence
  VARIANT 28: note = Xaa can be any amino acid other than serine
PAGLLDLRQGMFAQLVAQNVLLIDGPLXWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 460 - protein, 174 aa, synthetic construct
  REGION 1..174: note = synthetic sequence
  VARIANT 29: note = Xaa can be any amino acid other than tryptophan
PAGLLDLRQGMFAQLVAQNVLLIDGPLSXYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 461 - protein, 174 aa, synthetic construct
  REGION 1..174: note = synthetic sequence
  VARIANT 30: note = Xaa can be any amino acid other than tyrosine
PAGLLDLRQGMFAQLVAQNVLLIDGPLSWXSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 462 - protein, 174 aa, synthetic construct
  REGION 1..174: note = synthetic sequence
  VARIANT 31: note = Xaa can be any amino acid other than serine
PAGLLDLRQGMFAQLVAQNVLLIDGPLSWYXDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 463 - protein, 174 aa, synthetic construct
  REGION 1..174: note = synthetic sequence
  VARIANT 32: note = Xaa can be any amino acid other than aspartic acid
PAGLLDLRQGMFAQLVAQNVLLIDGPLSWYSXPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 464 - protein, 174 aa, synthetic construct
  REGION 1..174: note = synthetic sequence
  VARIANT 33: note = Xaa can be any amino acid other than proline
PAGLLDLRQGMFAQLVAQNVLLIDGPLSWYSDXGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 465 - protein, 174 aa, synthetic construct
  REGION 1..174: note = synthetic sequence
  VARIANT 34: note = Xaa can be any amino acid other than glycine
PAGLLDLRQGMFAQLVAQNVLLIDGPLSWYSDPXLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 466 - protein, 174 aa, synthetic construct
  REGION 1..174: note = synthetic sequence
  VARIANT 35: note = Xaa can be any amino acid other than leucine
PAGLLDLRQGMFAQLVAQNVLLIDGPLSWYSDPGXAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 467 - protein, 174 aa, synthetic construct
  REGION 1..174: note = synthetic sequence
  VARIANT 37: note = Xaa can be any amino acid other than glycine
PAGLLDLRQGMFAQLVAQNVLLIDGPLSWYSDPGLAXVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 468 - protein, 174 aa, synthetic construct
  REGION 1..174: note = synthetic sequence
  VARIANT 38: note = Xaa can be any amino acid other than valine
PAGLLDLRQGMFAQLVAQNVLLIDGPLSWYSDPGLAGXSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 469 - protein, 174 aa, synthetic construct
  REGION 1..174: note = synthetic sequence
  VARIANT 39: note = Xaa can be any amino acid other than serine
PAGLLDLRQGMFAQLVAQNVLLIDGPLSWYSDPGLAGVXLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 470 - protein, 174 aa, synthetic construct
  REGION 1..174: note = synthetic sequence
  VARIANT 40: note = Xaa can be any amino acid other than leucine
PAGLLDLRQGMFAQLVAQNVLLIDGPLSWYSDPGLAGVSXTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 471 - protein, 174 aa, synthetic construct
  REGION 1..174: note = synthetic sequence
  VARIANT 41: note = Xaa can be any amino acid other than threonine
PAGLLDLRQGMFAQLVAQNVLLIDGPLSWYSDPGLAGVSLXGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 472 - protein, 174 aa, synthetic construct
  REGION 1..174: note = synthetic sequence
  VARIANT 42: note = Xaa can be any amino acid other than glycine
PAGLLDLRQGMFAQLVAQNVLLIDGPLSWYSDPGLAGVSLTXGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 473 - protein, 174 aa, synthetic construct
  REGION 1..174: note = synthetic sequence
  VARIANT 43: note = Xaa can be any amino acid other than glycine
PAGLLDLRQGMFAQLVAQNVLLIDGPLSWYSDPGLAGVSLTGXLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 474 - protein, 174 aa, synthetic construct
  REGION 1..174: note = synthetic sequence
  VARIANT 44: note = Xaa can be any amino acid other than leucine
PAGLLDLRQGMFAQLVAQNVLLIDGPLSWYSDPGLAGVSLTGGXSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 475 - protein, 174 aa, synthetic construct
  REGION 1..174: note = synthetic sequence
  VARIANT 45: note = Xaa can be any amino acid other than serine
PAGLLDLRQGMFAQLVAQNVLLIDGPLSWYSDPGLAGVSLTGGLXYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 476 - protein, 174 aa, synthetic construct
  REGION 1..174: note = synthetic sequence
  VARIANT 46: note = Xaa can be any amino acid other than tyrosine
PAGLLDLRQGMFAQLVAQNVLLIDGPLSWYSDPGLAGVSLTGGLSXKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 477 - protein, 174 aa, synthetic construct
  REGION 1..174: note = synthetic sequence
  VARIANT 48: note = Xaa can be any amino acid other than glutamic acid
PAGLLDLRQGMFAQLVAQNVLLIDGPLSWYSDPGLAGVSLTGGLSYKXDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 478 - protein, 174 aa, synthetic construct
  REGION 1..174: note = synthetic sequence
  VARIANT 49: note = Xaa can be any amino acid other than aspartic acid
PAGLLDLRQGMFAQLVAQNVLLIDGPLSWYSDPGLAGVSLTGGLSYKEXTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 479 - protein, 174 aa, synthetic construct
  REGION 1..174: note = synthetic sequence
  VARIANT 50: note = Xaa can be any amino acid other than threonine
PAGLLDLRQGMFAQLVAQNVLLIDGPLSWYSDPGLAGVSLTGGLSYKEDXKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 480 - protein, 174 aa, synthetic construct
  REGION 1..174: note = synthetic sequence
  VARIANT 51: note = Xaa can be any amino acid other than lysine
PAGLLDLRQGMFAQLVAQNVLLIDGPLSWYSDPGLAGVSLTGGLSYKEDTXELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 481 - protein, 174 aa, synthetic construct
  REGION 1..174: note = synthetic sequence
  VARIANT 52: note = Xaa can be any amino acid other than glutamic acid
PAGLLDLRQGMFAQLVAQNVLLIDGPLSWYSDPGLAGVSLTGGLSYKEDTKXLVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 482 - protein, 174 aa, synthetic construct
  REGION 1..174: note = synthetic sequence
  VARIANT 64: note = Xaa can be any amino acid other than phenylalanine
PAGLLDLRQGMFAQLVAQNVLLIDGPLSWYSDPGLAGVSLTGGLSYKEDTKELVVAKAGVYYVXFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 483 - protein, 174 aa, synthetic construct
  REGION 1..174: note = synthetic sequence
  VARIANT 65: note = Xaa can be any amino acid other than phenylalanine
PAGLLDLRQGMFAQLVAQNVLLIDGPLSWYSDPGLAGVSLTGGLSYKEDTKELVVAKAGVYYVFXQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 484 - protein, 174 aa, synthetic construct
  REGION 1..174: note = synthetic sequence
  VARIANT 66: note = Xaa can be any amino acid other than glutamine
PAGLLDLRQGMFAQLVAQNVLLIDGPLSWYSDPGLAGVSLTGGLSYKEDTKELVVAKAGVYYVFFXLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 485 - protein, 174 aa, synthetic construct
  REGION 1..174: note = synthetic sequence
  VARIANT 67: note = Xaa can be any amino acid other than leucine
PAGLLDLRQGMFAQLVAQNVLLIDGPLSWYSDPGLAGVSLTGGLSYKEDTKELVVAKAGVYYVFFQXELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 486 - protein, 174 aa, synthetic construct
  REGION 1..174: note = synthetic sequence
  VARIANT 68: note = Xaa can be any amino acid other than glutamic acid
PAGLLDLRQGMFAQLVAQNVLLIDGPLSWYSDPGLAGVSLTGGLSYKEDTKELVVAKAGVYYVFFQLXLRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 487 - protein, 174 aa, synthetic construct
  REGION 1..174: note = synthetic sequence
  VARIANT 69: note = Xaa can be any amino acid other than leucine
PAGLLDLRQGMFAQLVAQNVLLIDGPLSWYSDPGLAGVSLTGGLSYKEDTKELVVAKAGVYYVFFQLEXRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 488 - protein, 174 aa, synthetic construct
  REGION 1..174: note = synthetic sequence
  VARIANT 70: note = Xaa can be any amino acid other than arginine
PAGLLDLRQGMFAQLVAQNVLLIDGPLSWYSDPGLAGVSLTGGLSYKEDTKELVVAKAGVYYVFFQLELXRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 489 - protein, 174 aa, synthetic construct
  REGION 1..174: note = synthetic sequence
  VARIANT 71: note = Xaa can be any amino acid other than arginine
PAGLLDLRQGMFAQLVAQNVLLIDGPLSWYSDPGLAGVSLTGGLSYKEDTKELVVAKAGVYYVFFQLELRXVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 490 - protein, 174 aa, synthetic construct
  REGION 1..174: note = synthetic sequence
  VARIANT 72: note = Xaa can be any amino acid other than valine
PAGLLDLRQGMFAQLVAQNVLLIDGPLSWYSDPGLAGVSLTGGLSYKEDTKELVVAKAGVYYVFFQLELRRXVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 491 - protein, 174 aa, synthetic construct
  REGION 1..174: note = synthetic sequence
  VARIANT 73: note = Xaa can be any amino acid other than valine
PAGLLDLRQGMFAQLVAQNVLLIDGPLSWYSDPGLAGVSLTGGLSYKEDTKELVVAKAGVYYVFFQLELRRVXAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 492 - protein, 174 aa, synthetic construct
  REGION 1..174: note = synthetic sequence
  VARIANT 75: note = Xaa can be any amino acid other than glycine
PAGLLDLRQGMFAQLVAQNVLLIDGPLSWYSDPGLAGVSLTGGLSYKEDTKELVVAKAGVYYVFFQLELRRVVAXEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 493 - protein, 174 aa, synthetic construct
  REGION 1..174: note = synthetic sequence
  VARIANT 76: note = Xaa can be any amino acid other than glutamic acid
PAGLLDLRQGMFAQLVAQNVLLIDGPLSWYSDPGLAGVSLTGGLSYKEDTKELVVAKAGVYYVFFQLELRRVVAGXGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 494 - protein, 174 aa, synthetic construct
  REGION 1..174: note = synthetic sequence
  VARIANT 77: note = Xaa can be any amino acid other than glycine
PAGLLDLRQGMFAQLVAQNVLLIDGPLSWYSDPGLAGVSLTGGLSYKEDTKELVVAKAGVYYVFFQLELRRVVAGEXSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 495 - protein, 174 aa, synthetic construct
  REGION 1..174: note = synthetic sequence
  VARIANT 78: note = Xaa can be any amino acid other than serine
PAGLLDLRQGMFAQLVAQNVLLIDGPLSWYSDPGLAGVSLTGGLSYKEDTKELVVAKAGVYYVFFQLELRRVVAGEGXGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 496 - protein, 174 aa, synthetic construct
  REGION 1..174: note = synthetic sequence
  VARIANT 104: note = Xaa can be any amino acid other than aspartic acid
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVXLPPASSEARNSAFGFQGRLLHLSAGQRLGVHLHTEARARHAWQLTQGATVLGLFRVTPEIPAGLPSPRSE

SEQ ID NO: 497 - protein, 174 aa, synthetic construct
  REGION 1..174: note = synthetic sequence
  VARIANT 105: note = Xaa can be any amino acid other than leucine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDXPPASSEARNSAFGFQGRLLHLSAGQRLGVHLHTEARARHAWQLTQGATVLGLFRVTPEIPAGLPSPRSE

SEQ ID NO: 498 - protein, 174 aa, synthetic construct
  REGION 1..174: note = synthetic sequence
  VARIANT 106: note = Xaa can be any amino acid other than proline
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLXPASSEARNSAFGFQGRLLHLSAGQRLGVHLHTEARARHAWQLTQGATVLGLFRVTPEIPAGLPSPRSE

SEQ ID NO: 499 - protein, 174 aa, synthetic construct
  REGION 1..174: note = synthetic sequence
  VARIANT 109: note = Xaa can be any amino acid other than serine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPAXSEARNSAFGFQGRLLHLSAGQRLGVHLHTEARARHAWQLTQGATVLGLFRVTPEIPAGLPSPRSE

SEQ ID NO: 500 - protein, 174 aa, synthetic construct
  REGION 1..174: note = synthetic sequence
  VARIANT 110: note = Xaa can be any amino acid other than serine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASXEARNSAFGFQGRLLHLSAGQRLGVHLHTEARARHAWQLTQGATVLGLFRVTPEIPAGLPSPRSE

SEQ ID NO: 501 - protein, 174 aa, synthetic construct
  REGION 1..174: note = synthetic sequence
  VARIANT 111: note = Xaa can be any amino acid other than glutamic acid
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSXARNSAFGFQGRLLHLSAGQRLGVHLHTEARARHAWQLTQGATVLGLFRVTPEIPAGLPSPRSE

SEQ ID NO: 502 - protein, 174 aa, synthetic construct
  REGION 1..174: note = synthetic sequence
  VARIANT 113: note = Xaa can be any amino acid other than arginine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEAXNSAFGFQGRLLHLSAGQRLGVHLHTEARARHAWQLTQGATVLGLFRVTPEIPAGLPSPRSE

SEQ ID NO: 503 - protein, 174 aa, synthetic construct
  REGION 1..174: note = synthetic sequence
  VARIANT 114: note = Xaa can be any amino acid other than asparagine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARXSAFGFQGRLLHLSAGQRLGVHLHTEARARHAWQLTQGATVLGLFRVTPEIPAGLPSPRSE

SEQ ID NO: 504 - protein, 174 aa, synthetic construct
  REGION 1..174: note = synthetic sequence
  VARIANT 115: note = Xaa can be any amino acid other than serine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNXAFGFQGRLLHLSAGQRLGVHLHTEARARHAWQLTQGATVLGLFRVTPEIPAGLPSPRSE

SEQ ID NO: 505 - protein, 174 aa, synthetic construct
  REGION 1..174: note = synthetic sequence
  VARIANT 117: note = Xaa can be any amino acid other than phenylalanine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAXGFQGRLLHLSAGQRLGVHLHTEARARHAWQLTQGATVLGLFRVTPEIPAGLPSPRSE

SEQ ID NO: 506 - protein, 174 aa, synthetic construct
  REGION 1..174: note = synthetic sequence
  VARIANT 130: note = Xaa can be any amino acid other than glutamine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGXRLGVHLHTEARARHAWQLTQGATVLGLFRVTPEIPAGLPSPRSE

SEQ ID NO: 507 - protein, 174 aa, synthetic construct
  REGION 1..174: note = synthetic sequence
  VARIANT 131: note = Xaa can be any amino acid other than arginine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQXLGVHLHTEARARHAWQLTQGATVLGLFRVTPEIPAGLPSPRSE

SEQ ID NO: 508 - protein, 174 aa, synthetic construct
  REGION 1..174: note = synthetic sequence
  VARIANT 132: note = Xaa can be any amino acid other than leucine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRXGVHLHTEARARHAWQLTQGATVLGLFRVTPEIPAGLPSPRSE

SEQ ID NO: 509 - protein, 174 aa, synthetic construct
  REGION 1..174: note = synthetic sequence
  VARIANT 133: note = Xaa can be any amino acid other than glycine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLXVHLHTEARARHAWQLTQGATVLGLFRVTPEIPAGLPSPRSE

SEQ ID NO: 510 - protein, 174 aa, synthetic construct
  REGION 1..174: note = synthetic sequence
  VARIANT 134: note = Xaa can be any amino acid other than valine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGXHLHTEARARHAWQLTQGATVLGLFRVTPEIPAGLPSPRSE

SEQ ID NO: 511 - protein, 174 aa, synthetic construct
  REGION 1..174: note = synthetic sequence
  VARIANT 135: note = Xaa can be any amino acid other than histidine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVXLHTEARARHAWQLTQGATVLGLFRVTPEIPAGLPSPRSE

SEQ ID NO: 512 - protein, 174 aa, synthetic construct
  REGION 1..174: note = synthetic sequence
  VARIANT 136: note = Xaa can be any amino acid other than leucine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHXHTEARARHAWQLTQGATVLGLFRVTPEIPAGLPSPRSE

SEQ ID NO: 513 - protein, 174 aa, synthetic construct
  REGION 1..174: note = synthetic sequence
  VARIANT 137: note = Xaa can be any amino acid other than histidine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLXTEARARHAWQLTQGATVLGLFRVTPEIPAGLPSPRSE

SEQ ID NO: 514 - protein, 174 aa, synthetic construct
  REGION 1..174: note = synthetic sequence
  VARIANT 138: note = Xaa can be any amino acid other than threonine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHXEARARHAWQLTQGATVLGLFRVTPEIPAGLPSPRSE

SEQ ID NO: 515 - protein, 174 aa, synthetic construct
  REGION 1..174: note = synthetic sequence
  VARIANT 139: note = Xaa can be any amino acid other than glutamic acid
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTXARARHAWQLTQGATVLGLFRVTPEIPAGLPSPRSE

SEQ ID NO: 516 - protein, 174 aa, synthetic construct
  REGION 1..174: note = synthetic sequence
  VARIANT 141: note = Xaa can be any amino acid other than arginine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEAXARHAWQLTQGATVLGLFRVTPEIPAGLPSPRSE

SEQ ID NO: 517 - protein, 174 aa, synthetic construct
  REGION 1..174: note = synthetic sequence
  VARIANT 143: note = Xaa can be any amino acid other than arginine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARAXHAWQLTQGATVLGLFRVTPEIPAGLPSPRSE

SEQ ID NO: 518 - protein, 174 aa, synthetic construct
  REGION 1..174: note = synthetic sequence
  VARIANT 144: note = Xaa can be any amino acid other than histidine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARXAWQLTQGATVLGLFRVTPEIPAGLPSPRSE

SEQ ID NO: 519 - protein, 174 aa, synthetic construct
  REGION 1..174: note = synthetic sequence
  VARIANT 146: note = Xaa can be any amino acid other than tryptophan
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAXQLTQGATVLGLFRVTPEIPAGLPSPRSE

SEQ ID NO: 520 - protein, 174 aa, synthetic construct
  REGION 1..174: note = synthetic sequence
  VARIANT 148: note = Xaa can be any amino acid other than leucine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQXTQGATVLGLFRVTPEIPAGLPSPRSE

SEQ ID NO: 521 - protein, 174 aa, synthetic construct
  REGION 1..174: note = synthetic sequence
  VARIANT 149: note = Xaa can be any amino acid other than threonine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLXQGATVLGLFRVTPEIPAGLPSPRSE

SEQ ID NO: 522 - protein, 174 aa, synthetic construct
  REGION 1..174: note = synthetic sequence
  VARIANT 150: note = Xaa can be any amino acid other than glutamine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTXGATVLGLFRVTPEIPAGLPSPRSE

SEQ ID NO: 523 - protein, 174 aa, synthetic construct
  REGION 1..174: note = synthetic sequence
  VARIANT 151: note = Xaa can be any amino acid other than glycine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQXATVLGLFRVTPEIPAGLPSPRSE

SEQ ID NO: 524 - protein, 174 aa, synthetic construct
  REGION 1..174: note = synthetic sequence
  VARIANT 153: note = Xaa can be any amino acid other than threonine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGAXVLGLFRVTPEIPAGLPSPRSE

SEQ ID NO: 525 - protein, 174 aa, synthetic construct
  REGION 1..174: note = synthetic sequence
  VARIANT 154: note = Xaa can be any amino acid other than valine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATXLGLFRVTPEIPAGLPSPRSE

SEQ ID NO: 526 - skipped

SEQ ID NO: 527 - protein, 133 aa, synthetic construct
  REGION 1..133: note = synthetic sequence
  VARIANT 42: note = Xaa can be any amino acid other than phenylalanine
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 528 - protein, 133 aa, synthetic construct
  REGION 1..133: note = synthetic sequence
  VARIANT 20: note = Xaa can be any amino acid other than aspartic acid
APTSSSTKKTQLQLEHLLLXLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 529 - protein, 133 aa, synthetic construct
  REGION 1..133: note = synthetic sequence
  VARIANT 15: note = Xaa can be any amino acid other than glutamic acid
APTSSSTKKTQLQLXHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 530 - protein, 133 aa, synthetic construct
  REGION 1..133: note = synthetic sequence
  VARIANT 16: note = Xaa can be any amino acid other than histidine
APTSSSTKKTQLQLEXLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 531 - protein, 133 aa, synthetic construct; identical to SEQ ID NO: 530
  REGION 1..133: note = synthetic sequence
  VARIANT 16: note = Xaa can be any amino acid other than histidine

SEQ ID NO: 532 - protein, 133 aa, synthetic construct
  REGION 1..133: note = synthetic sequence
  VARIANT 45: note = Xaa can be any amino acid other than tyrosine
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 533 - protein, 133 aa, synthetic construct
  REGION 1..133: note = synthetic sequence
  VARIANT 126: note = Xaa can be any amino acid other than glutamine
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 534 - protein, 133 aa, synthetic construct
  REGION 1..133: note = synthetic sequence
  VARIANT 16: note = Xaa can be any amino acid other than histidine
  VARIANT 42: note = Xaa can be any amino acid other than phenylalanine
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 535 - protein, 133 aa, synthetic construct
  REGION 1..133: note = synthetic sequence
  VARIANT 20: note = Xaa can be any amino acid other than aspartic acid
  VARIANT 42: note = Xaa can be any amino acid other than phenylalanine
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 536 - protein, 133 aa, synthetic construct
  REGION 1..133: note = synthetic sequence
  VARIANT 15: note = Xaa can be any amino acid other than glutamic acid
  VARIANT 20: note = Xaa can be any amino acid other than aspartic acid
  VARIANT 42: note = Xaa can be any amino acid other than phenylalanine
APTSSSTKKTQLQLXHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 537 - protein, 133 aa, synthetic construct
  REGION 1..133: note = synthetic sequence
  VARIANT 16: note = Xaa can be any amino acid other than histidine
  VARIANT 20: note = Xaa can be any amino acid other than aspartic acid
  VARIANT 42: note = Xaa can be any amino acid other than phenylalanine
APTSSSTKKTQLQLEXLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 538 - protein, 133 aa, synthetic construct
  REGION 1..133: note = synthetic sequence
  VARIANT 20: note = Xaa can be any amino acid other than aspartic acid
  VARIANT 42: note = Xaa can be any amino acid other than phenylalanine
  VARIANT 126: note = Xaa can be any amino acid other than glutamine
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 539 - protein, 133 aa, synthetic construct
  REGION 1..133: note = synthetic sequence
  VARIANT 20: note = Xaa can be any amino acid other than aspartic acid
  VARIANT 42: note = Xaa can be any amino acid other than phenylalanine
  VARIANT 45: note = Xaa can be any amino acid other than tyrosine
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 540 - protein, 133 aa, synthetic construct
  REGION 1..133: note = synthetic sequence
  VARIANT 16: note = Xaa can be any amino acid other than histidine
  VARIANT 20: note = Xaa can be any amino acid other than aspartic acid
  VARIANT 42: note = Xaa can be any amino acid other than phenylalanine
  VARIANT 45: note = Xaa can be any amino acid other than tyrosine
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 541 - protein, 133 aa, synthetic construct
  REGION 1..133: note = synthetic sequence
  VARIANT 20: note = Xaa can be any amino acid other than aspartic acid
  VARIANT 42: note = Xaa can be any amino acid other than phenylalanine
  VARIANT 45: note = Xaa can be any amino acid other than tyrosine
  VARIANT 126: note = Xaa can be any amino acid other than glutamine
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 542 - protein, 133 aa, synthetic construct
  REGION 1..133: note = synthetic sequence
  VARIANT 16: note = Xaa can be any amino acid other than histidine
  VARIANT 20: note = Xaa can be any amino acid other than aspartic acid
  VARIANT 42: note = Xaa can be any amino acid other than phenylalanine
  VARIANT 45: note = Xaa can be any amino acid other than tyrosine
  VARIANT 126: note = Xaa can be any amino acid other than glutamine
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 543 - protein, 133 aa, synthetic construct
  REGION 1..133: note = synthetic sequence
  VARIANT 16: note = Xaa can be any amino acid other than histidine
  VARIANT 42: note = Xaa can be any amino acid other than phenylalanine
  VARIANT 126: note = Xaa can be any amino acid other than glutamine
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 544 - protein, 9 aa, synthetic construct
  REGION 1..9: note = synthetic sequence
YPYDVPDYA

SEQ ID NO: 545 - protein, 8 aa, synthetic construct
  REGION 1..8: note = synthetic sequence
DYKDDDDK

SEQ ID NO: 546 - protein, 10 aa, synthetic construct
  REGION 1..10: note = synthetic sequence
EQKLISEEDL

SEQ ID NO: 547 - protein, 5 aa, synthetic construct
  REGION 1..5: note = synthetic sequence
HHHHH

SEQ ID NO: 548 - protein, 6 aa, synthetic construct
  REGION 1..6: note = synthetic sequence
HHHHHH

SEQ ID NO: 549 - protein, 10 aa, synthetic construct
  REGION 1..10: note = synthetic sequence
EQKLISEEDL

SEQ ID NO: 550 - protein, 8 aa, synthetic construct
  REGION 1..8: note = synthetic sequence
DYKDDDDK

SEQ ID NO: 551 - protein, 8 aa, synthetic construct
  REGION 1..8: note = synthetic sequence
WSHPQFEK

SEQ ID NO: 552 - protein, 9 aa, synthetic construct
  REGION 1..9: note = synthetic sequence
YPYDVPDYA

SEQ ID NO: 553 - protein, 5 aa, synthetic construct
  REGION 1..5: note = synthetic sequence
RYIRS

SEQ ID NO: 554 - protein, 4 aa, synthetic construct
  REGION 1..4: note = synthetic sequence
FHHT

SEQ ID NO: 555 - protein, 17 aa, synthetic construct
  REGION 1..17: note = synthetic sequence
WEAAAREACCRECCARA

SEQ ID NO: 556 - protein, 8 aa, synthetic construct
  REGION 1..8: note = synthetic sequence
LEVLFQGP

SEQ ID NO: 557 - protein, 7 aa, synthetic construct
  REGION 1..7: note = synthetic sequence
ENLYTQS

SEQ ID NO: 558 - protein, 5 aa, synthetic construct
  REGION 1..5: note = synthetic sequence
DDDDK

SEQ ID NO: 559 - protein, 4 aa, synthetic construct
  REGION 1..4: note = synthetic sequence
LVPR

SEQ ID NO: 560 - protein, 22 aa, synthetic construct
  REGION 1..22: note = synthetic sequence
GSGATNFSLLKQAGDVEENPGP

SEQ ID NO: 561 - protein, 5 aa, synthetic construct
  REGION 1..5: note = synthetic sequence
  VARIANT 4: note = Xaa is any amino acid other than proline
VPGXG

SEQ ID NO: 562 - protein, 10 aa, synthetic construct
  REGION 1..10: note = synthetic sequence
  VARIANT 6..10: note = The residues are repeated 1 time
GCGGSGGGGS

SEQ ID NO: 563 - protein, 10 aa, synthetic construct
  REGION 1..10: note = synthetic sequence
  VARIANT 6..10: note = The residues are repeated 2 times
GCGGSGGGGS

SEQ ID NO: 564 - protein, 10 aa, synthetic construct
  REGION 1..10: note = synthetic sequence
  VARIANT 6..10: note = The residues are repeated 3 times
GCGGSGGGGS

SEQ ID NO: 565 - protein, 10 aa, synthetic construct
  REGION 1..10: note = synthetic sequence
  VARIANT 6..10: note = The residues are repeated 4 times
GCGGSGGGGS

SEQ ID NO: 566 - protein, 10 aa, synthetic construct
  REGION 1..10: note = synthetic sequence
  VARIANT 6..10: note = The residues are repeated 5 times
GCGGSGGGGS

SEQ ID NO: 567 - protein, 10 aa, synthetic construct
  REGION 1..10: note = synthetic sequence
  VARIANT 6..10: note = The residues are repeated 6 times
GCGGSGGGGS

SEQ ID NO: 568 - protein, 10 aa, synthetic construct
  REGION 1..10: note = synthetic sequence
  VARIANT 6..10: note = The residues are repeated 7 times
GCGGSGGGGS

SEQ ID NO: 569 - protein, 10 aa, synthetic construct
  REGION 1..10: note = synthetic sequence
  VARIANT 6..10: note = The residues are repeated 8 times
GCGGSGGGGS

SEQ ID NO: 570 - protein, 10 aa, synthetic construct
  REGION 1..10: note = synthetic sequence
  VARIANT 6..10: note = The residues are repeated 9 times
GCGGSGGGGS

SEQ ID NO: 571 - protein, 10 aa, synthetic construct
  REGION 1..10: note = synthetic sequence
  VARIANT 6..10: note = The residues are repeated 10 times
GCGGSGGGGS

SEQ ID NO: 572 - protein, 10 aa, synthetic construct
  REGION 1..10: note = synthetic sequence
  VARIANT 6..10: note = The residues are repeated 1 time
CGGGSGGGGS

SEQ ID NO: 573 - protein, 10 aa, synthetic construct
  REGION 1..10: note = synthetic sequence
  VARIANT 6..10: note = The residues are repeated 2 times
CGGGSGGGGS

SEQ ID NO: 574 - protein, 10 aa, synthetic construct
  REGION 1..10: note = synthetic sequence
  VARIANT 6..10: note = The residues are repeated 3 times
CGGGSGGGGS

SEQ ID NO: 575 - protein, 10 aa, synthetic construct
  REGION 1..10: note = synthetic sequence
  VARIANT 6..10: note = The residues are repeated 4 times
CGGGSGGGGS

SEQ ID NO: 576 - protein, 10 aa, synthetic construct
  REGION 1..10: note = synthetic sequence
  VARIANT 6..10: note = The residues are repeated 5 times
CGGGSGGGGS

SEQ ID NO: 577 - protein, 10 aa, synthetic construct
  REGION 1..10: note = synthetic sequence
  VARIANT 6..10: note = The residues are repeated 6 times
CGGGSGGGGS

SEQ ID NO: 578 - protein, 10 aa, synthetic construct
  REGION 1..10: note = synthetic sequence
  VARIANT 6..10: note = The residues are repeated 7 times
CGGGSGGGGS

SEQ ID NO: 579 - protein, 10 aa, synthetic construct
  REGION 1..10: note = synthetic sequence
  VARIANT 6..10: note = The residues are repeated 8 times
CGGGSGGGGS

SEQ ID NO: 580 - protein, 10 aa, synthetic construct
  REGION 1..10: note = synthetic sequence
  VARIANT 6..10: note = The residues are repeated 9 times
CGGGSGGGGS

SEQ ID NO: 581 - protein, 10 aa, synthetic construct
  REGION 1..10: note = synthetic sequence
  VARIANT 6..10: note = The residues are repeated 10 times
CGGGSGGGGS

SEQ ID NO: 582 - protein, 10 aa, synthetic construct
  REGION 1..10: note = synthetic sequence
  VARIANT 6..10: note = The residues may be repeated 1, 2, or 3 times
GCGGSGGGGS

SEQ ID NO: 583 - protein, 10 aa, synthetic construct
  REGION 1..10: note = synthetic sequence
  VARIANT 6..10: note = The residues may be repeated 1, 2, 3, 4, 5, 6, 7, 8, or 9 times
GCGGSGGGGS

SEQ ID NO: 584 - protein, 13 aa, synthetic construct
  REGION 1..13: note = synthetic sequence
GCGGSGGGSGGGS

SEQ ID NO: 585 - protein, 15 aa, synthetic construct
  REGION 1..15: note = synthetic sequence
GCGGSGGGGSGGGGS

SEQ ID NO: 586 - protein, 341 aa, synthetic construct
  REGION 1..341: note = synthetic sequence
  VARIANT 84: note = In some cases, amino acid X1 at position 84 is an Ala. In some cases, amino acid 84 is a Cys.
  VARIANT 236: note = In some cases, amino acid X2 at position 236 is a Cys.
GSHSMRYFSTSVSRPGRGEPRFIAVGYVDDTQFVRFDSDAASQRMEPRAPWIEQEGPEYWDEETGKVKAHSQTDRENLRIALRXYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPXGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 587 - protein, 5 aa, synthetic construct
  REGION 1..5: note = synthetic sequence
GGCGS

SEQ ID NO: 588 - protein, 5 aa, synthetic construct
  REGION 1..5: note = synthetic sequence
GGGCS

SEQ ID NO: 589 - protein, 10 aa, synthetic construct
  REGION 1..10: note = synthetic sequence
  VARIANT 6..10: note = The residues may be repeated 1, 2, 3, 4, 5, 6, 7, 8, 9, or 10 times
GGGCSGGGGS

SEQ ID NO: 590 - protein, 5 aa, synthetic construct
  REGION 1..5: note = synthetic sequence
GGGGC

SEQ ID NO: 591 - protein, 10 aa, synthetic construct
  REGION 1..10: note = synthetic sequence
  VARIANT 6..10: note = The residues may be repeated 1, 2, 3, 4, 5, 6, 7, 8, 9, or 10 times
GGGGCGGGGS

SEQ ID NO: 592 - protein, 10 aa, synthetic construct
  REGION 1..10: note = synthetic sequence
  VARIANT 6..10: note = The residues may be repeated 1, 2, 3, 4, 5, 6, 7, 8, 9 or 10 times
GGCGSGGGGS

SEQ ID NO: 593 - protein, 945 aa, synthetic construct
  REGION 1..945: note = synthetic sequence
VVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 594 - protein, 966 aa, synthetic construct; identical to SEQ ID NO: 336
  REGION 1..966: note = synthetic sequence

SEQ ID NO: 595 - protein, 945 aa, synthetic construct; identical to SEQ ID NO: 593
  REGION 1..945: note = synthetic sequence

SEQ ID NO: 596 - protein, 946 aa, synthetic construct
  REGION 1..946: note = synthetic sequence
VVVGAVGVGKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 597 - protein, 946 aa, synthetic construct
  REGION 1..946: note = synthetic sequence
VVVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 598 - protein, 946 aa, synthetic construct
  REGION 1..946: note = synthetic sequence
VVVGAVGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 599 - protein, 645 aa, synthetic construct
  REGION 1..645: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 600 - protein, 645 aa, synthetic construct
  REGION 1..645: note = synthetic sequence
VVVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 601 - protein, 506 aa, synthetic construct
  REGION 1..506: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 602 - protein, 812 aa, synthetic construct
  REGION 1..812: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG